SEQUENCE LISTING (168 sequences)

SEQ ID NO: 1 - DNA, 12 bp, synthetic construct
  misc_feature 1..12: note = Synthetic
  source 1..12: mol_type = other DNA
acttgtttaagt

SEQ ID NO: 2 - protein, 4 aa, synthetic construct
  REGION 1..4: note = Synthetic
GGGS

SEQ ID NO: 3 - protein, 5 aa, synthetic construct
  REGION 1..5: note = Synthetic
GGGGS

SEQ ID NO: 4 - protein, 9 aa, synthetic construct
  REGION 1..9: note = Synthetic
GGSGGSGGS

SEQ ID NO: 5 - protein, 15 aa, synthetic construct
  REGION 1..15: note = Synthetic
GGGGSGGGGSGGGGS

SEQ ID NO: 6 - protein, 10 aa, synthetic construct
  REGION 1..10: note = Synthetic
GGGGSGGGGS

SEQ ID NO: 7 - protein, 20 aa, synthetic construct
  REGION 1..20: note = Synthetic
GGGGSGGGGSGGGGSGGGGS

SEQ ID NO: 8 - protein, 25 aa, synthetic construct
  REGION 1..25: note = Synthetic
GGGGSGGGGSGGGGSGGGGSGGGGS

SEQ ID NO: 9 - protein, 30 aa, synthetic construct
  REGION 1..30: note = Synthetic
GGGGSGGGGSGGGGSGGGGSGGGGSGGGGS

SEQ ID NO: 10 - protein, 35 aa, synthetic construct
  REGION 1..35: note = Synthetic
GGGGSGGGGSGGGGSGGGGSGGGGSGGGGSGGGGS

SEQ ID NO: 11 - protein, 40 aa, synthetic construct
  REGION 1..40: note = Synthetic
GGGGSGGGGSGGGGSGGGGSGGGGSGGGGSGGGGSGGGGS

SEQ ID NO: 12 - protein, 45 aa, synthetic construct
  REGION 1..45: note = Synthetic
GGGGSGGGGSGGGGSGGGGSGGGGSGGGGSGGGGSGGGGSGGGGS

SEQ ID NO: 13 - protein, 50 aa, synthetic construct
  REGION 1..50: note = Synthetic
GGGGSGGGGSGGGGSGGGGSGGGGSGGGGSGGGGSGGGGSGGGGSGGGGS

SEQ ID NO: 14 - protein, 55 aa, synthetic construct
  REGION 1..55: note = Synthetic
GGGGSGGGGSGGGGSGGGGSGGGGSGGGGSGGGGSGGGGSGGGGSGGGGSGGGGS

SEQ ID NO: 15 - protein, 60 aa, synthetic construct
  REGION 1..60: note = Synthetic
GGGGSGGGGSGGGGSGGGGSGGGGSGGGGSGGGGSGGGGSGGGGSGGGGSGGGGSGGGGS

SEQ ID NO: 16 - protein, 32 aa, synthetic construct
  REGION 1..32: note = Synthetic
LEPGEKPYKCPECGKSFSQSGALTRHQRTHTR

SEQ ID NO: 17 - protein, 20 aa, synthetic construct
  REGION 1..20: note = Synthetic
PKKKRKVEASSPKKRKVEAS

SEQ ID NO: 18 - skipped

SEQ ID NO: 19 - DNA, 27 bp, synthetic construct
  misc_feature 1..27: note = Synthetic
  source 1..27: mol_type = other DNA
ggtggtagtggagggagcggcggttca

SEQ ID NO: 20 - protein, 21 aa, synthetic construct
  REGION 1..21: note = Synthetic
GGSGGSGGSGGSGGSGGSGGS

SEQ ID NO: 21 - DNA, 72 bp, synthetic construct
  misc_feature 1..72: note = Synthetic
  source 1..72: mol_type = other DNA
ggtggaggaggctctggtggaggcggtagcggaggcggagggtcgggtggtagtggagggagcggcggttca

SEQ ID NO: 22 - DNA, 48 bp, synthetic construct
  misc_feature 1..48: note = Synthetic
  source 1..48: mol_type = other DNA
tcgggatctgagacgcctgggacctcggaatcggctacgcccgaaagt

SEQ ID NO: 23 - DNA, 192 bp, synthetic construct
  misc_feature 1..192: note = Synthetic
  source 1..192: mol_type = other DNA
gtggataacaaatttaacaaagaaatgtgggcggcgtgggaagaaattcgtaacctgccgaacctgaacggctggcagatgaccgcgtttattgcgagcctggtggatgatccgagccagagcgcgaacctgctggcggaagcgaaaaaactgaacgatgcgcaggcgccgaaaaccggcggtggttctggt

SEQ ID NO: 24 - DNA, 108 bp, synthetic construct
  misc_feature 1..108: note = Synthetic
  source 1..108: mol_type = other DNA
ggtggttctgccggtggctccggttctggctccagcggtggcagctctggtgcgtccggcacgggtactgcgggtggcactggcagcggttccggtactggctctggc

SEQ ID NO: 25 - protein, 10 aa, Simian virus 40
PKKKRKVEAS

SEQ ID NO: 26 - protein, 16 aa, synthetic construct
  REGION 1..16: note = Synthetic
KRPAATKKAGQAKKKK

SEQ ID NO: 27 - protein, 9 aa, Homo sapiens
PAAKRVKLD

SEQ ID NO: 28 - protein, 11 aa, Homo sapiens
RQRRNELKRSP

SEQ ID NO: 29 - protein, 38 aa, Homo sapiens
NQSSNFGPMKGGNFGGRSSGPYGGGGQYFAKPRNQGGY

SEQ ID NO: 30 - protein, 42 aa, Homo sapiens
RMRIZFKNKGKDTAELRRRRVEVSVELRKAKKDEQILKRRNV

SEQ ID NO: 31 - protein, 8 aa, Homo sapiens
VSRKRPRP

SEQ ID NO: 32 - protein, 8 aa, Homo sapiens
PPKKARED

SEQ ID NO: 33 - protein, 8 aa, Homo sapiens
PQPKKKPL

SEQ ID NO: 34 - protein, 12 aa, Mus sp.
SALIKKKKKMAP

SEQ ID NO: 35 - protein, 5 aa, Influenza virus
DRLRR

SEQ ID NO: 36 - protein, 7 aa, Influenza virus
PKQKKRK

SEQ ID NO: 37 - protein, 10 aa, Hepatitis delta virus
RKLKKKIKKL

SEQ ID NO: 38 - protein, 10 aa, Mus sp.
REKKKFLKRR

SEQ ID NO: 39 - protein, 20 aa, Homo sapiens
KRKGDEVDGVDEVAKKKSKK

SEQ ID NO: 40 - protein, 17 aa, Homo sapiens
RKCLQAGMNLEARKTKK

SEQ ID NO: 41 - DNA, 6106 bp, unidentified
  source 1..6106: mol_type = other DNA; note = AID-BLM-UGI
gcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgagagccgccaccatggacagcctcttgatgaaccggagggagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcatctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccacggaagaactttcaaagcctgggaagggctgcatgaaaattcagttcgtctctccagacagcttcggcgcatccttttgcccctgtatgaggttgatgacttacgagacgcatttcgtactagcggcagcgagactcccgggacctcagagtccgccacacccgaaagtatggctgctgttcctcaaaataatctacaggagcaactagaacgtcactcagccagaacacttaataataaattaagtctttcaaaaccaaaattttcaggtttcacttttaaaaagaaaacatcttcagataacaatgtatctgtaactaatgtgtcagtagcaaaaacacctgtattaagaaataaagatgttaatgttaccgaagacttttccttcagtgaacctctacccaacaccacaaatcagcaaagggtcaaggacttctttaaaaatgctccagcaggacaggaaacacagagaggtggatcaaaatcattattgccagatttcttgcagactccgaaggaagttgtatgcactacccaaaacacaccaactgtaaagaaatcccgggatactgctctcaagaaattagaatttagttcttcaccagattctttaagtaccatcaatgattgggatgatatggatgactttgatacttctgagacttcaaaatcatttgttacaccaccccaaagtcactttgtaagagtaagcactgctcagaaatcaaaaaagggtaagagaaacttttttaaagcacagctttatacaacaaacacagtaaagactgacttgcctccaccctcctctgaaagcgagcaaatagatttgactgaggaacagaaggatgactcagaatggttaagcagcgatgtgatttgcatcgatgatggccccattgctgaagtgcatataaatgaagatgctcaggaaagtgactctctgaaaactcatttggaagatgaaagagataatagcgaaaagaagaagaatttggaagaagctgaattacattcaactgagaaagttccatgtattgaatttgatgatgatgattatgatacggattttgttccaccttctccagaagaaattatttctgcttcttcttcctcttcaaaatgccttagtacgttaaaggaccttgacacctctgacagaaaagaggatgttcttagcacatcaaaagatcttttgtcaaaacctgagaaaatgagtatgcaggagctgaatccagaaaccagcacagactgtgacgctagacagataagtttacagcagcagcttattcatgtgatggagcacatctgtaaattaattgatactattcctgatgataaactgaaacttttggattgtgggaacgaactgcttcagcagcggaacataagaaggaaacttctaacggaagtagattttaataaaagtgatgccagtcttcttggctcattgtggagatacaggcctgattcacttgatggccctatggagggtgattcctgccctacagggaattctatgaaggagttaaatttttcacaccttccctcaaattctgtttctcctggggactgtttactgactaccaccctaggaaagacaggattctctgccaccaggaagaatctttttgaaaggcctttattcaatacccatttacagaagtcctttgtaagtagcaactgggctgaaacaccaagactaggaaaaaaaaatgaaagctcttatttcccaggaaatgttctcacaagcactgctgtgaaagatcagaataaacatactgcttcaataaatgacttagaaagagaaacccaaccttcctatgatattgataattttgacatagatgactttgatgatgatgatgactgggaagacataatgcataatttagcagccagcaaatcttccacagctgcctatcaacccatcaaggaaggtcggccaattaaatcagtatcagaaagactttcctcagccaagacagactgtcttccagtgtcatctactgctcaaaatataaacttctcagagtcaattcagaattatactgacaagtcagcacaaaatttagcatccagaaatctgaaacatgagcgtttccaaagtcttagttttcctcatacaaaggaaatgatgaagatttttcataaaaaatttggcctgcataattttagaactaatcagctagaggcgatcaatgctgcactgcttggtgaagactgttttatcctgatgccgactggaggtggtaagagtttgtgttaccagctccctgcctgtgtttctcctggggtcactgttgtcatttctcccttgagatcacttatcgtagatcaagtccaaaagctgacttccttggatattccagctacatatctgacaggtgataagactgactcagaagctacaaatatttacctccagttatcaaaaaaagacccaatcataaaactcctatatgtcactccagaaaagatctgtgcaagtaacagactcatttctactctggagaatctctatgagaggaagctcttggcacgttttgttattgatgaagcacattgtgtcagtcagtggggacatgattttcgtcaagattacaaaagaatgaatatgcttcgccagaagtttccttctgttccggtgatggctcttacggccacagctaatcccagggtacagaaggacatcctgactcagctgaagattctcagacctcaggtgtttagcatgagctttaacagacataatctgaaatactatgtattaccgaaaaagcctaaaaaggtggcatttgattgcctagaatggatcagaaagcaccacccatatgattcagggataatttactgcctctccaggcgagaatgtgacaccatggctgacacgttacagagagatgggctcgctgctcttgcttaccatgctggcctcagtgattctgccagagatgaagtgcagcagaagtggattaatcaggatggctgtcaggttatctgtgctacaattgcatttggaatggggattgacaaaccggacgtgcgatttgtgattcatgcatctctccctaaatctgtggagggttactaccaagaatctggcagagctggaagagatggggaaatatctcactgcctgcttttctatacctatcatgatgtgaccagactgaaaagacttataatgatggaaaaagatggaaaccatcatacaagagaaactcacttcaataatttgtatagcatggtacattactgtgaaaatataacagaatgcaggagaatacagcttttggcctactttggtgaaaatggatttaatcctgatttttgtaagaaacacccagatgtttcttgtgataattgctgtaaaacaaaggattataaaacaagagatgtgactgacgatgtgaaaagtattgtaagatttgttcaagaacatagttcatcacaaggaatgagaaatataaaacatgtaggtccttctggaagatttactatgaatatgctggtcgacattttcttggggagtaagagtgcaaaaatccagtcaggtatatttggaaaaggatctgcttattcacgacacaatgccgaaagactttttaaaaagctgatacttgacaagattttggatgaagacttatatatcaatgccaatgaccaggcgatcgcttatgtgatgctcggaaataaagcacaaactgtactaaatggcaatttaaaggtagactttatggaaacagaaaattccagcagtgtgaaaaaacaaaaagcgttagtagcaaaagtgtctcagagggaagagatggttaaaaaatgtcttggagaacttacagaagtctgcaaatctctggggaaagtttttggtgtccattacttcaatatttttaataccgtcactctcaagaagcttgcagaatctttatcttctgatcctgaggttttgcttcaaattgatggtgttactgaagacaaactggaaaaatatggtgcggaagtgatttcagtattacagaaatactctgaatggacatcgccagctgaagacagttccccagggataagcctgtccagcagcagaggccccggaagaagtgccgctgaggagcttgacgaggaaatacccgtatcttcccactactttgcaagtaaaaccagaaatgaaaggaagaggaaaaagatgccagcctcccaaaggtctaagaggagaaaaactgcttccagtggttccaaggcaaagggggggtctgccacatgtagaaagatatcttccaaaacgaaatcctccagcatcattggatccagttcagcctcacatacttctcaagcgacatcaggagccaatagcaaattggggattatggctccaccgaagcctataaatagaccgtttcttaagccttcatatgcattctcatctggtggttctactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctg

SEQ ID NO: 42 - DNA, 3256 bp, unidentified
  source 1..3256: mol_type = other DNA; note = AID-NS3h-UGI
gcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgagagccgccaccatggacagcctcttgatgaaccggagggagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcatctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccacggaagaactttcaaagcctgggaagggctgcatgaaaattcagttcgtctctccagacagcttcggcgcatccttttgcccctgtatgaggttgatgacttacgagacgcatttcgtactagcggcagcgagactcccgggacctcagagtccgccacacccgaaagtatgatggtggacttcatacccgttgagtctatggaaactaccatgcggtctccggtcttcacagacaactcaacccccccggctgtaccgcagacattccaagtggcacatctgcacgctcctactggcagcggcaagagcaccaaagtgccggctgcgtatgcagcccaagggtacaaggtgctcgtcctgaacccgtccgttgccgccaccttagggtttggggcgtatatgtccaaggcacacggtatcgaccctaacatcagaactggggtaaggaccattaccacgggcggctccattacgtactccacctatggcaagttccttgccgacggtggctgttctgggggcgcctatgacatcataatatgtgatgagtgccactcaactgactcgactaccatcttgggcatcggcacagtcctggaccaagcggagacggctggagcgcggctcgtcgtgctcgccaccgctacacctccgggatcggttaccgtgccacaccccaatatcgaggaaataggcctgtccaacaatggagagatccccttctatggcaaagccatccccattgaggccatcaagggggggaggcatctcattttctgccattccaagaagaaatgtgacgagctcgccgcaaagctgacaggcctcggactgaacgctgtagcatattaccggggccttgatgtgtccgtcataccgcctatcggagacgtcgttgtcgtggcaacagacgctctaatgacgggtttcaccggcgattttgactcagtgatcgactgcaatacatgtgtcacccagacagtcgacttcagcttggatcccaccttcaccattgagacgacgaccgtgccccaagacgcggtgtcgcgctcgcaacggcgaggtagaactggcaggggtaggagtggcatctacaggtttgtgactccaggagaacggccctcgggcatgttcgattcttcggtcctgtgtgagtgctatgacgcgggctgtgcttggtatgagctcacgcccgctgagacctcggttaggttgcgggcttacctaaatacaccagggttgcccgtctgccaggaccatctggagttctgggagagcgtcttcacaggcctcacccacatagatgcccacttcctgtcccagactaaacaggcaggagacaactttccttacctggtggcatatcaagctacagtgtgcgccagggctcaagctccacctccatcgtgggaccaaatgtggaagtgtctcatacggctgaaacctacactgcacgggccaacacccctgctgtataggctaggagccgtccaaaatgaggtcatcctcacacaccccataactaaatacatcatggcatgcatgtcggctgacctggaggtcgtcacttctggtggttctactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctg

SEQ ID NO: 43 - DNA, 4048 bp, unidentified
  source 1..4048: mol_type = other DNA; note = AID-PcrA-UGI
gcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgcgagagccgccaccatggacagcctcttgatgaaccggagggagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcatctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccacggaagaactttcaaagcctgggaagggctgcatgaaaattcagttcgtctctccagacagcttcggcgcatccttttgcccctgtatgaggttgatgacttacgagacgcatttcgtactagcggcagcgagactcccgggacctcagagtccgccacacccgaaagtatgatgaatgccctgctgaaccatatgaatacagaacaatccgaggcggtaaagaccacagaaggccccttgttgatcatggcgggggctgggagtggtaaaacgagggtccttactcaccgaatagcgtacttgcttgacgaaaaggacgtgagtccatataacgtgcttgccattaccttcacaaacaaggctgctagagagatgaaggaaagagtccaaaaacttgtcggtgaccaagcggaggtcatttggatgtctaccttccattctatgtgcgttcgcatacttcggcgagacgcggataggattgggatcgaacggaacttcacgataatagatcctacagatcaaaagtctgtaataaaagatgttctcaaaaatgagaatatagatagcaaaaaatttgaaccccgaatgttcataggtgccatatcaaacttgaagaacgaactcaaaacacctgcggacgcacaaaaggaagcaacagactaccacagtcagatggtcgcaactgtttattccggctaccaacgacagctgagtcggaatgaagcactggattttgatgatctgatcatgactactattaacctttttgaaagagtaccggaagtgttggaatattaccaaaacaaatttcaatatatccacgttgatgaataccaagatactaataaggcacagtatacattggtaaagctgctggcgtcaaagtttaaaaatctttgcgtggtcggggatagtgaccagagcatatacggttggcgcggcgccgacatacagaatatcttgtccttcgagaaagattatcctgaggcgaatacaatcttccttgagcagaattatagatctacaaaaactattttgaacgcggctaacgaagtaataaaaaataatagtgagcgaaagcctaaaggtctgtggacagctaacacaaatggtgaaaagattcattactacgaagcaatgactgaacgagacgaagcggagttcgtcatccgggaaataatgaaacaccaacgcaacggcaagaaataccaagacatggcaattctgtacaggaccaatgcgcaatccagagttctcgaagaaacctttatgaagagcaatatgccatacacgatggttggaggccaaaaattctatgataggaaagagatcaaagacctgctgagctacctccgaatcattgccaacagtaacgacgacatctcacttcaacggattattaacgtaccgaaacgcggggttggaccctcatcagttgagaaagttcaaaactatgcgttgcagaacaatatttccatgtttgacgctcttggagaagctgattttatcggcttgtcaaagaaagtaacccaggagtgtcttaacttttacgaactgatacaaagcctgataaaggaacaggaattccttgagatccacgagatcgtagatgaagttctgcaaaaatccggctatcgggaaatgttggaaagggagaacacgctcgaaagtaggtcaagactcgagaacatagatgagttcatgtcagtgcccaaagactatgaggagaatacgccccttgaagagcagtcattgatcaatttccttactgacctgtcactcgttgccgatattgacgaagctgacactgagaatggggtaacattgatgacgatgcacagtgctaagggattggagtttcccatagtcttcatcatgggtatggaggagtccctctttccacacattcgggcaatcaaatccgaggatgatcatgagatgcaagaggagcgcagaatctgttacgttgcgattacacgagcggaggaggttctttatattacacacgcaacgtctcggatgctctttggacgcccacagtcaaacatgccctctaggttccttaaggaaatacccgagagcctgctggagaaccatagctcaggtaagcggcagaccatacaaccaaaagctaagccgttcgccaaacgcggcttctcacagcgcacgactagcacgaagaagcaggtgctctccagcgattggaatgttggcgataaggttatgcacaaagcttggggagagggcatggtttccaatgtgaatgagaaaaatggatccatagagctcgacatcatctttaagagccagggcccaaagcgcctgctcgctcagttcgctccaatcgagaaaaaagaagactctggtggttctactaatctgtcagatattattgaaaaggagaccggtaagcaactggttatccaggaatccatcctcatgctcccagaggaggtggaagaagtcattgggaacaagccggaaagcgatatactcgtgcacaccgcctacgacgagagcaccgacgagaatgtcatgcttctgactagcgacgcccctgaatacaagccttgggctctggtcatacaggatagcaacggtgagaacaagattaagatgctctctggtggttctcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctg

SEQ ID NO: 44 - RNA, 20 nt, synthetic construct
  source 1..20: mol_type = other RNA
ggcccagactgagcacgtga

SEQ ID NO: 45 - RNA, 20 nt, synthetic construct
  source 1..20: mol_type = other RNA
tgaaagcatgatccgggacg

SEQ ID NO: 46 - RNA, 20 nt, synthetic construct
  source 1..20: mol_type = other RNA
tgaaagcagcaaagaggcac

SEQ ID NO: 47 - RNA, 20 nt, synthetic construct
  source 1..20: mol_type = other RNA
gaaagaaagctccaggtctg

SEQ ID NO: 48 - RNA, 20 nt, synthetic construct
  source 1..20: mol_type = other RNA
gattcctggtgccagaaaca

SEQ ID NO: 49 - RNA, 20 nt, synthetic construct
  source 1..20: mol_type = other RNA
gatgtctgcaggccagatga

SEQ ID NO: 50 - RNA, 21 nt, synthetic construct
  source 1..21: mol_type = other RNA
gccctccactagggcaagggt

SEQ ID NO: 51 - RNA, 21 nt, synthetic construct
  source 1..21: mol_type = other RNA
gccctccactagggcaagggt

SEQ ID NO: 52 - RNA, 20 nt, synthetic construct
  source 1..20: mol_type = other RNA
atgaagcactgtgggtacga

SEQ ID NO: 53 - RNA, 20 nt, synthetic construct
  source 1..20: mol_type = other RNA
gatgtctgcaggccagatga

SEQ ID NO: 54 - RNA, 20 nt, synthetic construct
  source 1..20: mol_type = other RNA
gattcctggtgccagaaaca

SEQ ID NO: 55 - RNA, 20 nt, synthetic construct
  source 1..20: mol_type = other RNA
ggcccagactgagcacgtga

SEQ ID NO: 56 - RNA, 20 nt, synthetic construct
  source 1..20: mol_type = other RNA
tgaaagcagcaaagaggcac

SEQ ID NO: 57 - RNA, 20 nt, synthetic construct
  source 1..20: mol_type = other RNA
gaaagaaagctccaggtctg

SEQ ID NO: 58 - RNA, 20 nt, synthetic construct
  source 1..20: mol_type = other RNA
aagctctttaaggtagaggg

SEQ ID NO: 59 - RNA, 20 nt, synthetic construct
  source 1..20: mol_type = other RNA
acacccccgtccgccatcag

SEQ ID NO: 60 - RNA, 20 nt, synthetic construct
  source 1..20: mol_type = other RNA
ataagaaatttagaattatc

SEQ ID NO: 61 - RNA, 20 nt, synthetic construct
  source 1..20: mol_type = other RNA
aagaaaggacagtcatgagt

SEQ ID NO: 62 - RNA, 20 nt, synthetic construct
  source 1..20: mol_type = other RNA
ccagactggactcaaacctt

SEQ ID NO: 63 - RNA, 20 nt, synthetic construct
  source 1..20: mol_type = other RNA
tgttcacattaaacaaaatt

SEQ ID NO: 64 - RNA, 20 nt, synthetic construct
  source 1..20: mol_type = other RNA
atttatcttcattaaagtta

SEQ ID NO: 65 - RNA, 20 nt, synthetic construct
  source 1..20: mol_type = other RNA
ccagatttgttaatgtaaac

SEQ ID NO: 66 - RNA, 20 nt, synthetic construct
  source 1..20: mol_type = other RNA
tatatgtgcttgattatgaa

SEQ ID NO: 67 - RNA, 20 nt, synthetic construct
  source 1..20: mol_type = other RNA
caaagtgtgcagagaaggtg

SEQ ID NO: 68 - RNA, 20 nt, synthetic construct
  source 1..20: mol_type = other RNA
tgtcttaggtcggaagtctg

SEQ ID NO: 69 - RNA, 20 nt, synthetic construct
  source 1..20: mol_type = other RNA
gacttgaagaggacagaagg

SEQ ID NO: 70 - DNA, 53 bp, synthetic construct
  misc_feature 1..53: note = Synthetic
  source 1..53: mol_type = other DNA
tcgtcggcagcgtcagatgtgtataagagacaggagctgcacatactagcccc

SEQ ID NO: 71 - DNA, 54 bp, synthetic construct
  misc_feature 1..54: note = Synthetic
  source 1..54: mol_type = other DNA
gtctcgtgggctcggagatgtgtataagagacagagggagcttggcatgagaaa

SEQ ID NO: 72 - DNA, 59 bp, synthetic construct
  misc_feature 1..59: note = Synthetic
  misc_feature 34..39: note = n is a, c, g, or t
  source 1..59: mol_type = other DNA
tcgtcggcagcgtcagatgtgtataagagacagnnnnnngcagaggaccagctaagagg

SEQ ID NO: 73 - DNA, 59 bp, synthetic construct
  misc_feature 1..59: note = Synthetic
  source 1..59: mol_type = other DNA
gtctcgtgggctcggagatgtgtataagagacagcccagtcactccaaagtgcagcagg

SEQ ID NO: 74 - DNA, 59 bp, synthetic construct
  misc_feature 1..59: note = Synthetic
  misc_feature 34..39: note = n is a, c, g, or t
  source 1..59: mol_type = other DNA
tcgtcggcagcgtcagatgtgtataagagacagnnnnnntgccaggatgccaatgagtt

SEQ ID NO: 75 - DNA, 59 bp, synthetic construct
  misc_feature 1..59: note = Synthetic
  source 1..59: mol_type = other DNA
gtctcgtgggctcggagatgtgtataagagacagcccagcactgcatgcaagagggaga

SEQ ID NO: 76 - DNA, 58 bp, synthetic construct
  misc_feature 1..58: note = Synthetic
  misc_feature 34..38: note = n is a, c, g, or t
  source 1..58: mol_type = other DNA
tcgtcggcagcgtcagatgtgtataagagacagnnnnntggtgatagtcatcccgggt

SEQ ID NO: 77 - DNA, 59 bp, synthetic construct
  misc_feature 1..59: note = Synthetic
  source 1..59: mol_type = other DNA
gtctcgtgggctcggagatgtgtataagagacagcccagcgtcatccttcagttctccc

SEQ ID NO: 78 - DNA, 55 bp, synthetic construct
  misc_feature 1..55: note = Synthetic
  source 1..55: mol_type = other DNA
tcgtcggcagcgtcagatgtgtataagagacagtgttccccagagtgacttttcc

SEQ ID NO: 79 - DNA, 58 bp, synthetic construct
  misc_feature 1..58: note = Synthetic
  source 1..58: mol_type = other DNA
gtctcgtgggctcggagatgtgtataagagacagttccactcatacagtggtagattt

SEQ ID NO: 80 - DNA, 53 bp, synthetic construct
  misc_feature 1..53: note = Synthetic
  source 1..53: mol_type = other DNA
tcgtcggcagcgtcagatgtgtataagagacaggggttttgccagactccaca

SEQ ID NO: 81 - DNA, 54 bp, synthetic construct
  misc_feature 1..54: note = Synthetic
  source 1..54: mol_type = other DNA
gtctcgtgggctcggagatgtgtataagagacagttgggactggagttgcttca

SEQ ID NO: 82 - DNA, 53 bp, synthetic construct
  misc_feature 1..53: note = Synthetic
  source 1..53: mol_type = other DNA
tcgtcggcagcgtcagatgtgtataagagacagaacaggaagttgggtaggga

SEQ ID NO: 83 - DNA, 54 bp, synthetic construct
  misc_feature 1..54: note = Synthetic
  source 1..54: mol_type = other DNA
gtctcgtgggctcggagatgtgtataagagacaggaggacagcagcagctcaaa

SEQ ID NO: 84 - DNA, 59 bp, synthetic construct
  misc_feature 1..59: note = Synthetic
  misc_feature 34..38: note = n is a, c, g, or t
  source 1..59: mol_type = other DNA
tcgtcggcagcgtcagatgtgtataagagacagnnnnnacaaacaagacagggaactgg

SEQ ID NO: 85 - DNA, 60 bp, synthetic construct
  misc_feature 1..60: note = Synthetic
  source 1..60: mol_type = other DNA
gtctcgtgggctcggagatgtgtataagagacagcccagctagaggggtgaggctgaaac

SEQ ID NO: 86 - DNA, 23 bp, synthetic construct
  misc_feature 1..23: note = Synthetic
  source 1..23: mol_type = other DNA
aactaaacagtcccactccatcc

SEQ ID NO: 87 - DNA, 24 bp, synthetic construct
  misc_feature 1..24: note = Synthetic
  source 1..24: mol_type = other DNA
gtatcttcatgcattctccacgcc

SEQ ID NO: 88 - DNA, 20 bp, synthetic construct
  misc_feature 1..20: note = Synthetic
  source 1..20: mol_type = other DNA
ggagaaacagagacaggccc

SEQ ID NO: 89 - DNA, 20 bp, synthetic construct
  misc_feature 1..20: note = Synthetic
  source 1..20: mol_type = other DNA
ccaggtttcgaagtggtggt

SEQ ID NO: 90 - DNA, 20 bp, synthetic construct
  misc_feature 1..20: note = Synthetic
  source 1..20: mol_type = other DNA
cccaccgggaacgaaagaga

SEQ ID NO: 91 - DNA, 22 bp, synthetic construct
  misc_feature 1..22: note = Synthetic
  source 1..22: mol_type = other DNA
gtctcccattagaccacaagca

SEQ ID NO: 92 - DNA, 63 bp, synthetic construct
  misc_feature 1..63: note = Synthetic
  misc_feature 34..43: note = n is a, c, g, or t
  source 1..63: mol_type = other DNA
tcgtcggcagcgtcagatgtgtataagagacagnnnnnnnnnnttgtgctccccactttggaa

SEQ ID NO: 93 - DNA, 68 bp, synthetic construct
  misc_feature 1..68: note = Synthetic
  misc_feature 35..44: note = n is a, c, g, or t
  source 1..68: mol_type = other DNA
gtctcgtgggctcggagatgtgtataagagacagnnnnnnnnnncctgttaatcaaggcaaactcacc

SEQ ID NO: 94 - DNA, 69 bp, synthetic construct
  misc_feature 1..69: note = Synthetic
  misc_feature 34..43: note = n is a, c, g, or t
  source 1..69: mol_type = other DNA
tcgtcggcagcgtcagatgtgtataagagacagnnnnnnnnnngactatatctttcatcccttcctccc

SEQ ID NO: 95 - DNA, 66 bp, synthetic construct
  misc_feature 1..66: note = Synthetic
  misc_feature 35..44: note = n is a, c, g, or t
  source 1..66: mol_type = other DNA
gtctcgtgggctcggagatgtgtataagagacagnnnnnnnnnncaactcttaaggccattgctcc

SEQ ID NO: 96 - DNA, 66 bp, synthetic construct
  misc_feature 1..66: note = Synthetic
  misc_feature 34..43: note = n is a, c, g, or t
  source 1..66: mol_type = other DNA
tcgtcggcagcgtcagatgtgtataagagacagnnnnnnnnnncccaatctacctgtgtcagttcc

SEQ ID NO: 97 - DNA, 65 bp, synthetic construct
  misc_feature 1..65: note = Synthetic
  misc_feature 35..44: note = n is a, c, g, or t
  source 1..65: mol_type = other DNA
gtctcgtgggctcggagatgtgtataagagacagnnnnnnnnnncctacccagcacaagactctg

SEQ ID NO: 98 - DNA, 19 bp, synthetic construct
  misc_feature 1..19: note = Synthetic
  source 1..19: mol_type = other DNA
ggagttggaagcgcgttac

SEQ ID NO: 99 - DNA, 20 bp, synthetic construct
  misc_feature 1..20: note = Synthetic
  source 1..20: mol_type = other DNA
caaaagcgacatggcaaacc

SEQ ID NO: 100 - DNA, 53 bp, synthetic construct
  misc_feature 1..53: note = Synthetic
  source 1..53: mol_type = other DNA
tcgtcggcagcgtcagatgtgtataagagacagtctgctctttttcccaggct

SEQ ID NO: 101 - DNA, 59 bp, synthetic construct
  misc_feature 1..59: note = Synthetic
  source 1..59: mol_type = other DNA
gtctcgtgggctcggagatgtgtataagagacagattaaggagaacaaaccttatgcac

SEQ ID NO: 102 - DNA, 53 bp, synthetic construct
  misc_feature 1..53: note = Synthetic
  source 1..53: mol_type = other DNA
tcgtcggcagcgtcagatgtgtataagagacagctcgtggtcattgaaccgct

SEQ ID NO: 103 - DNA, 57 bp, synthetic construct
  misc_feature 1..57: note = Synthetic
  source 1..57: mol_type = other DNA
gtctcgtgggctcggagatgtgtataagagacagagaaaggacagtcatgagttggt

SEQ ID NO: 104 - DNA, 2157 bp, synthetic construct
  misc_feature 1..2157: note = Synthetic
  source 1..2157: mol_type = other DNA
atggctgctgttcctcaaaataatctacaggagcaactagaacgtcactcagccagaacacttaataataaattaagtctttcaaaaccaaaattttcaggtttcacttttaaaaagaaaacatcttcagataacaatgtatctgtaactaatgtgtcagtagcaaaaacacctgtattaagaaataaagatgttaatgttaccgaagacttttccttcagtgaacctctacccaacaccacaaatcagcaaagggtcaaggacttctttaaaaatgctccagcaggacaggaaacacagagaggtggatcaaaatcattattgccagatttcttgcagactccgaaggaagttgtatgcactacccaaaacacaccaactgtaaagaaatcccgggatactgctctcaagaaattagaatttagttcttcaccagattctttaagtaccatcaatgattgggatgatatggatgactttgatacttctgagacttcaaaatcatttgttacaccaccccaaagtcactttgtaagagtaagcactgctcagaaatcaaaaaagggtaagagaaacttttttaaagcacagctttatacaacaaacacagtaaagactgacttgcctccaccctcctctgaaagcgagcaaatagatttgactgaggaacagaaggatgactcagaatggttaagcagcgatgtgatttgcatcgatgatggccccattgctgaagtgcatataaatgaagatgctcaggaaagtgactctctgaaaactcatttggaagatgaaagagataatagcgaaaagaagaagaatttggaagaagctgaattacattcaactgagaaagttccatgtattgaatttgatgatgatgattatgatacggattttgttccaccttctccagaagaaattatttctgcttcttcttcctcttcaaaatgccttagtacgttaaaggaccttgacacctctgacagaaaagaggatgttcttagcacatcaaaagatcttttgtcaaaacctgagaaaatgagtatgcaggagctgaatccagaaaccagcacagactgtgacgctagacagataagtttacagcagcagcttattcatgtgatggagcacatctgtaaattaattgatactattcctgatgataaactgaaacttttggattgtgggaacgaactgcttcagcagcggaacataagaaggaaacttctaacggaagtagattttaataaaagtgatgccagtcttcttggctcattgtggagatacaggcctgattcacttgatggccctatggagggtgattcctgccctacagggaattctatgaaggagttaaatttttcacaccttccctcaaattctgtttctcctggggactgtttactgactaccaccctaggaaagacaggattctctgccaccaggaagaatctttttgaaaggcctttattcaatacccatttacagaagtcctttgtaagtagcaactgggctgaaacaccaagactaggaaaaaaaaatgaaagctcttatttcccaggaaatgttctcacaagcactgctgtgaaagatcagaataaacatactgcttcaataaatgacttagaaagagaaacccaaccttcctatgatattgataattttgacatagatgactttgatgatgatgatgactgggaagacataatgcataatttagcagccagcaaatcttccacagctgcctatcaacccatcaaggaaggtcggccaattaaatcagtatcagaaagactttcctcagccaagacagactgtcttccagtgtcatctactgctcaaaatataaacttctcagagtcaattcagaattatactgacaagtcagcacaaaatttagcatccagaaatctgaaacatgagcgtttccaaagtcttagttttcctcatacaaaggaaatgatgaagatttttcataaaaaatttggcctgcataattttagaactaatcagctagaggcgatcaatgctgcactgcttggtgaagactgttttatcctgatgccgactggaggtggtaagagtttgtgttaccagctccctgcctgtgtttctcctggggtcactgttgtcatttctcccttgagatcactt

SEQ ID NO: 105 - DNA, 2094 bp, synthetic construct
  misc_feature 1..2094: note = Synthetic
  source 1..2094: mol_type = other DNA
atcgtagatcaagtccaaaagctgacttccttggatattccagctacatatctgacaggtgataagactgactcagaagctacaaatatttacctccagttatcaaaaaaagacccaatcataaaactcctatatgtcactccagaaaagatctgtgcaagtaacagactcatttctactctggagaatctctatgagaggaagctcttggcacgttttgttattgatgaagcacattgtgtcagtcagtggggacatgattttcgtcaagattacaaaagaatgaatatgcttcgccagaagtttccttctgttccggtgatggctcttacggccacagctaatcccagggtacagaaggacatcctgactcagctgaagattctcagacctcaggtgtttagcatgagctttaacagacataatctgaaatactatgtattaccgaaaaagcctaaaaaggtggcatttgattgcctagaatggatcagaaagcaccacccatatgattcagggataatttactgcctctccaggcgagaatgtgacaccatggctgacacgttacagagagatgggctcgctgctcttgcttaccatgctggcctcagtgattctgccagagatgaagtgcagcagaagtggattaatcaggatggctgtcaggttatctgtgctacaattgcatttggaatggggattgacaaaccggacgtgcgatttgtgattcatgcatctctccctaaatctgtggagggttactaccaagaatctggcagagctggaagagatggggaaatatctcactgcctgcttttctatacctatcatgatgtgaccagactgaaaagacttataatgatggaaaaagatggaaaccatcatacaagagaaactcacttcaataatttgtatagcatggtacattactgtgaaaatataacagaatgcaggagaatacagcttttggcctactttggtgaaaatggatttaatcctgatttttgtaagaaacacccagatgtttcttgtgataattgctgtaaaacaaaggattataaaacaagagatgtgactgacgatgtgaaaagtattgtaagatttgttcaagaacatagttcatcacaaggaatgagaaatataaaacatgtaggtccttctggaagatttactatgaatatgctggtcgacattttcttggggagtaagagtgcaaaaatccagtcaggtatatttggaaaaggatctgcttattcacgacacaatgccgaaagactttttaaaaagctgatacttgacaagattttggatgaagacttatatatcaatgccaatgaccaggcgatcgcttatgtgatgctcggaaataaagcacaaactgtactaaatggcaatttaaaggtagactttatggaaacagaaaattccagcagtgtgaaaaaacaaaaagcgttagtagcaaaagtgtctcagagggaagagatggttaaaaaatgtcttggagaacttacagaagtctgcaaatctctggggaaagtttttggtgtccattacttcaatatttttaataccgtcactctcaagaagcttgcagaatctttatcttctgatcctgaggttttgcttcaaattgatggtgttactgaagacaaactggaaaaatatggtgcggaagtgatttcagtattacagaaatactctgaatggacatcgccagctgaagacagttccccagggataagcctgtccagcagcagaggccccggaagaagtgccgctgaggagcttgacgaggaaatacccgtatcttcccactactttgcaagtaaaaccagaaatgaaaggaagaggaaaaagatgccagcctcccaaaggtctaagaggagaaaaactgcttccagtggttccaaggcaaagggggggtctgccacatgtagaaagatatcttccaaaacgaaatcctccagcatcattggatccagttcagcctcacatacttctcaagcgacatcaggagccaatagcaaattggggattatggctccaccgaagcctataaatagaccgtttcttaagccttcatatgcattctca

SEQ ID NO: 106 - DNA, 1198 bp, synthetic construct
  misc_feature 1..1198: note = Synthetic
  source 1..1198: mol_type = other DNA
tgccgccaccttagggtttggggcgtatatgtccaaggcacacggtatcgaccctaacatcagaactggggtaaggaccattaccacgggcggctccattacgtactccacctatggcaagttccttgccgacggtggctgttctgggggcgcctatgacatcataatatgtgatgagtgccactcaactgactcgactaccatcttgggcatcggcacagtcctggaccaagcggagacggctggagcgcggctcgtcgtgctcgccaccgctacacctccgggatcggttaccgtgccacaccccaatatcgaggaaataggcctgtccaacaatggagagatccccttctatggcaaagccatccccattgaggccatcaagggggggaggcatctcattttctgccattccaagaagaaatgtgacgagctcgccgcaaagctgacaggcctcggactgaacgctgtagcatattaccggggccttgatgtgtccgtcataccgcctatcggagacgtcgttgtcgtggcaacagacgctctaatgacgggtttcaccggcgattttgactcagtgatcgactgcaatacatgtgtcacccagacagtcgacttcagcttggatcccaccttcaccattgagacgacgaccgtgccccaagacgcggtgtcgcgctcgcaacggcgaggtagaactggcaggggtaggagtggcatctacaggtttgtgactccaggagaacggccctcgggcatgttcgattcttcggtcctgtgtgagtgctatgacgcgggctgtgcttggtatgagctcacgcccgctgagacctcggttaggttgcgggcttacctaaatacaccagggttgcccgtctgccaggaccatctggagttctgggagagcgtcttcacaggcctcacccacatagatgcccacttcctgtcccagactaaacaggcaggagacaactttccttacctggtggcatatcaagctacagtgtgcgccagggctcaagctccacctccatcgtgggaccaaatgtggaagtgtctcatacggctgaaacctacactgcacgggccaacacccctgctgtataggctaggagccgtccaaaatgaggtcatcctcacacaccccataactaaatacatcatggcatgcatgtcggctgacctggaggtcgtcact

SEQ ID NO: 107 - DNA, 2190 bp, synthetic construct
  misc_feature 1..2190: note = Synthetic
  source 1..2190: mol_type = other DNA
atgaatgccctgctgaaccatatgaatacagaacaatccgaggcggtaaagaccacagaaggccccttgttgatcatggcgggggctgggagtggtaaaacgagggtccttactcaccgaatagcgtacttgcttgacgaaaaggacgtgagtccatataacgtgcttgccattaccttcacaaacaaggctgctagagagatgaaggaaagagtccaaaaacttgtcggtgaccaagcggaggtcatttggatgtctaccttccattctatgtgcgttcgcatacttcggcgagacgcggataggattgggatcgaacggaacttcacgataatagatcctacagatcaaaagtctgtaataaaagatgttctcaaaaatgagaatatagatagcaaaaaatttgaaccccgaatgttcataggtgccatatcaaacttgaagaacgaactcaaaacacctgcggacgcacaaaaggaagcaacagactaccacagtcagatggtcgcaactgtttattccggctaccaacgacagctgagtcggaatgaagcactggattttgatgatctgatcatgactactattaacctttttgaaagagtaccggaagtgttggaatattaccaaaacaaatttcaatatatccacgttgatgaataccaagatactaataaggcacagtatacattggtaaagctgctggcgtcaaagtttaaaaatctttgcgtggtcggggatagtgaccagagcatatacggttggcgcggcgccgacatacagaatatcttgtccttcgagaaagattatcctgaggcgaatacaatcttccttgagcagaattatagatctacaaaaactattttgaacgcggctaacgaagtaataaaaaataatagtgagcgaaagcctaaaggtctgtggacagctaacacaaatggtgaaaagattcattactacgaagcaatgactgaacgagacgaagcggagttcgtcatccgggaaataatgaaacaccaacgcaacggcaagaaataccaagacatggcaattctgtacaggaccaatgcgcaatccagagttctcgaagaaacctttatgaagagcaatatgccatacacgatggttggaggccaaaaattctatgataggaaagagatcaaagacctgctgagctacctccgaatcattgccaacagtaacgacgacatctcacttcaacggattattaacgtaccgaaacgcggggttggaccctcatcagttgagaaagttcaaaactatgcgttgcagaacaatatttccatgtttgacgctcttggagaagctgattttatcggcttgtcaaagaaagtaacccaggagtgtcttaacttttacgaactgatacaaagcctgataaaggaacaggaattccttgagatccacgagatcgtagatgaagttctgcaaaaatccggctatcgggaaatgttggaaagggagaacacgctcgaaagtaggtcaagactcgagaacatagatgagttcatgtcagtgcccaaagactatgaggagaatacgccccttgaagagcagtcattgatcaatttccttactgacctgtcactcgttgccgatattgacgaagctgacactgagaatggggtaacattgatgacgatgcacagtgctaagggattggagtttcccatagtcttcatcatgggtatggaggagtccctctttccacacattcgggcaatcaaatccgaggatgatcatgagatgcaagaggagcgcagaatctgttacgttgcgattacacgagcggaggaggttctttatattacacacgcaacgtctcggatgctctttggacgcccacagtcaaacatgccctctaggttccttaaggaaatacccgagagcctgctggagaaccatagctcaggtaagcggcagaccatacaaccaaaagctaagccgttcgccaaacgcggcttctcacagcgcacgactagcacgaagaagcaggtgctctccagcgattggaatgttggcgataaggttatgcacaaagcttggggagagggcatggtttccaatgtgaatgagaaaaatggatccatagagctcgacatcatctttaagagccagggcccaaagcgcctgctcgctcagttcgctccaatcgagaaaaaagaagac

SEQ ID NO: 108 - DNA, 1181 bp, synthetic construct
  misc_feature 1..1181: note = Synthetic
  source 1..1181: mol_type = other DNA
atgaacttcctgtctgagcaactgctggcacacctgaacaaggagcagcaagaagctgtgcggaccaccgagggacctctgctgatcatggccggcgctggaagcggaaaaacaagagtgctgacacaccgcatcgcctacctgatggctgagaagcacgtggccccttggaacatcctggccattacctttacaaacaaggccgctagagagatgagagaaagagtgcagagcctcctgggaggcgccgccgaggacgtgtggatcagcaccttcgccagcatggccgtgcggatcctgagaagagatatcgacagaatcggcatcaaccggaacttcagcatcctggacccaacagaccagctgagcgtgatgaaaaccatcctgaaagaaaagaacatcgaccccaagaaattcgagcctagaacaatcctgggcacaatcagcgccgccaagaatgaactgctgcctccagaacagtttgcgaagcgggcctccacctactatgagaaagtggtgtctgacgtctaccaggagtatcagcagcggctgctcaggtgtcacagccttgacttcgatgatctgatcatgaccacaatccagctgtttgaccgggtccccgacgtgctccactactaccaatacaagtttcagtacatccacatcgatgagtaccaggacacaaacagagcccaatacaccctggtgaaaaagctggctgagcggttccagaacatcgccgccgtgggcgacgccgatcagtctatctacagatggcggggcgccgacatccagaacatcctgagcttcgaaagagattaccccaacgccaaagtgatcctgctggagcaaaattaccggagcacgaagcgcatcctgcaggccgcaaacgaggtgatcgagcacaacgtgaacagaaagcctaagcggatctggaccgagaatcctgagggcaagcccatcctgtactacgaggccatgaacgaagccgacgaggcccagttcgtggccggcagaatcagagaggccgtggagcgcggcgagagaagataccgagacttcgccgtgctgtacagaaccaatgcccagtccagagtcatggaagagatgctgctgaaggccaacatcccttaccagatagtgggcggcgtgaagttctacgacagaaaggaaatcaagga

SEQ ID NO: 109 - DNA, 723 bp, synthetic construct
  misc_feature 1..723: note = Synthetic
  source 1..723: mol_type = other DNA
atggcgaagatccacatggtccttcagggtaaaggtggggtcggaaaaagcgcaatcgcggcgatcatagcccagtacaagatggacaaaggtcagacgccgctttgtatagatacagatccagtcaatgccacgtttgagggatataaggcccttaatgtacgacgccttaacatcatggctggggatgagatcaacagccgcaactttgatacactggtcgagctcatcgcgcccaccaaagatgacgtagtaatcgacaacggtgcctcatcttttgttcctctgtcacactatctcatatcaaatcaagtaccggcactcctccaggagatgggacatgagctcgtgatacataccgtggtaactggcggccaggcattgcttgacactgtaagtgggttcgcccagcttgccagccagtttccagctgaagctctgtttgtcgtgtggctgaatccgtactggggtccaattgaacacgaagggaagtcattcgagcaaatgaaagcttacactgctaataaggctagggtctcaagcattatccaaatcccagcccttaaggaggagacatacggacgagacttctccgatatgctgcaagaacggttgactttcgaccaggccctcgcagacgaatctttgactataatgacccggcagaggcttaaaatagtgagacgcggccttttcgaacaattggacgccgcagccgttctg

SEQ ID NO: 110 - DNA, 2160 bp, synthetic construct
  misc_feature 1..2160: note = Synthetic
  source 1..2160: mol_type = other DNA
atggacgtatcttaccttttggattcattgaatgacaaacagcgcgaagctgtagctgccccaagatccaacctcttggtgttggcgggtgctggctcagggaagacccgagttcttgtacacagaatcgcgtggcttatgtctgttgaaaattgcagcccttactctataatggccgtcacgtttacgaataaggcagctgcggaaatgcgccatcgaattgggcagcttatgggcacttcacaaggtggcatgtgggtgggaacttttcacgggctcgctcatcggctgttgcgcgcacatcacatggacgcgaacctgcctcaagattttcagatcctcgattccgaagatcaattgcgccttctgaaaagactgatcaaggcaatgaacctggatgaaaagcaatggccaccccgacaggcaatgtggtacataaacagccaaaaagacgagggtcttcgaccacatcatatccaaagttatggtaaccctgttgaacaaacatggcagaaggtttatcaggcgtaccaggaagcctgtgaccgagcgggtcttgtcgattttgcagaactgctcctcagagcacatgagttgtggctcaataaacctcacatccttcagcattatagggaaagattcacgaatatactggttgatgagtttcaggatacaaacaatatccaatatgcttggattagacttctcgcaggagacacgggtaaggtgatgatcgtcggtgacgatgaccaatcaatttacggatggcggggtgcccaggtggaaaacatacaaagatttcttaacgacttccctggggcggaaacgattaggttggagcagaattatcggagtacttctaacatactcagtgcagctaacgcactcatcgagaacaacaacggccgactgggaaagaagctttggacagatggcgctgacggtgagccgatatcactgtattgcgcgtttaacgagcttgacgaggcccgcttcgtcgtcaatagaataaaaacctggcaagataatgggggggcgctcgctgagtgcgctattttgtatcggagtaacgcgcaaagccgggttttggaggaagcacttcttcaggcatctatgccataccggatatatggaggaatgcgattttttgaacgccaggagatcaaagatgcgcttagttatcttcgacttattgcgaatagaaatgatgatgcggcctttgagcgggttgtcaatacacccacacgcgggataggtgacagaactctggatgttgtcaggcaaacatctagagaccggcaactcaccctttggcaggcatgtagagaactgctccaggaaaaagcactggctggccgcgcggcatcagcactgcaaaggtttatggagctgattgacgctcttgcacaggaaacagcggacatgcccttgcatgtccaaacggatagagtgatcaaagattcaggtcttcgcacgatgtatgaacaagaaaaaggggagaaagggcagactaggatagagaacctcgaagaattggttacagctacccgccagttttcatacaatgaggaagacgaggacctgatgccactccaagctttcttgtcccacgctgcgctcgaagccggcgagggtcaagctgatacctggcaggatgcggtacaactgatgaccctccactcagccaagggtctggaattccctcaagtgtttatcgtcgggatggaggagggtatgttcccatcccagatgtctttggacgaaggaggacgactcgaggaggagcggcgactcgcctatgttggtgtaacccgagcgatgcagaaactcacgttgacgtatgcagagacgcgcaggttgtatgggaaggaggtgtaccaccgaccctctaggttcattggcgaacttcctgaagaatgtgtggaagaagtacgcctgcgggctacggtatctaggcccgttagccatcaacggatggggactccaatggtggagaatgactcaggctataagctgggccaaagggtccgccacgcgaaatttggtgagggcaccatcgtaaacatggaaggaagtggtgaacattcaaggcttcaagtagcctttcagggacaagggattaagtggcttgtggccgcatacgcgcgactcgagagcgtg

SEQ ID NO: 111 - DNA, 1182 bp, synthetic construct
  misc_feature 1..1182: note = Synthetic
  source 1..1182: mol_type = other DNA
atgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaa

SEQ ID NO: 112 - DNA, 1182 bp, synthetic construct
  misc_feature 1..1182: note = Synthetic
  source 1..1182: mol_type = other DNA
atgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtggacttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtggggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaa

SEQ ID NO: 113 - DNA, 1182 bp, synthetic construct
  misc_feature 1..1182: note = Synthetic
  source 1..1182: mol_type = other DNA
atgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtgaggagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaa

SEQ ID NO: 114 - DNA, 1182 bp, synthetic construct
  misc_feature 1..1182: note = Synthetic
  source 1..1182: mol_type = other DNA
atgcccaagaagaagccgacgcccatccagctgaacccggcccccgacggctctgcagttaacgggaccagctctgcggagaccaacttggaggccttgcagaagaagctggaggagctagagcttgatgagcagcagcgaaagcgccttgaggcctttcttacccagaagcagaaggtgggagaactgaaggatgacgactttgagaagatcagtgagctgggggctggcaatggcggtgtggtgttcaaggtctcccacaagccttctggcctggtcatggccagaaagctaattcatctggagatcaaacccgcaatccggaaccagatcataagggagctgcaggttctgcatgagtgcaactctccgtacatcgtgggcttctatggtgcgttctacagcgatggcgagatcagtatctgcatggagcacatggatggaggttctctggatcaagtcctgaagaaagctggaagaattcctgaacaaattttaggaaaagttagcattgctgtaataaaaggcctgacatatctgagggagaagcacaagatcatgcacagagatgtcaagccctccaacatcctagtcaactcccgtgggaagatcaagctctgtgactttggggtcagcgggcagctcatcgactccatggccaactccttcgtgggcacaaggtcctacatgtcgccagaaagactccaggggactcattactctgtgcagtcagacatctggagcatgggactgtctctggtagagatggcggttgggaggtatcccatccctcctccagatgccaaggagctggagctgatgtttgggtgccaggtggaaggagatgcggctgagaccccacccaggccaaggacccccgggaggccccttagctcatacggaatggacagccgacctcccatggcaatttttgagttgttggattacatagtcaacgagcctcctccaaaactgcccagtggagtgttcagtctggaatttcaagattttgtgaataaatgcttaataaaaaaccccgcagagagagcagatttgaagcaactcatggttcatgcttttatcaagagatctgatgctgaggaagtggattttgcaggttggctctgctccaccatcggccttaaccagcccagcacaccaacccatgctgctggcgtctaa

SEQ ID NO: 115 - DNA, 1199 bp, synthetic construct
  misc_feature 1..1199: note = Synthetic
  source 1..1199: mol_type = other DNA
atgggtcgctttgacagggaggtagatattggaattcctgatgctacaggacgcttagagattcttcagatccataccaagaacatgaagctggcagatgagtggacctggaacaggtgaagtgatgatgatggctgaccaggcgttacagtgtctctaggcagttgctgggaactggctagagacataaggttaagatgtgaggagatgggttttgatttctggacaggggaaaggaagtaatctgagatgaatccaggaaatgaagcttcgacaccgagctcgcctttgacaactgtcttgttacaaatgtttggtccttgcttctttaacctcttagtaaagcctttgattgtcttttcactgttggagacacagttgcagaagatgaagtggtttgtgagattgaaactgacaagcaaggcagcgagggcagaggaagtcttctaacatgcggtgacgtggaggagaatcccggccctgtcagtaaaggtgaagaactgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggtatggatgaattatataaataa

SEQ ID NO: 116 - DNA, 1244 bp, synthetic construct
  misc_feature 1..1244: note = Synthetic
  source 1..1244: mol_type = other DNA
atgggtcgctttgacagggaggtagatattggaattcctgatgctacaggacgcttagagattcttcagatccataccaagaacatgaagctggcagatgatgtggacctggaacaggtgaagtgatgatgatggctgaccaggcgttacagtgtctctaggcagttgctgggaactggctagagacataaggttaagatgtgaggagatgggttttgatttctggacaggggaaaggaagtaatctgagattgaatccaggaaatgaagcttcgacaccgagctcgcctttgacaactgtcttgttacaaatgtttggtccttgcttctttaacctcttagtaaagtttgtgtattctagattaccacagttccagagacaatgctggcacaaggcttccagcccatcctgtcacactgacacggagaatgaaatcgtcctgcctctgggctccttagatcagcaaggcagcgagggcagaggaagtcttctaacatgcggtgacgtggaggagaatcccggccctgtcagtaaaggtgaagaactgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggtatggatgaattatataaataa

SEQ ID NO: 117 - RNA, 20 nt, synthetic construct
  source 1..20: mol_type = other RNA
ggggccactagggacaggat

SEQ ID NO: 118 - RNA, 20 nt, synthetic construct
  source 1..20: mol_type = other RNA
tagaagcccacgatgtacgg

SEQ ID NO: 119 - RNA, 20 nt, synthetic construct
  source 1..20: mol_type = other RNA
cccacgggagttgactagga

SEQ ID NO: 120 - RNA, 20 nt, synthetic construct
  source 1..20: mol_type = other RNA
cttgatctccccacgggagt

SEQ ID NO: 121 - RNA, 20 nt, synthetic construct
  source 1..20: mol_type = other RNA
ctgtacaaacttgatgactt

SEQ ID NO: 122 - RNA, 20 nt, synthetic construct
  source 1..20: mol_type = other RNA
atagataacatggatgagta

SEQ ID NO: 123 - RNA, 20 nt, synthetic construct
  source 1..20: mol_type = other RNA
gagtatgtccgtaacacaac

SEQ ID NO: 124 - RNA, 20 nt, synthetic construct
  source 1..20: mol_type = other RNA
attaagattgtacaacagat

SEQ ID NO: 125 - RNA, 20 nt, synthetic construct
  source 1..20: mol_type = other RNA
tggtattaagattgtacaac

SEQ ID NO: 126 - RNA, 20 nt, synthetic construct
  source 1..20: mol_type = other RNA
cagaaagttcggaccatcag

SEQ ID NO: 127 - RNA, 20 nt, synthetic construct
  source 1..20: mol_type = other RNA
agcagaaagttcggaccatc

SEQ ID NO: 128 - RNA, 20 nt, synthetic construct
  source 1..20: mol_type = other RNA
tcctttctgacgtctcaccc

SEQ ID NO: 129 - RNA, 20 nt, synthetic construct
  source 1..20: mol_type = other RNA
tatcagacagctgcagcagc

SEQ ID NO: 130 - RNA, 20 nt, synthetic construct
  source 1..20: mol_type = other RNA
gaccacagacttccgaccta

SEQ ID NO: 131 - RNA, 194 nt, synthetic construct
  source 1..194: mol_type = other RNA
gaacaaaccttatgcacatagttttagagctagaaatagcaagttaaaataaggctagtccgttatcaacttgaaaaagtggcaccgagtcggtgctgcaaacttgatgacttagttcgtccatatgtgcataaggttccgaaagattgacgcggttctatctagttacgcgttaaaccaactagaaatttttt

SEQ ID NO: 132 - RNA, 184 nt, synthetic construct
  source 1..184: mol_type = other RNA
gtacttgtgaaagttattgatgttttagagctagaaatagcaagttaaaataaggctagtccgttatcaacttgaaaaagtggcaccgagtcggtgcgtttgcacagtatcctatcaataactttcactccctttattgacgcggttctatctagttacgcgttaaaccaactagaaatttttt

SEQ ID NO: 133 - RNA, 202 nt, synthetic construct
  source 1..202: mol_type = other RNA
gaagctctagctgttgtgttagttttagagctagaaatagcaagttaaaataaggctagtccgttatcaacttgaaaaagtggcaccgagtcggtgcacctgatgtagataacatggatgagtatgtccgtaacacaacagctagataataacattgacgcggttctatctagttacgcgttaaaccaactagaaatttttt

SEQ ID NO: 134 - RNA, 200 nt, synthetic construct
  source 1..200: mol_type = other RNA
gaagctctagctgttgtgttagttttagagctagaaatagcaagttaaaataaggctagtccgttatcaacttgaaaaagtggcaccgagtcggtgcgatgtggataacatggatgagtatgtccgtaacacaacagctagagcgaaatattttgacgcggttctatctagttacgcgttaaaccaactagaaatttttt

SEQ ID NO: 135 - RNA, 202 nt, synthetic construct
  source 1..202: mol_type = other RNA
gaagctctagctgttgtgttagttttagagctagaaatagcaagttaaaataaggctagtccgttatcaacttgaaaaagtggcaccgagtcggtgcacctgatgtggataacatggatgagtatgtccgtaacacaacagctagataaatattttgacgcggttctatctagttacgcgttaaaccaactagaaatttttt

SEQ ID NO: 136 - RNA, 200 nt, synthetic construct
  source 1..200: mol_type = other RNA
gaagctctagctgttgtgttagttttagagctagaaatagcaagttaaaataaggctagtccgttatcaacttgaaaaagtggcaccgagtcggtgcgatatggataacatggatgagtatgtccgtaacacaacagctagagcacagaaccttgacgcggttctatctagttacgcgttaaaccaactagaaatttttt

SEQ ID NO: 137 - RNA, 202 nt, synthetic construct
  source 1..202: mol_type = other RNA
gaagctctagctgttgtgttagttttagagctagaaatagcaagttaaaataaggctagtccgttatcaacttgaaaaagtggcaccgagtcggtgcacctgatatggataacatggatgagtatgtccgtaacacaacagctagaaacaacacttgacgcggttctatctagttacgcgttaaaccaactagaaatttttt

SEQ ID NO: 138 - RNA, 180 nt, synthetic construct
  source 1..180: mol_type = other RNA
gaagctctagctgttgtgttagttttagagctagaaatagcaagttaaaataaggctagtccgttatcaacttgaaaaagtggcaccgagtcggtgctgagtgtgtccgtaacacaacagctagtaacaaatttgacgcggttctatctagttacgcgttaaaccaactagaaatttttt

SEQ ID NO: 139 - RNA, 180 nt, synthetic construct
  source 1..180: mol_type = other RNA
gtcctggcaagcgagacacacgttttagagctagaaatagcaagttaaaataaggctagtccgttatcaacttgaaaaagtggcaccgagtcggtgcatcttaacaccagtgtgtctcgcttgcaacccttcttgacgcggttctatctagttacgcgttaaaccaactagaaatttttt

SEQ ID NO: 140 - RNA, 180 nt, synthetic construct
  source 1..180: mol_type = other RNA
gtcctggcaagcgagacacacgttttagagctagaaatagcaagttaaaataaggctagtccgttatcaacttgaaaaagtggcaccgagtcggtgcatctcaacaccagtgtgtctcgcttgcaattcttcttgacgcggttctatctagttacgcgttaaaccaactagaaatttttt

SEQ ID NO: 141 - RNA, 199 nt, synthetic construct
  source 1..199: mol_type = other RNA
gtgtgcaaaagcaagaagtccgttttagagctagaaatagcaagttaaaataaggctagtccgttatcaacttgaaaaagtggcaccgagtcggtgcatcttaacaccagtgtgtctcgcttgccaggacttcttgcttttgcttctaaatttgacgcggttctatctagttacgcgttaaaccaactagaaatttttt

SEQ ID NO: 142 - RNA, 124 nt, synthetic construct
  source 1..124: mol_type = other RNA
tgtcttaggtcggaagtctggttttagagctagaaatagcaagttaaaataaggctagtccgttatcaacttgaaaaagtggcaccgagtcggtgcaacagggaccacagacttccgacctaag

SEQ ID NO: 143 - RNA, 124 nt, synthetic construct
  source 1..124: mol_type = other RNA
tgtcttaggtcggaagtctggttttagagctagaaatagcaagttaaaataaggctagtccgttatcaacttgaaaaagtggcaccgagtcggtgcaacagggatcacagacttccgacctaag

SEQ ID NO: 144 - RNA, 124 nt, synthetic construct
  source 1..124: mol_type = other RNA
tgtcttaggtcggaagtctggttttagagctagaaatagcaagttaaaataaggctagtccgttatcaacttgaaaaagtggcaccgagtcggtgcaacagggactacagacttccgacctaag

SEQ ID NO: 145 - RNA, 124 nt, synthetic construct
  source 1..124: mol_type = other RNA
tgtcttaggtcggaagtctggttttagagctagaaatagcaagttaaaataaggctagtccgttatcaacttgaaaaagtggcaccgagtcggtgcaacagggaccatagacttccgacctaag

SEQ ID NO: 146 - RNA, 124 nt, synthetic construct
  source 1..124: mol_type = other RNA
tgtcttaggtcggaagtctggttttagagctagaaatagcaagttaaaataaggctagtccgttatcaacttgaaaaagtggcaccgagtcggtgcaacagggattatagacttccgacctaag

SEQ ID NO: 147 - RNA, 20 nt, synthetic construct
  source 1..20: mol_type = other RNA
gctgatgtctcctacacttg

SEQ ID NO: 148 - RNA, 20 nt, synthetic construct
  source 1..20: mol_type = other RNA
gaacaaaccttatgcacata

SEQ ID NO: 149 - RNA, 20 nt, synthetic construct
  source 1..20: mol_type = other RNA
tgctgttgtagcctctgccc

SEQ ID NO: 150 - RNA, 20 nt, synthetic construct
  source 1..20: mol_type = other RNA
tgctgttgtagcctctgccc

SEQ ID NO: 151 - RNA, 20 nt, synthetic construct
  source 1..20: mol_type = other RNA
tgctgttgtagcctctgccc

SEQ ID NO: 152 - RNA, 20 nt, synthetic construct
  source 1..20: mol_type = other RNA
tgctgttgtagcctctgccc

SEQ ID NO: 153 - RNA, 20 nt, synthetic construct
  source 1..20: mol_type = other RNA
tgctgttgtagcctctgccc

SEQ ID NO: 154 - RNA, 20 nt, synthetic construct
  source 1..20: mol_type = other RNA
gctgttgtagcctctgccct

SEQ ID NO: 155 - RNA, 20 nt, synthetic construct
  source 1..20: mol_type = other RNA
acttctaagatgtggcaaga

SEQ ID NO: 156 - RNA, 20 nt, synthetic construct
  source 1..20: mol_type = other RNA
acttctaagatgtggcaaga

SEQ ID NO: 157 - RNA, 20 nt, synthetic construct
  source 1..20: mol_type = other RNA
gcaataaagaaggaatgccc

SEQ ID NO: 158 - RNA, 40 nt, synthetic construct
  source 1..40: mol_type = other RNA
agggaccacagacttccgacctaagacaatttccggagac

SEQ ID NO: 159 - DNA, 28 bp, synthetic construct
  misc_feature 1..28: note = Synthetic
  source 1..28: mol_type = other DNA
gcttatcagacagctgcagcagcaggat

SEQ ID NO: 160 - DNA, 31 bp, synthetic construct
  misc_feature 1..31: note = Synthetic
  source 1..31: mol_type = other DNA
aatccagggtgagacgtcagaaaggaagtaa

SEQ ID NO: 161 - RNA, 40 nt, synthetic construct; identical to SEQ ID NO: 158
  source 1..40: mol_type = other RNA

SEQ ID NO: 162 - RNA, 40 nt, synthetic construct
  source 1..40: mol_type = other RNA
agggactatagacttccgacctaagacaatttccggagac

SEQ ID NO: 163 - RNA, 40 nt, synthetic construct
  source 1..40: mol_type = other RNA
agggattatagacttccgacctaagacaatttccggagac

SEQ ID NO: 164 - RNA, 40 nt, synthetic construct
  source 1..40: mol_type = other RNA
agggaccatagacttccgacctaagacaatttccggagac

SEQ ID NO: 165 - RNA, 40 nt, synthetic construct; identical to SEQ ID NO: 158
  source 1..40: mol_type = other RNA

SEQ ID NO: 166 - RNA, 40 nt, synthetic construct; identical to SEQ ID NO: 163
  source 1..40: mol_type = other RNA

SEQ ID NO: 167 - RNA, 40 nt, synthetic construct; identical to SEQ ID NO: 162
  source 1..40: mol_type = other RNA

SEQ ID NO: 168 - RNA, 40 nt, synthetic construct; identical to SEQ ID NO: 164
  source 1..40: mol_type = other RNA

(19) United States
(12) Patent Application Publication
(10) Pub. No.: US 2025/0346885 A1
(43) Pub. Date: Nov. 13, 2025

(54) SYSTEMS AND METHODS FOR TARGETED CONTINUOUS GENOME MUTAGENESIS

(71) Applicants: The Broad Institute, Inc., Cambridge, MA (US); President and Fellows of Harvard College, Cambridge, MA (US)

(72) Inventors: Dawn (Xi) CHEN, Cambridge, MA (US); Fei CHEN, Cambridge, MA (US); George WYTHES, Cambridge, MA (US)

(73) Assignees: THE BROAD INSTITUTE, INC., Cambridge, MA (US); PRESIDENT AND FELLOWS OF HARVARD COLLEGE, Cambridge, MA (US)

(21) Appl. No.: 19/271,555

(22) Filed: Jul. 16, 2025

Related U.S. Application Data
(63) Continuation of application No. PCT/US2024/011869, filed on Jan. 17, 2024.
(60) Provisional application No. 63/439,469, filed on Jan. 17, 2023.

(51) Int. Cl. C12N 15/10; C12N 9/22; C12N 9/78; C12N 9/90; C12N 15/11

(52) U.S. Cl. CPC C12N 15/1024; C12N 9/226; C12N 9/78; C12N 9/90; C12N 15/11; C12N 2310/20

(57) ABSTRACT

The disclosure provides for compositions, systems, and methods for long-range targeted mutagenesis. In particular, the disclosure provides engineered compositions comprising a programmable nickase configured to introduce a single-strand nick in double-stranded DNA (dsDNA) at one or more targeted nick sites; a helicase configured to unwind a portion of the dsDNA at the one or more targeted nick sites; and a deaminase configured to introduce one or more base edits within the portion of unwound dsDNA. Also provided are vector and delivery systems comprising one or more polynucleotides encoding the components of the compositions, as well as modified cells, cell populations, animal models, pharmaceutical compositions, and kits comprising the compositions.

### CROSS-REFERENCE TO RELATED APPLICATIONS

This application claims the benefit of U.S. Provisional Application No. 63/439,469, filed Jan. 17, 2023. The entire contents of the above-identified application is hereby fully incorporated by reference.

### STATEMENT REGARDING FEDERALLY SPONSORED RESEARCH

This invention was made with government support under Grant Nos. MH121289 and NS132135 awarded by the National Institutes of Health. The government has certain rights in the invention.

### TECHNICAL FIELD

The subject matter disclosed herein is generally directed to systems and methods for targeted continuous genome mutagenesis and directed continuous evolution.

### REFERENCE TO AN ELECTRONIC SEQUENCE LISTING

The contents of the electronic sequence listing (“BROD-5680WP_ST26.xml”; Size is 215,734 bytes and it was created on Jan. 17, 2024) is herein incorporated by reference in its entirety.

### BACKGROUND

A fundamental challenge of genomics is to chart the impact of billions of bases in a genome (e.g. ˜3 billion in the human genome) on protein function and gene regulation. Therefore, a critical goal is to develop strategies for mutagenizing genomic sequences systematically and at high throughput. In particular, saturation mutagenesis of single genomic loci could emulate the natural evolution process to reveal sequence-structure relationships, gain-of-function, and loss-of-function phenotypes. By performing such mutagenesis and selection in a stepwise and/or continuous process, this evolutionary process could be directed to generate enhanced protein functions, gene expression, or cell fitness.

However, targeted mutagenesis in the endogenous mammalian genome remains difficult for three primary reasons. First, many existing tools require exogenous overexpression of the gene of interest on a plasmid or vector (e.g., deep mutational scanning, VEGAS). This is sensitive to gene dosage and cannot be used to evolve noncoding regions in their native chromatin contexts. Second, some tools (e.g., TRACE, TRIDENT) require integrating exogenous sequences into the genome, which leads to experimental complexity and constraints throughput. Third, existing tools targeting the endogenous genome are either non-specific (e.g., alkylators that introduce genome-wide mutations) or confined to narrow genomic windows (e.g., CRISPR base-editors, CRISPR-X, or TAM). Whereas CRISPR base-editor screens have been used to interrogate protein function and regulatory elements, they are limited in the base positions that can be targeted with high efficiency and can lead to artificial variants linkage due to the base editor mutating multiple bases in the editing window.

### SUMMARY OF THE INVENTION

The present disclosure provides compositions, vector systems, delivery systems, and methods for targeted mutagenesis. In one embodiment, the composition for targeted mutagenesis, comprises a programmable nickase configured to introduce a single-strand nick in double-stranded DNA (dsDNA) at one or more targeted nick sites; a helicase configured to unwind a portion of the dsDNA at the one or more targeted nick sites; and a deaminase configured to introduce one or more base edits within the portion of unwound dsDNA.

In one embodiment, the composition for targeted mutagenesis comprises a programmable nickase comprising a Cas nickase (nCas) and one or more guide molecules capable of forming a complex with the nCas and directing sequence-specific binding of the complex to the one or more targeted nick sites. In another embodiment, the nCas comprises a Type II or Type V Cas.

In one embodiment, the composition for targeted mutagenesis comprises a programmable nickase comprising an OMEGA nickase and one or more @RNA molecules capable of forming a complex with the OMEGA nickase and directing sequence-specific binding of the complex to the one or more targeted nick sites. In another embodiment, the OMEGA nickase comprises an IscB nickase, an IsrB nickase, an IshB nickase, a TnpB nickase, or a Fanzor nickase.

In one embodiment, the composition for targeted mutagenesis comprises a helicase that exhibits a processivity range of greater than or equal to 200 base pairs. In one embodiment, the helicase is selected from the group comprising BLM, NS3, PcrA, PcrA M6, RepX, TraI, DNA2, Srs2, RecG, PriA, UvrD. In one embodiment, the composition for targeted mutagenesis comprises a helicase that exhibits a processivity range of less than 200 base pairs. In one embodiment, the helicase is selected from the group comprising UvrD, Rep, and Sgs1.

In one embodiment, the composition for targeted mutagenesis comprises a deaminase that is linked to or other otherwise capable of associating with the helicase. In one embodiment, the deaminase and helicase are further linked to or capable of associating with the programmable nickase.

In an embodiment, the deaminase functions as a cytidine deaminase, an adenosine deaminase, or both.

In one embodiment, the composition for targeted mutagenesis comprises a cytidine deaminase. In one embodiment, the cytidine deaminase is selected from the group comprising AID APOBEC, and TadA.

In an embodiment, the composition for targeted mutagenesis further comprises a uracil DNA glycosylase (UGI). In one embodiment, the UGI is linked to or otherwise capable of associating with the cytidine deaminase.

In one embodiment, the composition for targeted mutagenesis comprises an adenosine deaminase. In one embodiment, the adenosine deaminase is selected from the group comprising TadA, ADAR, and ADAT.

In one embodiment, the present disclosure provides a vector system comprising one or more polynucleotides encoding the programmable nickase, helicase, and deaminase, of any of the various embodiments of the composition for targeted mutagenesis.

In one embodiment, the present disclosure provides a delivery system comprising any of the various embodiments of the composition for targeted mutagenesis or any of the various embodiments of the vector system.

In one embodiment, the present disclosure provides a modified cell comprising any of the various embodiments of the composition for targeted mutagenesis, any of the various embodiments of the vector system, or any of the various embodiments of the delivery system.

In one embodiment, the present disclosure provides an animal model comprising one or more of the modified cell.

In one embodiment, the present disclosure provides a cell population comprising one or more of the modified cell.

In one embodiment, the present disclosure provides a kit comprising any of the various embodiments of the composition for targeted mutagenesis or any of the various embodiments of the vector system, and a pharmaceutically acceptable carrier.

In one embodiment, the present disclosure provides a method of targeted mutagenesis comprising delivering to a cell or population of cells any of the various embodiments of the composition for targeted mutagenesis, any of the various embodiments of the vector system, or any of the various embodiments of the delivery system, and a pharmaceutically acceptable carrier.

In one embodiment a method of targeted continuous mutagenesis comprises delivering the targeted mutagenesis compositions disclosed herein to a population of cells, wherein the one or more programmable nickases are configured to introduce a nick site(s) at one at one or more genomic regions to be diversified by continuous mutagenesis and wherein the helicase unwinds dsDNA starting at the nick site and the deaminase introduces point mutations via base edits in DNA unwound by the helicase. In an embodiment, the helicase unwinds a portion of dsDNA between approximately 1000 bp-5000 bp from the nick site, and multiple point mutations are made within the portion of unwound dsDNA. In an embodiment, the method further comprises sequencing DNA isolated from the cell or cell population to identify mutations introduced in the one or more genomic regions. In one embodiment, the one or more genomic regions to be diversified comprise one or more exons of a protein, and the method further comprises functionally screening the diversified proteins to select for a change in one or more functions. In one embodiment, the one or more functions comprise enhanced stability, increased catalytic efficiency, altered substrate specificity, improved substrate binding affinity, new enzymatic activity, or a combination thereof. In one embodiment, one or more genomic regions to be diversified encode a functional polynucleotide, and the method further comprises functionally screening the functional polynucleotide to select for a change in one or more functions. In one embodiment, the functional polynucleotide is a ribozyme, an aptamer, a guide RNA or Omega RNA. In one embodiment, the functional polynucleotide is a ribozyme, an aptamer, a guide RNA or Omega RNA, and the one or more functions are increased catalytic efficiency, new catalytic activity, altered substrate specificity, improved substrate binding affinity, or a combination thereof.

In one embodiment, a method for identifying mutations conferring resistance to therapeutic agents comprises diversifying one or more target regions by delivering to a sample cell population the targeted mutagenesis compositions disclosed herein, selecting for resistance mutations by exposing the sample cell population to one or more therapeutic agents to be screened and isolating DNA from surviving cells and identifying one or more resistance mutations by sequencing. In one embodiment, the method may further comprise validating the one or more resistance mutations by introducing the one or more resistance mutations into a wild type cell; and selecting for enriched allele frequencies of the one or more resistance mutations after exposure to the one or more therapeutic molecules to define a final set of one or more resistance mutations.

In one embodiment, a method for identifying mutations associated with alternative splicing events comprises introducing into a sample cell population a splicing reporter configured to produce a detectable signal in the presence of an alternative splicing event, diversifying one or more target regions by introducing into the sample cell population the targeted mutagenesis compositions disclosed herein, selecting cells having alternative splicing event(s) based on expression of the detectable signal from the splicing reporter; isolating DNA from cells having alternative splicing events; and sequencing the one or target regions to identify a set of mutations associated with alternative splicing events. In an embodiment, the splicing reporter comprises a portion of an endogenous intron and downstream exon fused to a constant upstream exon and a downstream fluorescent protein reporter such that correct splicing results in a frameshift in an opening reading of the fluorescent protein reporter suppressing fluorescence, while an incorrect splicing event permits expression of the fluorescent protein reporter. In one embodiment, the method may further comprise validating the one or more mutations by introducing the one or more mutations into a wild type cell population; selecting for cells enriched in GFP expression, and sequencing DNA from cells enriched in GFP expression to identify the one or more mutations associated with incorrect splicing events to define a validated set of mutations associated with incorrect splicing events.

In an embodiment, a method for identifying functional variants within non-coding gene regulatory elements may comprise diversifying one or more non-coding gene regulatory elements by delivering to a sample cell population the targeted mutagenesis compositions disclosed herein, inducing expression of one or more genes regulated by the one or more non-coding gene regulatory elements, selecting cells from the sample cell population exhibiting increased expression of the one or more genes, and sequencing DNA from the cells exhibiting increased expression of the one or more genes to identify a set of candidate mutations associated with functional variants within non-coding gene regulatory elements. In one embodiment, the method comprises further validating the one or more functional variants by introducing the set of candidate mutations into a population of wild-type cells, selecting for cells enriched in expression of the one or more genes, sequencing DNA from cells enriched in expression of the one or more genes to define a validated set of functional variants, and sequencing DNA from the cells exhibiting increased expression of the one or more genes to identify a set of candidate mutations associated with functional variants within non-coding gene regulatory elements.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows profiling of base editing of endogenous MEK1 locus by helicase-deaminase fusion proteins in the presence of nCas9. The gray vertical line represents the single guide RNA (sgRNA) nick site of the endogenous MEK1 gene.

FIG. 2 shows long-range profiling of base editing of endogenous MEK1 locus by helicase-deaminase fusion proteins in the presence of nCas9. The gray vertical line represents the single guide RNA (sgRNA) nick site of the endogenous MEK 1 gene.

FIG. 3 shows profiling of base editing of four different endogenous loci by helicase-deaminase fusion proteins in the presence of nCas9.

FIG. 4A-4G show an overview of the helicase-assisted continuous editing (HACE) system. FIG. 4A shows a schematic of a HACE editor (HE), which is a fusion protein of helicase and base-editing enzymes. A CRISPR-guided nickase (Cas9 nickase) binds with a sgRNA to generate a single-stranded DNA nick at the target genomic DNA target. FIG. 4B shows a schematic model for HACE system. A CRISPR-guided nickase targets a specific genomic position to create a nick site. A HACE editor (HE) loads to this site. As the helicase translocates along the DNA, the base editor introduces random point mutations (shown as circles). FIG. 4C shows a schematic of HACE experimental workflow, involving co-transfection of a HE plasmid (PcrA helicase variant (PcrA M6) fused with a hyperactive mutant of activation-induced cytidine deaminase (AID*Δ) and uracil DNA glycosylase inhibitor (UGI)), a nCas9 (D10A) plasmid, and sgRNA plasmid(s). Editing efficiency is assessed 72 hours post-transfection following genomic DNA extraction and amplicon sequencing. FIG. 4D shows the mutation rate per base across a ˜1-kb target region in the presence (left) and absence (right) of nCas9. The vertical dashed line shows the nick site. Data are mean of technical replicates (n=3)±s.d. FIG. 4E shows the mutation rate at the target loci (HEK3). The average mutation rate is calculated for both C>T and G>A modes for the region downstream of the nick site, excluding the sgRNA spacer region. Data are mean of technical replicates (n=3)±s.d. Significance is determined via unpaired two-tailed t-test between +/−nCas9 samples. ***P<0.001. FIG. 4F shows the mutation rate across genomic targets using loci-specific sgRNAs. The mutation rate is a sum of both C>T and G>A modes for the region downstream of the nick site, excluding the sgRNA spacer region. Data are mean of technical replicates (n=3)±s.d. FIG. 4G shows the average mutation rate for multiplex sgRNA targeting. Two sets of sgRNAs (three sgRNA per set, see Table 4) are independently co-transfected with other HACE components. The mutation rate at each sgRNA target loci is depicted in the heatmap.

FIG. 5A-5G show the HACE system is modular and flexible. FIG. 5A shows the modular components of the HACE system. Each component can be independently substituted to control for editing efficiency, mode, and range. FIG. 5B shows the mutation rate at the HEK3, TNF, and IL6 loci for HEs with different helicase variants and the nCas9 (D10A) or nCas9 (H840A) nickase variant compared with (−)nCas9 condition. Significance is determined via unpaired two-tailed t-test between control and nCas9 samples with multiple-testing correction. n.s.: not significant. *P<0.05. **P<0.01. ***P<0.001. ****P<0.0001. FIG. 5C shows the mutation rate per base across a ˜1-kb target region for HEs with different helicase variants and the nCas9 (D10A) nickase variant. The vertical dashed line shows the nick site. Data are mean of technical replicates (n=3)±s.d. FIG. 5D shows the mutation rate per base across a ˜1-kb target region for HEs with different helicase variants and the nCas9 (H840A) nickase variant. The vertical dashed line shows the nick site. Data are mean of technical replicates (n=3)±s.d. FIG. 5E shows the average G>A mutation rate at the CD209 loci for HEs with different deaminase variants fused to the PcrA M6 helicase. Significance is determined via unpaired two-tailed t-test between AID and rAPOBEC1 groups. n.s.: not significant. FIG. 5F shows the average T>C mutation rate at the CD209 loci for HEs with different deaminase variants fused to the PcrA-M6 helicase. Significance is determined via unpaired two-tailed t-test between AID and TadA groups. ***P<0.001. FIG. 5G shows the mutation rate at the HEK3 loci for HEs with different variants with and without UGI fusion. All data are mean of technical replicates (n=3)±s.d.

FIG. 6A-6F show how HACE enables the identification of MEK1 inhibitor-resistance mutations in the endogenous genome. FIG. 6A shows a workflow of HACE MEK 1 inhibitor resistance screen. A375 cells are transfected with HACE and diversified for 3 days. The genomic diversified cells are selected for 20 days. Genomes of resistant clones are harvested and sequenced by amplicon sequencing. FIG. 6B shows the location of sgRNAs for HACE screen. Exons 2, 3, and 6 (highlighted in gray) are targeted for HACE diversification. Each exon-specific sgRNA (highlighted bar) is placed ˜100 bp upstream of the target exon. FIG. 6C shows fold enrichment of MEK1 cDNA sequence in trametinib-treated (left) and selumetinib-treated (right) samples. FIG. 6D shows the enrichment of mutations installed via base editing targeting G128D (sg383) and E203K (sg607-1/2) post trametinib or selumetinib treatment. Samples are sequenced 14 days post-selection by amplicon sequencing. Significance is determined via an unpaired two-tailed t-test between control and drug-selected samples. *P<0.05. **P<0.01. ***P<0.001. ****P<0.0001. FIG. 6E shows MAPK-ERK signaling activity as measured by luciferase SRE reporter activity for G128D, G202E, and E203K mutants (mean±s.e.m., n=3 independent experiments). FIG. 6F shows the structure of MEK1 in complex with trametinib (PDB: 7JUR).

FIG. 7A-7H show identification of variants in SF3B1 that result in alternative 3′ branch point usage using HACE. FIG. 7A shows the structure of SF3B1 (left). HEAT repeats (HD) 4-8 are highlighted in dark gray (PDB: 6EN4). Differential splicing patterns can result from mutations in SF3B1 (right). FIG. 7B shows a schematic of the splicing reporter construct used for testing SF3B1-dependent splicing pattern. The plasmid reporter consists of a constitutively expressed mCherry, and a minigene splicing GFP reporter VCP exon 10 fused with DLST exon 6 with a downstream GFP. Correct splicing will not generate GFP expression, while SF3B1-dependent altered splicing will lead to GFP expression. FIG. 7C shows a histogram of GFP signal measured by flow cytometry between isogenic K562 SF3B1WT and SF3B1K700E cells. Cells were gated for mCherry expression. FIG. 7D shows a schematic of SF3B1 mutagenesis screen using HACE HACE components and splicing reporter plasmids were co-transfected in HEK293FT cells. Mutagenesis was allowed to occur for 72 h, and then cells were sorted for GFP expression. The editing rate for each sorted group was assessed following genomic DNA extraction and amplicon sequencing. FIG. 7E shows fold enrichment of individual bases in the SF3B1 cDNA sequence after selection across two biological replicates. Validated mutations are highlighted in dark gray. FIG. 7F shows normalized reporter activity fold change in mutations installed via base editing (mean±s.d., n=3). Significance is determined via an unpaired two-tailed t-test between control and edited samples. ***P<0.001 for all comparisons. The sgRNA sequences and their target bases are listed in Table 14. FIG. 7G shows normalized reporter activity fold change in mutations installed via prime editing (mean±s.d., n=3). Significance is determined via an unpaired two-tailed t-test between control and edited samples. **P<0.01 for all comparisons. The epegRNA sequences and their target bases are listed in Table 15. FIG. 7H shows the structure of SF3B1 in complex with pre-mRNA. Validated mutations are shown are labeled and annotated. The structure was an overlay of PDB structures 6AHD and 5IFE.

FIG. 8A-8H show single-base tuning of cis-regulatory elements via HACE identifies transcriptional regulation of CD69 by RUNX1/2. FIG. 8A shows a schematic of experimental workflow. The CD69 enhancer region in K562 cells was identified using ATAC-seq data and targeted via HACE sgRNAs. HACE+ K562 cells were diversified for 6 days, then stimulated with PMA/ionomycin to induce CD69 expression. Cells are sorted into CD69high and CD69low populations, and the editing rate was profiled using amplicon sequencing. FIG. 8B shows per base enrichment of C>T or G>A edits in CD69high cells relative to CD69low cells. The top most enriched C>T (dark gray and annotated and G>A (medium gray and annotated) variants in the CD69low population are annotated. Each data point represents mean±s.e.m. (n=2). FIG. 8C shows fold enrichment of individual bases in the CD69 enhancer region across 2 biological replicates. Validated bases are highlighted in dark gray and annotated (C>T) or medium gray and annotated (G>A). FIG. 8D shows a sequence of chr12:9764990 9765029. RUNX motif boxed. A sgRNA (sg4995) with NG-PAM was used to target multiple cytosines in the RUNX motif. (SEQ ID NO: 158). FIG. 8E shows a bar plot depicting the proportion of CD69high cells in SpG-CBE-sgCtrl (gray) and SpG-CBE-sg4995 (light gray) after stimulation on day 4 post-transfection. Significance is determined via unpaired two-tailed t-test between groups (***P<0.001). Data are from 3 independent experiments each with 3-4 technical replicates, mean±s.e.m. FIG. 8F shows frequency of different incurred base edit combinations in sg4995-transfected K562 cells in CD69low and CD69high populations. FIG. 8G shows the pegRNA templates for single base dissection around chr12: 9764992-9764999. The hypothesized changes in phenotype are annotated. FIG. 8H shows the proportion of CD69high post-stimulation for cells edited with different pegRNAs on day 4 post transfection. Significance is determined via unpaired two-tailed t-test between WT and edited groups. **P<0.01. ***P<0.001. ****P<0.0001.

FIG. 9A-9F show characterization of the HACE system. FIG. 9A shows a schematic of direction of helicase translocation relative to position of sgRNA. Vertical dashed line represents the location of the nick. The non-target strand (DNA strand that does not bind sgRNA) is depicted in light gray. The helicase translocates in the 3′ to 5′ direction relative to the non-target strand. FIG. 9B shows the average mutation rate across diverse base transition and transversion modes for the region downstream of the nick site. FIG. 9C shows average G>A mutation rates before and after sgRNA spacer. FIG. 9D shows a G>A mutation rate for two sets of three sgRNAs each. Significance is determined via paired two-tailed t-test between the presence and absence of sgRNAs. *P<0.05. **P<0.01. FIG. 9E shows a G>A mutation rate over the course of 96 h with transfected HE, nCas9, and sgRNA. (FIG. 9F) Mean mutations per contiguous read over time points. All data are mean of technical replicates (n=3)±s.d.

FIG. 10A-10E show HACE has long range and activity across diverse helicases and deaminases. FIG. 10A shows a mutation rate at the HEK3 loci for HEs with different helicase variants and the nCas9 (D10A) nickase variant. Data are mean of technical replicates (n=3)±s.d. FIG. 10B shows a mutation rate per base across a ˜1-kb target region at the TNF loci for HEs with different helicase variants and either the nCas9 (D10A) or nCas9 (H840A) nickase variant. The vertical dashed line shows the nick site. Data are mean of technical replicates (n=3)±s.d. FIG. 10C shows a mutation rate per base across a ˜1-kb target region at the IL6 loci for HEs with different helicase variants and either the nCas9 (D10A) or nCas9 (H840A) nickase variant. The vertical dashed line shows the nick site. Data are mean of technical replicates (n=3)±s.d. FIG. 10D shows the local mutation rate per every 100 bp window across a ˜1-kb region from the nick site for HEs with different helicase variants and either the nCas9 (D10A) or nCas9 (H840A) nickase variants. The local mutation rate is the average across 3 target loci. FIG. 10E shows the average G>A and T>C mutation rate at 5 different genomic loci for HEs with different deaminase variants fused to the PcrA M6 helicase.

FIG. 11A-11B show evaluation of HACE toxicity and off target editing. FIG. 11A shows cell viability of HEK293FT cells after transfection of HEs with different helicase variants or AID alone. All experiments were transfected with nCas9 (D10A) and a sgRNA targeting the MAP2K1 loci. Data are mean of technical replicates (n=3)±s.d. P-values for conditions with significant difference between control and treated groups (unpaired two-tailed t-test) are annotated above the bars. *P<0.05. ***P<0.001. FIG. 11B shows an analysis of exome-wide off-target editing. Scatter plots show the average C>T mutation rate for 100 kb genomic bins in cells transfected with AID alone or HE constructs with different helicases compared with control cells transfected with nCas9 (D10A) only. Sites are colored by FDR-adjusted P value (grayscale bar, right). Experiments were generated from two independent replicates.

FIG. 12 shows identification and validation of mutations leading to MEK1-inhibitor resistance. Scatter plot shows the mutant vs. reference allele frequency for A375 cells selected with either trametinib (left) or selumetinib (left) compared to control cells. Sites are shaded by Bonferroni-corrected P value (grayscale bar, right). Significant CDS base positions are annotated.

FIG. 13A-13G shows SF3B1 minigene reporter and validation of SF3B1 mutations via base and prime editing. FIG. 13A shows a histogram of GFP signal measured by flow cytometry between isogenic K562 SF3B1WT and SF3B1K700E cells for two different minigene reporter constructs. Cells were gated for mCherry expression. FIG. 13B shows RNA base pileup for 2 minigene reporter constructs nucleofected into isogenic K562 SF3B1WT and SF3B1K700E cells. The location of intron-exon junctions is annotated in black. The sequence that is retained by alternative 3′ss is annotated in black. FIG. 13C shows fold enrichment of SF3B1 cDNA sequence in GFP+ vs GFP− samples. The mutations with >10-fold base enrichment are shaded by whether they have been observed >=3 times in clinical samples. FIG. 13D shows representative images of minigene GFP reporter expression in control cells vs cells with candidate mutations introduced by base editing (sg1668, Y623C). FIG. 13E shows the editing rate in mutations installed via base editing. Data are mean of technical replicates (n=3)±s.d. FIG. 13F shows the editing rate in mutations installed via prime editing. Data are mean of technical replicates (n=3)±s.d. FIG. 13G shows correlation of editing rate to normalized fold-change for minigene reporter (p=0.880). Data are mean of technical replicates (n=3)±s.d.

FIG. 14A-14C show HACE mutagenesis and validation on the CD69 enhancer region. FIG. 14A shows a per base mutation rate across the core region of CD69 enhancer for CD69low and CD69high sorted populations. G>A and C>T transitions are colored dark gray and medium gray, respectively. Light gray dots represent the editing rate from control groups. Each data point represents mean±s.e.m. (n=3). FIG. 14B shows a sequence of the sg4948 target site, with the GATA motif boxed (top) (SEQ ID NO: 159). The proportion of CD69high cells after stimulation on day 7 post-transfection in base-edited cells using sg4948 is compared to control cells as quantified by flow cytometry (bottom). FIG. 14C shows a sequence of the sg4879 target site, with the GATA motif boxed (top) (SEQ ID NO: 160). The proportion of CD69high cells after stimulation on day 7 post-transfection in base-edited cells using sg4879 is compared to control cells as quantified by flow cytometry (bottom). Significance is determined via unpaired two-tailed t-test between control and edited groups. **P<0.01. ***P<0.001.

FIG. 15A-15H show RUNX1/2 regulates CD69 expression via the CD69 enhancer. FIG. 15A shows flow cytometry and a bar plot depicts the proportion of CD69high cells after stimulation in control, RUNX1-overexpression (OE), and RUNX2-OE groups three days post-transfection. Data are from 2 independent experiments, each with 3 technical replicates, mean±SEM. FIG. 15B shows a flow cytometry and a bar plot depicts the proportion of CD69high cells after stimulation in control, RUNX1-shRNA, and RUNX2-shRNA groups three days post-transfection. Data are from 2 independent experiments, each with 3 technical replicates, mean±s.e.m. FIG. 15C shows a proportion of CD69high post-stimulation for cells targeted with control sgRNA (sgCtrl) or sg4995 with SpG-CBE base editor 4 days post-transfection. FIG. 15D shows top alleles with different combinations of C>T mutations in cells targeted with base editing and sg4995 as quantified by amplicon sequencing in both CD69low and CD69high populations (SEQ ID NOS: 158, 161-168). FIG. 15E shows a ratio of CD69high to CD69low for sequencing read proportions for different base edit combinations as depicted in FIG. 15D. FIG. 15F shows representative flow cytometry plots depicting the proportion of CD69high after stimulation in cell populations targeted with different epegRNAs using prime editing. FIG. 15G shows a frequency of perfect homologous recombination rate (% HDR) in cell populations targeted with different epegRNAs using prime editing. FIG. 15H show a ratio of % HDR between CD69high and CD69low for editing quantified in FIG. 15G. For all comparisons, significance is determined via an unpaired two-tailed t-test between control and edited groups. *P<0.05. **P<0.01. ***P<0.001.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosure. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the disclosure. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

The embodiments disclosed herein provide compositions and methods for performing continuous mutagenesis on endogenous loci in their native chromatin context. The embodiments disclosed herein provide several advantageous properties, including (1) a long mutagenesis range (>200 bp); (2) the capacity to incur multiple, potentially interacting mutations across a region of interest; (3) a continuous and tunable mutation rate for sampling variant space and exploring fitness landscape changes; and (4) a generalizable technical framework to target genomic loci of interest individually and in combination.

In one aspect, the compositions comprise a programmable nickase configured to introduce a single-strand nick in dsDNA at one or more targeted nick sites; a helicase configured to unwind a portion of the dsDNA at the one or more targeted nick sites; and a deaminase configured to introduce one or more base edits within the portion of unwound dsDNA. The programmable nickase, which can be programmed to target a specific site on the locus of interest, creates a single-strand break at the target site. This enables the helicase to begin unwinding the dsDNA at the target site, displacing the cleaved single strand, and establishing the beginning of the editing window (i.e., the portion of the locus of interest to be edited by the system). As the helicase unwinds the dsDNA, the deaminase begins introducing base edits into the displaced single strand along the editing window propagated by the helicase. These components can be modular, allowing for the use of helicases exhibiting varying degrees of processivity (i.e., the average number of base pairs unwound by the helicase in a single binding event) in combination with different types of deaminases (e.g., cytidine deaminases, adenosine deaminases). This modularity provides for a composition capable of performing targeted continuous mutagenesis for applications including directed evolution (e.g., engineering biomolecular function) and probing the function of single nucleotide polymorphisms across varying genomic ranges (e.g., within a specific exon or an entire locus).

The present disclosure further provides vector systems comprising one or more polynucleotides encoding the components of the compositions, as well as delivery systems comprising the compositions and vector systems. The present disclosure also provides modified cells, cell populations, animal models, and kits comprising the compositions.

### Compositions for Targeted and Continuous Mutagenesis

The present disclosure provides compositions and systems for targeted mutagenesis, comprising a programmable nickase configured to introduce a single-strand nick in double-stranded DNA (dsDNA) at one or more targeted nick sites; a helicase configured to unwind a portion of the dsDNA at the one or more targeted nick sites; and a deaminase configured to introduce one or more base edits within the portion of unwound dsDNA.

The present disclosure introduces helicase assisted continuous editing (HACE), which combines long range editing of entire loci with the advantages in sequence programmability inherent to programmable gene editing tools. HACE utilizes a programmable nickase to direct the loading of a helicase and deaminase for targeted hypermutation of the downstream genomic sequence. In one embodiment, the helicase and deaminase are linked together using a polypeptide or chemical linker, or a fusion protein. Example methods for generating a combined helicase-deaminase are disclosed herein. In one embodiment, the helicase and deaminase may be further linked to or fused with the programmable nickase.

### Programmable Nickases

In example embodiments, the compositions and systems herein comprise one or more programmable nickases. A nickase is a nuclease that cuts only a single strand of a double-stranded target polynucleotide such as dsDNA. The nickase may be a naturally occurring nickase or may be obtained by engineering of a double-stranded nuclease, for example by mutating at least one nuclease domain, such that it only cuts a single strand of a target polynucleotide. Programmable nucleases which may be engineered to function as nickases include, but are not limited to, TALENs, Zn Fingers, meganucleases, Cas nucleases, and OMEGA nucleases.

### CRISPR-Cas Nickases

The compositions and systems herein may comprise a programmable nickase comprising one or more components of a CRISPR-Cas system. The one or more components of the CRISPR-Cas system may comprise one or more Cas proteins (used interchangeably herein with “CRISPR protein,” “CRISPR enzyme,” “CRISPR-Cas protein,” “CRISPR-Cas enzyme,” “Cas,” “Cas effector,” “Cas effector protein,” “CRISPR effector,” or “CRISPR effector protein”), a fragment thereof, or a mutated form thereof; and one or more guide molecules capable of forming a complex with the Cas protein. The one or more Cas proteins may be a Cas nickase (nCas, used interchangeably herein with “nicking Cas”), which introduces a single-strand nick in double-stranded (dsDNA) at one or more targeted nick sites. In some examples, the nCas comprises one or more Class 2 (e.g., Type II and Type V) CRISPR-Cas proteins.

Example Type II CRISPR-Cas nickases are known in the art (Ran et al., Genome engineering using the CRISPR-Cas9 system, Nature Protocols 8, 2281-2308 (2013) (doi: 10.1038/nprot.2013.143); Xue et al., CRISPR-mediated direct mutation of cancer genes in the mouse liver, Nature 514, 380-384 (2014) (doi: 10.1038/nature13589); Yamano et al., Crystal Structure of Cpf1 in Complex with Guide RNA and Target DNA, Cell 165, 949-962 (2016) (doi: 10.1016/j.cell.2016.04.003)). Likewise, Type V CRISPR-Cas nickases are known in the art (Zetsche et al., Cpf1 is a single RNA-guided endonuclease of a class 2 CRISPR-Cas system Cell 163, 759-771 (2015) (doi: 10.1016/j.cell.2015.09.038); Yamano et al., 2016; Kim et al., Highly precise genome editing using enhanced CRISPR-Cas12a nickase module, BioRxiv, 2022 (doi: 10.1101/2022.08.27.505535)).

In general, CRISPR-Cas nickases may be generated by mutating one of the catalytic domains. For example, the Type II CRISPR-Cas effector protein from Streptococcus pyogenes may be mutated in the RuvC domain to generate a Cas9 nickase (Yamano et al., 2016). Similarly, Acidaminococcus Type V, Cas12a CRISPR-Cas nickases may be generated by inactivating the Nuc domain (Xue et al., 2014; Yamano et al., 2016). Accordingly, nickases suitable for use in the present disclosure may also be obtained by similar modification to one or more nuclease domains.

In the context of CRISPR-Cas nickases, the site of the single-stranded nick at one or more targeted nick sites is determined by at least two elements, a protospacer adjacent motif (PAM) sequence and a guide molecule.

### Protospacer Adjacent Motif (PAM)

The PAM is a short DNA sequence, usually 2-6 base pairs in length, adjacent to the region in a target polynucleotide targeted for cleavage by the CRISPR-Cas system. The PAM is generally found 3-4 nucleotides from the nick site. Different Cas proteins may recognize different PAM sequences. For example, the Cas9 from Streptococcus pyogenes recognizes a 5′-NGG-3′ PAM, the Cas9 from Staphylococcus aureus Cas9 recognizes a 5′-NNGRR(N)-3′ PAM, and Cas12a generally recognizes a 5′-TTTV-3′, where V is a A, C, or G. It is also possible to engineer Cas proteins to recognize different PAMs. See e.g. Kleinstiver et al., Engineered CRISPR-Cas9 nucleases with altered PAM specificities, Nature 523, 481-485 (2015) (doi: 10.1038/nature14592); Gao et al., Engineered Cpf1 variants with altered PAM specificities increase genome targeting range, Nature Biotechnology 35, 789-792 (2017) (doi: 10.1038/nbt.3900); Ma et al., Engineer chimeric Cas9 to expand PAM recognition based on evolutionary information, Nature Communications 10, Article number: 560 (2019) (doi: 10.1038/s41467-019-08395-8); Toth et al., Improved LbCas12a variants with altered PAM specificities further broaden the genome targeting range of Cas12a nucleases, Nucleic Acids Research 48, 3722-3733 (2020) (doi: 10.1093/nar/gkaa110). Accordingly, selection of the appropriate CRISPR-Cas system may be dependent on the availability of a PAM near the intended one or more targeted nick sites.

The PAM or PAM-like motif (used interchangeably herein with “protospacer flanking site,” “protospacer flanking sequence,” and “PFS”) directs binding of the Cas effector protein complex as disclosed herein to the one or more targeted nick sites of interest. In an embodiment, the PAM may be a 5′ PAM (i.e., located upstream of the 5′ end of the protospacer). In other embodiments, the PAM may be a 3′ PAM (i.e., located downstream of the 5′ end of the protospacer). In a preferred embodiment, the Cas effector protein may recognize a 3′ PAM. In an embodiment, the Cas effector protein may recognize a 3′ PAM which is 5′H, wherein His A, C or U.

### Guide Molecules

The terms “guide molecule,” “guide RNA,” and “guide polynucleotide” refer to polynucleotides capable of guiding a Cas or nCas to a target genomic locus and are used interchangeably as in foregoing cited documents such as International Patent Publication No. WO 2014/093622 (PCT/US2013/074667). In general, a guide molecule is any polynucleotide sequence having sufficient complementarity with a target polynucleotide sequence to hybridize with the target sequence or target nick site and direct sequence-specific binding of a CRISPR complex to the target sequence or target nick site. The guide molecule may comprise any type of polynucleotide. In some example embodiments, the guide molecule comprises an RNA sequence, or guide RNA (gRNA).

In an embodiment, the guide molecule comprises a guide sequence and a scaffold. When the guide sequence and scaffold are part of the same single molecule, the molecule may be referred to as a single guide molecule or single guide RNA (sgRNA). As used herein, the term “guide sequence” and “spacer” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In an embodiment, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide molecule may be selected to target any target nucleic acid sequence. The target sequence may be any DNA or RNA sequence. In an embodiment, the target sequence may be double-stranded DNA (dsDNA) or single-stranded DNA (ssDNA). In an embodiment, the target sequence may be chromosomal DNA. In an embodiment, the target sequence may be plasmid DNA, circularized DNA, or linear DNA. In an embodiment, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA).

In an embodiment, a guide molecule, guide RNA, or crRNA may comprise, consist essentially of, or consist of a direct repeat (DR) sequence and a guide sequence or spacer sequence. In an embodiment, the guide RNA or crRNA may comprise, consist essentially of, or consist of a direct repeat sequence fused or linked to a guide sequence or spacer sequence. In an embodiment, the direct repeat sequence may be located upstream (i.e., 5′) from the guide sequence or spacer sequence. In other embodiments, the direct repeat sequence may be located downstream (i.e., 3′) from the guide sequence or spacer sequence.

In an embodiment, the crRNA comprises a stem loop, preferably a single stem loop. In an embodiment, the direct repeat sequence forms a stem loop, preferably a single stem loop.

In an embodiment, the spacer length of the guide RNA is from 15 to 35 nt. In an embodiment, the spacer length of the guide RNA is at least 15 nucleotides. In an embodiment, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In an embodiment, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In an embodiment, the tracr sequence is about or more than about 5, 6, 7, 8, 9, 10, 11, 12, 13, 14, 15, 16, 17, 18, 19, 20, 25, 30, 40, 50, or more nucleotides in length. In an embodiment, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In an embodiment, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In an embodiment, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In an embodiment, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 98.5% or 98% or 97.5% or 97% or 96.5% or 96% or 95.5% or 95% or 94.5% complementarity between the sequence and the guide.

In an embodiment according to the disclosure, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this disclosure. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333], which is incorporated herein by reference. Additional guide sequence modifications are described in detail below.

In an embodiment, guides of the disclosure comprise non-naturally occurring nucleic acids and/or non-naturally occurring nucleotides and/or nucleotide analogs, and/or chemical modifications. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the disclosure, a guide nucleic acid comprises ribonucleotides and non-ribonucleotides. In one such embodiment, a guide comprises one or more ribonucleotides and one or more deoxyribonucleotides. In an embodiment of the disclosure, the guide comprises one or more non-naturally occurring nucleotide or nucleotide analog such as a nucleotide with phosphorothioate linkage, boranophosphate linkage, a locked nucleic acid (LNA) nucleotide comprising a methylene bridge between the 2′ and 4′ carbons of the ribose ring, or bridged nucleic acids (BNA). Other examples of modified nucleotides include 2′-O-methyl analogs, 2′-deoxy analogs, 2-thiouridine analogs, N6-methyladenosine analogs, or 2′-fluoro analogs. Further examples of modified bases include, but are not limited to, 2-aminopurine, 5-bromo-uridine, pseudouridine (Ψ), N¹-methylpseudouridine (me¹Ψ′), 5-methoxyuridine (5moU), inosine, 7-methylguanosine. Examples of guide RNA chemical modifications include, without limitation, incorporation of 2′-O-methyl (M), 2′-O-methyl-3′-phosphorothioate (MS), phosphorothioate (PS), S-constrained ethyl (cEt), or 2′-O-methyl-3′-thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33 (9): 985-9, doi: 10.1038/nbt.3290, published online 29 Jun. 2015; Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33 (9): 985-989; Li et al., Nature Biomedical Engineering, 2017, 1, 0066 DOI: 10.1038/s41551-017-0066). In an embodiment, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In an embodiment, a guide comprises ribonucleotides in a region that binds to a target DNA and one or more deoxyribonucleotides and/or nucleotide analogs in a region that binds to Cas9, Cpf1, or C2c1. In an embodiment of the disclosure, deoxyribonucleotides and/or nucleotide analogs are incorporated in engineered guide structures, such as, without limitation, 5′ and/or 3′ end, stem-loop regions, and the seed region. In an embodiment, the modification is not in the 5′-handle of the stem-loop regions. Chemical modification in the 5′-handle of the stem-loop region of a guide may abolish its function (see Li, et al., Nature Biomedical Engineering, 2017, 1:0066). In an embodiment, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In an embodiment, 3-5 nucleotides at either the 3′ or the 5′ end of a guide is chemically modified. In an embodiment, only minor modifications are introduced in the seed region, such as 2′-F modifications. In an embodiment, 2′-F modification is introduced at the 3′ end of a guide. In an embodiment, three to five nucleotides at the 5′ and/or the 3′ end of the guide are chemically modified with 2′-O-methyl (M), 2′-O-methyl-3′-phosphorothioate (MS), S-constrained ethyl (cEt), or 2′-O-methyl-3′-thioPACE (MSP). Such modification can enhance genome editing efficiency (see Hendel et al., Nat. Biotechnol. (2015) 33 (9): 985-989). In an embodiment, all of the phosphodiester bonds of a guide are substituted with phosphorothioates (PS) for enhancing levels of gene disruption. In an embodiment, more than five nucleotides at the 5′ and/or the 3′ end of the guide are chemically modified with 2′-O-Me, 2′-F or S-constrained ethyl (cEt). Such chemically modified guides can mediate enhanced levels of gene disruption (see Ragdarm et al., 0215, PNAS, E7110-E7111). In an embodiment of the disclosure, a guide is modified to comprise a chemical moiety at its 3′ and/or 5′ end. Such moieties include, but are not limited to, amine, azide, alkyne, thio, dibenzocyclooctyne (DBCO), or Rhodamine. In an embodiment, the chemical moiety is conjugated to the guide by a linker, such as an alkyl chain. In an embodiment, the chemical moiety of the modified guide can be used to attach the guide to another molecule, such as DNA, RNA, protein, or nanoparticles. Such chemically modified guides can be used to identify or enrich cells genetically edited by a CRISPR system (see Lee et al., eLife, 2017, 6: e25312, DOI: 10.7554).

In an embodiment, the CRISPR system as provided herein can make use of a crRNA or analogous polynucleotide comprising a guide sequence, wherein the polynucleotide is an RNA, a DNA or a mixture of RNA and DNA, and/or wherein the polynucleotide comprises one or more nucleotide analogs. The sequence can comprise any structure, including but not limited to a structure of a native crRNA, such as a bulge, a hairpin, or a stem loop structure. In an embodiment, the polynucleotide comprising the guide sequence forms a duplex with a second polynucleotide sequence, which can be an RNA or a DNA sequence.

In an embodiment, use is made of chemically modified guide RNAs. Examples of guide RNA chemical modifications include, without limitation, incorporation of 2′-O-methyl (M), 2′-O-methyl 3′phosphorothioate (MS), or 2′-O-methyl 3′thioPACE (MSP) at one or more terminal nucleotides. Such chemically modified guide RNAs can comprise increased stability and increased activity as compared to unmodified guide RNAs, though on-target vs. off-target specificity is not predictable. (See, Hendel, 2015, Nat Biotechnol. 33 (9): 985-9, doi: 10.1038/nbt.3290, published online 29 Jun. 2015). Chemically modified guide RNAs further include, without limitation, RNAs with phosphorothioate linkages and locked nucleic acid (LNA) nucleotides comprising a methylene bridge between the 2′ and 4′ carbons of the ribose ring.

In an embodiment, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In an embodiment, a guide sequence is less than about 75, 50, 45, 40, 35, 30, 25, 20, 15, 12, or fewer nucleotides in length. Preferably the guide sequence is 10 to 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay. Similarly, cleavage of a target RNA may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In an embodiment, the modification to the guide is a chemical modification, an insertion, a deletion, or a split. In an embodiment, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N¹-methylpseudouridine (me¹Ψ), 5-methoxyuridine (5moU), inosine, 7-methylguanosine, 2′-O-methyl-3′-phosphorothioate (MS), S-constrained ethyl (cEt), phosphorothioate (PS), or 2′-O-methyl-3′-thioPACE (MSP). In an embodiment, the guide comprises one or more of phosphorothioate modifications. In an embodiment, at least 1, 2, 3, 4, 5, 6, 7, 8, 9, 10, 11, 12, 13, 14, 15, 16, 17, 18, 19, 20, or 25 nucleotides of the guide are chemically modified. In an embodiment, one or more nucleotides in the seed region are chemically modified. In an embodiment, one or more nucleotides in the 3′-terminus are chemically modified. In an embodiment, none of the nucleotides in the 5′-handle is chemically modified. In an embodiment, the chemical modification in the seed region is a minor modification, such as incorporation of a 2′-fluoro analog. In a specific embodiment, one nucleotide of the seed region is replaced with a 2′-fluoro analog. In an embodiment, 5 or 10 nucleotides in the 3′-terminus are chemically modified. Such chemical modifications at the 3′-terminus of the Cpf1 CrRNA improve gene cutting efficiency (see Li, et al., Nature Biomedical Engineering, 2017, 1:0066). In a specific embodiment, 5 nucleotides in the 3′-terminus are replaced with 2′-fluoro analogues. In a specific embodiment, 10 nucleotides in the 3′-terminus are replaced with 2′-fluoro analogues. In a specific embodiment, 5 nucleotides in the 3′-terminus are replaced with 2′-O-methyl (M) analogs.

In an embodiment, the loop of the 5′-handle of the guide is modified. In an embodiment, the loop of the 5′-handle of the guide is modified to have a deletion, an insertion, a split, or chemical modifications. In an embodiment, the loop comprises 3, 4, or 5 nucleotides. In an embodiment, the loop comprises the sequence of UCUU, UUUU, UAUU, or UGUU.

### Class 2 Systems

The compositions, systems, and methods described in greater detail elsewhere herein can be designed and adapted for use with Class 2 CRISPR-Cas systems. Thus, in an embodiment, the CRISPR-Cas system is a Class 2 CRISPR-Cas system. In an embodiment, the Class 2 system can be a Type II or Type V system, which are described in Makarova et al. “Evolutionary classification of CRISPR-Cas systems: a burst of class 2 and derived variants” Nature Reviews Microbiology, 18:67-81 (February 2020), incorporated herein by reference.

Type II and Type V systems differ in the domain organization of their Cas effector complexes. Type II Cas effector proteins (e.g., Cas9) contain two nuclease domains that are each responsible for the cleavage of one strand of the target DNA, with the HNH nuclease inserted inside the Ruv-C like nuclease domain sequence. The Type V Cas effector proteins (e.g., Cas12) contain only a RuvC-like nuclease domain that cleaves both strands.

In an embodiment, the Class 2 system is a Type II system. In an embodiment, the Type II CRISPR-Cas system is a II-A CRISPR-Cas system. In an embodiment, the Type II CRISPR-Cas system is a II-B CRISPR-Cas system. In an embodiment, the Type II CRISPR-Cas system is a II-C1 CRISPR-Cas system. In an embodiment, the Type II CRISPR-Cas system is a II-C2 CRISPR-Cas system. In an embodiment, the Type II system is a Cas9 system. In an embodiment, the Type II system includes a Cas9.

In an embodiment, the Class 2 system is a Type V system. In an embodiment, the Type V CRISPR-Cas system is a V-A CRISPR-Cas system. In an embodiment, the Type V CRISPR-Cas system is a V-B1 CRISPR-Cas system. In an embodiment, the Type V CRISPR-Cas system is a V-B2 CRISPR-Cas system. In an embodiment, the Type V CRISPR-Cas system is a V-C CRISPR-Cas system. In an embodiment, the Type V CRISPR-Cas system is a V-D CRISPR-Cas system. In an embodiment, the Type V CRISPR-Cas system is a V-E CRISPR-Cas system. In an embodiment, the Type V CRISPR-Cas system is a V-F1 CRISPR-Cas system. In an embodiment, the Type V CRISPR-Cas system is a V-F1 (V-U3) CRISPR-Cas system. In an embodiment, the Type V CRISPR-Cas system is a V-F2 CRISPR-Cas system. In an embodiment, the Type V CRISPR-Cas system is a V-F3 CRISPR-Cas system. In an embodiment, the Type V CRISPR-Cas system is a V-G CRISPR-Cas system. In an embodiment, the Type V CRISPR-Cas system is a V-H CRISPR-Cas system. In an embodiment, the Type V CRISPR-Cas system is a V-I CRISPR-Cas system. In an embodiment, the Type V CRISPR-Cas system is a V-K (V-U5) CRISPR-Cas system. In an embodiment, the Type V CRISPR-Cas system is a V-U1 CRISPR-Cas system. In an embodiment, the Type V CRISPR-Cas system is a V-U2 CRISPR-Cas system. In an embodiment, the Type V CRISPR-Cas system is a V-U4 CRISPR-Cas system. In an embodiment, the Type V CRISPR-Cas system includes a Cas12a (Cpf1), Cas12b (C2c1), Cas12c (C2c3), Cas12d (CasY), Cas12e (CasX), Cas14, and/or CasΦ.

### OMEGA (Obligate Mobile Element-Guided Activity) Nickases

OMEGA (Obligate Mobile Element-Guided Activity) nucleases are a class of RNA-guided nucleases encoded in a distinct family of IS200/IS605 transposons and are likely ancestors of Cas9 and Cas12 nucleases (Altae-Tran et al., The widespread IS200/IS605 transposon family encodes diverse programmable RNA-guided endonucleases. Science 374, 57-65 (2021)). These nucleases include the transposon-encoded proteins IscB (and its homologs IsrB and IshB) TnpB, and Fanzor, and use a non-coding RNA sequence (termed “OMEGA RNA” or “@RNA”) as a guide to target and cleave dsDNA. OMEGA nucleases can be reprogrammed to bind to varying target sites by using different guide RNAs specific for those sites.

OMEGA nucleases may also be mutated in one or more of their nuclease domains to generate an OMEGA nickase, which generates a single-strand nick at one or more targeted nick sites of the locus of interest. The site of the single-stranded nick at one or more targeted nick sites is determined by at least two elements, a target adjacent motif (TAM) sequence and an ΦRNA.

In an embodiment, the programmable nickase comprises an OMEGA nickase and one or more ΦRNA molecules capable of forming a complex with the OMEGA nickase and directing sequence-specific binding of the complex to the one or more targeted nick sites. In an embodiment, the OMEGA nickase may comprise an IscB nickase, an IsrB nickase, an IshB nickase, or a TnpB nickase.

### IscB Nucleases and Homologs Thereof

In an embodiment, the programmable nickase disclosed herein may comprise an OMEGA nickase from an IscB system. The IscB system comprises an IscB protein and a nucleic acid component capable of forming a complex with the IscB protein and directing the complex to a target polynucleotide or targeted nick site. The IscB systems include the homolog IsrB and IshB systems. The nucleic acid component may also be referred to herein as a hRNA or ωRNA. IscB proteins, and homologs thereof, are considerably smaller than other RNA-guided nucleases. As such, IscB proteins, and homologs thereof, represent a novel class of RNA-guided nucleases that do not suffer from the delivery size limitations of other larger single-effector, RNA-guided nucleases, such as Type II and Type V CRISPR-Cas systems. Due to their smaller size, IscB proteins, and homologs thereof, may be combined with other functional domains (e.g., nucleobase deaminases, reverse transcriptases, transposases, ligases, topoisomerases, serine, and threonine recombinases, etc.) and still be packaged in conventional delivery systems like certain adenovirus and lentivirus based viral vectors. Thus, among other improvements, the IscB systems and homologs thereof disclosed herein allow more flexible and effective strategies to manipulate and modify target polynucleotides. IscB nucleases and OMEGA systems are further described in Altae-Tran et al., The widespread IS200/605 transposon family encodes diverse programmable RNA-guided endonucleases, Science. 2021 October; 374 (6563): 57-65, which is incorporated by reference herein in its entirety.

In an embodiment, the programmable nickase may comprise an IscB nickase. IscB proteins comprise a PLMP domain, RuvC domains, and an HNH domain. In one embodiment, the IscB is an ωRNA-guided nickase. In one embodiment, the ωRNA-guided IscB nicks a DNA target. In one embodiment, the DNA target is a dsDNA, and the nick occurs on the non-target strand of the dsDNA target. In an embodiment, the IscB nicks the dsDNA in a guide and TAM specific manner.

In an embodiment, the programmable nickase may comprise an IsrB nickase. As noted above, IsrB proteins are homologs of IscB proteins. IsrB polypeptides comprise a PLMP domain and RuvC domains but do not comprise an HNH domain. The IsrB proteins may be about 200 to about 500 amino acids in length, about 250 to about 450 amino acids in length, or about 300 to about 400 amino acids in length. In one embodiment, the IsrB is an ωRNA-guided nickase. In one embodiment, the ωRNA-guided IsrB nicks a DNA target. In one embodiment, the DNA target is a dsDNA, and the nick occurs on the non-target strand of the dsDNA target. In an embodiment, the IsrB nicks the dsDNA in a guide and TAM specific manner.

In an embodiment, the programmable nickase may comprise an IshB nickase. As noted above, IshB proteins are homologs of IscB proteins. IshB proteins are generally smaller than IscB and IsrB proteins and contain only a PLMP domain and HNH domain, but no RuvC domains. The IshB proteins may be about 150 to about 235 amino acids in length, about 160 to about 220 amino acids in length, about 170 to about 200 amino acids in length, about 170 to about 190 amino acids in length, or about 175 to 185 amino acids in length. In one embodiment, the IshB is an @RNA-guided nickase. In one embodiment, the @RNA-guided IshB nicks a DNA target. In one embodiment, the DNA target is a dsDNA, and the nick occurs on the non-target strand of the dsDNA target. In an embodiment, the IshB nicks the dsDNA in a guide and TAM specific manner.

### TnpB Nucleases

In an embodiment, the programmable nickase may comprise a TnpB nickase. TnpB proteins are characterized by the presence of RuvC domains and a zinc finger domain. The TnpB proteins are between 175 and 800 amino acids in size, between 200 and 790 amino acids in size, between 200 and 780 amino acids in size, between 200 and 770 amino acids in size, between 200 and 760 amino acids in size, between 200 and 750 amino acids in size, between 200 and 740 amino acids in size, between 200 and 730 amino acids in size, between 200 and 720 amino acids in size, between 200 and 710 amino acids in size, between 200 and 700 amino acids in size, between 200 and 690 amino acids in size, between 200 and 680 amino acids in size, between 200 and 670 amino acids in size, between 200 and 660 amino acids in size, between 200 and 650 amino acids in size, between 200 and 640 amino acids in size, between 200 and 630 amino acids in size, between 200 and 620 amino acids in size, between 200 and 610 amino acids in size, between 200 and 600 amino acids in size, between 200 and 590 amino acids in size, between 200 and 580 amino acids in size, between 200 and 570 amino acids in size, between 200 and 560 amino acid, between 200 and 550 amino acids, between 200 and 540 amino acids, between 200 and 530 amino acids, between 200 and 520 amino acids, between 200 and 510 amino acids, between 200 and 500 amino acids, between 200 and 490 amino acids, between 200 and 480 amino acids, between 200 and 470 amino acids, between 200 and 460 amino acids, between 200 and 450 amino acids, between 200 and 440 amino acids, between 200 and 430 amino acids, between 200 and 420 amino acids, between 200 and 410 amino acids, between 210 and 500 amino acids, between 220 and 500 amino acids, between 230 and 500 amino acids, between 240 and 500 amino acids, between 250 and 500 amino acids, between 260 and 500 amino acids, between 270 and 500 amino acids, between 280 and 500 amino acids, between 290 and 500 amino acids, between 300 and 500 amino acids, between 250 and 470 amino acids, between 250 and 480 amino acids, between 250 and 490 amino acids, or between 250 and 600 amino acids. In one embodiment, the TnpB polypeptide is between 300 and 500 amino acids, or between 350 and 450 amino acids. In one embodiment, the TnpB is an ORNA-guided nickase. In one embodiment, the @RNA-guided TnpB nicks a DNA target. In one embodiment, the DNA target is a dsDNA, and the nick occurs on the non-target strand of the dsDNA target. In an embodiment, the TnpB nicks the dsDNA in a guide and TAM specific manner.

The TnpB proteins also encompass homologs or orthologs of TnpB proteins. The terms “ortholog” and “homolog” are well known in the art. By means of further guidance, a “homolog” of a protein as used herein is a protein of the same species which performs the same or a similar function as the protein it is a homolog of. Homologous proteins may but need not be structurally related, or are only partially structurally related. An “ortholog” of a protein as used herein is a protein of a different species which performs the same or a similar function as the protein it is an ortholog of. Orthologous proteins may but need not be structurally related or are only partially structurally related. In particular embodiments, the homolog or ortholog of a TnpB polypeptide such as referred to herein has a sequence homology or identity of at least 80%, at least 85%, at least 90%, at least 95% with a TnpB polypeptide. In further embodiments, the homolog or ortholog of a TnpB polypeptide has a sequence identity of at least 80%, at least 85%, at least 90%, or at least 95% with a wildtype TnpB polypeptide. A homolog or ortholog may be identified according to its domain structure and/or function. Sequence alignments conducted as described herein, as well as folding studies and domain predictions as taught herein can aid in the identification of a homolog or ortholog with the structural and functional characteristics identifying TnpB polypeptides, particularly those with conserved residues, including catalytic residues, and domains of TnpB polypeptides.

### Fanzor Nucleases

In one embodiment, the programmable nickase may be a Fanzor nickase. Fanzors are eukaryotic programmable RNA-guided endonucleases and also utilize an @RNA. Saito et al. “Fanzor is a eukaryotic programmable RNA-guided endonuclease” Nature 2023, 620 (7974): 660-668; Jiang et al. “Programmable RNA-guided DNA endonucleases are widespread in eukaryotes and their viruses.” Sci Adv. 2023; 9 (39); WO 2023/114872, “Reprogrammable Fanzor Polynucleotides and Uses Thereof” Jun. 22, 2023.

In an embodiment, the programmable nickase may comprise an Fanzor nickase. The Fanzor nickase may comprise one or more inactivating mutations in one nuclease domain while retaining nuclease function in a second nuclease domain. In one embodiment, the Fanzor is an ωRNA-guided nickase. In one embodiment, the ωRNA-guided Fanzor nicks a DNA target. In one embodiment, the DNA target is a dsDNA, and the nick occurs on the non-target strand of the dsDNA target. In an embodiment, the Fanzor nicks the dsDNA in a guide and TAM specific manner.

### ωRNA Molecules

The systems herein may further comprise one or more hRNA molecules, which are referred to herein interchangeably as ωRNA. The hRNA complex can comprise a guide sequence and a scaffold that interacts with the IscB protein. An hRNA molecule may form a complex with IscB protein nuclease or IscB protein, or homolog thereof, and direct the complex to bind with a target sequence. In an embodiment, the hRNA molecule is a single molecule comprising a scaffold sequence and a spacer sequence. In an embodiment, the spacer is 5′ of the scaffold sequence. In an embodiment, the hRNA molecule may further comprise a conserved nucleic acid sequence between the scaffold and spacer portions.

In an embodiment, the hRNA scaffold comprises a spacer sequence and a conserved nucleotide sequence. The hRNA scaffold typically comprises conserved regions, with the scaffold comprising 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 115, 125, 135, 145, 155, 165, 175, 185, 195, 205, 215, 225, 235, 245, 255, 265, 275, 285, 295, 305, 315, 325, 335, 345, or 355 or more nt. In an aspect, the hRNA scaffold comprises one conserved nucleotide sequence. In embodiments, the conserved nucleotide sequence is on or near a 5′ end of the scaffold. In embodiments, the scaffold may comprise a short 3-4 base pair nexus, a conserved nexus hairpin and a large multi-stem loop region that may consist of two interconnected multi-stem loops. The scaffold hRNA may further comprise a spacer, which can be re-programmed to direct site-specific binding to a target sequence of a target polynucleotide. The spacer may also be referred to herein as part of the hRNA scaffold or as gRNA and may comprise an engineered heterologous sequence.

In an embodiment, the spacer length of the hRNA is from 10 to 150 nt. In an embodiment, the spacer length of the guide RNA is at least 15 nucleotides. In an embodiment, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer. In certain example embodiments, the guide sequence is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 nt.

In an embodiment, the hRNA spacer length is from 15 to 50 nt. In an embodiment, the spacer length of the hRNA is at least 15 nucleotides. In an embodiment, the spacer length is from 15 to 50 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt, from 34 to 40 nt, e.g., 34, 35, 36, 37, 38, 39, 40, from 35 to 39, from 36 to 38 nt long, about 37 nt, or longer.

In an embodiment, the sequence of the hRNA molecule is selected to reduce the degree of secondary structure within the hRNA molecule. In an embodiment, about or less than about 75%, 50%, 40%, 30%, 25%, 20%, 15%, 10%, 5%, 1%, or fewer of the nucleotides of the nucleic acid-targeting hRNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example of a folding algorithm is the online webserver RNAfold, developed at Institute for Theoretical Chemistry at the University of Vienna, using the centroid structure prediction algorithm (see e.g., A. R. Gruber et al., 2008, Cell 106 (1): 23-24; and PA Carr and GM Church, 2009, Nature Biotechnology 27 (12): 1151-62).

As used herein, a heterologous hRNA molecule is an hRNA molecule that is not derived from the same species as the IscB protein nuclease, or comprises a portion of the molecule, e.g. spacer, that is not derived from the same species as the IscB polypeptide nuclease, e.g. IscB protein. For example, a heterologous hRNA molecule of a IscB polypeptide nuclease derived from species A comprises a polynucleotide derived from a species different from species A, or an artificial polynucleotide.

In a particular embodiment, the hRNA comprises a guide sequence linked to a conserved nucleotide sequence, wherein the conserved nucleotide sequence may comprise one or more stem loops or optimized secondary structures. In particular embodiments, the conserved nucleotide sequence has a minimum length of 16 nts and a single stem loop. In further embodiments the conserved nucleotide sequence has a length longer than 16 nts, preferably more than 17 nts, and has more than one stem loop or optimized secondary structures. In particular embodiments, the guide sequence may be linked to all or part of the natural conserved nucleotide sequence. In particular embodiments, certain aspects of the guide architecture can be modified, for example by addition, subtraction, or substitution of features, whereas certain other aspects of guide architecture are maintained. Preferred locations for engineered guide modifications, including but not limited to insertions, deletions, and substitutions include guide termini and regions of the guide that are exposed when complexed with IscB polypeptide nuclease and/or target, for example the tetraloop and/or loop2.

In an embodiment, a loop in the guide RNA is provided. This may be a stem loop or a tetra loop. The loop is preferably GAAA, but it is not limited to this sequence or indeed to being only 4 bp in length. Indeed, preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG.

In an embodiment, the hRNA forms a stem loop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In an embodiment, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the conserved nucleotide sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, sulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In an embodiment, these stem-loop forming sequences can be chemically synthesized. In an embodiment, the chemical synthesis uses automated, solid-phase oligonucleotide synthesis machines with 2′-acetoxyethyl orthoester (2′-ACE) (Scaringe et al., J. Am. Chem. Soc. (1998) 120:11820-11821; Scaringe, Methods Enzymol. (2000) 317:3-18) or 2′-thionocarbamate (2′-TC) chemistry (Dellinger et al., J. Am. Chem. Soc. (2011) 133:11540-11546; Hendel et al., Nat. Biotechnol. (2015) 33:985-989).

The repeat: anti-repeat duplex will be apparent from the secondary structure of the hRNA. It may be typically a first complementary stretch after (in 5′ to 3′ direction) the poly U tract and before the tetraloop; and a second complementary stretch after (in 5′ to 3′ direction) the tetraloop and before the poly A tract. The first complementary stretch (the “repeat”) is complementary to the second complementary stretch (the “anti-repeat”). As such, they Watson-Crick base pair to form a duplex of dsRNA when folded back on one another. As such, the anti-repeat sequence is the complementary sequence of the repeat and in terms to A-U or C-G base pairing, but also in terms of the fact that the anti-repeat is in the reverse orientation due to the tetraloop.

In an embodiment of the disclosure, modification of guide architecture comprises replacing bases in stem loop 2. For example, in an embodiment, “actt” (“acuu” in RNA) and “aagt” (“aagu” in RNA) bases in stemloop2 are replaced with “cgcc” and “gcgg”. In an embodiment, “actt” and “aagt” bases in stemloop2 are replaced with complementary GC-rich regions of 4 nucleotides. In an embodiment, the complementary GC-rich regions of 4 nucleotides are “cgcc” and “gcgg” (both in 5′ to 3′ direction). In an embodiment, the complementary GC-rich regions of 4 nucleotides are “gcgg” and “cgcc” (both in 5′ to 3′ direction). Other combinations of C and G in the complementary GC-rich regions of 4 nucleotides will be apparent including CCCC and GGGG.

In one aspect, the stemloop 2, e.g., “ACTTgtttAAGT” (SEQ ID NO: 1) can be replaced by any “XXXXgtttYYYY”, e.g., where XXXX and YYYY represent any complementary sets of nucleotides that together will base pair to each other to create a stem.

As used herein, the term “spacer” may also be referred to as a “guide sequence.” In an embodiment, the degree of complementarity of the guide sequence to a given target sequence, when optimally aligned using a suitable alignment algorithm, is about or more than 50%, 60%, 75%, 80%, 85%, 90%, 95%, 97.5%, 99%, or more. In an embodiment, the hRNA molecule comprises a guide sequence that may be designed to have at least one mismatch with the target sequence, such that an RNA duplex is formed between the sequence and the target sequence. Accordingly, the degree of complementarity is less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In an embodiment, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). The ability of a sequence (within a nucleic acid-targeting guide sequence) to direct sequence-specific binding of a nucleic acid-targeting complex to a target nucleic acid sequence may be assessed by any suitable assay. For example, the components of a hRNA system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the sequence to be tested and a control sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting hRNA may be selected to target any target nucleic acid sequence.

A hRNA sequence, and hence a nucleic acid-targeting guide, may be selected to target any target nucleic acid sequence. The target sequence may be DNA. The target sequence may be any RNA sequence. In an embodiment, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In an embodiment, the hRNA molecule comprises non-naturally occurring nucleic acids and/or non-naturally occurring nucleotides and/or nucleotide analogs, and/or chemical modifications. Preferably, these non-naturally occurring nucleic acids and non-naturally occurring nucleotides are located outside the hRNA sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the disclosure, a hRNA nucleic acid comprises ribonucleotides and non-ribonucleotides. In one such embodiment, a hRNA comprises one or more ribonucleotides and one or more deoxyribonucleotides. In an embodiment of the disclosure, the hRNA comprises one or more non-naturally occurring nucleotide or nucleotide analog such as a nucleotide with phosphorothioate linkage, locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of hRNA chemical modifications include, without limitation, incorporation of 2′-O-methyl (M), 2′-O-methyl 3′phosphorothioate (MS), S-constrained ethyl (cEt), or 2′-O-methyl 3′thioPACE (MSP) at one or more terminal nucleotides. Such chemically modified hRNAs can comprise increased stability and increased activity as compared to unmodified hRNAs, though on-target vs. off-target specificity is not predictable. (See, Hendel, 2015, Nat Biotechnol. 33 (9): 985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33 (9): 985-989; Li et al., Nature Biomedical Engineering, 2017, 1, 0066 DOI: 10.1038/s41551-017-0066). In an embodiment, the 5′ and/or 3′ end of a hRNA is modified by a variety of functional moieties including fluorescent dyes, polyethylene glycol, cholesterol, proteins, or detection tags. (See Kelly et al., 2016, J. Biotech. 233:74-83). In an embodiment, a hRNA comprises ribonucleotides in a region that binds to a target sequence and one or more deoxyribonucleotides and/or nucleotide analogs in a region that binds to the IscB polypeptide nuclease. In an embodiment, deoxyribonucleotides and/or nucleotide analogs are incorporated in engineered hRNA structures. In an embodiment, 3-5 nucleotides at either the 3′ or the 5′ end of a hRNA is chemically modified. In an embodiment, only minor modifications are introduced in the seed region, such as 2′-F modifications. In an embodiment, 2′-F modification is introduced at the 3′ end of a hRNA. In an embodiment, three to five nucleotides at the 5′ and/or the 3′ end of the hRNA are chemically modified with 2′-O-methyl (M), 2′-O-methyl 3′ phosphorothioate (MS), S-constrained ethyl (cEt), or 2′-O-methyl 3′ thioPACE (MSP). Such modification can enhance genome editing efficiency (see Hendel et al., Nat. Biotechnol. (2015) 33 (9): 985-989). In an embodiment, all of the phosphodiester bonds of a hRNA are substituted with phosphorothioates (PS) for enhancing levels of gene disruption. In an embodiment, more than five nucleotides at the 5′ and/or the 3′ end of the hRNA are chemically modified with 2′-O-Me, 2′-F or S-constrained ethyl (cEt). Such chemically modified hRNA can mediate enhanced levels of gene disruption (see Ragdarm et al., 0215, PNAS, E7110-E7111). In an embodiment of the disclosure, a hRNA is modified to comprise a chemical moiety at its 3′ and/or 5′ end. Such moieties include, but are not limited to amine, azide, alkyne, thio, dibenzocyclooctyne (DBCO), or Rhodamine. In an embodiment, the chemical moiety is conjugated to the hRNA by a linker, such as an alkyl chain. In an embodiment, the chemical moiety of the modified hRNA can be used to attach the hRNA to another molecule, such as DNA, RNA, protein, or nanoparticles. Such chemically modified hRNA can be used to identify or enrich cells genetically edited by a IscB polypeptide nuclease and related systems (see Lee et al., eLife, 2017, 6: e25312, DOI: 10.7554).

In a particular embodiment, the conserved nucleotide sequence may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In embodiments, the IscB polypeptide utilizes the hRNA scaffold comprising a polynucleotide sequence that facilitates the interaction with the IscB protein, allowing for sequence specific binding and/or targeting of the guide sequence with the target polynucleotide. Chemical synthesis of the hRNA scaffold is contemplated, using covalent linkage using various bioconjugation reactions, loops, bridges, and non-nucleotide links via modifications of sugar, internucleotide phosphodiester bonds, purine and pyrimidine residues. Sletten et al., Angew. Chem. Int. Ed. (2009) 48:6974-6998; Manoharan, M. Curr. Opin. Chem. Biol. (2004) 8:570-9; Behlke et al., Oligonucleotides (2008) 18:305-19; Watts, et al., Drug. Discov. Today (2008) 13:842-55; Shukla, et al., ChemMedChem (2010) 5:328-49; chemical synthesis using automated, solid-phase oligonucleotide synthesis machines with 2′-acetoxyethyl orthoester (2′-ACE) (Scaringe et al., J. Am. Chem. Soc. (1998) 120:11820-11821; Scaringe, Methods Enzymol. (2000) 317:3-18) or 2′-thionocarbamate (2′-TC) chemistry (Dellinger et al., J. Am. Chem. Soc. (2011) 133:11540-11546; Hendel et al., Nat. Biotechnol. (2015) 33:985-989).

In an embodiment, the scaffold and spacer may be designed as two separate molecules that can hybridize or covalently join into a single molecule. Covalent linkage can be via a linker (e.g., a non-nucleotide loop) that comprises a moiety such as spacers, attachments, bioconjugates, chromophores, reporter groups, dye labeled RNAs, and non-naturally occurring nucleotide analogues. More specifically, suitable spacers for purposes of this disclosure include, but are not limited to, polyethers (e.g., polyethylene glycols, polyalcohols, polypropylene glycol or mixtures of ethylene and propylene glycols), polyamines group (e.g., spennine, spermidine and polymeric derivatives thereof), polyesters (e.g., poly(ethyl acrylate)), polyphosphodiesters, alkylenes, and combinations thereof. Suitable attachments include any moiety that can be added to the linker to add additional properties to the linker, such as but not limited to, fluorescent labels. Suitable bioconjugates include, but are not limited to, peptides, glycosides, lipids, cholesterol, phospholipids, diacyl glycerols and dialkyl glycerols, fatty acids, hydrocarbons, enzyme substrates, steroids, biotin, digoxigenin, carbohydrates, polysaccharides. Suitable chromophores, reporter groups, and dye-labeled RNAs include, but are not limited to, fluorescent dyes such as fluorescein and rhodamine, chemiluminescent, electrochemiluminescent, and bioluminescent marker compounds. The design of example linkers conjugating two RNA components are also described in WO 2004/015075.

The linker (e.g., a non-nucleotide loop) can be of any length. In an embodiment, the linker has a length equivalent to about 0-16 nucleotides. In an embodiment, the linker has a length equivalent to about 0-8 nucleotides. In an embodiment, the linker has a length equivalent to about 0-4 nucleotides. In an embodiment, the linker has a length equivalent to about 2 nucleotides. Example linker design is also described in International Patent Application Publication No. WO 2011/008730.

### Helicases

The term “helicase” refers here to any protein, polypeptide, or one or more functional domains of a protein or polypeptide that is capable of unwinding a double stranded nucleic acid enzymatically. For example, helicases are enzymes that are found in all organisms and in all processes that involve nucleic acid such as replication, recombination, repair, transcription, translation, and RNA splicing. (Kornberg and Baker, DNA Replication, W. H. Freeman and Company (2^{nd} ed. (1992)), especially chapter 11). Within the context of the compositions described herein, the helicase unwinds the dsDNA (beginning at the nick generated at the targeted nick site by the programmable nickase), displacing a single strand of DNA and propagating an editing window within which the deaminase introduces base edits along the displaced strand. Helicases exhibiting varying processivity ranges may be used. As used herein, the term “processivity” (also used interchangeably herein with “processivity range”) refers to the average number of base pairs unwound by the helicase in a single binding event, in the absence of DNA single-stranded binding proteins, before the helicase detaches from the nucleic acid. For example, a DNA helicase having a processivity range of 100 base pairs will unwind an average of 100 base pairs of double-stranded DNA before detaching from the DNA. A helicase exhibiting a long processivity range (e.g., greater than or equal to 200 base pairs) may be used to broaden the editing window for directed evolution applications (e.g., engineering new proteins and biomolecular function). A helicase exhibiting a shorter processivity range (e.g., less than 200 base pairs) may be desirable where modifications within a narrower editing window are beneficial (e.g., analysis of single nucleotide polymorphisms within an exon).

Any helicase that translocates along DNA or RNA in a 5′ to 3′ direction or in the opposite 3′ to 5′ direction may be used in present embodiments of the disclosure. This includes helicases obtained from prokaryotes, viruses, archaea, and eukaryotes or recombinant forms of naturally occurring enzymes as well as analogues or derivatives having the specified activity. Examples of naturally occurring DNA helicases, described by Kornberg and Baker in chapter 11 of their book, DNA Replication, W. H. Freeman and Company (2^{nd} ed. (1992)), include E. coli helicase I, II, III, & IV, Rep, DnaB, PriA, PcrA, T4 Gp41helicase, T4 Dda helicase, T7 Gp4 helicases, SV40 Large T antigen, yeast RAD. Additional helicases include RecQ helicase (Harmon and Kowalczykowski, J. Biol. Chem. 276:232-243 (2001)), thermostable UvrD helicases from T. tengcongensis (disclosed herein, Example XII) and T. thermophilus (Collins and Mccarthy, Extremophiles. 7:35-41. (2003)), thermostable DnaB helicase from T. aquaticus (Kaplan and Steitz, J. Biol. Chem. 274:6889-6897 (1999)), and MCM helicase from archaeal and eukaryotic organisms ((Grainge et al., Nucleic Acids Res. 31:4888-4898 (2003)).

A traditional definition of a helicase is an enzyme that catalyzes the reaction of separating, unzipping, or unwinding the helical structure of nucleic acid duplexes (DNA, RNA, or hybrids) into single-stranded components, using nucleoside triphosphate (NTP) hydrolysis as the energy source (such as ATP). However, it should be noted that not all helicases fit this definition anymore. A more general definition is that they are motor proteins that move along the single-stranded or double stranded nucleic acids (usually in a certain direction, 3′ to 5′ or 5 to 3, or both), i.e. translocases, that can or cannot unwind the duplexed nucleic acid encountered. In addition, some helicases simply bind and “melt” the duplexed nucleic acid structure without an apparent translocase activity.

Helicases exist in all living organisms and function in all aspects of nucleic acid metabolism. Helicases are classified based on the amino acid sequences, directionality, oligomerization state and nucleic-acid type and structure preferences. The most common classification method was developed based on the presence of certain amino acid sequences, called motifs. According to this classification helicases are divided into 6 superfamilies: SF1, SF2, SF3, SF4, SF5, and SF6. SF1 and SF2 helicases do not form a ring structure around the nucleic acid, whereas SF3 to SF6 do. Superfamily classification is not dependent on the classical taxonomy.

DNA helicases are responsible for catalyzing the unwinding of double-stranded DNA (dsDNA) molecules to their respective single-stranded nucleic acid (ssDNA) forms. Although structural and biochemical studies have shown how various helicases can translocate on ssDNA directionally, consuming one ATP per nucleotide, the mechanism of nucleic acid unwinding and how the unwinding activity is regulated remains unclear and controversial (T. M. Lohman, E. J. Tomko, C. G. Wu, “Non-hexameric DNA helicases and translocases: mechanisms and regulation,” Nat Rev Mol Cell Biol 9:391-401 (2008)). Since helicases can potentially unwind all nucleic acids encountered, understanding how their unwinding activities are regulated can lead to harnessing helicase functions for biotechnology applications.

The disclosure comprises use of any suitable helicase known in the art. These include, but are not necessarily limited to, UvrD helicase, Srs2 helicase, CRISPR-Cas3 helicase, E. coli helicase I, E. coli helicase II, E. coli helicase III, E. coli helicase IV, Rep helicase, DnaB helicase, PriA helicase, PcrA helicase, T4 Gp41 helicase, T4 Dda helicase, SV40 Large T antigen, yeast RAD helicase, RecD helicase, RecG helicase RecQ helicase, thermostable T. tengcongensis UvrD helicase, thermostable T. thermophilus UvrD helicase, thermostable T. aquaticus DnaB helicase, Dda helicase, papilloma virus E1 helicase, archaeal MCM helicase, eukaryotic MCM helicase, and T7 Gp4 helicase.

Helicases exhibiting varying processivity ranges may be used advantageously as components of the compositions described herein. Helicases may be categorized exhibiting “long-range processivity” or “short-range processivity.” As used herein, the term “long-range processivity” (also used interchangeably herein with “long processivity range”) describes a helicase exhibiting a processivity range of greater than or equal to 200 base pairs. As used herein, the term “short-range processivity” (also used interchangeably herein with “short processivity range”) describes a helicase exhibiting a processivity range of less than 200 base pairs. In an embodiment, the compositions described herein may comprise a helicase exhibiting a processivity range of greater than or equal to 200 base pairs. In an embodiment, the helicase may be selected from the group comprising BLM (processivity range of over 200 base pairs (Brosh et al., Journal of Biological Chemistry, Vol. 275, No. 31, 4 Aug. 2000, pp. 23500-23508; Xue et al., Nucleic Acids Rs. 2019 Dec. 2; 47 (21): 11225-11237.)), NS3h (processivity range of up to about 500 base pairs (Gwack et al., Eur. J. Biochem. 250, 47-54 (1997))), PcrA (processivity range of up to about 3,000 base pairs (Chisty et al., Nucleic Acids Research, Volume 41, Issue 9, 1 May 2013, Pages 5050-5023)), RepX (processivity of up to about 6,000 base pairs (Arslan et al., Science. 2015 Apr. 17; 348 (6232): 344-347)), TraI (processivity of at least 850 base pairs (Sikora et al., Journal of Biological Chemistry, Vol. 281, No. 47, pp. 36110-36116, Nov. 24, 2006)), DNA2 (processivity of up to about 6,000 base pairs (Pinto et al., eLife, 2016; 5: e18574. DOI: 10.7554/eLife.18574)), Srs2, RecG, and PriA. In an embodiment, the compositions described herein may comprise a helicase exhibiting a processivity range of less than 200 base pairs. In an embodiment, the helicase may be selected from the group comprising UvrD (processivity of about 30-40 base pairs (Meiners et al., J Biol Chem. 2014. June 13; 289 (24): 17100-17110)), Rep (processivity of about 30-50 base pairs (Arslan et al., Science. 2015 Apr. 17; 348 (6232): 344-347)), and Sgs1 (processivity of about 100 base pairs (Kasaciunaite et al., The EMBO Journal (2019) 38: e101516)).

In an embodiment, the helicase is linked to or otherwise capable of associating with the deaminase and/or the programmable nickase. The term “associating with” or “associated with” is used herein in relation to the physical association between the components (i.e., programmable nickase, helicase, deaminase) of the compositions described herein. The term may be used with respect to how one molecule ‘associates’ with another, for example, between an adaptor protein and a functional domain, or between a Cas protein and other components of a gene editing system. In the case of such non-covalent protein-protein interactions, this association may be viewed in terms of recognition in the way an antibody recognizes an epitope. Alternatively, one protein may be associated with another protein via a covalent interaction, such as a protein-protein fusion. Fusion typically occurs by addition of the amino acid sequence of one protein to the amino acid sequence of another, for instance via splicing together of the nucleotide sequences that encode each protein or subunit. Alternatively, this association via protein-protein fusion may be viewed as binding between two molecules by direct linkage. In this case, the fusion protein may include a linker between the two subunits of interest (i.e., between the enzyme and the functional domain or between the adaptor protein and the functional domain). Thus, in one embodiment, the helicase may be associated with the deaminase via a non-covalent protein-protein interaction. In another embodiment, the helicase may be associated with the deaminase via a covalent protein-protein fusion. In another embodiment, the helicase may be associated with the deaminase via a covalent linker. In another embodiment, the associated helicase and deaminase may be further associated with the programmable nickase via a non-covalent protein-protein interaction. In another embodiment, the associated helicase and deaminase may be further associated with the programmable nickase via covalent protein-protein interaction. In another embodiment, the associated helicase and deaminase may be further associated with the programmable nickase via a covalent linker.

### Deaminases

In an embodiment, the compositions described herein comprise a deaminase configured to introduce one or more base edits within the portion of dsDNA unwound by the helicase. As used herein, the term “deaminase” (also used interchangeably herein with “deaminase protein” and “deaminase enzyme”) refers to a protein, polypeptide, or one or more functional domain(s) of a protein or polypeptide that catalyzes the removal of an amino group from a molecule. Within the context of the compositions described herein, the deaminase introduces base edits along the single-strand DNA displaced by the unwinding activity of the helicase. In an embodiment, the deaminase comprises a cytidine deaminase. In an embodiment, the deaminase comprises an adenosine deaminase.

### Cytidine Deaminases

As used herein, the term “cytidine deaminase” (also used interchangeably herein with “cytidine deaminase protein” and “cytidine deaminase enzyme”) refers to a protein, polypeptide, or one or more functional domain(s) of a protein or polypeptide that is capable of catalyzing a hydrolytic deamination reaction that converts a cytosine to a uracil. In an embodiment, the cytidine deaminase catalyzes this reaction on cytosine comprised within DNA. In an embodiment, the cytidine deaminase catalyzes this reaction on cytosine comprised within RNA.

Cytidine deaminases that can be used with the compositions described herein include, but are not limited to, an apolipoprotein B mRNA-editing complex (APOBEC) deaminase, an activation-induced deaminase (AID), or a cytidine deaminase 1 (CDA1). In particular embodiments, the deaminase in an APOBEC1 deaminase, an APOBEC2 deaminase, an APOBEC3A deaminase, an APOBEC3B deaminase, an APOBEC3C deaminase, and APOBEC3D deaminase, an APOBEC3E deaminase, an APOBEC3F deaminase, an APOBEC3G deaminase, an APOBEC3H deaminase, or an APOBEC4 deaminase.

In the compositions and systems of the present disclosure, the cytidine deaminase is capable of targeting cytosine in single-stranded DNA. In an embodiment, the cytidine deaminase may edit the single DNA strand that is displaced from the unwinding of the DNA duplex catalyzed by the helicase. In an embodiment, the cytidine deaminase may contain mutations that alter the editing window such as those disclosed in Kim et al., Nat Biotechnol. 2017 April; 35 (4): 371-376 (doi: 10.1038/nbt.3803).

In an embodiment, the cytidine deaminase is derived from one or more metazoa species, including but not limited to, mammals, birds, frogs, squids, fish, flies, and worms. In an embodiment, the cytidine deaminase is a human, primate, cow, dog, rat, or mouse cytidine deaminase.

In an embodiment, the cytidine deaminase is a human APOBEC, including hAPOBEC1 or hAPOBEC3. In an embodiment, the cytidine deaminase is a human AID.

In an embodiment, the cytidine deaminase comprises human APOBEC1 full protein (hAPOBEC1) or the deaminase domain thereof (hAPOBEC1-D) or a C-terminally truncated version thereof (hAPOBEC-T). In an embodiment, the cytidine deaminase is an APOBEC family member that is homologous to hAPOBEC1, hAPOBEC-D, or hAPOBEC-T. In an embodiment, the cytidine deaminase comprises human AID1 full protein (hAID) or the deaminase domain thereof (hAID-D) or a C-terminally truncated version thereof (hAID-T). In an embodiment, the cytidine deaminase is an AID family member that is homologous to hAID, hAID-D or hAID-T. In an embodiment, the hAID-T is a hAID which is C-terminally truncated by about 20 amino acids.

In an embodiment, the cytidine deaminase comprises the wild-type amino acid sequence of a cytosine deaminase. In an embodiment, the cytidine deaminase comprises one or more mutations in the cytosine deaminase sequence, such that the editing efficiency, and/or substrate editing preference of the cytosine deaminase is changed according to specific needs.

Certain mutations of APOBEC1 and APOBEC3 proteins have been described in Kim et al., Nat Biotechnol. 2017 April; 35 (4): 371-376 (doi: 10.1038/nbt.3803); and Harris et al. Mol. Cell (2002) 10:1247-1253, each of which is incorporated herein by reference in its entirety.

In one embodiment, the deaminase is a cytidine deaminase. In one embodiment, the cytidine deaminase is an activation induced deaminase (AID). In one embodiment, the AID is a hyperactive mutant (AID*Δ). Hess et al. Nat Methods. 2016, 13 (12): 1036-1042. In one embodiment, the deaminase is a tRNA-specific adenosine deaminase cytidine deaminase (TadACBE). In one embodiment, the deaminase is TadA-8e. Richter et al. Nat. Biotechnol. 38, 901 (2020). In one embodiment, the TadACBE is a dual base editor that performs both cytosine and adenine base editing, for example TadDE. Neugebauer et al. Nat. Biotechnol. 41, 673-685 (2023).

In an embodiment, the cytidine deaminase is an adenosine deaminase that has been engineered by directed evolution to function as a cytidine deaminase. See, e.g., Abudayyeh et al. Science. 2019, 365 (6451): 382-386.

In an embodiment, the cytidine deaminase is linked to or otherwise capable of associating with the helicase. Thus, in one embodiment, the cytidine deaminase may be associated with the helicase via a non-covalent protein-protein interaction. In another embodiment, the cytidine deaminase may be associated with the helicase via a covalent protein-protein fusion. In another embodiment, the cytidine deaminase may be associated with the helicase via a covalent linker. In another embodiment, the associated helicase and cytidine deaminase may be further associated with the programmable nickase via a non-covalent protein-protein interaction. In another embodiment, the associated helicase and cytidine deaminase may be further associated with the programmable nickase via covalent protein-protein interaction. In another embodiment, the associated helicase and cytidine deaminase may be further associated with the programmable nickase via a covalent linker.

In an embodiment, the cytidine deaminase may be used in combination with a uracil DNA glycosylase inhibitor (UGI). Uracil DNA glycosylase is an enzyme that catalyzes the removal of uracil in cellular DNA and initiates base excision repair, which usually reverts the uracil: guanine pair to a cytosine: guanine pair (Kim et al., Nat Biotechnol. 2017 April; 35 (4): 371-376 (doi: 10.1038/nbt.3803); and Harris et al. Mol. Cell (2002) 10:1247-1253; Kunz et al., Cell Mol Life Sci. 2009; 66:1021-1038 (doi: 10.1007/s00018-009-8739-9)). UGI, a protein derived from B. subtilis bacteriophage PBS1, has been used in cytidine deaminase base editors to inhibit the activity of uracil DNA glycosylase (Kim et al., 2017). Thus, in an embodiment, the compositions described herein comprising a cytidine deaminase may further comprise a UGI. In an embodiment, the UGI is linked to or otherwise capable of associating with the cytidine deaminase. In one embodiment, the UGI may be associated with the cytidine deaminase via a non-covalent protein-protein interaction. In another embodiment, the UGI may be associated with the cytidine deaminase via a covalent protein-protein fusion. In another embodiment, the UGI may be associated with the cytidine deaminase via a covalent linker.

### Adenosine Deaminases

As used herein, the term “adenosine deaminase” (also used herein interchangeably with “adenosine deaminase protein” and “adenosine deaminase enzyme”) refers to a protein, polypeptide, or one or more functional domain(s) of a protein or polypeptide that is capable of catalyzing a hydrolytic deamination reaction that converts an adenine to a hypoxanthine. In an embodiment, the adenosine deaminase catalyzes this reaction on adenine comprised within DNA. In an embodiment, the adenosine deaminase catalyzes this reaction on adenine comprised within RNA.

Adenosine deaminases that can be used with the compositions described herein include, but are not limited to, adenosine deaminases that act on RNA (ADAR), adenosine deaminases that act on transfer RNA (ADAT), transfer RNA adenosine deaminase A (TadA), and other adenosine deaminase domain-containing (ADAD) family members. According to the present disclosure, the adenosine deaminase is capable of targeting adenine in RNA/DNA and RNA duplexes. Indeed, Zheng et al. (Nucleic Acids Res. 2017, 45 (6): 3369-3377) demonstrate that ADARs can carry out adenosine to inosine editing reactions on RNA/DNA and RNA/RNA duplexes. In particular embodiments, the adenosine deaminase has been modified to increase its ability to edit DNA in an RNA/DNAn RNA duplex as detailed herein below.

In an embodiment, the adenosine deaminase is derived from one or more metazoa species, including but not limited to, mammals, birds, frogs, squids, fish, flies, and worms. In an embodiment, the adenosine deaminase is a human, squid, or Drosophila adenosine deaminase.

In an embodiment, the adenosine deaminase is a human ADAR, including hADAR1, hADAR2, hADAR3. In an embodiment, the adenosine deaminase is a Caenorhabditis elegans ADAR protein, including ADR-1 and ADR-2. In an embodiment, the adenosine deaminase is a Drosophila ADAR protein, including dAdar. In an embodiment, the adenosine deaminase is a squid Loligo pealeii ADAR protein, including sqADAR2a and sqADAR2b. In an embodiment, the adenosine deaminase is a human ADAT protein. In an embodiment, the adenosine deaminase is a Drosophila ADAT protein. In an embodiment, the adenosine deaminase is a human ADAD protein, including TENR (hADAD1) and TENRL (hADAD2).

In an embodiment, the adenosine deaminase protein recognizes and converts one or more target adenosine residue(s) in a double-stranded nucleic acid substrate into inosine residue(s). In an embodiment, the double-stranded nucleic acid substrate is an RNA-DNA hybrid duplex. In an embodiment, the adenosine deaminase protein recognizes a binding window on the double-stranded substrate. In an embodiment, the binding window contains at least one target adenosine residue(s). In an embodiment, the binding window is in the range of about 3 bp to about 100 bp. In an embodiment, the binding window is in the range of about 5 bp to about 50 bp. In an embodiment, the binding window is in the range of about 10 bp to about 30 bp. In an embodiment, the binding window is about 1 bp, 2 bp, 3 bp, 5 bp, 7 bp, 10 bp, 15 bp, 20 bp, 25 bp, 30 bp, 40 bp, 45 bp, 50 bp, 55 bp, 60 bp, 65 bp, 70 bp, 75 bp, 80 bp, 85 bp, 90 bp, 95 bp, or 100 bp.

In an embodiment, the adenosine deaminase protein comprises one or more deaminase domains. Not intended to be bound by theory, it is contemplated that the deaminase domain functions to recognize and convert one or more target adenosine (A) residue(s) contained in a double-stranded nucleic acid substrate into inosine (I) residue(s). In an embodiment, the deaminase domain comprises an active center. In an embodiment, the active center comprises a zinc ion. In an embodiment, during the A-to-I editing process, base pairing at the target adenosine residue is disrupted, and the target adenosine residue is “flipped” out of the double helix to become accessible by the adenosine deaminase. In an embodiment, amino acid residues in or near the active center interact with one or more nucleotide(s) 5′ to a target adenosine residue. In an embodiment, amino acid residues in or near the active center interact with one or more nucleotide(s) 3′ to a target adenosine residue. In an embodiment, amino acid residues in or near the active center further interact with the nucleotide complementary to the target adenosine residue on the opposite strand. In an embodiment, the amino acid residues form hydrogen bonds with the 2′ hydroxyl group of the nucleotides.

In an embodiment, the adenosine deaminase comprises human ADAR2 full protein (hADAR2) or the deaminase domain thereof (hADAR2-D). In an embodiment, the adenosine deaminase is an ADAR family member that is homologous to hADAR2 or hADAR2-D.

Particularly, in an embodiment, the homologous ADAR protein is human ADAR1 (hADAR1) or the deaminase domain thereof (hADAR1-D). In an embodiment, glycine 1007 of hADAR1-D corresponds to glycine 487 hADAR2-D, and glutamic acid 1008 of hADAR1-D corresponds to glutamic acid 488 of hADAR2-D.

In an embodiment, the adenosine deaminase comprises the wild-type amino acid sequence of hADAR2-D. In an embodiment, the adenosine deaminase comprises one or more mutations in the hADAR2-D sequence, such that the editing efficiency, and/or substrate editing preference of hADAR2-D is changed according to specific needs.

Certain mutations of hADAR1 and hADAR2 proteins have been described in Kuttan et al., Proc Natl Acad Sci USA. (2012) 109 (48): E3295-304; Want et al. ACS Chem Biol. (2015) 10 (11): 2512-9; and Zheng et al. Nucleic Acids Res. (2017) 45 (6): 3369-337, each of which is incorporated herein by reference in its entirety.

In an embodiment, the adenosine deaminase is linked to or otherwise capable of associating with the helicase. Thus, in one embodiment, the adenosine deaminase may be associated with the helicase via a non-covalent protein-protein interaction. In another embodiment, the adenosine deaminase may be associated with the helicase via a covalent protein-protein fusion. In another embodiment, the adenosine deaminase may be associated with the helicase via a covalent linker. In another embodiment, the associated helicase and adenosine deaminase may be further associated with the programmable nickase via a non-covalent protein-protein interaction. In another embodiment, the associated helicase and adenosine deaminase may be further associated with the programmable nickase via covalent protein-protein interaction. In another embodiment, the associated helicase and adenosine deaminase may be further associated with the programmable nickase via a covalent linker.

### Linkers

The term “associating with” or “associated with” may be used herein in relation to the physical association between the components (i.e., programmable nickase, helicase, deaminase) of the compositions described herein. The term may be used with respect to how one molecule ‘associates’ with another, for example, between an adaptor protein and a functional domain, or between a Cas protein and other components of a gene editing system. In the case of such non-covalent protein-protein interactions, this association may be viewed in terms of recognition in the way an antibody recognizes an epitope. Alternatively, one protein may be associated with another protein via a covalent interaction, such as a protein-protein fusion. Fusion typically occurs by addition of the amino acid sequence of one protein to the amino acid sequence of another, for instance via splicing together of the nucleotide sequences that encode each protein or subunit. Alternatively, this association via protein-protein fusion may be viewed as binding between two molecules by direct linkage. In this case, the fusion protein may include a linker between the two subunits of interest (i.e., between the enzyme and the functional domain or between the adaptor protein and the functional domain). Thus, in one embodiment, the helicase may be associated with deaminase and/or programmable nickase via a non-covalent protein-protein interaction. In another embodiment, the helicase may be associated with the deaminase and/or programmable nickase via covalent protein-protein fusion. In another embodiment, the helicase may be associated with the deaminase and/or programmable nickase via a covalent linker.

The protein components of the systems described herein (e.g., transposases, Cas proteins, tyrosine recombinases, etc.) may be associated via a linker. The term “linker” refers to a molecule which joins the proteins to form a fusion protein. Generally, such molecules have no specific biological activity other than to join or to preserve some minimum distance or other spatial relationship between the proteins. However, in an embodiment, the linker may be selected to influence some property of the linker and/or the fusion protein such as the folding, net charge, or hydrophobicity of the linker.

Suitable linkers for use in the methods herein include straight or branched-chain carbon linkers, heterocyclic carbon linkers, or peptide linkers. However, as used herein the linker may also be a covalent bond (carbon-carbon bond or carbon-heteroatom bond). In particular embodiments, the linker is used to separate the Cas protein and the transposase by a distance sufficient to ensure that each protein retains its required functional property. A peptide linker sequence may adopt a flexible extended conformation and may not exhibit a propensity for developing an ordered secondary structure. In an embodiment, the linker can be a chemical moiety which can be monomeric, dimeric, multimeric, or polymeric. In an embodiment, the linker comprises amino acids. Example amino acids in flexible linkers include Gly, Asn and Ser. Accordingly, in an embodiment, the linker comprises a combination of one or more of Gly, Asn and Ser amino acids. Other near neutral amino acids, such as Thr and Ala, also may be used in the linker sequence. Exemplary linkers are disclosed in Maratea et al. (1985), Gene 40:39-46; Murphy et al. (1986) Proc. Nat'l. Acad. Sci. USA 83:8258-62; U.S. Pat. Nos. 4,935,233; and 4,751,180.

For example, GlySer linkers GGS, GGGS (SEQ ID NO: 2) or GSG can be used. GGS, GSG, GGGS (SEQ ID NO: 2) or GGGGS (SEQ ID NO: 3) linkers can be used in repeats of 3 (such as (GGS)₃ (SEQ ID NO: 4), (GGGGS) 3 (SEQ ID NO: 5)) or 5, 6, 7, 9 or even 12 or more, to provide suitable lengths. In some cases, the linker may be (GGGGS)₃₋₁₅, For example, in some cases, the linker may be (GGGGS)₃₋₁₁, e.g., GGGGS (SEQ ID NO: 3), (GGGGS)₂ (SEQ ID NO: 6), (GGGGS)₃ (SEQ ID NO: 5), (GGGGS)₄ (SEQ ID NO: 7), (GGGGS)₅ (SEQ ID NO: 8), (GGGGS)₆ (SEQ ID NO: 9), (GGGGS)₇ (SEQ ID NO: 10), (GGGGS)₈ (SEQ ID NO: 11), (GGGGS)₉ (SEQ ID NO: 12), (GGGGS)₁₀ (SEQ ID NO: 13), or (GGGGS)₁₁ (SEQ ID NO: 14).

In particular embodiments, linkers such as (GGGGS)₃ (SEQ ID NO: 5) are preferably used herein. (GGGGS)₆ (SEQ ID NO: 9), (GGGGS)₉ (SEQ ID NO: 12) or (GGGGS)₁₂ (SEQ ID NO: 15) may be used as alternatives. Other alternatives include (GGGGS)₁ (SEQ ID NO: 3), (GGGGS)₂ (SEQ ID NO: 6), (GGGGS)₄ (SEQ ID NO: 7), (GGGGS)₅ (SEQ ID NO: 8), (GGGGS)₇ (SEQ ID NO: 10), (GGGGS)₈ (SEQ ID NO: 11), (GGGGS)₁₀ (SEQ ID NO: 13), or (GGGGS)₁₁ (SEQ ID NO: 14). In yet a further embodiment, LEPGEKPYKCPECGKSFSQSGALTRHQRTHTR (SEQ ID NO: 16) is used as a linker. In yet an additional embodiment, the linker is an XTEN linker. In an embodiment, the Cas protein is linked to the deaminase protein or its catalytic domain by means of an LEPGEKPYKCPECGKSFSQSGALTRHQRTHTR (SEQ ID NO: 16) linker. In some embodiments, the Cas protein is linked C-terminally to the N-terminus of a deaminase protein or its catalytic domain by means of an LEPGEKPYKCPECGKSFSQSGALTRHQRTHTR (SEQ ID NO: 16) linker. In addition, N- and C-terminal NLSs can also function as linker (e.g., PKKKRKVEASSPKKRKVEAS (SEQ ID NO: 17)).

Examples of linkers are shown in the Table 1 below.

TABLE 1

| | |
|---|---|
| GGS | GGTGGTAGT (SEQ ID NO: 18) |
| GGSx3 (9) | GGTGGTAGTGGAGGGAGCGGCGGTTCA (SEQ ID NO: 19) |
| (SEQ ID NO: | |
| 4) | |
| GGSx7 (21) | ggtggaggaggctctggtggaggcggtagcggaggcggagggtcgGGTGGTAGTGGAGGG |
| SEQ ID NO: | AGCGGCGGTTCA (SEQ ID NO: 21) |
| 20) | |
| XTEN | TCGGGATCTGAGACGCCTGGGACCTCGGAATCGGCTACGCCCGAA |
| | AGT (SEQ ID NO: 22) |
| Z- | Gtggataacaaatttaacaaagaaatgtgggcggcgtgggaagaaattcgtaacctgccgaacctgaacggc |
| EGFR_Short | tggcagatgaccgcgtttattgcgagcctggtggatgatccgagccagagcgcgaacctgctggcggaagcg |
| | aaaaaactgaacgatgcgcaggcgccgaaaaccggcggtggttctggt (SEQ ID NO: 23) |
| GSAT | Ggtggttctgccggtggctccggttctggctccagcggtggcagctctggtgcgtccggcacgggtactgcg |
| | ggtggcactggcagcggttccggtactggctctggc (SEQ ID NO: 24) |

Linkers may be used between the guide RNAs and the functional domain (activator or repressor), or between the Cas protein and the transposase(s). The linkers may be used to engineer appropriate amounts of “mechanical flexibility”.

In an embodiment, the one or more functional domains are controllable, e.g., inducible.

### Nuclear Localization Signals

In an embodiment, the systems and compositions herein further comprise one or more nuclear localization signals (NLSs). The NLS may be capable of driving the accumulation of the components, e.g., Cas and/or transposase(s) to a desired amount in the nucleus of a cell.

In an embodiment, at least one nuclear localization signal (NLS) is attached to the Cas and/or transposase(s). In an embodiment, one or more C-terminal or N-terminal NLSs are attached (and hence nucleic acid molecule(s) coding for the Cas and/or transposase(s) can include coding for NLS(s) so that the expressed product has the NLS(s) attached or connected). In some embodiments, a C-terminal NLS is attached for optimal expression and nuclear targeting in eukaryotic cells, e.g., human cells.

The NLS may be monopartite. In certain cases, the NLS may be bipartite. These types of NLSs can be further classified as either monopartite or bipartite. The two basic amino acid clusters in bipartite NLSs are separated by a short spacer sequence (e.g., about 10 amino acids), while monopartite NLSs are not. In some cases, one or more monopartite NSLs is attached to the Cas and/or transposase(s). In certain cases, one or more bipartite NSLs is attached to the Cas and/or transposase(s). In some cases, one or more monopartite NSLs and one or more bipartite NSLs are attached to the Cas and/or transposase(s).

Non-limiting examples of NLSs include an NLS sequence derived from: the NLS of the SV40 virus large T-antigen, having the amino acid sequence PKKKRKV (SEQ ID NO: 25); the NLS from nucleoplasmin (e.g., the nucleoplasmin bipartite NLS with the sequence KRPAATKKAGQAKKK (SEQ ID NO: 26)); the c-myc NLS having the amino acid sequence PAAKRVKLD (SEQ ID NO: 27) or RQRRNELKRS (SEQ ID NO: 28); the hRNPA1 M9 NLS having the sequence NQSSNFGPMKGGNFGGRSSGPYGGGGQYFAKPRNQGGY (SEQ ID NO: 29); the sequence RMRIZFKNKGKDTAELRRRRVEVSVELRKAKKDEQILKRRNV (SEQ ID NO: 30) of the IBB domain from importin-alpha; the sequences VSRKRPRP (SEQ ID NO: 31) and PPKKARED (SEQ ID NO: 32) of the myoma T protein; the sequence PQPKKKPL (SEQ ID NO: 33) of human p53; the sequence SALIKKKKKMAP (SEQ ID NO: 34) of mouse c-abl IV; the sequences DRLRR (SEQ ID NO: 35) and PKQKKRK (SEQ ID NO: 36) of the influenza virus NS1; the sequence RKLKKKIKKL (SEQ ID NO: 37) of the Hepatitis virus delta antigen; the sequence REKKKFLKRR (SEQ ID NO: 38) of the mouse Mx1 protein; the sequence KRKGDEVDGVDEVAKKKSKK (SEQ ID NO: 39) of the human poly(ADP-ribose) polymerase; and the sequence RKCLQAGMNLEARKTKK (SEQ ID NO: 40) of the steroid hormone receptors (human) glucocorticoid.

In an embodiment, a NLS is a heterologous NLS. For example, the NLS is not naturally present in the molecule (e.g., Cas and/or transposase(s)) to which it is attached.

In general, strength of nuclear localization activity may derive from the number of NLSs in the nucleic acid-targeting effector protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI).

In an embodiment, a vector described herein (e.g., those comprising polynucleotides encoding Cas proteins, transposase(s), tyrosine recombinases, etc.) comprise one or more nuclear localization sequences (NLSs), such as about or more than about 1, 2, 3, 4, 5, 6, 7, 8, 9, 10, or more NLSs. For example, vectors may comprise one or more NLSs not naturally present in the Cas and/or transposase(s). For example, the NLS may be present in the vector 5′ and/or 3′ of the Cas and/or transposase(s) sequence. In an embodiment, the Cas and/or transposase(s) comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In an embodiment, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus.

In an embodiment, other localization tags may be fused to the components of the systems described herein, such as without limitation for localizing to particular sites in a cell, such as organelles, such as mitochondria, plastids, chloroplast, vesicles, golgi, (nuclear or cellular) membranes, ribosomes, nucleolus, ER, cytoskeleton, vacuoles, centrosome, nucleosome, granules, centrioles, etc.

### Vectors and Delivery

The present disclosure also provides delivery systems for introducing components of the systems and compositions herein to cells, tissues, organs, or organisms. A delivery system may comprise one or more delivery vehicles and/or cargos.

In an embodiment, the delivery systems may be used to introduce the components of the systems and compositions to plant cells. For example, the components may be delivered to plants using electroporation, microinjection, aerosol beam injection of plant cell protoplasts, biolistic methods, DNA particle bombardment, and/or Agrobacterium-mediated transformation. Examples of methods and delivery systems for plants include those described in Fu et al., Transgenic Res. 2000 February; 9 (1): 11-9; Klein R M, et al., Biotechnology. 1992; 24:384-6; Casas A M et al., Proc Natl Acad Sci USA. 1993 Dec. 1; 90 (23): 11212-11216; and U.S. Pat. No. 5,563,055, Davey M R et al., Plant Mol Biol. 1989 September; 13 (3): 273-85, which are incorporated by reference herein in their entireties.

### Physical Delivery

In an embodiment, the cargos may be introduced to cells by physical delivery methods. Examples of physical methods include microinjection, electroporation, and hydrodynamic delivery. Both nucleic acid and proteins may be delivered using such methods. For example, Cas protein may be prepared in vitro, isolated, (refolded, purified if needed), and introduced to cells.

### Microinjection

Microinjection of the cargo directly to cells can achieve high efficiency, e.g., above 90% or about 100%. In an embodiment, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Plasmids comprising coding sequences for Cas proteins and/or guide RNAs, mRNAs, and/or guide RNAs, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to deliver sgRNA directly to the nucleus and Cas-encoding mRNA to the cytoplasm, e.g., facilitating translation and shuttling of Cas to the nucleus.

Microinjection may be used to generate genetically modified animals. For example, gene editing cargos may be injected into zygotes to allow for efficient germline modification. Such an approach can yield normal embryos and full-term mouse pups harboring the desired modification(s). Microinjection can also be used to transiently up- or down-regulate a specific gene within the genome of a cell, e.g., using CRISPRa and CRISPRi.

### Electroporation

In an embodiment, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to or into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles in accordance with the present disclosure may comprise a greatest dimension (e.g., diameter) of less than 100 microns (μm). In an embodiment, the delivery vehicles have a greatest dimension of less than 10 μm. In an embodiment, the delivery vehicles may have a greatest dimension of less than 2000 nanometers (nm). In an embodiment, the delivery vehicles may have a greatest dimension of less than 1000 nanometers (nm). In an embodiment, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, or less than 50 nm. In an embodiment, the delivery vehicles may have a greatest dimension ranging between 25 nm and 200 nm.

In an embodiment, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles). Nanoparticles may also be used to deliver the compositions and systems to plant cells, e.g., as described in WO 2008/042156, US 2013/0185823, and WO 2015/089419.

### Vectors

The systems, compositions, and/or delivery systems may comprise one or more vectors. The present disclosure also includes vector systems. A vector system may comprise one or more vectors. In an embodiment, a vector refers to a nucleic acid molecule capable of transporting another nucleic acid to which it has been linked. Vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques. Certain vectors may be capable of autonomous replication in a host cell into which they are introduced (e.g., bacterial vectors having a bacterial origin of replication and episomal mammalian vectors). Some vectors (e.g., non-episomal mammalian vectors) are integrated into the genome of a host cell upon introduction into the host cell, and thereby are replicated along with the host genome. In certain examples, vectors may be expression vectors, e.g., capable of directing the expression of genes to which they are operatively-linked. In some cases, the expression vectors may be for expression in eukaryotic cells. Common expression vectors of utility in recombinant DNA techniques are often in the form of plasmids.

In one embodiment the programmable nickase maybe encoded on one vector and the helicase and the deaminase may be encoded together on a separate vector, either separately or as a fusion protein. Example vectors are disclosed in the Example section below.

### Regulatory Elements

A vector may comprise one or more regulatory elements. The regulatory element(s) may be operably linked to coding sequences of the programmable nickase, helicase, and/or deaminase. The term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). In certain examples, a vector may comprise: a first regulatory element operably linked to a nucleotide sequence encoding a protein, and a second regulatory element operably linked to a nucleotide sequence encoding a guide RNA.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter.

### Viral Vectors

The cargos may be delivered by viruses. In an embodiment, viral vectors are used. A viral vector may comprise virally-derived DNA or RNA sequences for packaging into a virus (e.g., retroviruses, replication defective retroviruses, adenoviruses, replication defective adenoviruses, and adeno-associated viruses). Viral vectors also include polynucleotides carried by a virus for transfection into a host cell. Viruses and viral vectors may be used for in vitro, ex vivo, and/or in vivo deliveries.

### Adeno Associated Virus (AAV)

The systems and compositions herein may be delivered by adeno associated virus (AAV). AAV vectors may be used for such delivery. AAV, of the Dependovirus genus and Parvoviridae family, is a single stranded DNA virus. In an embodiment, AAV may provide a persistent source of the provided DNA, as AAV delivered genomic material can exist indefinitely in cells, e.g., either as exogenous DNA or, with some modification, be directly integrated into the host DNA. In an embodiment, AAV do not cause or relate with any diseases in humans. The virus itself is able to efficiently infect cells while provoking little to no innate or adaptive immune response or associated toxicity.

Examples of AAV that can be used herein include AAV-1, AAV-2, AAV-3, AAV-4, AAV-5, AAV-6, AAV-8, and AAV-9. The type of AAV may be selected with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. AAV-2-based vectors were originally proposed for CFTR delivery to CF airways, other serotypes such as AAV-1, AAV-5, AAV-6, and AAV-9 exhibit improved gene transfer efficiency in a variety of models of the lung epithelium. Examples of cell types targeted by AAV are described in Grimm, D. et al, J. Virol. 82:5887-5911 (2008)), and shown as follows:

TABLE 2

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

AAV particles may be created in HEK 293 T cells. Once particles with specific tropism have been created, they are used to infect the target cell line much in the same way that native viral particles do. This may allow for persistent presence of CRISPR-Cas components in the infected cell type, and what makes this version of delivery particularly suited to cases where long-term expression is desirable. Examples of doses and formulations for AAV that can be used include those described in U.S. Pat. Nos. 8,454,972 and 8,404,658.

Various strategies may be used for delivery of the systems and compositions herein with AAVs. In some examples, coding sequences of Cas and gRNA may be packaged directly onto one DNA plasmid vector and delivered via one AAV particle. In some examples, AAVs may be used to deliver gRNAs into cells that have been previously engineered to express Cas. In some examples, coding sequences of Cas and gRNA may be made into two separate AAV particles, which are used for co-transfection of target cells. In some examples, markers, tags, and other sequences may be packaged in the same AAV particles as coding sequences of Cas and/or gRNAs.

### Lentiviruses

The systems and compositions herein may be delivered by lentiviruses. Lentiviral vectors may be used for such delivery. Lentiviruses are complex retroviruses that have the ability to infect and express their genes in both mitotic and post-mitotic cells.

Examples of lentiviruses include human immunodeficiency virus (HIV), which may use its envelope glycoproteins of other viruses to target a broad range of cell types; minimal non-primate lentiviral vectors based on the equine infectious anemia virus (EIAV), which may be used for ocular therapies. In an embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the nucleic acid-targeting system herein.

Lentiviruses may be pseudo-typed with other viral proteins, such as the G protein of vesicular stomatitis virus. In doing so, the cellular tropism of the lentiviruses can be altered to be as broad or narrow as desired. In some cases, to improve safety, second- and third-generation lentiviral systems may split essential genes across three plasmids, which may reduce the likelihood of accidental reconstitution of viable viral particles within cells.

In some examples, leveraging the integration ability, lentiviruses may be used to create libraries of cells comprising various genetic modifications, e.g., for screening and/or studying genes and signaling pathways.

### Adenoviruses

The systems and compositions herein may be delivered by adenoviruses. Adenoviral vectors may be used for such delivery. Adenoviruses include nonenveloped viruses with an icosahedral nucleocapsid containing a double stranded DNA genome. Adenoviruses may infect dividing and non-dividing cells. In an embodiment, adenoviruses do not integrate into the genome of host cells, which may be used for limiting off-target effects of CRISPR-Cas systems in gene editing applications.

### Viral Vehicles for Delivery to Plants

The systems and compositions may be delivered to plant cells using viral vehicles. In particular embodiments, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

### Non-Viral Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, gold nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes.

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of Cas and/or gRNA) and/or RNA molecules (e.g., mRNA of Cas, gRNAs). In certain cases, LNPs may be used for delivering RNP complexes of Cas/gRNA.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol) 2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, December 2011).

### Liposomes

In an embodiment, a lipid particle may be liposome. Liposomes are spherical vesicle structures composed of a uni- or multilamellar lipid bilayer surrounding internal aqueous compartments and a relatively impermeable outer lipophilic phospholipid bilayer. In an embodiment, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

### Stable Nucleic-Acid-Lipid Particles (SNALPs)

In an embodiment, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol) 2000) carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-CDMA, and 1,2-dilinoleyloxy-3-(N;N-dimethyl)aminopropane (DLinDMA)

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

### Lipoplexes/Polyplexes

In an embodiment, the delivery vehicles comprise lipoplexes and/or polyplexes. Lipoplexes may bind to negatively charged cell membranes and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca21p (e.g., forming DNA/Ca²⁺ microcomplexes), polyethenimine (PEI) (e.g., branched PEI), and poly(L-lysine) (PLL).

### Cell Penetrating Peptides

In an embodiment, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPPs may also be used to deliver RNPs.

CPPs may be used to deliver the compositions and systems to plants. In some examples, CPPs may be used to deliver the components to plant protoplasts, which are then regenerated to plant cells and further to plants.

### DNA Nanoclews

In an embodiment, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aid in the self-assembly of the structure. The sphere may then be loaded with a payload. An example of DNA nanoclew is described in Sun W et al, J Am Chem Soc. 2014 Oct. 22; 136 (42): 14722-5; and Sun W et al, Angew Chem Int Ed Engl. 2015 Oct. 5; 54 (41): 12029-33. DNA nanoclew may have a palindromic sequence to be partially complementary to the gRNA within the Cas:gRNA ribonucleoprotein complex. A DNA nanoclew may be coated, e.g., coated with PEI to induce endosomal escape.

### Gold Nanoparticles

In an embodiment, the delivery vehicles comprise gold nanoparticles (also referred to AuNPs or colloidal gold). Gold nanoparticles may form complexes with cargos, e.g., Cas:gRNA RNP. Gold nanoparticles may be coated, e.g., coated in a silicate and an endosomal disruptive polymer, PAsp (DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901.

iTOP

In an embodiment, the delivery vehicles comprise iTOP. iTOP refers to a combination of small molecules that drive the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In an embodiment, the delivery vehicles may comprise polymer-based particles (e.g., nanoparticles). In an embodiment, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In an embodiment, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data., doi: 10.13140/RG.2.2.23912.16642.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6: e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptides (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In an embodiment, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and deliver various types of biomolecules, such as proteins, carbohydrates, lipids, and nucleic acids, and complexes thereof (e.g., RNPs). Examples of exosomes include those described in Schroeder A, et al., J Intern Med. 2010 January; 267 (1): 9-21; El-Andaloussi S, et al., Nat Protoc. 2012 December; 7 (12): 2112-26; Uno Y, et al., Hum Gene Ther. 2011 June; 22 (6): 711-9; Zou W, et al., Hum Gene Ther. 2011 April; 22 (4): 465-75.

In some examples, the exosome may form a complex (e.g., by binding directly or indirectly) to one or more components of the cargo. In certain examples, a molecule of an exosome may be fused with a first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

### Modified Cells and Organisms

Described herein are modified cells, cell populations, and organisms that can be modified by the engineered CRISPR-Cas system of the present disclosure. The modified cells, cell populations, and organisms can have an insertion of one or more polynucleotides, deletion of one or more polynucleotides, mutation of one or more polynucleotides, or a combination thereof. The modification can result in activation of one or more genes, inactivation of one or more genes, modulation of one or more genes, or a combination thereof. Cells, including cells in an organism, can be modified in vitro, in situ, ex vivo, or in vivo. In an embodiment, the modification is insertion or deletion of a polynucleotide, gene, or allele of interest. In an embodiment, the polynucleotide, gene, or allele of interest is associated with a genetic disease or condition.

### Modified Cells

Also described herein are modified cells and cell populations that can be modified by an embodiment of a targeted continuous mutagenesis composition described in greater detail elsewhere herein. In an embodiment, the cell is a eukaryotic cell. In an embodiment, the eukaryotic cell is a mammalian cell. In an embodiment, the eukaryotic cell is a non-human mammalian cell. In an embodiment, the cell is a human cell. In an embodiment, the cell is a plant cell. In an embodiment, the cell is a fungal cell. In an embodiment, the cell is a prokaryotic cell. The cells can be modified in vitro, ex vivo, or in vivo. The cells can be modified by delivering a polynucleotide modifying agent or system described in greater detail elsewhere herein or a component thereof into a cell by a suitable delivery mechanism. Suitable delivery methods and techniques include but are not limited to, transfection via a vector, transduction with viral particles, electroporation, endocytic methods, and others, which are described elsewhere herein and will be appreciated by those of ordinary skill in the art in view of this disclosure.

The modified cells can be further optionally cultured and/or expanded in vitro or ex vivo using any suitable cell culture techniques or conditions, which unless specified otherwise herein, will be appreciated by one of ordinary skill in the art in view of this disclosure. In an embodiment, the cells can be modified, optionally cultured and/or expanded, and administered to a subject in need thereof. In an embodiment, cells can be isolated from a subject, subsequently modified and optionally cultured and/or expanded, and administered back to the subject, such as in a cell therapy. In an embodiment, the cell therapy is an adoptive cell therapy. Such administration can be referred to as autologous administration. In an embodiment, cells can be isolated from a first subject, subsequently modified, optionally cultured and/or expanded, and administered to a second subject, where the first subject and the second subject are different. Such administration can be referred to as non-autologous administration.

In an embodiment, the modified cells can be used as a bioreactor for production of a bioproduct. In an embodiment, engineered compositions of the present disclosure introduce a gene or polynucleotide or otherwise modify the cell to produce one or more bioproducts. In an embodiment, the engineered compositions of the present disclosure are used to modify a producer cell so as to improve production of a bioproduct.

### Organisms

Also described herein are modified organisms. In an embodiment, the modified organisms can include one or more modified cells as are described elsewhere herein. In an embodiment, the modified organism is a non-human mammal. In an embodiment, the modified organism is a modified plant. In an embodiment, the modified organism is an insect. In an embodiment, the modified organism is a fungus. The modified organisms can be generated using the compositions described herein. Methods of making modified organisms are described in greater detail elsewhere herein.

The systems and methods described herein can be used in non-animal organisms, e.g., plants, fungi to generate modified non-animal organisms. The system and methods described can be used to generate non-human animal organisms. The system and methods described herein can be used to modify non-germline cells in a human. In an embodiment, the modification is expression of a polynucleotide of interest, gene of interest, and/or allele of interest.

### Non-Human Animals

The systems and methods may be used to generate modified non-human animals and cells thereof. In an aspect, the disclosure provides a non-human eukaryotic organism; preferably a multicellular eukaryotic organism, comprising a eukaryotic host cell according to any of the described embodiments. In other aspects, the disclosure provides a eukaryotic organism; preferably a multicellular eukaryotic organism, comprising a eukaryotic host cell according to any of the described embodiments. The organism in an embodiment of these aspects may be an animal; for example, a mammal. Also, the organism may be an arthropod such as an insect. The present disclosure may also be extended to other agricultural applications such as, for example, farm and production animals. For example, pigs have many features that make them attractive as biomedical models, especially in regenerative medicine. In particular, pigs with severe combined immunodeficiency (SCID) may provide useful models for regenerative medicine, xenotransplantation (discussed also elsewhere herein), and tumor development and will aid in developing therapies for human SCID patients. Lee et al., (Proc Natl Acad Sci USA. 2014 May 20; 111 (20): 7260-5) utilized a reporter-guided transcription activator-like effector nuclease (TALEN) system to generated targeted modifications of recombination activating gene (RAG) 2 in somatic cells at high efficiency, including some that affected both alleles. Such techniques and modifications can be adapted for and used with the modifying agent(s) and systems thereof described herein to generate a modified non-human animal or cell thereof.

The methods of Lee et al., (Proc Natl Acad Sci USA. 2014 May 20; 111 (20): 7260-5) may be applied to the present disclosure analogously as follows. Mutated pigs are produced by targeted insertion for example in RAG2 in fetal fibroblast cells followed by SCNT and embryo transfer. Constructs coding for CRISPR Cas and a reporter are electroporated into fetal-derived fibroblast cells. After 48 h, transfected cells expressing the green fluorescent protein are sorted into individual wells of a 96-well plate at an estimated dilution of a single cell per well. Targeted modification of RAG2 are screened by amplifying a genomic DNA fragment flanking any CRISPR Cas cutting sites followed by sequencing the PCR products. After screening and ensuring lack of off-site mutations, cells carrying targeted modification of RAG2 are used for SCNT. The polar body, along with a portion of the adjacent cytoplasm of oocyte, presumably containing the metaphase II plate, are removed, and a donor cell are placed in the perivitelline. The reconstructed embryos are then electrically porated to fuse the donor cell with the oocyte and then chemically activated. The activated embryos are incubated in Porcine Zygote Medium 3 (PZM3) with 0.5 μM Scriptaid (S7817; Sigma-Aldrich) for 14-16 h. Embryos are then washed to remove the Scriptaid and cultured in PZM3 until they were transferred into the oviducts of surrogate pigs. Such techniques and modifications can be adapted for and used with the targeted continuous mutagenesis systems described herein to generate a modified non-human animal or cell thereof.

The modified non-human animals described herein can be a platform to model a disease or disorder of an animal, including but not limited to mammals. In some of these embodiments, the mammal can be a human. In an embodiment, such models and platforms are rodent based, in non-limiting examples rat or mouse. Such models and platforms can take advantage of distinctions among and comparisons between inbred rodent strains. In an embodiment, such models and platforms include primate, horse, cattle, sheep, goat, swine, dog, cat or bird-based, for example to directly model diseases and disorders of such animals or to create modified and/or improved lines of such animals. Advantageously, in an embodiment, an animal-based platform or model is created to mimic a human disease or disorder. For example, the similarities of swine to humans make swine an ideal platform for modeling human diseases. Compared to rodent models, development of swine models has been costly and time intensive. On the other hand, swine and other animals are much more similar to humans genetically, anatomically, physiologically, and pathophysiologically. The present disclosure provides a high efficiency platform for targeted mutagenesis to be used in such animal platforms and models. Though ethical standards block development of human models and, in many cases, models based on non-human primates, the present disclosure is used with in vitro systems including, but not limited to, cell culture systems, three dimensional models and systems, and organoids to mimic, model, and investigate genetics, anatomy, physiology and pathophysiology of structures, organs, and systems of humans. The platforms and models provide manipulation of single or multiple targets.

### Methods for Targeted Continuous Long-Range Mutagenesis

The compositions disclosed herein may be used in a method of continuous mutagenesis, a process whereby mutations are continuously introduced into a genome, or gene over time. Continuous mutagenesis may be used in functional genetics study to understand the roles of specific genes or sequences in an organism. By observing the effects of different mutations, and mutation combinations, scientists can infer the function of a mutated gene or non-coding region. Continuous mutagenesis may also be used to study resistance to therapeutic molecules by introducing mutations that enable survival of cells upon exposure to therapeutic molecules. Understanding the mutations that lead to resistance can in turn allow for screening and design of more effective therapeutic molecules. Continuous mutagenesis may also be used to evolve proteins or nucleic acids towards a desired trait. By repeatedly mutating and selecting for certain properties (like increased enzymatic activity or binding affinity), molecules can be engineered with enhanced or novel functions. All of these applications, and more, are enabled by the compositions of the present disclosure. In each of the disclosed and claimed methods it will be understood that multiple programable nickase, helicase, and deaminase combinations may be used concurrently or sequentially. Different combinations may allow for fine tuning as to the locations that can be targeted, length of the mutagenesis window, and the rate of base edits introduced to generate the desire level of diversification in a given target region.

### Continuous Targeted Mutagenesis

In one embodiment, a method of targeted continuous mutagenesis comprises delivering to a cell population a HACE composition as described above. The programmable nickases are configured to introduce a nick site at one or more locations (e.g., genomic locations) where continuous mutagenesis is desired. The nickase may be directed to the target strand or the non-target strand of DNA. In the context of CRISPR-Cas and OMEGA systems, the target strand refers to the strand of DNA that contains the sequence complementary to and pairs with the guide RNA or ωRNA, and the non-target strand is the strand that does not directly pair with the guide RNA or ωRNA. The helicase then unwinds a portion of the dsDNA starting at the nick site. Introduction of mutations, which may be random, are made on a strand of DNA in the unwound portion of the dsDNA by the nucleotide deaminase. Nucleotide deaminases introduce mutations by converting base pairs. For example, a cytidine deaminase converts cytosine to uracil (which retains thymine base pairing properties in DNA), and an adenosine deaminase converts adenosine to inosine (which is read as guanine during DNA replication and base pairs with cytosine). In an embodiment, the helicase and nucleotide deaminase are linked or fused together. In summary, the programmable nickase allows targeting to a specific genomic region where mutation is desired. The helicase and nucleotide deaminase then work in combination to generate multiple edits (mutations) across an extended mutagenic window created by the winding activity of the helicase. In an embodiment, the mutagenic window is within between 500 to 5000 bp, 500 to 725 bp, 500 to 1000 bp, 725 to 1000 bp, 1000 to 1100 bp, 1000 to 1200 bp, 1000 to 1300 bp, 1000 to 1400 bp, 1000 to 1500 bp, 1000 to 1600 bp, 1000 to 1700 bp, 1000 to 1800 bp, 1000 to 1900 bp, 1000 to 2000 bp, 1000 to 3000 bp, 1000 to 4000 bp, or 1000 to 5000 bp. Diversification may be allowed to proceed over 1, 2, 3, 4, 5, 6, 7, 8, 9, 10, 11, 12, 13, or 14 days.

More than one programmable nickase may be used to target multiple locations for continuous mutagenesis. The programmable nickase may the same type of programmable nickase e.g., a HACE may comprise different nickases, e.g., a TALEN and CRISPR-Cas. Where a CRISPR-Cas system or OMEGA system is used, multiple gRNA or @RNAs may be used to target multiple locations in a multiplex fashion.

The method of targeted continuous mutagenesis may further comprise isolating DNA from the cell population and sequencing the DNA to identify the mutations in the one or more regions targeted for continuous mutagenesis using the compositions described herein. In one embodiment, amplicon sequencing is used to sequence the one or more regions targeted for continuous mutagenesis, which may also be referred to herein as “diversification”.

### Directed Evolution of Polypeptides and Polynucleotides

In an embodiment, the method of targeted continuous mutagenesis may be used to direct evolution of a polypeptide or polynucleotide having enhanced or novel characteristics. For example, one or more functions that directed evolution may be used to obtain included enhanced stability, increased enzymatic efficiency, altered substrate (target) binding specificity, improved substrate (target) binding affinity, new enzymatic activity relative to a non-evolved wild type version of the polypeptide or polynucleotide, or a combination thereof. Accordingly, in one embodiment the programmable nuclease is configured to introduce the mutagenesis window in a dsDNA sequence encoding the polypeptide or polynucleotide to be evolved. Selection of the site will depend on the functionality to be evolved. For example, an exon encoding a particular enzymatic function may be targeted if the goal is to evolve a polypeptide with enhanced or novel catalytic activity. Likewise, a region comprising a domain responsible for substrate binding may be targeted if the goal is to alter or enhance substrate binding. The length of the mutagenesis window also dictates configuration of the programmable nickase. The nicking site targeted by the programmable nickase needs to be close enough to the region to be edited such that it falls with the editing window of the helicase i.e., the length of dsDNA that can be unwound by the helicase activity.

After the continuous mutagenesis step is complete, a functional screen may be applied to screen for the desired characteristic. A number of functional screens are known in the art and selection of the appropriate screen may depend on the desired trait. One of ordinary skill in the art can select the appropriate functional screen base on the trait to be selected for. By way of example, but not limitation, the following papers describe functional screens that were paired with directed evolution to select for functional characteristics of interest: Festa et al. utilized directed evolution to develop new laccases, using random mutagenesis to select mutants with improved activity and stability compared to the wild-type enzyme. Festa et al. Proteins, 2008, 72 (1): 25-3; Waltenspühl et al. presented an engineering strategy to enhance GPCRs properties. This was applied to the human oxytocin receptor, resulting in variants with improved production levels, Waltenspühl et al. Scientific Reports, 2021, 11 (8630); Xue et al. increased the expression and activity of the transglutaminase enzyme from Streptomyces mobaraensis in E. coli through directed evolution, Xue et al. Food Biotechnology 2020, 34 (1): 42-61; Gielen et al. described a microfluidic workflow enabling ultrahigh-throughput screening of single-cell lysates, useful in directed evolution and functional metagenomics, Gielan et al. Protein Engineering. Methods in Molecular Biology, vol 1685. Humana Press, New York, NY; Throckmorton et al. used directed evolution and genetic selection to analyze the specificity code of the adenylation domain of EntF, an NRPS involved in enterobactin biosynthesis, identifying new specificity codes for L-Ser recognition, Throckmorton et al. ACS Chem. Biol. 2019, 14 (9): 2044-2054; Sago et al. demonstrated that changes in the chemical composition of nanoparticles can significantly impact their targeting ability, which might negate the need for active targeting ligands, Sago et al. J. Am. Chem. Soc. 2018, 140 (49): 17095-17105; Yin et al. engineered a mutant fungal laccase, PIE5, with an optimum pH at an alkaline condition, showing improved indigo dye decolorization capabilities compared to the parental laccase, which could be beneficial for certain industrial applications, Yin et al. AMB Expr. 2019, 9, 151; Saito et al. combined molecular evolution with machine learning to guide mutagenesis, altering the fluorescence of a green fluorescent protein into yellow, demonstrating the creation of proteins with longer wavelengths than the reference yellow fluorescent protein, Saito et al. ACS Synth. Biol. 2018, 7 (9): 2014-2022; Xiang et al. improved the specific activity and pH stability of xylanase XynHBN188A, resulting in a mutant with 2.8-fold higher specific activity and better pH stability than the wild type, Xiang et al., Bioresour. Bioprocess. 6, 25 (2019). doi.org/10.1186/s40643-019-0262-8; and Gonzalez-Perez and Alcalde discussed the generation of improved versatile peroxidase (VP) variants that have industrial applications, such as enhanced expression, activity, and stability, David Gonzalez-Perez & Miguel Alcalde, Biocatalysis and Biotransformation, 2018, 36:1, 1-11. Accordingly, similar functional screens may be combined with the targeted continuous evolution methods disclosed herein to select for desired engineered functional traits.

Following selection of cells enriched for the desired functional traits, DNA may be isolated from the selected cells to identify mutations associated with the desired functional traits. Further validation of the identified mutations may be obtained by introducing the one or more identified mutations into a wild-type cell, for example using a gene editing technique such as base editing or prime editing and rescreening for the desired function activity to validate which mutation or combination of mutations is associated with the desired characteristic. Example validation steps are disclosed in the Examples section below.

### Resistance to Therapeutic Agent Screening

In an embodiment, a method for identifying resistance mutations to therapeutic agents may comprise diversifying one or more target loci for one or more genes by delivering to a sample cell population the HACE compositions to introduce several mutations into the one or more target loci. The one or more target loci may be coding or non-coding. In an embodiment, the one or more target loci may be an exon or an intron in a gene known or suspected of being associated with drug resistance. Diversification may be allowed to proceed over 1, 2, 3, 4, 5, 6, 7, 8, 9, 10, 11, 12, 13, or 14 days. Mutations that confer a survival benefit, in this instance resistance to a given therapeutic agent, are then selected for by exposing the sample cell to the given therapeutic agents to be screened. Any type of therapeutic agent may be screened including but not limited to, small molecules, to siRNA, gene editors, and antibodies. One of ordinary skill in the art will be able to select the appropriate duration for the selection step based on the type of therapeutic agent or combination of therapeutics agents to be screened. DNA from surviving cells is then isolated and sequenced to identify for mutated alleles significantly enriched post-drug selection. Cell viability may be accessed using standard techniques known in the art and an example technique is described below in the Examples. The mutation rate (allele frequency) may be calculated for both pre- and post-selection samples. Significantly enriched mutations may be identified by comparing the base counts between pre- and post-selection samples using a Fisher's exact test. In an embodiment, a significantly enriched allele has a p-value less than 0.05. In an embodiment, a significantly enriched allele has a p-value less than 0.01.

Further validation of the identified mutations may be obtained by introducing the one or more identified mutations into a wild-type cell, for example using a gene editing technique such as base editing or prime editing and rescreening for the desired function activity to validate which mutation or combination of mutations is associated with resistance to the therapeutic agent. As used herein a wild-type cell is a cell that does not comprise the mutations to be validated prior to their introduction via gene editing. Example validation steps are disclosed in the Examples section below.

Identifying Mutations Associated with Alternative Splicing Events

In an embodiment, a method of identifying mutations associated with incorrect splicing events may comprise introducing into a sample cell population a splicing reporter configured to produce a detectable signal in the presence of an alternative splicing event. The alternative splicing event may result in a different protein, a protein of altered function (i.e. either increased or decreased activity), or a non-functional protein. The method may be used to identify mutations in proteins associated with splicing regulation that can lead to alternative splicing events. In an embodiment, the splicing reporter may comprise a portion of an endogenous intron and downstream exon fused to a constant upstream exon and a downstream fluorescent protein reporter such that results in a frameshift in an opening reading of the fluorescent protein reporter suppressing fluorescence, while an incorrect splicing event permits GFP expression and fluorescence.

The one or more target regions associated with alternative splicing events, e.g., proteins associated with splicing regulation, are diversified using the HACE compositions disclosed herein. Diversification may be allowed to proceed over 1, 2, 3, 4, 5, 6, 7, 8, 9, 10, 11, 12, 13, or 14 days. Cells containing an alternative splicing event are then selected by detecting cells expressing the detectable signal from the splicing reporter. For example, cells may be sorted, based on fluorescent protein expression into two bins (fluorescence negative and fluorescence positive). DNA may then be isolated from cells in the fluorescence positive bin and sequenced to identify mutations at the one or more target locations that are associated with the detected alternative splicing event. Mutations may be selected based on fold enrichment which may be calculated by dividing the mutation rate in the fluorescent positive samples by that of the fluorescent negative samples. Significantly enriched mutations may be determined using a Fisher's exact test. In an embodiment, a significantly enriched allele has a p-value less than 0.05. In an embodiment, a significantly enriched allele has a p-value less than 0.01. In an embodiment, significantly enriched mutations may show a log 2 fold change greater than 1.

Further validation of the identified mutations may be obtained by introducing the one or more identified mutations into a wild-type cell, for example using a gene editing technique such as base editing or prime editing and rescreening for the desired function activity to validate which mutation or combination of mutations is associated with the alternative splicing event. As used herein a wild-type cell is a cell that does not comprise the mutations to be validated prior to their introduction via gene editing. Example validation steps are disclosed in the Examples section below.

Identifying Functional Variants within Non-Coding Gene Regulatory Elements

In an embodiment, a method of identifying a functional variant within non-coding gene regulatory elements may comprise diversifying one or more non-coding gene regulatory elements by delivering to a sample cell population the HACE compositions disclosed herein. The non-coding gene regulatory element may comprise a promoter, an enhancer, silencers, insulators, locus control regions, 5′ and 3′ untranslated regions, introns, microRNA (miRNA) and small interfering RNA (siRNA) binding sites, response elements, or a combination thereof. Diversification may be allowed to proceed over 1, 2, 3, 4, 5, 6, 7, 8, 9, 10, 11, 12, 13, or 14 days.

Expression in the gene or genes controlled by the non-coding gene regulatory element is then induced. Cells are then selected based on detecting increased expression of the one or more genes. One of ordinary skill in the art can select the proper induction conditions and technique for measuring gene expression using known techniques in the art and according to the gene expression to be detected. DNA is then isolated from cells both exhibiting high and low expression. An example of selecting high and low expression using FACs is disclosed in the example below. To identify enriched bases, the t % C→T or % G→A of each group may be calculated for both high expression and low expression groups (% high or % low). Then the log 2 odds ratio of high expression versus low expression may be calculated as log₂OR=log 2 [(% high/(1−% high))/(% low/(1−% low))]. The correlation of technical replicates was plotted using GraphPad Prism 10.0. The top hits are recorded in Table 13.

Further validation of the identified mutations may be obtained by introducing the one or more identified mutations into a wild-type cell, for example using a gene editing technique such as base editing or prime editing and rescreening for the desired function activity to validate which mutation or combination of mutations is associated with functional variation in a non-coding gene regulatory element. As used herein a wild-type cell is a cell that does not comprise the mutations to be validated prior to their introduction via gene editing. Example validation steps are disclosed in the Examples section below.

### KITS

Any of the compounds, compositions, formulations, particles, or cells, described herein or a combination thereof can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, or cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In an embodiment, the combination kit also includes instructions printed on or otherwise contained in a tangible medium of expression. The instructions can provide information regarding the content of the compounds, compositions, formulations, particles, or cells, described herein or a combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In an embodiment, the instructions can provide directions for administering the compounds, compositions, formulations, particles, and cells described herein or a combination thereof to a subject in need thereof.

### EXAMPLES

Further embodiments are illustrated in the following Examples which are given for illustrative purposes only and are not intended to limit the scope of the disclosure.

### Example 1—Helicases Fused to Deaminase AID Promote Base Editing of Endogenous Loci in HEK293FT Cells

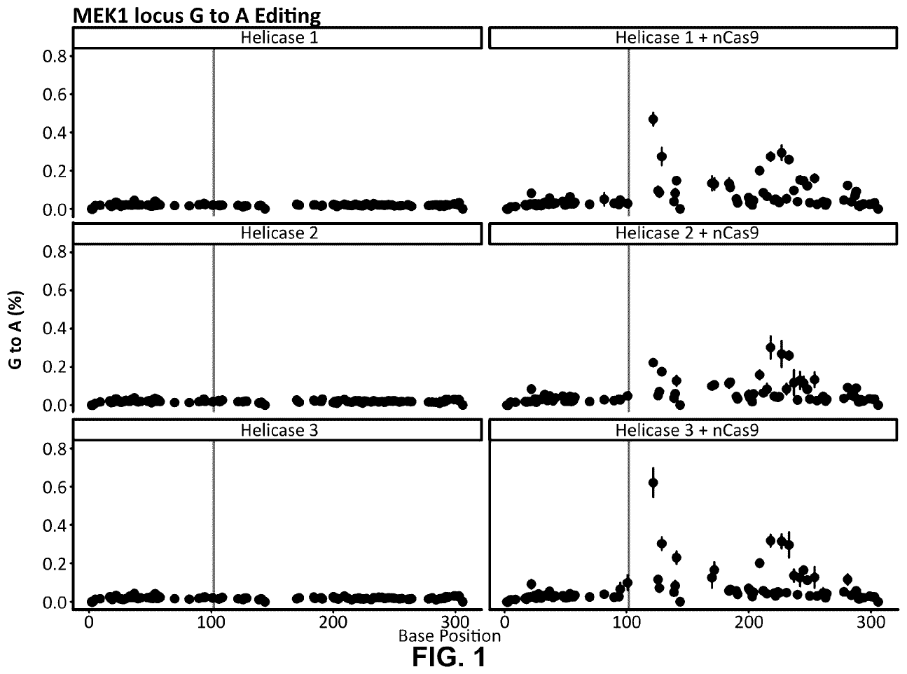

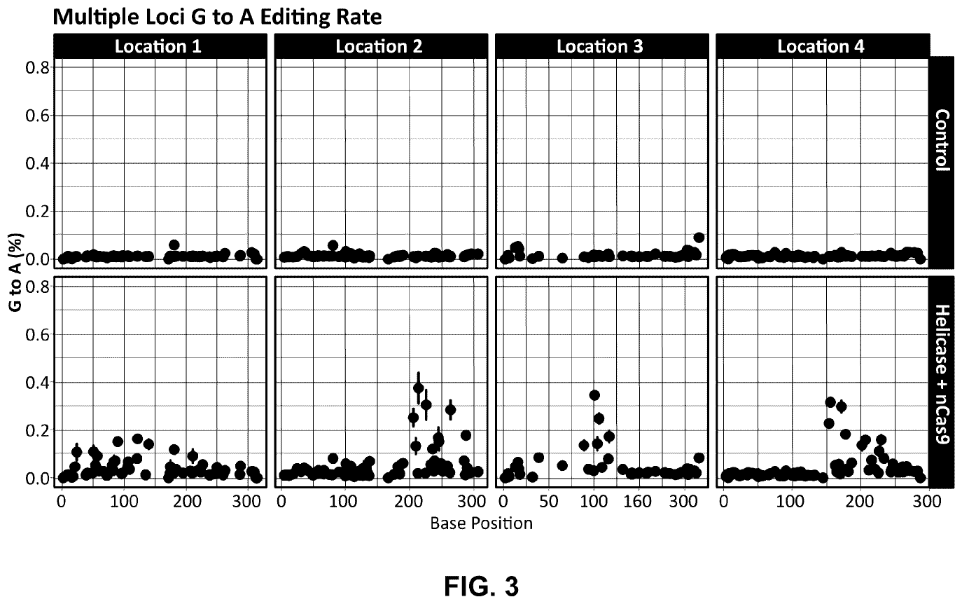

The helicases BLM, NS3h, and PcrA were each evaluated for their ability to promote base editing in the presence of a deaminase and Cas9 nickase (nCas9). Each helicase was fused to cytidine deaminase AID and uracil DNA glycosylase inhibitor (UGI) to generate a helicase fusion. Compositions comprising either (a) a helicase fusion only or (b) a helicase fusion, nCas9, and single guide RNA (sgRNA) targeting an endogenous locus in HEK293FT cells, were prepared. Each composition was incubated with HEK293FT cells and the frequency of G-to-A substitutions of the target locus was assayed (FIGS. 1 and 3). Nucleotide sequences for each of the helicase fusions can be found in Table 3, below.

TABLE 3
Helicase Fusion Nucleotide Sequences

| Helicase Fusion | Nucleotide Sequence (5′ to 3′) |
|---|---|
| AID-BLM-UGI | GCGTTGACATTGATTATTGACTAGTTATTAATAGTAATCAATTA |
| (SEQ ID NO: 41) | CGGGGTCATTAGTTCATAGCCCATATATGGAGTTCCGCGTTAC |
| | ATAACTTACGGTAAATGGCCCGCCTGGCTGACCGCCCAACGAC |
| | CCCCGCCCATTGACGTCAATAATGACGTATGTTCCCATAGTAA |
| | CGCCAATAGGGACTTTCCATTGACGTCAATGGGTGGAGTATTT |
| | ACGGTAAACTGCCCACTTGGCAGTACATCAAGTGTATCATATG |
| | CCAAGTACGCCCCCTATTGACGTCAATGACGGTAAATGGCCCG |
| | CCTGGCATTATGCCCAGTACATGACCTTATGGGACTTTCCTACT |
| | TGGCAGTACATCTACGTATTAGTCATCGCTATTACCATGGTGAT |
| | GCGGTTTTGGCAGTACATCAATGGGCGTGGATAGCGGTTTGAC |
| | TCACGGGGATTTCCAAGTCTCCACCCCATTGACGTCAATGGGA |
| | GTTTGTTTTGGCACCAAAATCAACGGGACTTTCCAAAATGTCGT |
| | AACAACTCCGCCCCATTGACGCAAATGGGCGGTAGGCGTGTAC |
| | GGTGGGAGGTCTATATAAGCAGAGCTGGTTTAGTGAACCGTCA |
| | GATCCGCTAGAGATCCGCGGCCGCGAGAGCCGCCACCATGGAC |
| | AGCCTCTTGATGAACCGGAGGGAGTTTCTTTACCAATTCAAAA |
| | ATGTCCGCTGGGCTAAGGGTCGGCGTGAGACCTACCTGTGCTA |
| | CGTAGTGAAGAGGCGTGACAGTGCTACATCCTTTTCACTGGAC |
| | TTTGGTTATCTTCGCAATAAGAACGGCTGCCACGTGGAATTGCT |
| | CTTCCTCCGCTACATCTCGGACTGGGACCTAGACCCTGGCCGCT |
| | GCTACCGCGTCACCTGGTTCATCTCCTGGAGCCCCTGCTACGAC |
| | TGTGCCCGACATGTGGCCGACTTTCTGCGAGGGAACCCCAACC |
| | TCAGTCTGAGGATCTTCACCGCGCGCCTCTACTTCTGTGAGGAC |
| | CGCAAGGCTGAGCCCGAGGGGCTGCGGCGGCTGCACCGCGCC |
| | GGGGTGCAAATAGCCATCATGACCTTCAAAGATTATTTTTACT |
| | GCTGGAATACTTTTGTAGAAAACCACGGAAGAACTTTCAAAGC |
| | CTGGGAAGGGCTGCATGAAAATTCAGTTCGTCTCTCCAGACAG |
| | CTTCGGCGCATCCTTTTGCCCCTGTATGAGGTTGATGACTTACG |
| | AGACGCATTTCGTACTAGCGGCAGCGAGACTCCCGGGACCTCA |
| | GAGTCCGCCACACCCGAAAGTATGGCTGCTGTTCCTCAAAATA |
| | ATCTACAGGAGCAACTAGAACGTCACTCAGCCAGAACACTTAA |
| | TAATAAATTAAGTCTTTCAAAACCAAAATTTTCAGGTTTCACTT |
| | TTAAAAAGAAAACATCTTCAGATAACAATGTATCTGTAACTAA |
| | TGTGTCAGTAGCAAAAACACCTGTATTAAGAAATAAAGATGTT |
| | AATGTTACCGAAGACTTTTCCTTCAGTGAACCTCTACCCAACAC |
| | CACAAATCAGCAAAGGGTCAAGGACTTCTTTAAAAATGCTCCA |
| | GCAGGACAGGAAACACAGAGAGGTGGATCAAAATCATTATTG |
| | CCAGATTTCTTGCAGACTCCGAAGGAAGTTGTATGCACTACCC |
| | AAAACACACCAACTGTAAAGAAATCCCGGGATACTGCTCTCAA |
| | GAAATTAGAATTTAGTTCTTCACCAGATTCTTTAAGTACCATCA |
| | ATGATTGGGATGATATGGATGACTTTGATACTTCTGAGACTTCA |
| | AAATCATTTGTTACACCACCCCAAAGTCACTTTGTAAGAGTAA |
| | GCACTGCTCAGAAATCAAAAAAGGGTAAGAGAAACTTTTTTAA |
| | AGCACAGCTTTATACAACAAACACAGTAAAGACTGACTTGCCT |
| | CCACCCTCCTCTGAAAGCGAGCAAATAGATTTGACTGAGGAAC |
| | AGAAGGATGACTCAGAATGGTTAAGCAGCGATGTGATTTGCAT |
| | CGATGATGGCCCCATTGCTGAAGTGCATATAAATGAAGATGCT |
| | CAGGAAAGTGACTCTCTGAAAACTCATTTGGAAGATGAAAGAG |
| | ATAATAGCGAAAAGAAGAAGAATTTGGAAGAAGCTGAATTAC |
| | ATTCAACTGAGAAAGTTCCATGTATTGAATTTGATGATGATGA |
| | TTATGATACGGATTTTGTTCCACCTTCTCCAGAAGAAATTATTT |
| | CTGCTTCTTCTTCCTCTTCAAAATGCCTTAGTACGTTAAAGGAC |
| | CTTGACACCTCTGACAGAAAAGAGGATGTTCTTAGCACATCAA |
| | AAGATCTTTTGTCAAAACCTGAGAAAATGAGTATGCAGGAGCT |
| | GAATCCAGAAACCAGCACAGACTGTGACGCTAGACAGATAAG |
| | TTTACAGCAGCAGCTTATTCATGTGATGGAGCACATCTGTAAA |
| | TTAATTGATACTATTCCTGATGATAAACTGAAACTTTTGGATTG |
| | TGGGAACGAACTGCTTCAGCAGCGGAACATAAGAAGGAAACT |
| | TCTAACGGAAGTAGATTTTAATAAAAGTGATGCCAGTCTTCTT |
| | GGCTCATTGTGGAGATACAGGCCTGATTCACTTGATGGCCCTA |
| | TGGAGGGTGATTCCTGCCCTACAGGGAATTCTATGAAGGAGTT |
| | AAATTTTTCACACCTTCCCTCAAATTCTGTTTCTCCTGGGGACT |
| | GTTTACTGACTACCACCCTAGGAAAGACAGGATTCTCTGCCAC |
| | CAGGAAGAATCTTTTTGAAAGGCCTTTATTCAATACCCATTTAC |
| | AGAAGTCCTTTGTAAGTAGCAACTGGGCTGAAACACCAAGACT |
| | AGGAAAAAAAAATGAAAGCTCTTATTTCCCAGGAAATGTTCTC |
| | ACAAGCACTGCTGTGAAAGATCAGAATAAACATACTGCTTCAA |
| | TAAATGACTTAGAAAGAGAAACCCAACCTTCCTATGATATTGA |
| | TAATTTTGACATAGATGACTTTGATGATGATGATGACTGGGAA |
| | GACATAATGCATAATTTAGCAGCCAGCAAATCTTCCACAGCTG |
| | CCTATCAACCCATCAAGGAAGGTCGGCCAATTAAATCAGTATC |
| | AGAAAGACTTTCCTCAGCCAAGACAGACTGTCTTCCAGTGTCA |
| | TCTACTGCTCAAAATATAAACTTCTCAGAGTCAATTCAGAATTA |
| | TACTGACAAGTCAGCACAAAATTTAGCATCCAGAAATCTGAAA |
| | CATGAGCGTTTCCAAAGTCTTAGTTTTCCTCATACAAAGGAAAT |
| | GATGAAGATTTTTCATAAAAAATTTGGCCTGCATAATTTTAGA |
| | ACTAATCAGCTAGAGGCGATCAATGCTGCACTGCTTGGTGAAG |
| | ACTGTTTTATCCTGATGCCGACTGGAGGTGGTAAGAGTTTGTGT |
| | TACCAGCTCCCTGCCTGTGTTTCTCCTGGGGTCACTGTTGTCAT |
| | TTCTCCCTTGAGATCACTTATCGTAGATCAAGTCCAAAAGCTGA |
| | CTTCCTTGGATATTCCAGCTACATATCTGACAGGTGATAAGACT |
| | GACTCAGAAGCTACAAATATTTACCTCCAGTTATCAAAAAAAG |
| | ACCCAATCATAAAACTCCTATATGTCACTCCAGAAAAGATCTG |
| | TGCAAGTAACAGACTCATTTCTACTCTGGAGAATCTCTATGAG |
| | AGGAAGCTCTTGGCACGTTTTGTTATTGATGAAGCACATTGTGT |
| | CAGTCAGTGGGGACATGATTTTCGTCAAGATTACAAAAGAATG |
| | AATATGCTTCGCCAGAAGTTTCCTTCTGTTCCGGTGATGGCTCT |
| | TACGGCCACAGCTAATCCCAGGGTACAGAAGGACATCCTGACT |
| | CAGCTGAAGATTCTCAGACCTCAGGTGTTTAGCATGAGCTTTA |
| | ACAGACATAATCTGAAATACTATGTATTACCGAAAAAGCCTAA |
| | AAAGGTGGCATTTGATTGCCTAGAATGGATCAGAAAGCACCAC |
| | CCATATGATTCAGGGATAATTTACTGCCTCTCCAGGCGAGAAT |
| | GTGACACCATGGCTGACACGTTACAGAGAGATGGGCTCGCTGC |
| | TCTTGCTTACCATGCTGGCCTCAGTGATTCTGCCAGAGATGAAG |
| | TGCAGCAGAAGTGGATTAATCAGGATGGCTGTCAGGTTATCTG |
| | TGCTACAATTGCATTTGGAATGGGGATTGACAAACCGGACGTG |
| | CGATTTGTGATTCATGCATCTCTCCCTAAATCTGTGGAGGGTTA |
| | CTACCAAGAATCTGGCAGAGCTGGAAGAGATGGGGAAATATC |
| | TCACTGCCTGCTTTTCTATACCTATCATGATGTGACCAGACTGA |
| | AAAGACTTATAATGATGGAAAAAGATGGAAACCATCATACAA |
| | GAGAAACTCACTTCAATAATTTGTATAGCATGGTACATTACTGT |
| | GAAAATATAACAGAATGCAGGAGAATACAGCTTTTGGCCTACT |
| | TTGGTGAAAATGGATTTAATCCTGATTTTTGTAAGAAACACCC |
| | AGATGTTTCTTGTGATAATTGCTGTAAAACAAAGGATTATAAA |
| | ACAAGAGATGTGACTGACGATGTGAAAAGTATTGTAAGATTTG |
| | TTCAAGAACATAGTTCATCACAAGGAATGAGAAATATAAAACA |
| | TGTAGGTCCTTCTGGAAGATTTACTATGAATATGCTGGTCGACA |
| | TTTTCTTGGGGAGTAAGAGTGCAAAAATCCAGTCAGGTATATT |
| | TGGAAAAGGATCTGCTTATTCACGACACAATGCCGAAAGACTT |
| | TTTAAAAAGCTGATACTTGACAAGATTTTGGATGAAGACTTAT |
| | ATATCAATGCCAATGACCAGGCGATCGCTTATGTGATGCTCGG |
| | AAATAAAGCACAAACTGTACTAAATGGCAATTTAAAGGTAGAC |
| | TTTATGGAAACAGAAAATTCCAGCAGTGTGAAAAAACAAAAA |
| | GCGTTAGTAGCAAAAGTGTCTCAGAGGGAAGAGATGGTTAAA |
| | AAATGTCTTGGAGAACTTACAGAAGTCTGCAAATCTCTGGGGA |
| | AAGTTTTTGGTGTCCATTACTTCAATATTTTTAATACCGTCACT |
| | CTCAAGAAGCTTGCAGAATCTTTATCTTCTGATCCTGAGGTTTT |
| | GCTTCAAATTGATGGTGTTACTGAAGACAAACTGGAAAAATAT |
| | GGTGCGGAAGTGATTTCAGTATTACAGAAATACTCTGAATGGA |
| | CATCGCCAGCTGAAGACAGTTCCCCAGGGATAAGCCTGTCCAG |
| | CAGCAGAGGCCCCGGAAGAAGTGCCGCTGAGGAGCTTGACGA |
| | GGAAATACCCGTATCTTCCCACTACTTTGCAAGTAAAACCAGA |
| | AATGAAAGGAAGAGGAAAAAGATGCCAGCCTCCCAAAGGTCT |
| | AAGAGGAGAAAAACTGCTTCCAGTGGTTCCAAGGCAAAGGGG |
| | GGGTCTGCCACATGTAGAAAGATATCTTCCAAAACGAAATCCT |
| | CCAGCATCATTGGATCCAGTTCAGCCTCACATACTTCTCAAGCG |
| | ACATCAGGAGCCAATAGCAAATTGGGGATTATGGCTCCACCGA |
| | AGCCTATAAATAGACCGTTTCTTAAGCCTTCATATGCATTCTCA |
| | TCTGGTGGTTCTACTAATCTGTCAGATATTATTGAAAAGGAGA |
| | CCGGTAAGCAACTGGTTATCCAGGAATCCATCCTCATGCTCCC |
| | AGAGGAGGTGGAAGAAGTCATTGGGAACAAGCCGGAAAGCGA |
| | TATACTCGTGCACACCGCCTACGACGAGAGCACCGACGAGAAT |
| | GTCATGCTTCTGACTAGCGACGCCCCTGAATACAAGCCTTGGG |
| | CTCTGGTCATACAGGATAGCAACGGTGAGAACAAGATTAAGAT |
| | GCTCTCTGGTGGTTCTCCCAAGAAGAAGAGGAAAGTCTAACCG |
| | GTCATCATCACCATCACCATTGAGTTTAAACCCGCTGATCAGCC |
| | TCGACTGTGCCTTCTAGTTGCCAGCCATCTGTTGTTTGCCCCTC |
| | CCCCGTGCCTTCCTTGACCCTGGAAGGTGCCACTCCCACTGTCC |
| | TTTCCTAATAAAATGAGGAAATTGCATCGCATTGTCTGAGTAG |
| | GTGTCATTCTATTCTGGGGGGTGGGGTGGGGCAGGACAGCAAG |
| | GGGGAGGATTGGGAAGACAATAGCAGGCATGCTGGGGATGCG |
| | GTGGGCTCTATGGCTTCTG |
| AID-NS3h-UGI | GCGTTGACATTGATTATTGACTAGTTATTAATAGTAATCAATTA |
| (SEQ ID NO: 42) | CGGGGTCATTAGTTCATAGCCCATATATGGAGTTCCGCGTTAC |
| | ATAACTTACGGTAAATGGCCCGCCTGGCTGACCGCCCAACGAC |
| | CCCCGCCCATTGACGTCAATAATGACGTATGTTCCCATAGTAA |
| | CGCCAATAGGGACTTTCCATTGACGTCAATGGGTGGAGTATTT |
| | ACGGTAAACTGCCCACTTGGCAGTACATCAAGTGTATCATATG |
| | CCAAGTACGCCCCCTATTGACGTCAATGACGGTAAATGGCCCG |
| | CCTGGCATTATGCCCAGTACATGACCTTATGGGACTTTCCTACT |
| | TGGCAGTACATCTACGTATTAGTCATCGCTATTACCATGGTGAT |
| | GCGGTTTTGGCAGTACATCAATGGGCGTGGATAGCGGTTTGAC |
| | TCACGGGGATTTCCAAGTCTCCACCCCATTGACGTCAATGGGA |
| | GTTTGTTTTGGCACCAAAATCAACGGGACTTTCCAAAATGTCGT |
| | AACAACTCCGCCCCATTGACGCAAATGGGCGGTAGGCGTGTAC |
| | GGTGGGAGGTCTATATAAGCAGAGCTGGTTTAGTGAACCGTCA |
| | GATCCGCTAGAGATCCGCGGCCGCGAGAGCCGCCACCATGGAC |
| | AGCCTCTTGATGAACCGGAGGGAGTTTCTTTACCAATTCAAAA |
| | ATGTCCGCTGGGCTAAGGGTCGGCGTGAGACCTACCTGTGCTA |
| | CGTAGTGAAGAGGCGTGACAGTGCTACATCCTTTTCACTGGAC |
| | TTTGGTTATCTTCGCAATAAGAACGGCTGCCACGTGGAATTGCT |
| | CTTCCTCCGCTACATCTCGGACTGGGACCTAGACCCTGGCCGCT |
| | GCTACCGCGTCACCTGGTTCATCTCCTGGAGCCCCTGCTACGAC |
| | TGTGCCCGACATGTGGCCGACTTTCTGCGAGGGAACCCCAACC |
| | TCAGTCTGAGGATCTTCACCGCGCGCCTCTACTTCTGTGAGGAC |
| | CGCAAGGCTGAGCCCGAGGGGCTGCGGCGGCTGCACCGCGCC |
| | GGGGTGCAAATAGCCATCATGACCTTCAAAGATTATTTTTACT |
| | GCTGGAATACTTTTGTAGAAAACCACGGAAGAACTTTCAAAGC |
| | CTGGGAAGGGCTGCATGAAAATTCAGTTCGTCTCTCCAGACAG |
| | CTTCGGCGCATCCTTTTGCCCCTGTATGAGGTTGATGACTTACG |
| | AGACGCATTTCGTACTAGCGGCAGCGAGACTCCCGGGACCTCA |
| | GAGTCCGCCACACCCGAAAGTATGATGGTGGACTTCATACCCG |
| | TTGAGTCTATGGAAACTACCATGCGGTCTCCGGTCTTCACAGA |
| | CAACTCAACCCCCCCGGCTGTACCGCAGACATTCCAAGTGGCA |
| | CATCTGCACGCTCCTACTGGCAGCGGCAAGAGCACCAAAGTGC |
| | CGGCTGCGTATGCAGCCCAAGGGTACAAGGTGCTCGTCCTGAA |
| | CCCGTCCGTTGCCGCCACCTTAGGGTTTGGGGCGTATATGTCCA |
| | AGGCACACGGTATCGACCCTAACATCAGAACTGGGGTAAGGA |
| | CCATTACCACGGGCGGCTCCATTACGTACTCCACCTATGGCAA |
| | GTTCCTTGCCGACGGTGGCTGTTCTGGGGGCGCCTATGACATC |
| | ATAATATGTGATGAGTGCCACTCAACTGACTCGACTACCATCTT |
| | GGGCATCGGCACAGTCCTGGACCAAGCGGAGACGGCTGGAGC |
| | GCGGCTCGTCGTGCTCGCCACCGCTACACCTCCGGGATCGGTT |
| | ACCGTGCCACACCCCAATATCGAGGAAATAGGCCTGTCCAACA |
| | ATGGAGAGATCCCCTTCTATGGCAAAGCCATCCCCATTGAGGC |
| | CATCAAGGGGGGGAGGCATCTCATTTTCTGCCATTCCAAGAAG |
| | AAATGTGACGAGCTCGCCGCAAAGCTGACAGGCCTCGGACTGA |
| | ACGCTGTAGCATATTACCGGGGCCTTGATGTGTCCGTCATACC |
| | GCCTATCGGAGACGTCGTTGTCGTGGCAACAGACGCTCTAATG |
| | ACGGGTTTCACCGGCGATTTTGACTCAGTGATCGACTGCAATA |
| | CATGTGTCACCCAGACAGTCGACTTCAGCTTGGATCCCACCTTC |
| | ACCATTGAGACGACGACCGTGCCCCAAGACGCGGTGTCGCGCT |
| | CGCAACGGCGAGGTAGAACTGGCAGGGGTAGGAGTGGCATCT |
| | ACAGGTTTGTGACTCCAGGAGAACGGCCCTCGGGCATGTTCGA |
| | TTCTTCGGTCCTGTGTGAGTGCTATGACGCGGGCTGTGCTTGGT |
| | ATGAGCTCACGCCCGCTGAGACCTCGGTTAGGTTGCGGGCTTA |
| | CCTAAATACACCAGGGTTGCCCGTCTGCCAGGACCATCTGGAG |
| | TTCTGGGAGAGCGTCTTCACAGGCCTCACCCACATAGATGCCC |
| | ACTTCCTGTCCCAGACTAAACAGGCAGGAGACAACTTTCCTTA |
| | CCTGGTGGCATATCAAGCTACAGTGTGCGCCAGGGCTCAAGCT |
| | CCACCTCCATCGTGGGACCAAATGTGGAAGTGTCTCATACGGC |
| | TGAAACCTACACTGCACGGGCCAACACCCCTGCTGTATAGGCT |
| | AGGAGCCGTCCAAAATGAGGTCATCCTCACACACCCCATAACT |
| | AAATACATCATGGCATGCATGTCGGCTGACCTGGAGGTCGTCA |
| | CTTCTGGTGGTTCTACTAATCTGTCAGATATTATTGAAAAGGAG |
| | ACCGGTAAGCAACTGGTTATCCAGGAATCCATCCTCATGCTCC |
| | CAGAGGAGGTGGAAGAAGTCATTGGGAACAAGCCGGAAAGCG |
| | ATATACTCGTGCACACCGCCTACGACGAGAGCACCGACGAGAA |
| | TGTCATGCTTCTGACTAGCGACGCCCCTGAATACAAGCCTTGG |
| | GCTCTGGTCATACAGGATAGCAACGGTGAGAACAAGATTAAG |
| | ATGCTCTCTGGTGGTTCTCCCAAGAAGAAGAGGAAAGTCTAAC |
| | CGGTCATCATCACCATCACCATTGAGTTTAAACCCGCTGATCA |
| | GCCTCGACTGTGCCTTCTAGTTGCCAGCCATCTGTTGTTTGCCC |
| | CTCCCCCGTGCCTTCCTTGACCCTGGAAGGTGCCACTCCCACTG |
| | TCCTTTCCTAATAAAATGAGGAAATTGCATCGCATTGTCTGAGT |
| | AGGTGTCATTCTATTCTGGGGGGTGGGGTGGGGCAGGACAGCA |
| | AGGGGGAGGATTGGGAAGACAATAGCAGGCATGCTGGGGATG |
| | CGGTGGGCTCTATGGCTTCTG |
| AID-PcrA-UGI | GCGTTGACATTGATTATTGACTAGTTATTAATAGTAATCAATTA |
| (SEQ ID NO: 43) | CGGGGTCATTAGTTCATAGCCCATATATGGAGTTCCGCGTTAC |
| | ATAACTTACGGTAAATGGCCCGCCTGGCTGACCGCCCAACGAC |
| | CCCCGCCCATTGACGTCAATAATGACGTATGTTCCCATAGTAA |
| | CGCCAATAGGGACTTTCCATTGACGTCAATGGGTGGAGTATTT |
| | ACGGTAAACTGCCCACTTGGCAGTACATCAAGTGTATCATATG |
| | CCAAGTACGCCCCCTATTGACGTCAATGACGGTAAATGGCCCG |
| | CCTGGCATTATGCCCAGTACATGACCTTATGGGACTTTCCTACT |
| | TGGCAGTACATCTACGTATTAGTCATCGCTATTACCATGGTGAT |
| | GCGGTTTTGGCAGTACATCAATGGGCGTGGATAGCGGTTTGAC |
| | TCACGGGGATTTCCAAGTCTCCACCCCATTGACGTCAATGGGA |
| | GTTTGTTTTGGCACCAAAATCAACGGGACTTTCCAAAATGTCGT |
| | AACAACTCCGCCCCATTGACGCAAATGGGCGGTAGGCGTGTAC |
| | GGTGGGAGGTCTATATAAGCAGAGCTGGTTTAGTGAACCGTCA |
| | GATCCGCTAGAGATCCGCGGCCGCGAGAGCCGCCACCATGGAC |
| | AGCCTCTTGATGAACCGGAGGGAGTTTCTTTACCAATTCAAAA |
| | ATGTCCGCTGGGCTAAGGGTCGGCGTGAGACCTACCTGTGCTA |
| | CGTAGTGAAGAGGCGTGACAGTGCTACATCCTTTTCACTGGAC |
| | TTTGGTTATCTTCGCAATAAGAACGGCTGCCACGTGGAATTGCT |
| | CTTCCTCCGCTACATCTCGGACTGGGACCTAGACCCTGGCCGCT |
| | GCTACCGCGTCACCTGGTTCATCTCCTGGAGCCCCTGCTACGAC |
| | TGTGCCCGACATGTGGCCGACTTTCTGCGAGGGAACCCCAACC |
| | TCAGTCTGAGGATCTTCACCGCGCGCCTCTACTTCTGTGAGGAC |
| | CGCAAGGCTGAGCCCGAGGGGCTGCGGCGGCTGCACCGCGCC |
| | GGGGTGCAAATAGCCATCATGACCTTCAAAGATTATTTTTACT |
| | GCTGGAATACTTTTGTAGAAAACCACGGAAGAACTTTCAAAGC |
| | CTGGGAAGGGCTGCATGAAAATTCAGTTCGTCTCTCCAGACAG |
| | CTTCGGCGCATCCTTTTGCCCCTGTATGAGGTTGATGACTTACG |
| | AGACGCATTTCGTACTAGCGGCAGCGAGACTCCCGGGACCTCA |
| | GAGTCCGCCACACCCGAAAGTATGATGAATGCCCTGCTGAACC |
| | ATATGAATACAGAACAATCCGAGGCGGTAAAGACCACAGAAG |
| | GCCCCTTGTTGATCATGGCGGGGGCTGGGAGTGGTAAAACGAG |
| | GGTCCTTACTCACCGAATAGCGTACTTGCTTGACGAAAAGGAC |
| | GTGAGTCCATATAACGTGCTTGCCATTACCTTCACAAACAAGG |
| | CTGCTAGAGAGATGAAGGAAAGAGTCCAAAAACTTGTCGGTG |
| | ACCAAGCGGAGGTCATTTGGATGTCTACCTTCCATTCTATGTGC |
| | GTTCGCATACTTCGGCGAGACGCGGATAGGATTGGGATCGAAC |
| | GGAACTTCACGATAATAGATCCTACAGATCAAAAGTCTGTAAT |
| | AAAAGATGTTCTCAAAAATGAGAATATAGATAGCAAAAAATTT |
| | GAACCCCGAATGTTCATAGGTGCCATATCAAACTTGAAGAACG |
| | AACTCAAAACACCTGCGGACGCACAAAAGGAAGCAACAGACT |
| | ACCACAGTCAGATGGTCGCAACTGTTTATTCCGGCTACCAACG |
| | ACAGCTGAGTCGGAATGAAGCACTGGATTTTGATGATCTGATC |
| | ATGACTACTATTAACCTTTTTGAAAGAGTACCGGAAGTGTTGG |
| | AATATTACCAAAACAAATTTCAATATATCCACGTTGATGAATA |
| | CCAAGATACTAATAAGGCACAGTATACATTGGTAAAGCTGCTG |
| | GCGTCAAAGTTTAAAAATCTTTGCGTGGTCGGGGATAGTGACC |
| | AGAGCATATACGGTTGGCGCGGCGCCGACATACAGAATATCTT |
| | GTCCTTCGAGAAAGATTATCCTGAGGCGAATACAATCTTCCTT |
| | GAGCAGAATTATAGATCTACAAAAACTATTTTGAACGCGGCTA |
| | ACGAAGTAATAAAAAATAATAGTGAGCGAAAGCCTAAAGGTC |
| | TGTGGACAGCTAACACAAATGGTGAAAAGATTCATTACTACGA |
| | AGCAATGACTGAACGAGACGAAGCGGAGTTCGTCATCCGGGA |
| | AATAATGAAACACCAACGCAACGGCAAGAAATACCAAGACAT |
| | GGCAATTCTGTACAGGACCAATGCGCAATCCAGAGTTCTCGAA |
| | GAAACCTTTATGAAGAGCAATATGCCATACACGATGGTTGGAG |
| | GCCAAAAATTCTATGATAGGAAAGAGATCAAAGACCTGCTGA |
| | GCTACCTCCGAATCATTGCCAACAGTAACGACGACATCTCACT |
| | TCAACGGATTATTAACGTACCGAAACGCGGGGTTGGACCCTCA |
| | TCAGTTGAGAAAGTTCAAAACTATGCGTTGCAGAACAATATTT |
| | CCATGTTTGACGCTCTTGGAGAAGCTGATTTTATCGGCTTGTCA |
| | AAGAAAGTAACCCAGGAGTGTCTTAACTTTTACGAACTGATAC |
| | AAAGCCTGATAAAGGAACAGGAATTCCTTGAGATCCACGAGAT |
| | CGTAGATGAAGTTCTGCAAAAATCCGGCTATCGGGAAATGTTG |
| | GAAAGGGAGAACACGCTCGAAAGTAGGTCAAGACTCGAGAAC |
| | ATAGATGAGTTCATGTCAGTGCCCAAAGACTATGAGGAGAATA |
| | CGCCCCTTGAAGAGCAGTCATTGATCAATTTCCTTACTGACCTG |
| | TCACTCGTTGCCGATATTGACGAAGCTGACACTGAGAATGGGG |
| | TAACATTGATGACGATGCACAGTGCTAAGGGATTGGAGTTTCC |
| | CATAGTCTTCATCATGGGTATGGAGGAGTCCCTCTTTCCACACA |
| | TTCGGGCAATCAAATCCGAGGATGATCATGAGATGCAAGAGG |
| | AGCGCAGAATCTGTTACGTTGCGATTACACGAGCGGAGGAGGT |
| | TCTTTATATTACACACGCAACGTCTCGGATGCTCTTTGGACGCC |
| | CACAGTCAAACATGCCCTCTAGGTTCCTTAAGGAAATACCCGA |
| | GAGCCTGCTGGAGAACCATAGCTCAGGTAAGCGGCAGACCAT |
| | ACAACCAAAAGCTAAGCCGTTCGCCAAACGCGGCTTCTCACAG |
| | CGCACGACTAGCACGAAGAAGCAGGTGCTCTCCAGCGATTGGA |
| | ATGTTGGCGATAAGGTTATGCACAAAGCTTGGGGAGAGGGCAT |
| | GGTTTCCAATGTGAATGAGAAAAATGGATCCATAGAGCTCGAC |
| | ATCATCTTTAAGAGCCAGGGCCCAAAGCGCCTGCTCGCTCAGT |
| | TCGCTCCAATCGAGAAAAAAGAAGACTCTGGTGGTTCTACTAA |
| | TCTGTCAGATATTATTGAAAAGGAGACCGGTAAGCAACTGGTT |
| | ATCCAGGAATCCATCCTCATGCTCCCAGAGGAGGTGGAAGAAG |
| | TCATTGGGAACAAGCCGGAAAGCGATATACTCGTGCACACCGC |
| | CTACGACGAGAGCACCGACGAGAATGTCATGCTTCTGACTAGC |
| | GACGCCCCTGAATACAAGCCTTGGGCTCTGGTCATACAGGATA |
| | GCAACGGTGAGAACAAGATTAAGATGCTCTCTGGTGGTTCTCC |
| | CAAGAAGAAGAGGAAAGTCTAACCGGTCATCATCACCATCACC |
| | ATTGAGTTTAAACCCGCTGATCAGCCTCGACTGTGCCTTCTAGT |
| | TGCCAGCCATCTGTTGTTTGCCCCTCCCCCGTGCCTTCCTTGAC |
| | CCTGGAAGGTGCCACTCCCACTGTCCTTTCCTAATAAAATGAG |
| | GAAATTGCATCGCATTGTCTGAGTAGGTGTCATTCTATTCTGGG |
| | GGGTGGGGTGGGGCAGGACAGCAAGGGGGAGGATTGGGAAGA |
| | CAATAGCAGGCATGCTGGGGATGCGGTGGGCTCTATGGCTTCT |
| | G |

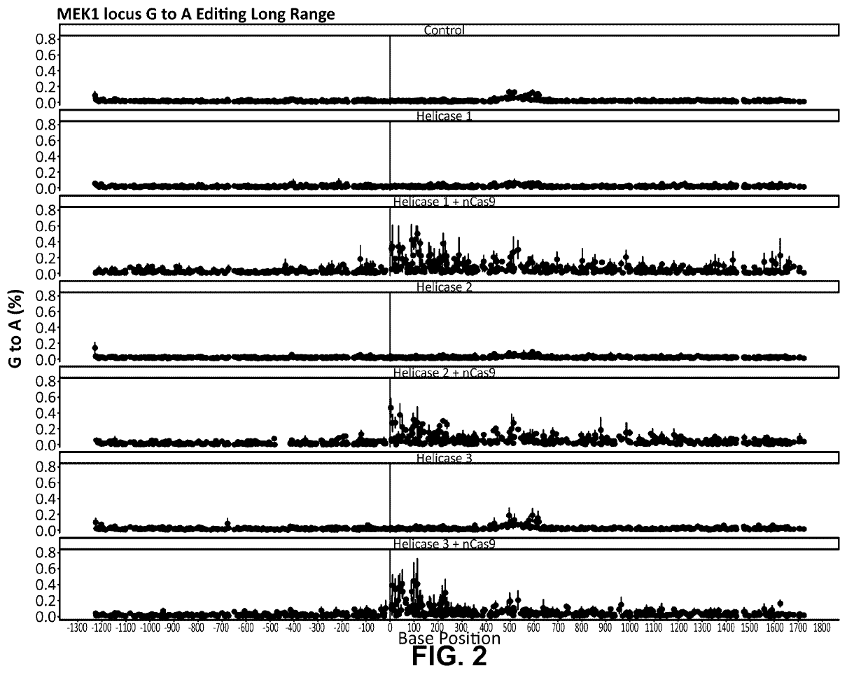

No G-to-A substitutions were observed in HEK293FT cells incubated with the compositions comprising only the helicase fusions, suggesting no background off-target editing occurred (FIG. 1-3). Elevated levels of G-to-A substitutions were observed in HEK293FT cells incubated with compositions comprising the helicase fusion, nCas9, and sgRNA targeting the MEK1 locus (FIG. 1). The range of these substitutions was observed as far as more than 1,500 base pairs from the nick site (FIG. 2). The helicase fusions were also observed to promote G-to-A substitutions across multiple loci when incubated with nCas9 and sgRNAs targeting those loci (FIG. 3).

### Example 2

### HACE Design and Implementation

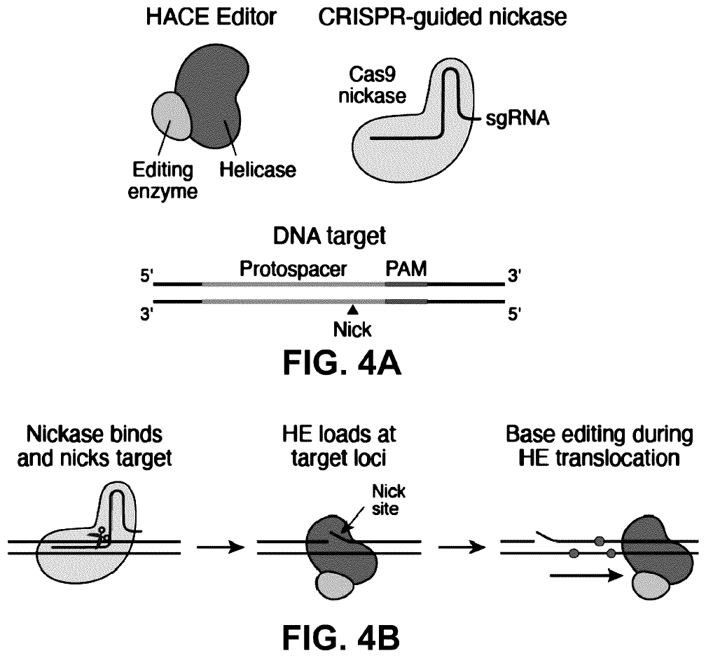

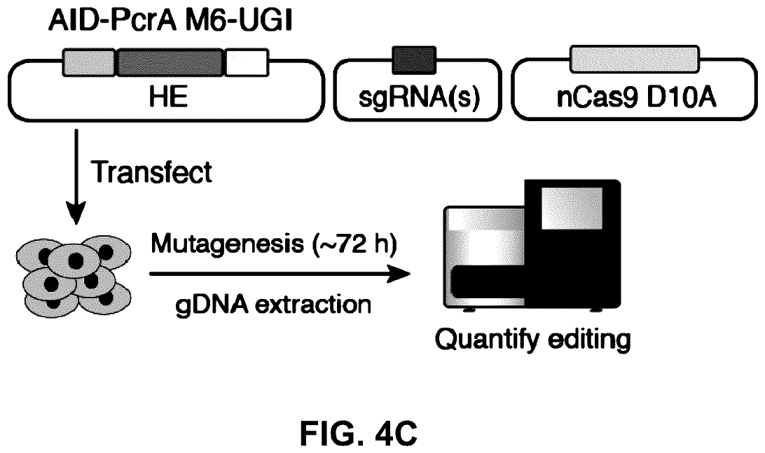

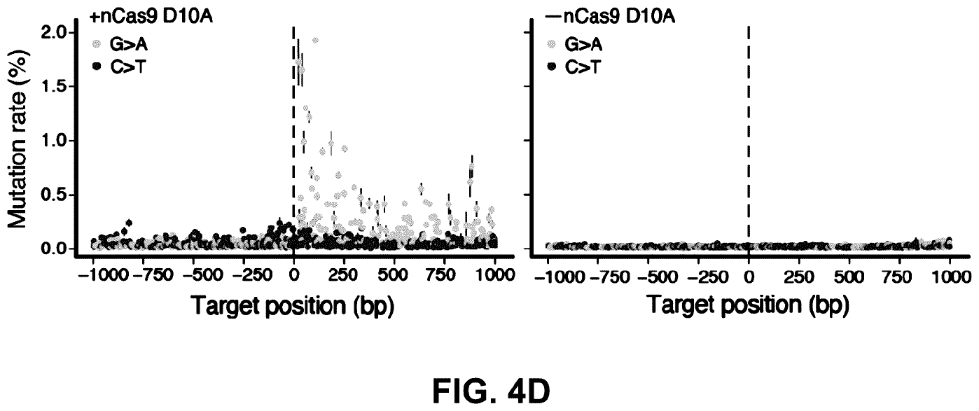

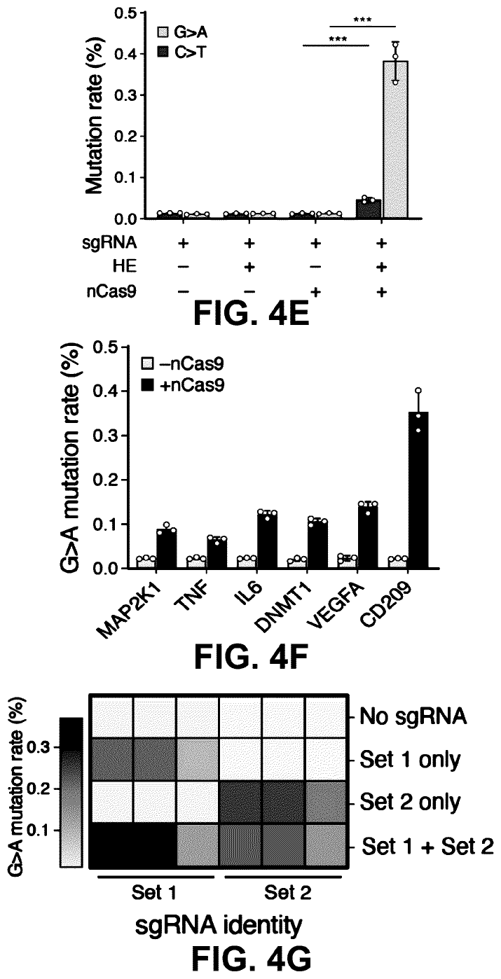

An example HACE Editor (HE) protein was constructed by fusing a hyperactive mutant of activation-induced cytidine deaminase (AID*Δ) with a Geobacillus stearothermophilus PcrA helicase that was previously optimized for processivity (PcrA M6). A uracil DNA glycosylase inhibitor (UGI), which has been shown to facilitate C: G>T: A mutations, at the 3′ end of the HE (FIG. 4C), was also appended to the fusion. To generate a targeted single-stranded nick, a SpCas9 nickase (nCas9; D10A) and corresponding single-guide RNA (sgRNA) were used. After the nCas9 creates a nick, the HE may load at the nick, start unwinding the DNA, and generate random mutations in the process.

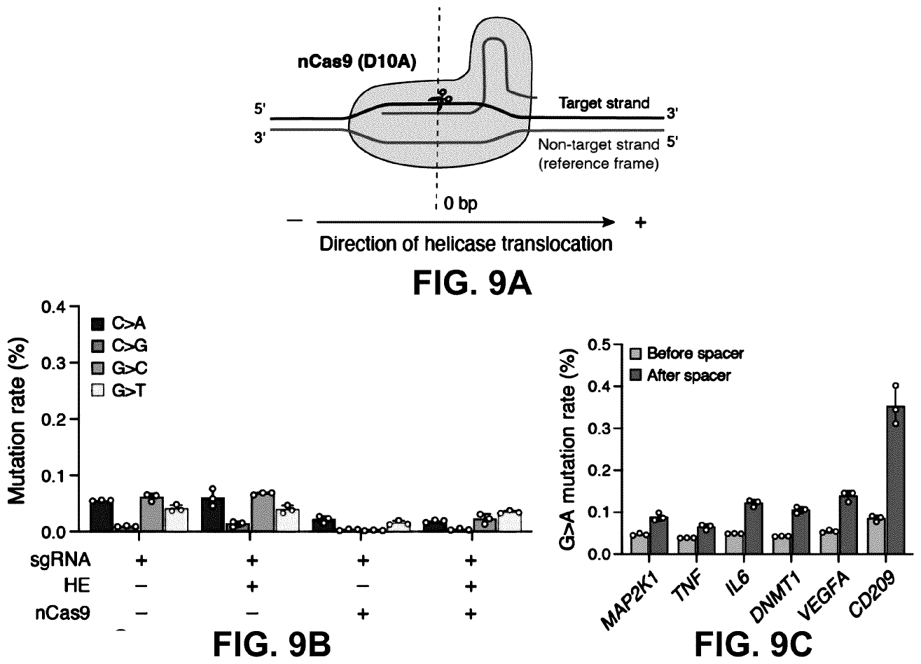

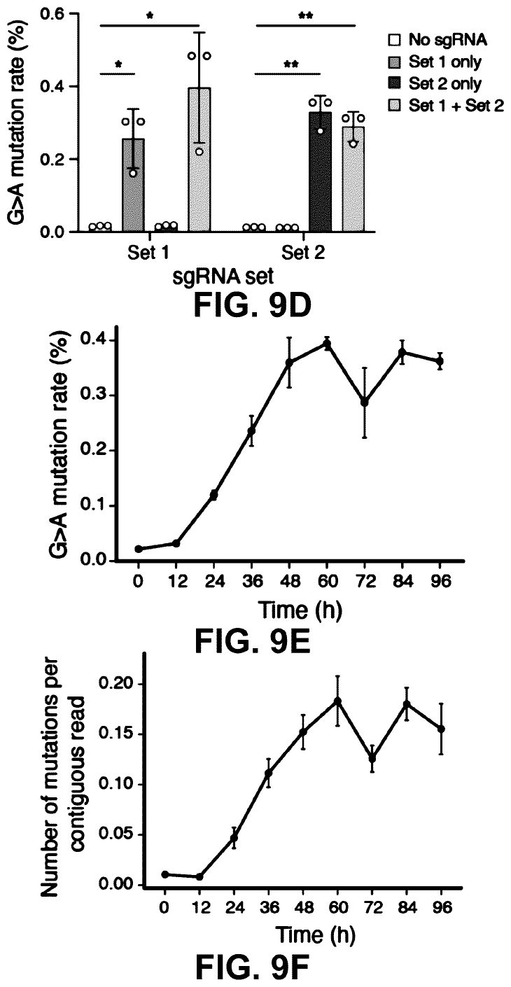

Separate plasmids expressing HE, nCas9, and a sgRNA targeting the HEK3 locus were transfected into HEK293FT cells. Cells were collected 72 hours after transfection, and editing rates were evaluated by amplicon sequencing. Directional editing was observed from the nick-site in the presence of the HE, nCas9, and sgRNA (FIG. 4D). Importantly, elevated mutation levels were not observed in cells transfected with only HE or only with nCas9 and sgRNA, suggesting that editing is driven by the HE and is guide-specific. Using the non-target strand of the Cas9 (strand that is not bound by the sgRNA) that runs from 3′ to 5′ as the frame of reference (FIG. 9A), a significant increase in mutation rates across a ˜1000 bp window downstream of the nick site, but not upstream, suggested that editing occurs in a directional manner (FIG. 4D). A strand bias with G>A substitutions occurring at a higher rate than C>T substitutions was detected, which likely reflects preferential repair of mismatches on the nicked strand. The direction of editing corresponds to helicase loading on the non-nicked strand and translocating in the 3′ to 5′ direction, which is consistent with known mechanisms of PcrA helicases. The editing rate downstream of the nick site was quantified, and an average G>A mutation rate of 0.38% per base and an average C>T mutation rate of 0.046% per base was observed (FIG. 4E, Methods), representing a significantly higher mutation rate than cells transfected with nCas9 or HE only (unpaired t-test, P<0.001 for +/−nCas9 in both C>T and G>A groups). This also is a significantly elevated mutation rate as compared to the replication error rate of human cells. The rates of other transition and transversion mutation modes were comparable to the background, providing further support for the specificity and targeting of the fusion protein (FIG. 9B).

### HACE Flexibility and Programmability

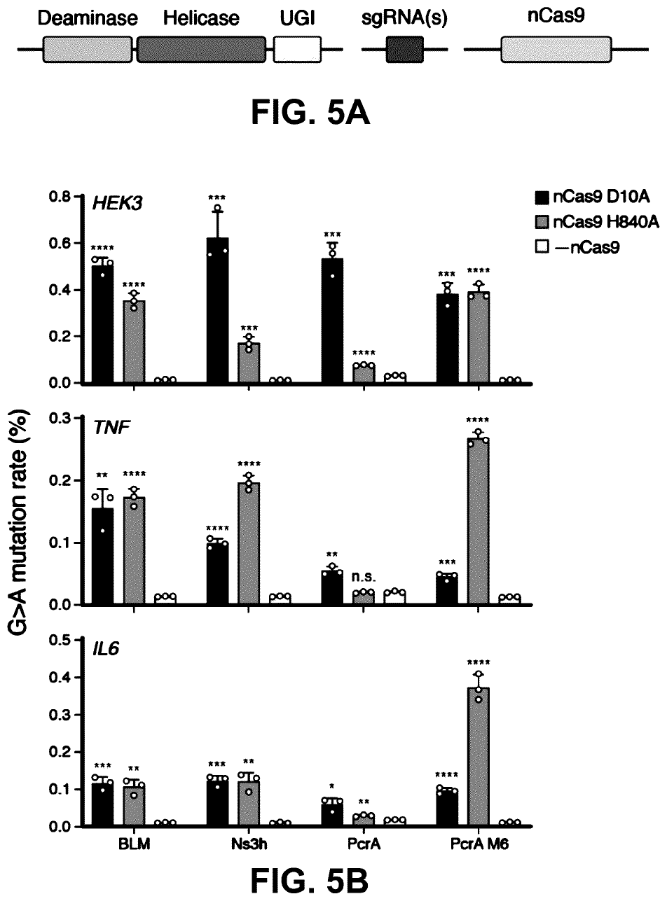

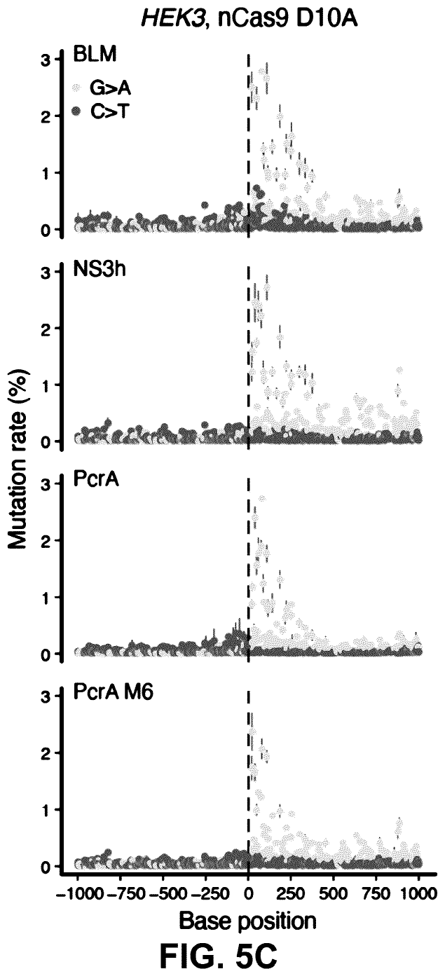

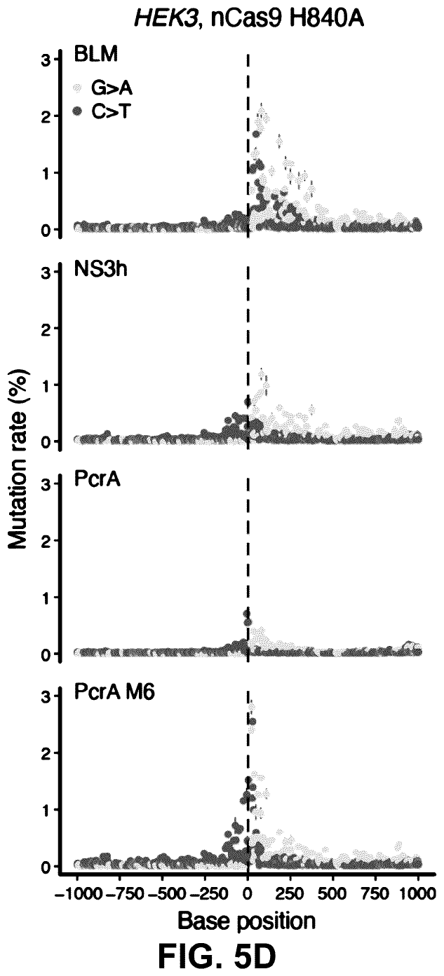

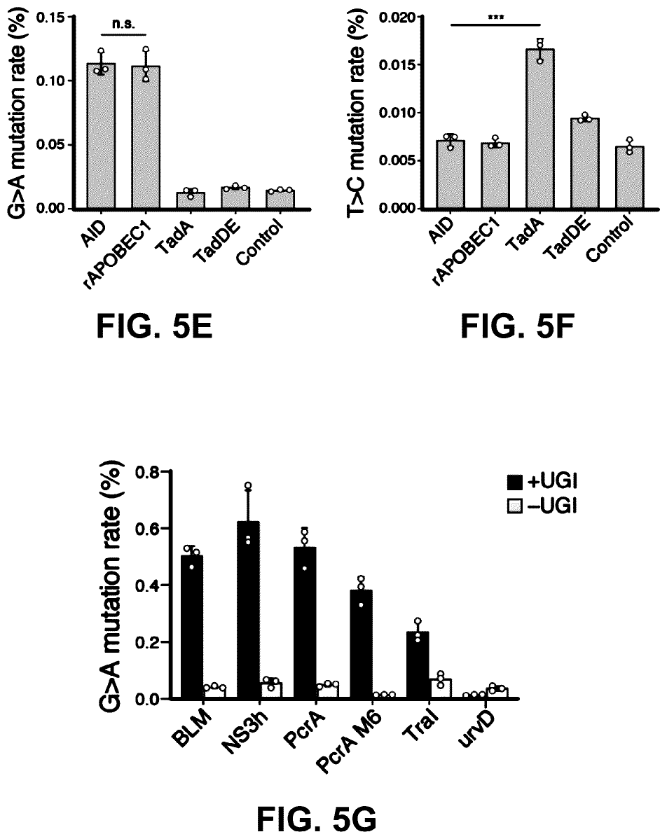

In one embodiment, HACE constructs were tested with different helicase enzymes. Elevated mutation rates were detected using either target-strand nickase (nCas9 D10A) or non-target-strand nickase (nCas9 H840A), with all constructs showing significant editing across the three loci for at least one nickase variant (FIG. 5B). The preference of the target and non-target nickase is loci dependent, though the BLM helicase appears to prefer both nickase variants equally (FIG. 5B).

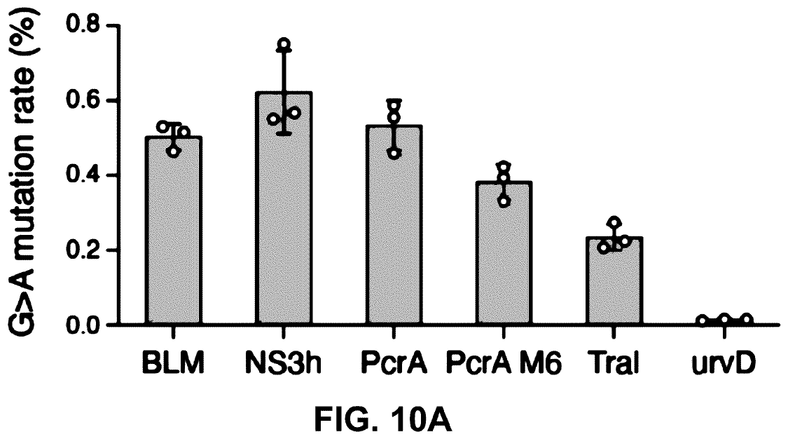

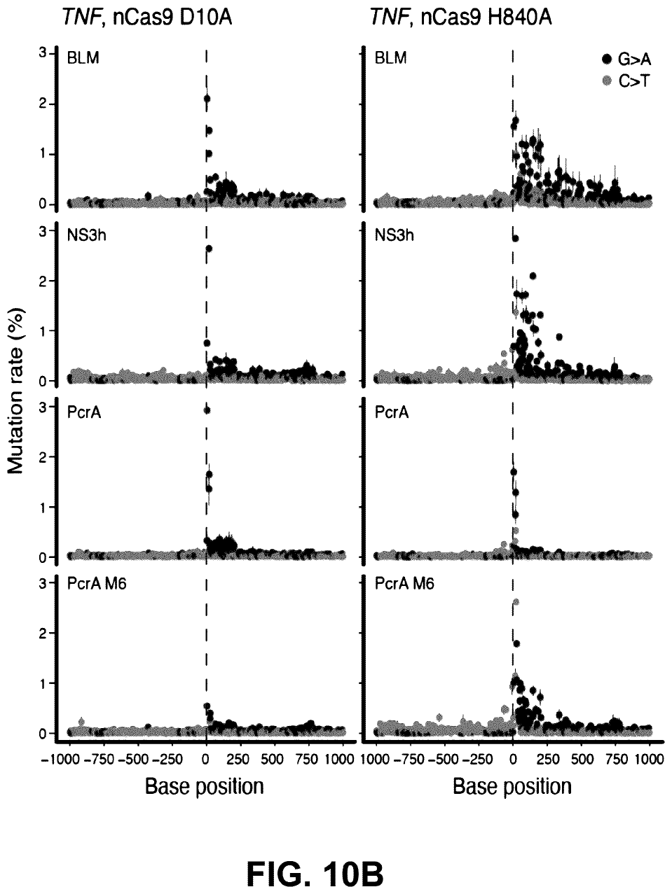

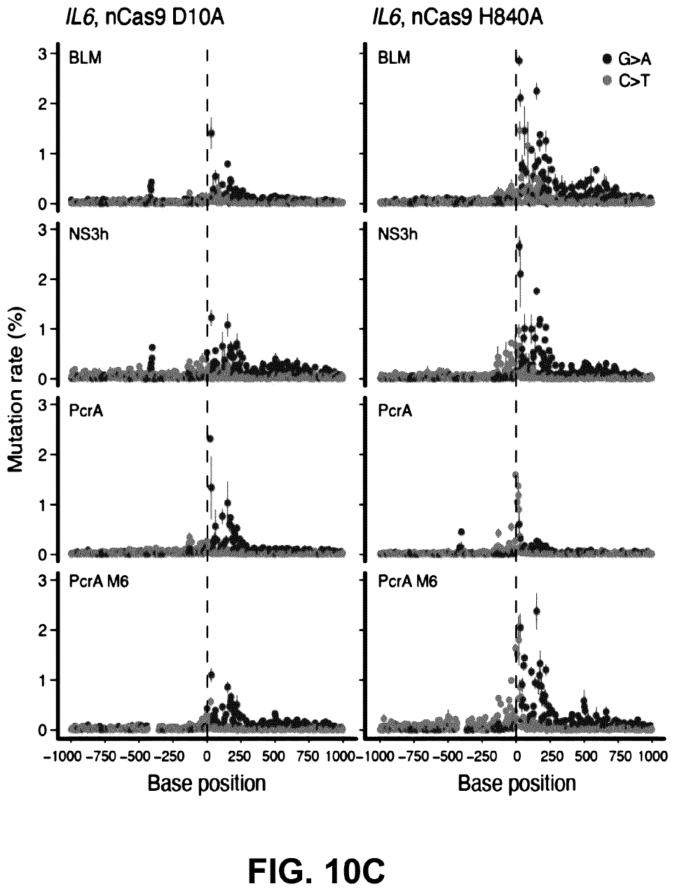

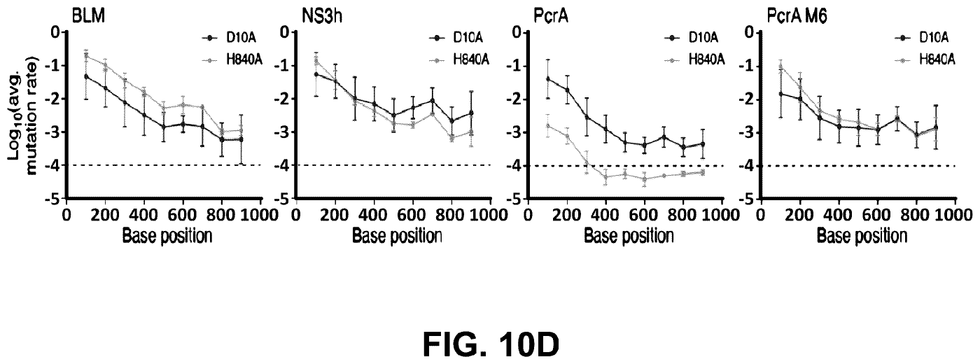

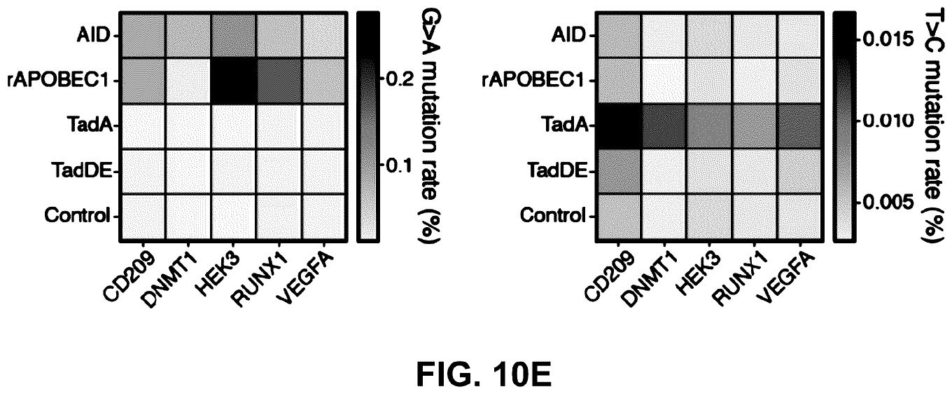

The editing range of different HE constructs was then characterized. For each helicase, nickase, and loci combination, the respective HE, nCas9, and sgRNA combination was transfected into HEK293FT cells, genomic DNA was harvested after three days, and then a 1000 bp window was amplified by PCR for sequencing. The target-strand and non-target strand nickase had similar long range editing performance (FIG. 5C-D, FIG. 10B-C). Across a ˜1000 bp window around the nick, it was observed that the helicases still preferred to translocate in the downstream direction for the non-target strand nickase (nCas9 H840A), even though the nick was now on the opposite strand. Thus, the nCas9 binding and DNA-sgRNA duplex might obstruct the helicase loading on the non-nicked single-stranded DNA.

In another embodiment, the local average mutation rate was calculated in 100 bp bins for each genomic loci (FIG. 10D). A decreasing mutation rate was observed as a function of distance from the nick for all helicases profiled with both nickase variants. BLM, NS3h, and PcrA M6 helicases all demonstrated elevated editing (>10-3 G>A mutation rate per base) within 500 bp from the nick site. The mutation rate of PcrA M6 stabilized past 500 bp (at ˜10-3 G>A mutation rate per base), suggesting long, consistent, long-range editing up to 1000 bp away from the nick site. This range is an order of magnitude longer than previous Cas9-directed editing tools.

In another embodiment, different base-editing systems for HACE were explored. HEs were fused with diverse deaminases including (1) other cytosine deaminase enzymes that introduce C>T and G>A substitutions (rAPOBEC1 (17)), (2) adenosine deaminase enzymes that introduce A>G and T>C substitutions (TadA-8e (18)), and (3) an engineered dual base editor that can perform both cytosine and adenine base editing (TadDE). These constructs were tested in HEK293T cells and mutation rates were quantified by amplicon sequencing (FIG. 5E-F). It was observed that rAPOBEC1 performs comparably to AID*Δ in introducing G>A base edits (unpaired t-test, P>0.05). TadA was able to induce T>C edits at a significantly higher rate than G>A editors (unpaired t-test, P<0.001). On the other hand, the dual TadDE editor only induced minor levels of G>A and T>C editing. The deaminases rAPOBEC1 and TadA introduced mutations across diverse genomic loci (FIG. 10E), demonstrating that HACE utilizing different deaminase fusions can introduce diverse programmable base editing modes.

In another embodiment, it was found that the fusion of uracil glycosylase inhibitor (UGI) significantly elevated the editing levels for AID (FIG. 5G), consistent with reports from previous cytidine-base editor studies. The results demonstrate that HACE editing rates can be tuned by varying the helicase and nickase variants used, making it suitable for diverse applications.

### HACE is Minimally Perturbative to Mammalian Cells

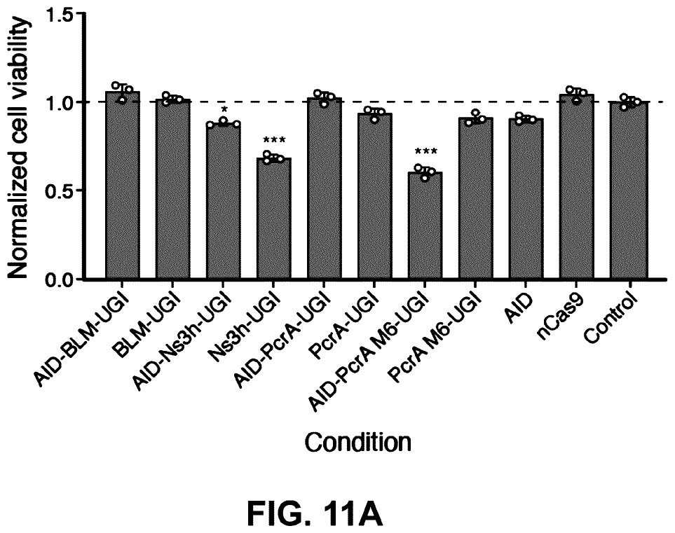

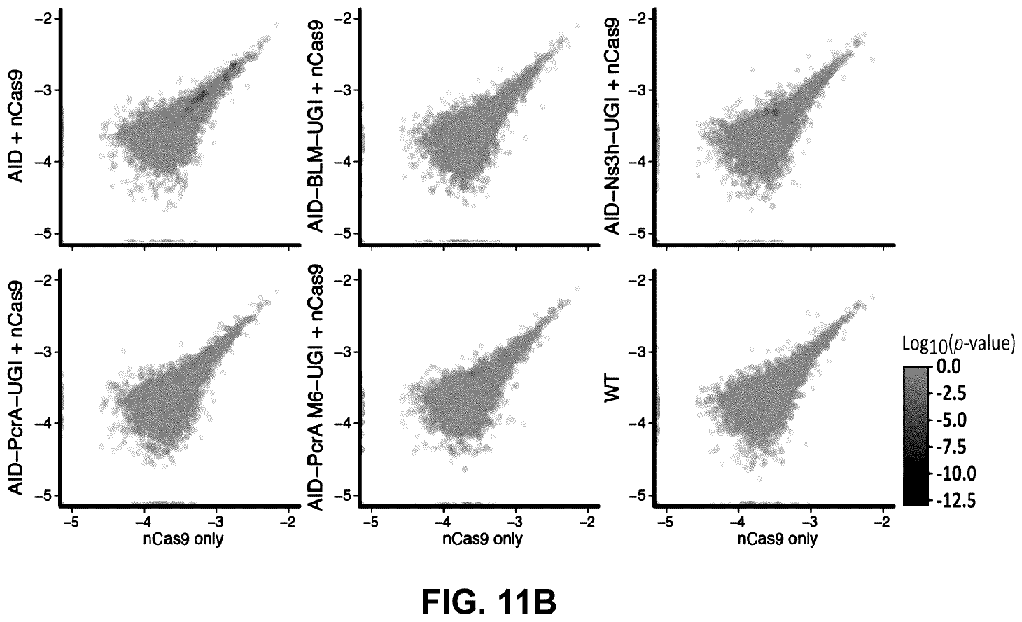

Additionally, HACE constructs are well tolerated in transfection experiments. The effects of different HACE constructs on cell viability were then quantified. To do so, the cell viability was quantified using a luciferase-based ATP-assay (CellTiter-Glo) across various helicase constructs both with and without deaminase along with a loci-targeting sgRNA and nCas9 (FIG. 11A). It was found that HEs constructed with BLM and PcrA helicases did not result in a significant decrease in cell viability (unpaired t-test, p>0.05 for each group). However, AID-NS3h-UGI leads to decrease in cell viability (unpaired t-test, p<0.05), which is possibly related to the toxicity of NS3h helicases since it also acts on RNA. AID-PcrA M6-UGI also significantly decreases cell viability (unpaired t-test, p<0.001), while PcrA M6 alone did not affect cell viability (unpaired t-test, p=0.118). These results suggest that helicases used for HACE are well tolerated for cell viability.

In another embodiment, it was explored whether HACE generated elevated mutation rates in non-targeted parts of the genome. To do so, whole exome sequencing of cells expressing different HE variants and AID overexpression at high coverage (average ˜1000× coverage across the exome) was performed. To increase the statistical power to detect elevated mutation rates, the genome was binned into 100 kb bins and the editing rate of each bin was calculated, then compared to the editing rate between HE variants and control cells (FIG. 10B). Of the 16621 genomic bins, it was observed that overexpression of AID alone generated the most significant off-target bins (16 bins, Fisher's Exact Test). 5 bins were detected with elevated editing rates for AID-NS3h-UGI, 2 for AID-PcrA-UGI and AID-PcrA M6-UGI, respectively, and 1 for AID-BLM-UGI. The bins with elevated editing rates in AID-NS3h-UGI overlapped with bins identified from other conditions, indicating common off-target sites across helicases.

### HACE Enables the Identification of MEK1 Inhibitor-Resistance Mutations

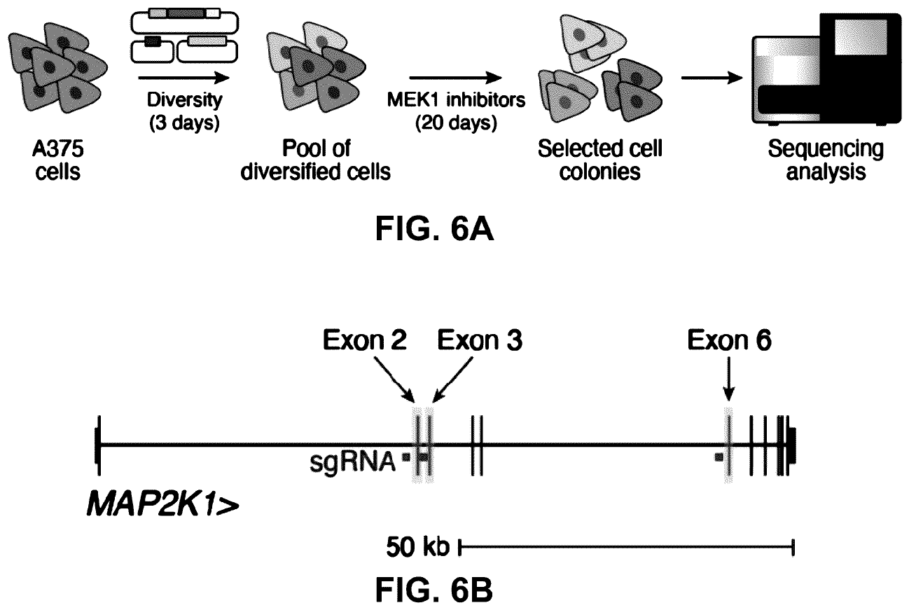

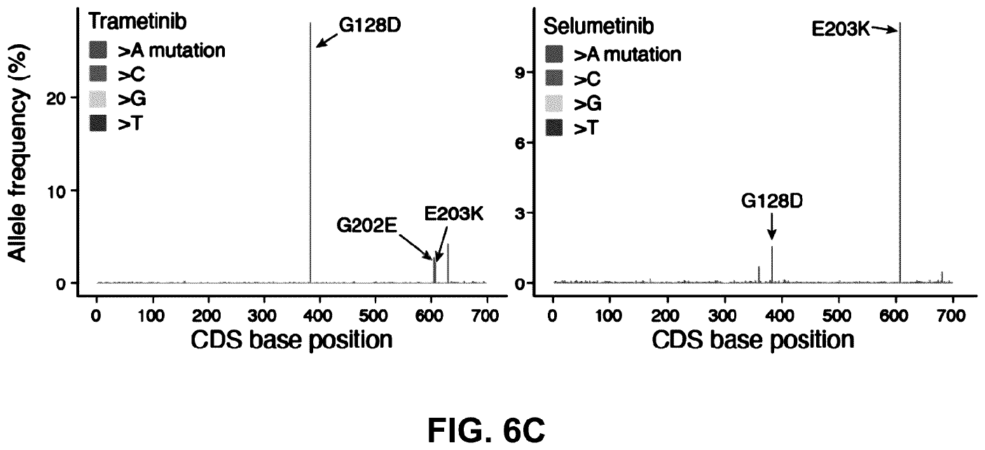

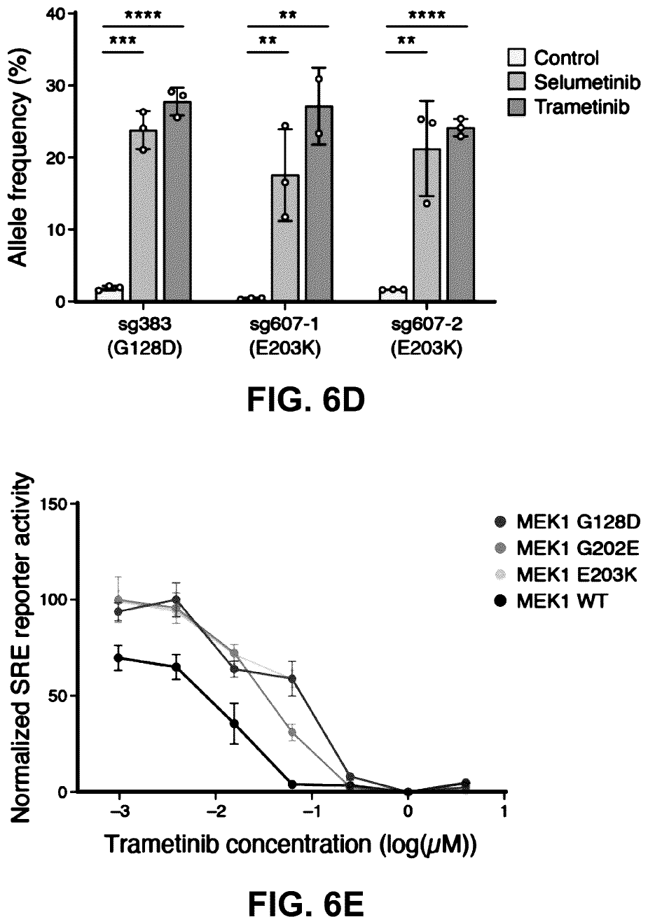

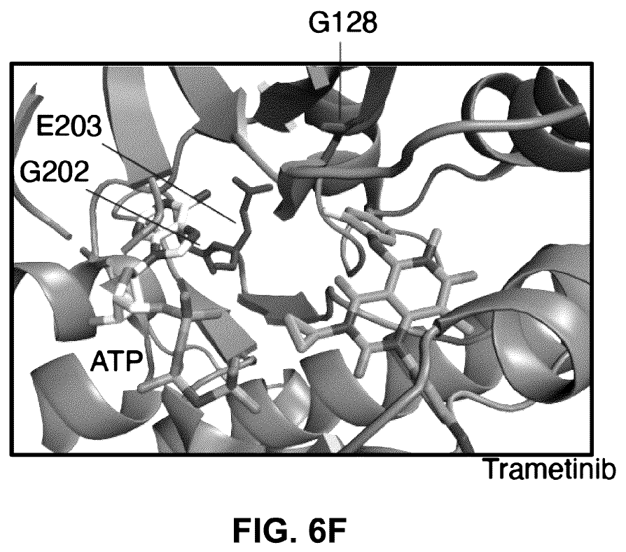

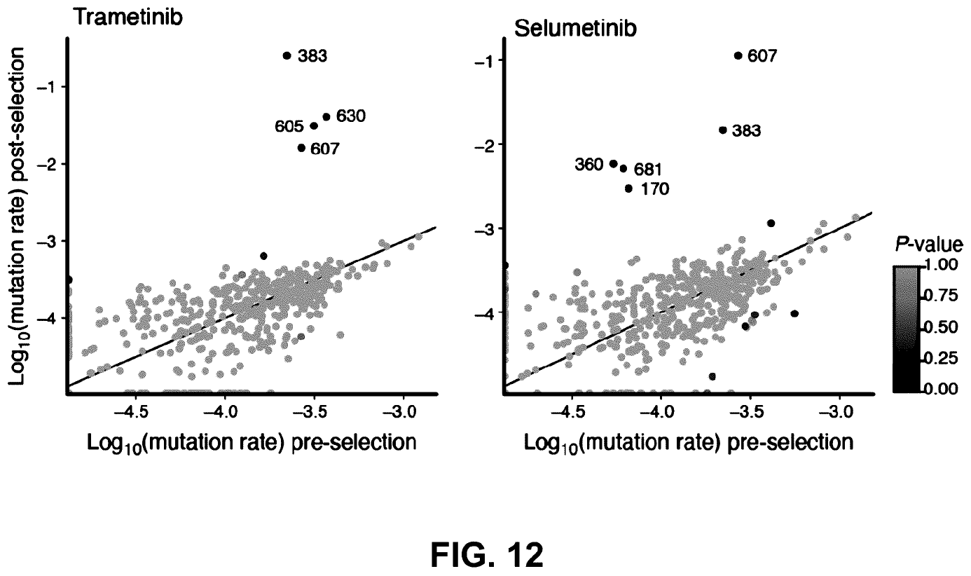

In another embodiment, HACE enables the identification of MEK1 inhibitor resistance mutations. In the coding genome, HACE was first applied to screen for mutations within mitogen-activated protein kinase kinase 1 (MEK1 kinase, also known as MAP2K1) that promote resistance to small-molecule drug inhibition. MEK inhibitors target the MAPK/ERK pathway, which is aberrantly upregulated in one-third of all cancers. Using HACE, exons of the MEK1 gene were diversified in A375 cells, a melanoma line sensitive to MEK inhibition, for three days, then cells were selected for resistance to two MEK1 inhibitors-selumetinib and trametinib (FIG. 6A). Exons 2, 3, and 6 were targeted, which contain previously identified mutation hotspots. Since the mutagenesis range of HACE is long, it was only needed to design one sgRNA per exon. Each exon-specific sgRNA ˜100 bp was placed upstream of the exon within the intronic region (FIG. 6B). By comparing allele frequencies between pre- and post-drug selection samples and identifying alleles that are significantly enriched post-drug selection, three candidate mutations were identified that conferred resistance to trametinib (G128D, G202E, and E203K) and two candidate mutations that conferred resistance to selumetinib (G128D and E203K) were identified (FIG. 6C, FIG. 12). Two of the mutations, G128D and E203K, conferred resistance to both selumetinib and trametinib.

To validate top mutation candidates, sgRNAs were designed to introduce mutations individually into A375 cells using base editing, then selected edited cells with either selumetinib or trametinib for 14 days. The allele frequencies of introduced mutations pre- and post-selection were evaluated by amplicon sequencing (FIG. 6D). Significant enrichment of G128D (sg383) and E203K (sg607-1 and sg607-2) post-selection with both inhibitors was observed. Due to the artificial linkage in base editing between G202E and E203K, the 605G>A (G202E) mutation could not be introduced by base editing. Candidates that conferred resistance to trametinib were further validated using a luciferase serum response element (SRE) reporter assay of MAPK-ERK signaling activity via exogenous overexpression of candidate MEK1 mutants (FIG. 6E). All three mutations individually increased trametinib resistance (IC50=68.0 nM, 46.1 nM, and 46.1 nM for G128D, G202E, and E203K, respectively, vs. 5.28 nM for wild-type). Structural analysis revealed that G128D is in the ligand-binding pocket. This mutation may function by inducing conformational changes of the binding pocket via steric interactions (FIG. 6F).

HACE Enables the Identification of Variants in SF3B1 that Result in Alternative 3′ Branch Point Usage

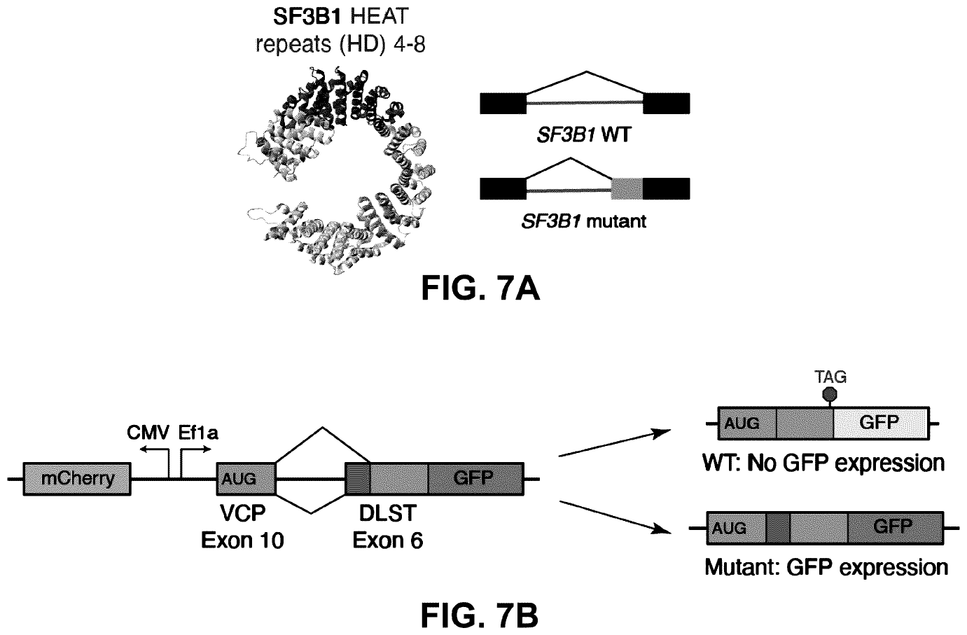

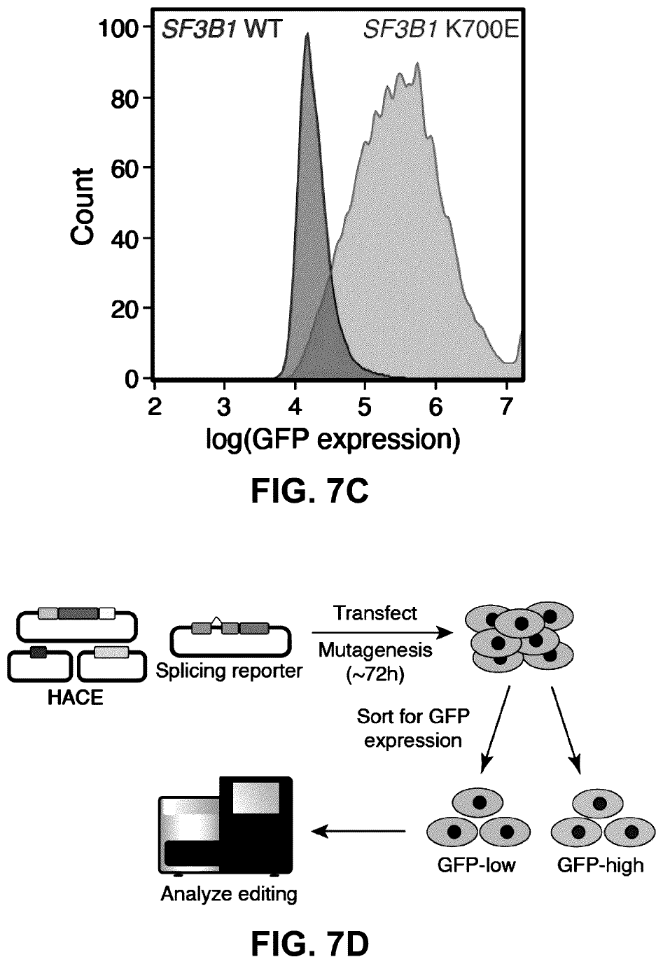

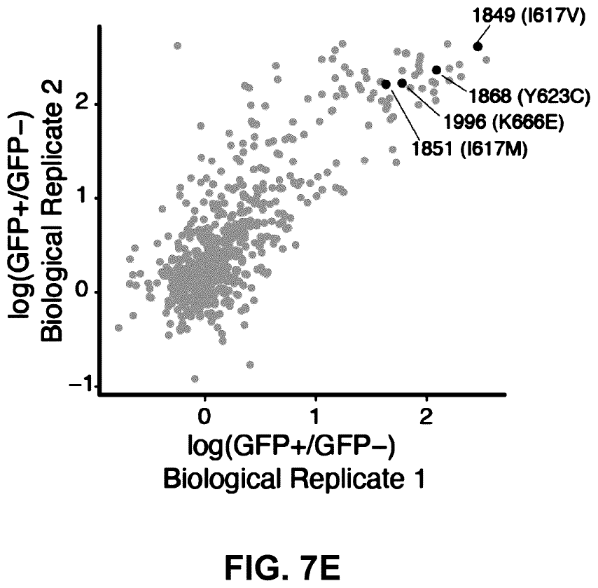

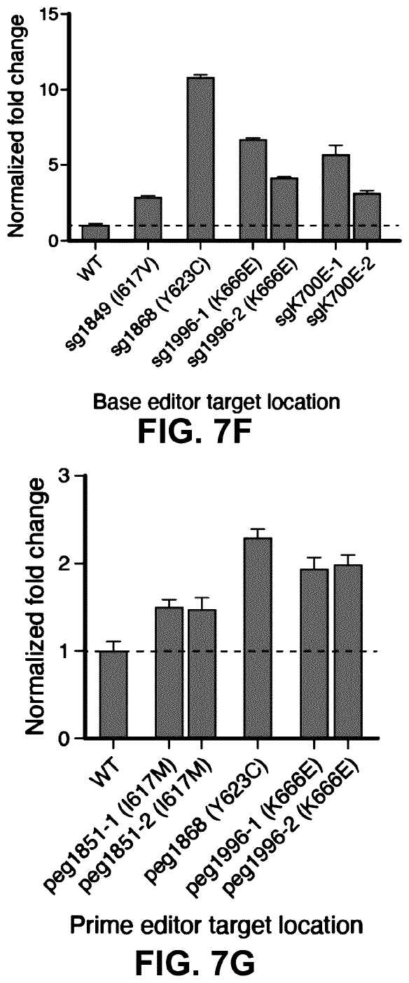

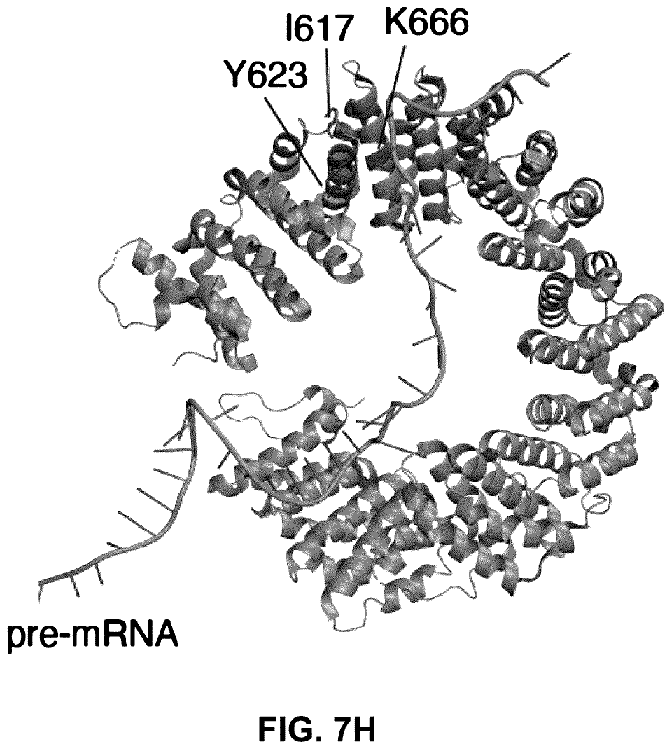

In another embodiment, HACE was applied to explore the function of individual variants in splicing factor 3B subunit 1 (SF3B1) for splicing regulation. Mutations in RNA splicing factors occur in many cancer types and are especially prevalent in hematopoietic malignancies. SF3B1 is the most frequently mutated splicing factor in cancer. It is a member of the U2 small nuclear RNP (snRNP) complex and binds to the branch point nucleotide in the pre-catalytic spliceosome. Pan-cancer analysis of SF3B1 mutations has identified hotspot mutations clustered within the C-terminal HEAT repeat domains 4-8 that display an alternative 3′ splice site (ss) usage signature (FIG. 7A). This mis-splicing occurs through the recognition of a different branch point sequence during 3′ss selection and results in global splicing changes associated with tumorigenesis. However, most known mutations identified from bioinformatic analysis of clinical samples have not been functionally validated for their effect on splicing.

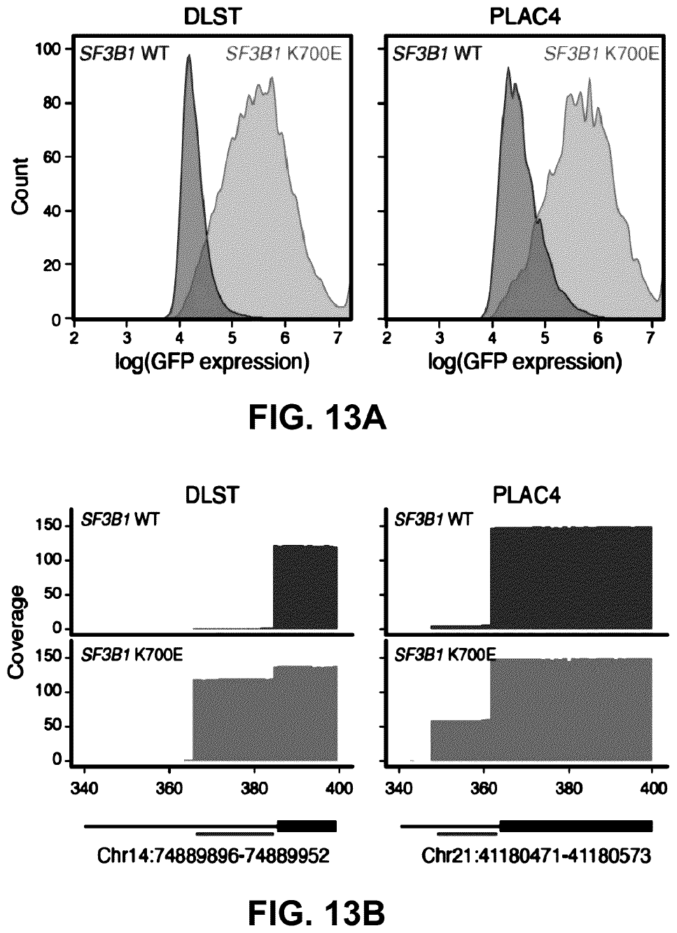

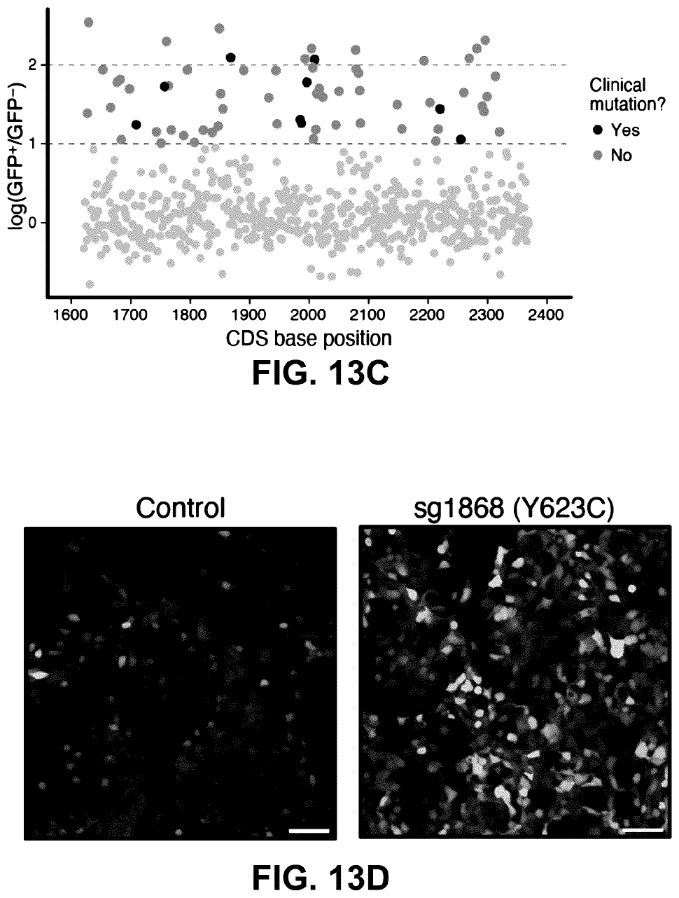

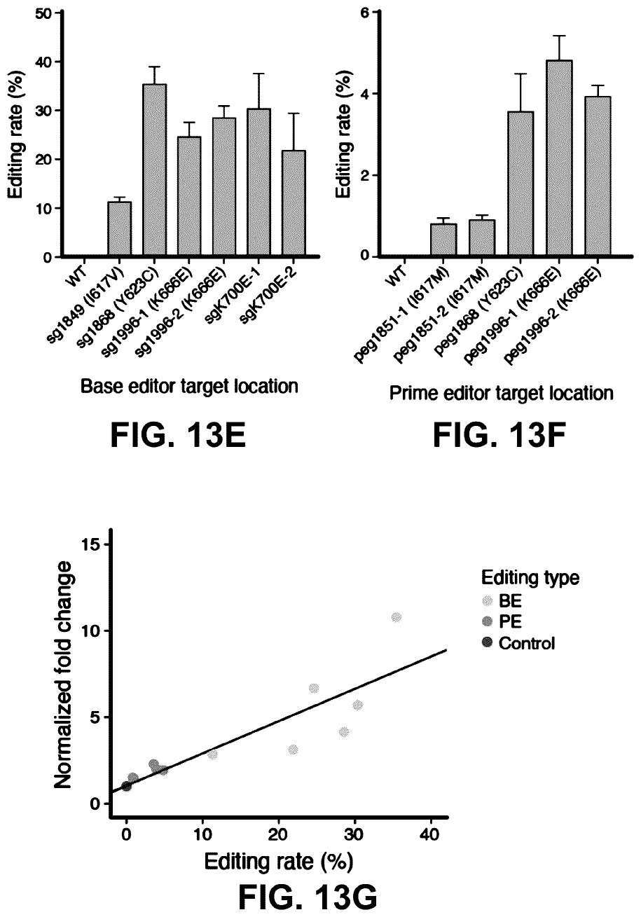

In another embodiment, validation of screening for mutations in SF3B1 that functionally lead to mis-splicing, it was first sought to construct a minigene reporter that could distinguish between wild-type SF3B1 (SF3B1WT) and mutated SF3B1 splicing patterns. These patterns display alternative 3′ss usage characteristics of hematopoietic malignancies. First, the RNA-seq data from isogenic K562 cells containing either SF3B1WT or mutant SF3B1 (SF3B1K700E, a mutation known to induce the alternative 3′ss phenotype) were compared. Splicing events were shortlisted that were significantly differentially spliced between WT and mutant cells and minigene reporters were constructed from two of the top sequences to test their ability to functionally distinguish between SF3B1WT and SF3B1K700E-induced mis-splicing. To do so, the last 150 bp of the endogenous intron and its downstream exon for each sequence were extracted and a minigene was constructed by fusing it to a constant upstream exon and a downstream GFP reporter (FIG. 7B). To validate the splicing reporter constructs, each construct was transfected into isogenic SF3B1WT or SF3B1K700E K562 cells and mutant-dependent protein expression was measured by flow cytometry. Both reporters showed mutant-dependent specificity, showing elevated GFP expression in SF3B1K700E cells compared to SF3B1WT cells (FIG. 13A). Alternative 3′ss usage drives the reporter expression using targeted RNA-seq (FIG. 13B). The reporter with the most significant mutant-dependent specificity derived from DLST Exon 6 was selected to proceed with a SF3B1 variant screen using HACE (FIG. 7C).

In another embodiment, to perform the screening using HACE, the exons 13-17 of the SF3B1 gene were diversified in HEK293FT cells for 3 days. To widen the genetic search space, HACE editors were used that can cover both C:G>T:A and A:T>G:C mutation modes (AID*Δ-PcrA M6-UGI and TadA-8e-PcrA M6-UGI). The minigene reporter was transfected into diversified cells, the cells were sorted into two bins (GFP− and GFP+) based on the GFP:mCherry ratio, and high-throughput sequencing for cells in each bin was performed (FIG. 7D). Enriched mutations were identified by comparing the fold-change between GFP− and GFP+ cells (FIG. 7E). A high degree of replicate correlation for enriched variants between two independent biological replicates (Pearson's p=0.795) was observed. The highly enriched variants (>10-fold enrichment) were also compared to mutations observed in clinical datasets and nine variants were found that occurred at high frequency clinically (FIG. 13C). To validate the candidates that displayed the highest enrichment levels, the candidate mutations were introduced individually into HEK293FT cells using base editing in cells that were co-expressing the minigene reporter. Then the fold change in GFP:mCherry ratio compared to unedited cells was quantified. It was found that three of the mutations (1617V, Y623C, K666E) led to a significant increase in reporter fold-change (unpaired t-test, p<0.001 for all base editing groups compared with control, FIG. 7F, FIG. 13D). Targeted amplicon sequencing was also performed for each cell population to validate editing at each target base (FIG. 13E). Two of the mutations Y623C and K666E, have been observed in clinical datasets and are highly enriched in hematopoietic tumor samples. K666E has been previously validated for its effect on alternative 3′ss usage. An additional mutation, 1617V/M, not previously observed in clinical datasets was also observed. These top candidate mutations were further validated via prime-editing and reporter fold change was measured (FIG. 7G, FIG. 13F). Despite the low editing efficiency, significantly increased splicing reporter fold changes were observed for the mutations as compared to WT cells (unpaired t-test, p<0.01 for all prime editing groups compared with control). Overall, the editing rate for candidate mutations that affect SF3B1 alternative 3′ss usage across validation experiments correlates well with the minigene reporter fold change (FIG. 13G). Analysis of the protein structure of these mutations found that the mutations are all located at the edge of the HEAT repeat helices of the SF3B1 protein structure, which matches the pattern of hotspot mutations previously found from pan-cancer analysis (FIG. 7H).

### Mutagenesis of Noncoding Regulatory Elements Uncovers Functional Bases and Variants

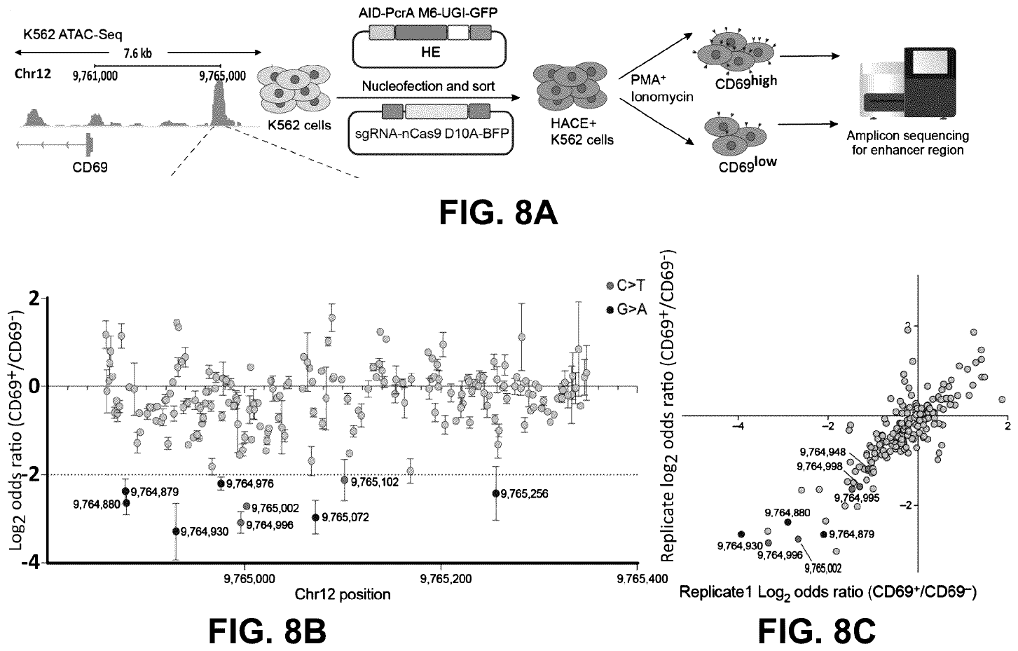

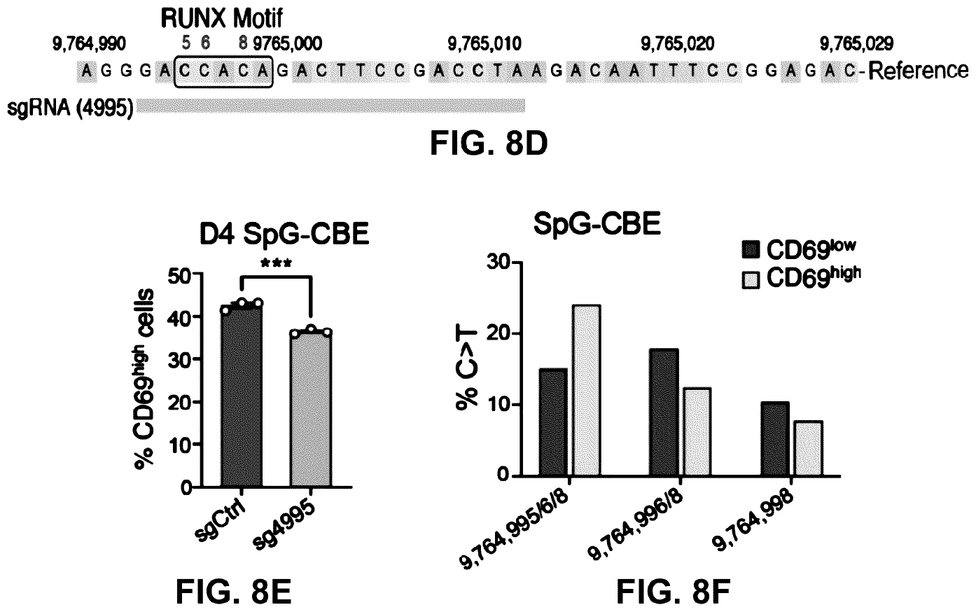

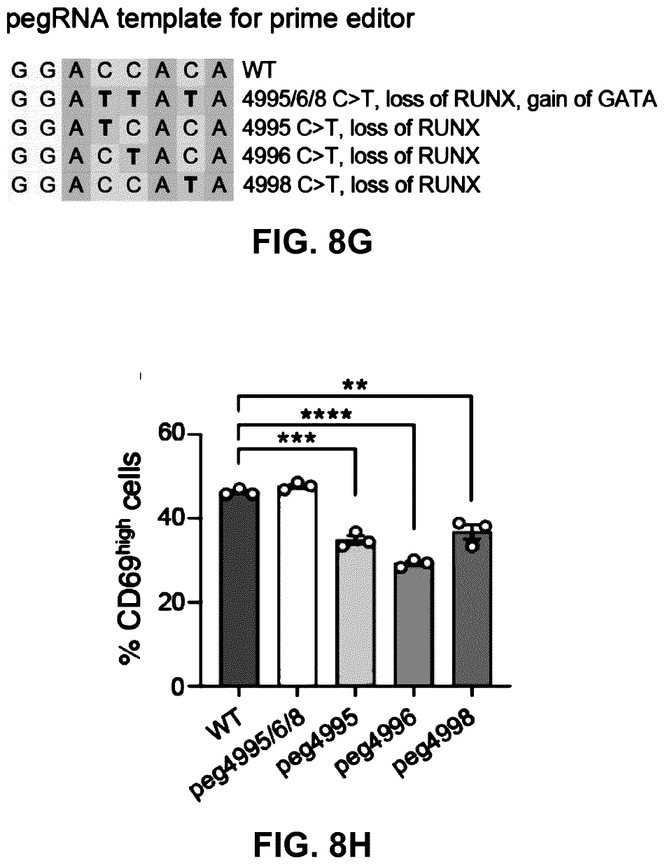

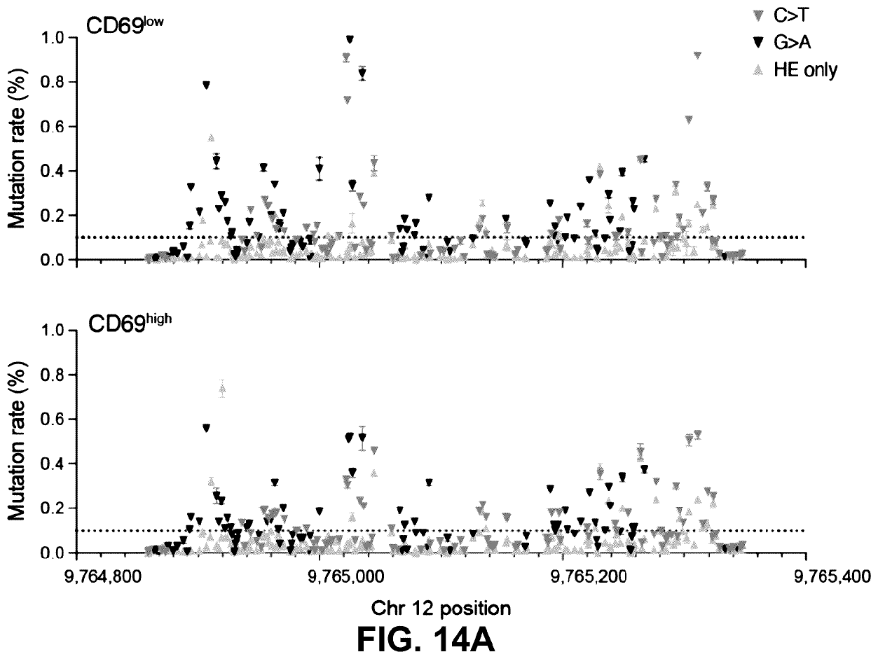

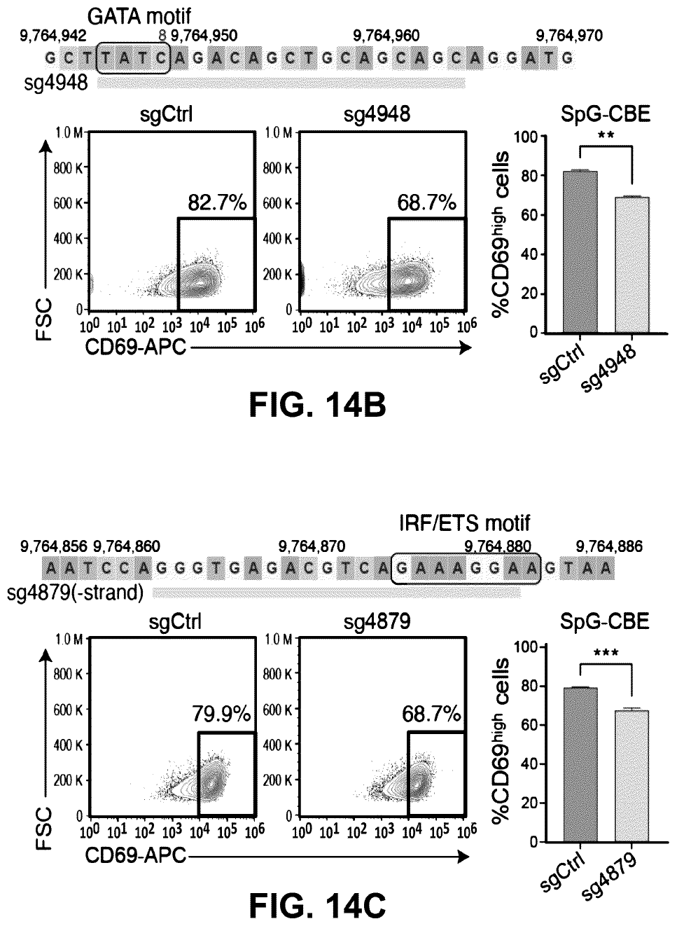

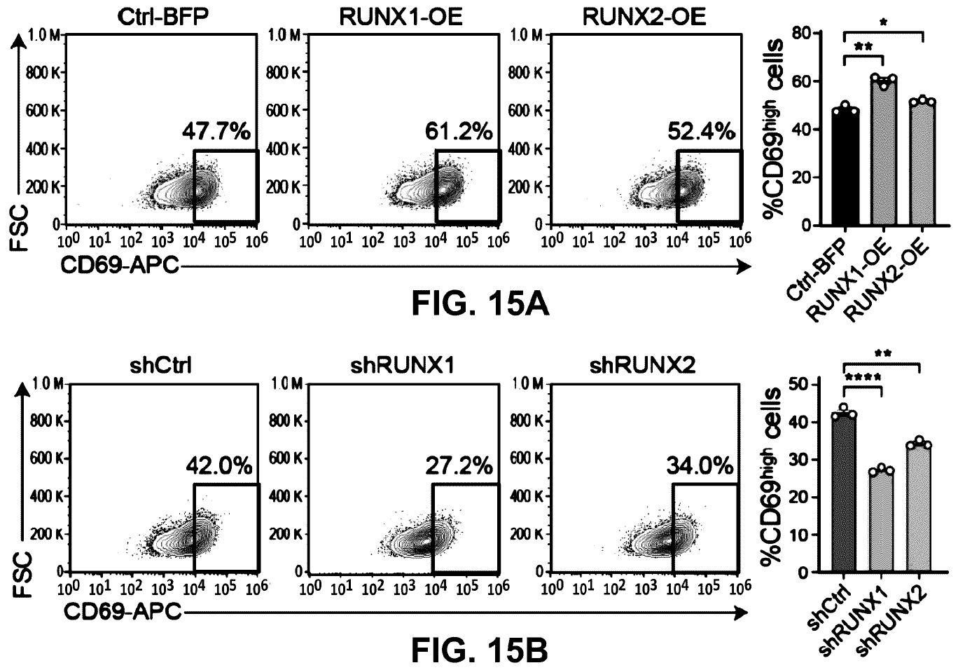

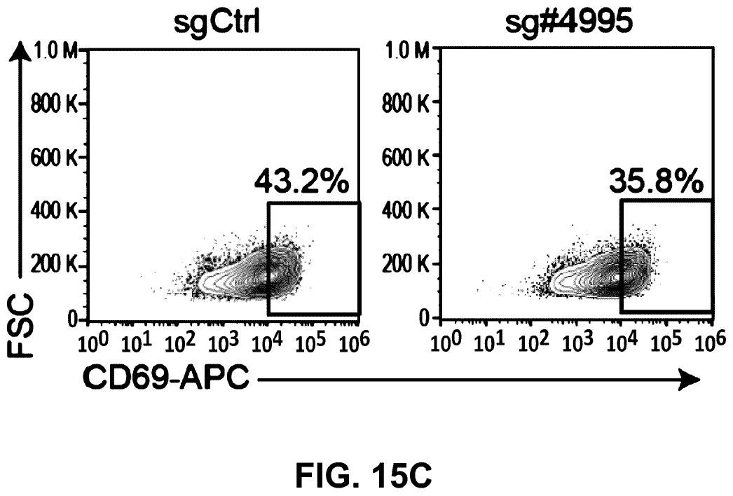

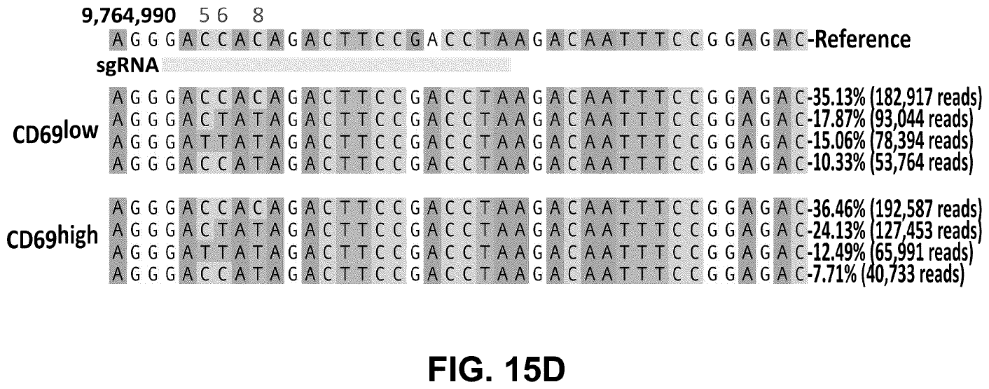

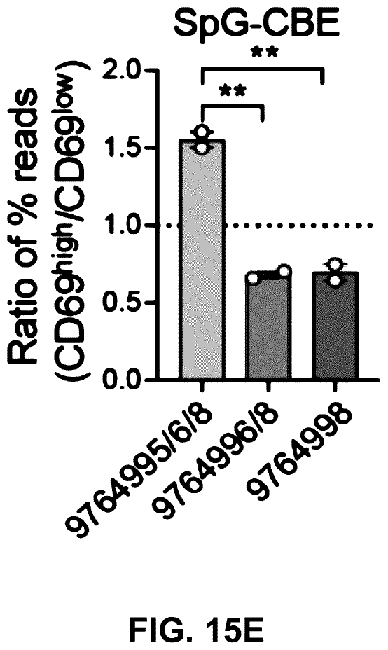

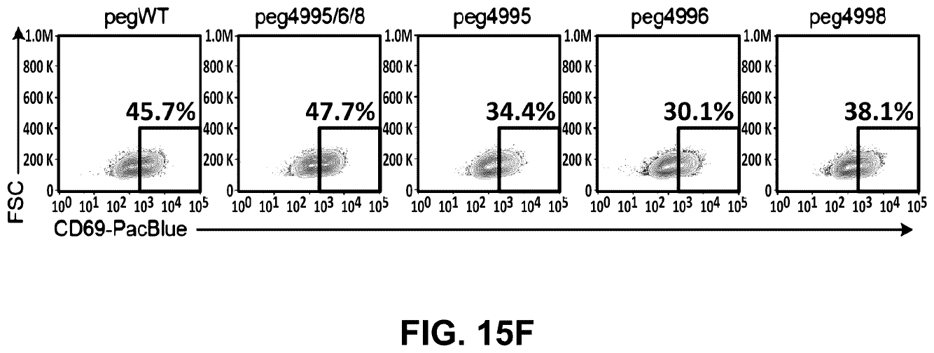

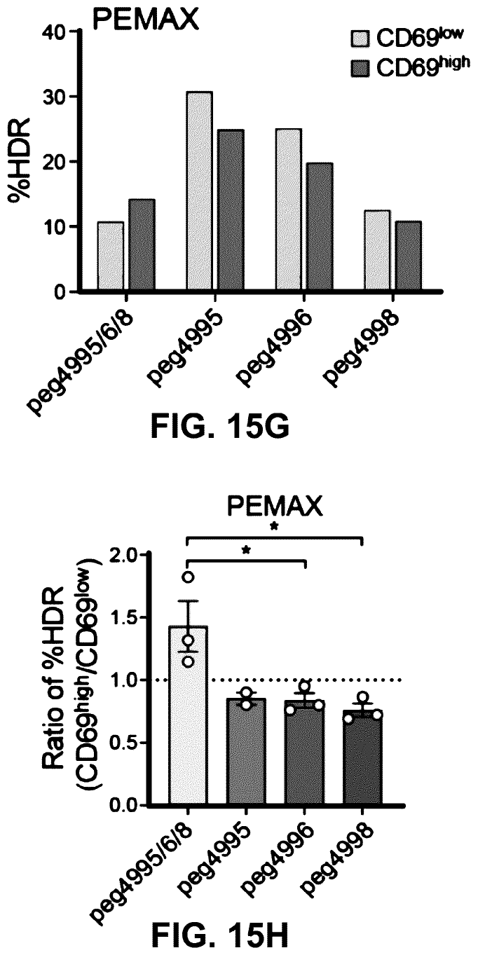

In another embodiment, HACE was targeted to an enhancer region that regulates CD69, a membrane-bound lectin receptor gene that contributes to immune cell tissue residency. Three sgRNAs were designed targeting the core region of the CD69 enhancer. K562 cells were infected with these nCas9-sgRNAs and HE (AID-PcrA-M6-UGI) constructs. After 6 days, the cells were stimulated with PMA/ionomycin to induce CD69 expression and sorted based on CD69 surface expression. Mutations were assessed by amplifying and sequencing the targeted region in CD69low and CD69high subsets (FIG. 8A, FIG. 14A). Multiple individual bases reduced CD69 activation, with most of them located in motifs of immune-related transcription factors (FIG. 8B). The base enrichment pattern was highly consistent across biological replicates (Pearson's p=0.845), confirming the robustness of the screen (FIG. 8C). The HACE screen was validated by evaluating mutations that were located in three immune-related transcription factor motifs via base editing. K562 cells were infected with a base editor construct and sgRNAs designed to incur the corresponding edits, the cells were activated, and CD69 expression was analyzed by flow cytometry. It was first confirmed that a C>T transition at base Chr12:9764948 (shortened as the last four digits “4948”, same nomenclature used below) significantly suppressed CD69 induction upon stimulation (FIG. 14B). The base editing tiling screen previously identified this artificial variant, which disrupts a GATA transcription factor motif. G>A transitions were also validated at both bases 4879 and 4880 that suppress CD69 induction (FIG. 14C). These variants lie within a predicted IRF/ETS or IRF/STAT transcription CD69 expression during stimulation. Three closely adjacent screen hits were focused on: C>T transitions at positions 4995, 4996, and 4998. Motif analysis suggests these variants are located within the core motif region (“CCACA”) recognized by RUNX family transcription factors (FIG. 8D). RUNX1 and RUNX2 are both expressed in K562 cells and have previously been implicated in CD69 expression. Indeed, CD69 expression increased when either RUNX1 or RUNX2 was overexpressed (FIG. 15A), and CD69 expression levels decreased when RUNX1 or RUNX2 was knocked down using shRNA (FIG. 15B). These results support a role for a RUNX1/2 circuit in driving CD69 induction in K562 cells. To validate the data, a sgRNA targeting the 4995-4998 region was designed and C>T mutations were introduced using an NG-PAM cytidine base editor (FIG. 8D). It was confirmed that the base edits reduced CD69 expression four days post-editing (FIG. 8E, FIG. 15C). To evaluate the impact of different combinations of variants in this window, CD69low and CD69high populations were sorted and targeted amplicon sequencing was performed. It was found that edited alleles with a single mutation at position 4998 or paired mutations at positions 4996/4998 were enriched in the CD69low population, consistent with an adverse effect on CD69 induction. This loss-of-function likely reflects the ablation of the coincident RUNX motif. However, edited alleles with C>T mutations at all three positions (4995/4996/4998) were enriched in the CD69high population (FIG. 8F, FIG. 15D-E). A likely explanation for this discordance is that the concurrent triple mutation not only disrupts the RUNX motif but also creates a GATA motif (“GATT”) at positions 4993-4996. GATA factors, including GATA1 and GATA2, are highly expressed in K562 cells and associated with transcriptional induction. These data suggest that de novo motif creation and GATA recruitment underlie the increased CD69 induction associated with the triple mutant allele (FIG. 8G). The findings support the potential of combinatorial base editing to create gain-of-function regulatory sequences but also highlight complications related to artificial linkage between adjacent variants. To definitively address this limitation, prime editing was used to introduce either individual or triple mutations at this locus. It was found that the triple mutation (4995/4996/4998) increased CD69 induction (FIG. 8H, FIG. 15F), while the respective single mutations reduced induction. Amplicon sequencing of sorted cells further confirmed that the CD69high population is enriched for the triple mutant allele but depleted for the respective single mutants (FIG. 15G-H). Hence, the application of HACE, followed by base and prime editing validation, revealed single nucleotide variants and combinatorial mutations capable of significantly modulating the activity of the CD69 immune enhancer through ablation or creation of transcription factor motifs.

### Materials and Methods

### Plasmids and Oligonucleotides

The guide sequences used for HACE mutagenesis (Table 4) are closed by Gibson assembly or Golden Gate assembly. The oligos used in this study for sequencing (Table 5) were purchased from Integrated DNA technologies (IDT) or Azenta/GENEWIZ. The Cas9 nickase plasmids were derived from plasmids pSpCas9(BB)-2A-GFP (Addgene 48138) and pCMV-PEmax-P2A-GFP (Addgene 180020). Plasmids expressing sgRNAs and pegRNAs were cloned by Gibson assembly or Golden Gate assembly. HACE Editor plasmids were cloned by Gibson assembly of PCR products. Individual helicases were either subcloned from plasmids (pEGFP-BLM—Addgene 110299; pET22B_SA_PcrA—Addgene 102999; pCMV-Tag1-NS3—Addgene 17645) or synthesized by Integrated DNA Technologies after mammalian codon optimization. The helicases tested are summarized in Table 6, and sequences of individual helicases tested are listed in Table 7. All new plasmids generated during this study will be deposited on Addgene.

### Mammalian Cell Culture

HEK293FT cells (Thermo Fisher—R70007) and A375 cells (ATCC, CRL-1619) were cultured in Dulbecco's Modified Eagle Medium with GlutaMAX (Thermo Fisher Scientific 10564011) supplemented with 10% (v/v) fetal bovine serum (FBS, Sigma-Aldrich F4135) and 1× penicillin-streptomycin (Thermo Fisher Scientific 15140122). Adherent cells were maintained at confluency below 80%-90% at 37° C. and 5% CO₂.

K562 cells (ATCC, CCL-243) were cultured in RPMI 1640 medium with GlutaMax (Thermo Fisher-61870036) supplemented with 10% (v/v) FBS and 1× penicillin-streptomycin. Suspended cells were maintained at confluency below 1.5×10⁶ cells/ml at 37° C. and 5% CO₂. For stimulation experiments, 50 ng/ml Phorbol12-myristate13-acetate (PMA, Sigma-Alrich, P8139) and 500 ng/ml ionomycin calcium salt from Streptomyces conglobatus (ionomycin, Sigma-Aldrich, I0634) are added to the cell culture media to simulate the cells for 2-3 hours.

### Transfection of HACE Plasmid and Genomic DNA Preparation for HACE Editing Assays

The day before transfection, 10,000 HEK293FT cells were seeded per well on 96-well plates (Corning). Then, 16-24 hours after seeding, cells were transfected at approximately 70% confluency with 0.3 μL of TransIT-LT1 (Mirus Bio) according to the manufacturer's specifications. Each well was transfected with 40 ng of HACE editor plasmid, 40 ng of Cas9 nickase plasmid, and 16 ng of sgRNA plasmid were delivered to each well unless otherwise specified. For control conditions, HACE editor plasmid and/or Cas9 nickase plasmid were substituted with the same amount of pUC19 plasmid. Cells were cultured for 3 days after transfection. DNA was collected from transfected cells by removal of medium, resuspension in 50 μL of QuickExtract (Lucigen), and incubation at 65° C. for 15 min, 68° C. for 15 min, and 98° C. for 10 min. After thermocycling, lysate was used directly in downstream PCR reactions as per manufacturer protocol.

### High-Throughput DNA Sequencing of Genomic DNA Samples

For short amplicon (˜300 bp) sequencing for HEK293T and A375 cells, the target region was amplified from genomic DNA samples using Phusion U Hot Start PCR master mix (ThermoFisher Scientific, F562) in a 20 μL reaction. The following program was used: 98° C. for 30 s; 28 cycles of 98° C. for 10 s, 65° C. for 30 s, 72° C. for 30 s; 72° C. for 2 min, then 4° C. forever. Barcodes and adapters for Illumina sequencing were added in a subsequent PCR amplification using Q5 High-Fidelity Hot-Start Polymerase Master Mix (2×, New England Biolabs). Amplicons were pooled and prepared for sequencing on a NextSeq (Illumina) with paired-end reads (read1, 160 bp; index1, 8 bp; index2, 8 bp; read2, 160 bp). Reads were demultiplexed and analyzed with appropriate pipelines.

For long amplicon (˜2000 bp) sequencing, the targeted region was amplified using Phusion U Hot Start PCR master mix in a 20 μL reaction. The following program was used: 98° C. for 30 s; 28 cycles of 98° C. for 10 s, 65° C. for 30 s, 72° C. for 2 min; 72° C. for 5 min, then 4° C. forever. PCR products were purified using Magnetic Ampure XP beads (Beckman Coulter) using a 1:1 bead solution:DNA solution ratio to select the PCR fragments. Purified PCR products were eluted in 20 μL of water. The concentration of each sample was measured by Qubit (Thermo Fisher Scientific). The sequencing library was prepared following the Nextera XT Kit protocol (Illumina) using Ing of purified amplicon DNA per sample as starting material and half of the recommended amount of each kit reagent. Sequencing was performed on a NextSeq (Illumina) with paired-end reads (read1,100 bp; index1, 8 bp; index2, 8 bp; read2, 100 bp).

### Quantification of Editing Rate

Raw fastq reads obtained from sequencing were quality trimmed using BBduk(1) (BBMap v38.93) with the options “qtrim=r1 trimq=28 maq=25”. Next, all bases with quality scores below 28 were masked to N using seqtk v1.3 (2). The filtered reads were aligned to the reference sequence using Bowtie2(3) (version 2.3.4.3). The pileup at each base was calculated using a custom Python script.

To calculate the mutation rate, it was filtered for base positions with a sequencing coverage of at least 10,000. Bases that had a higher than 5% mutation rate in the control condition were masked since this either indicated that it was a variant or an artifact from sequence alignment. The average G>A editing rate was calculated by extracting all positions where “G” was the reference base, then taking the average of the per base G>A editing rate. The editing rate for other base transition and transversion modes was calculated similarly.

To calculate the local editing rate, the alignment was centered such that the nick site is centered at base position 0. For every base position, the local G>A editing rate was calculated by extracting all the “G” bases within a 100 bp window (50 bp upstream and 50 bp downstream) and then taking the average of all per G>A editing rates.

### Cell Viability Measurements

Cell viability was measured using the CellTiter-Glo Luminescent Cell Viability Assay (Promega) following the manufacturer's protocols. Briefly, HEK293FT cells were seeded at a density of 10,000 cells per 100 μL per well in a 96-well plate in biological triplicates. The following day, cells were transfected with respective HACE plasmids according to the above protocol. Cell viability was measured 72 h after transfection. Luminescence readings were performed using a SpectraMax M5 (Molecular Devices) plate reader.

### Whole Exome Sequencing and Off-Target Analysis

The day prior to transfection, 50,000 HEK293FT cells were seeded in a 24-well plate. The following day, individual HE constructs were transfected together with a sgRNA targeting the MAP2K1 locus. Genomic DNA was extracted from cells 3 days post-transfection using the Zymo Quick-DNA Miniprep Kit (Cat D3024). Amplicon sequencing was performed at the MAP2K1 loci to confirm that there is HACE-dependent editing at the target loci in each condition. The whole genome DNA sequencing library was prepared using the NEBNext Ultra II FS DNA Library Prep Kit for Illumina (Cat E7805S). Exome sequences were enriched using the xGen Exome Hybridization Panel (IDT 10005152) following the manufacturer's protocols. Exome libraries were sequenced on a NovaSeq X (Illumina) with paired-end reads (read1,150 bp; index1, 8 bp; index2, 8 bp; read2, 150 bp) at a minimum of 100 million reads per sample.

The sequencing output was demultiplexed using bcl2fastq, and the paired-end reads were aligned to the reference genome hg38 using HISAT2 v2.2.1 (4). Aligned reads from each replicate were subsampled using reformat.sh (BBMap v38.93) and 100 million aggregated reads per replicate for each condition were used for further analysis. The HEK293FT-specific single-nucleotide polymorphisms (SNPs) were determined following the GATK4 variant calling workflow for germline short variant discovery (gatk.broadinstitute.org/hc/en-us/articles/360035535932-Germline-short-variant-discovery-SNPs-Indels-) on wild-type HEK293FT exome libraries (>50× coverage). In brief, the aligned reads were de-duplicated using Picard v2.27.5. HaplotypeCaller (GATK4) was used for calling variants and known variants, in dbSNP version 138 were used during base-quality recalibration. The chromosomal coordinates where SNPs were detected were excluded from subsequent analysis.

To quantify the per-base editing rate of the exome, the base pileup at each base was calculated using samtools mpileup (v 1.15.1), followed by post processing using mpileup2readcounts (github.com/IARCbioinfo/mpileup2readcounts) Bases with less than 50 total read depth were excluded from subsequent analysis. The genome was binned into 100 kb bins using bcftools v1.15.1. The off-target C>T editing rates for each genomic bin were obtained using a custom R script by counting the number of C and T bases in each bin. Fisher's exact test was used to quantify significant changes in editing for each bin relative to cells transfected with only nCas9, using the FDR correction to adjust for multiple hypothesis testing. Significant off-target sites are listed in Table 8.

### MEK1 Inhibitor-Resistance Screen

A375 cells were diversified for 3 days by transfection of HE variant AID-PcrA M6-UGI, nCas9 D10A, and sgRNAs targeting exons 2, 3, and 6 of the MEK1 gene using TransIT-2020 (Mirus Bio). Approximately 5 million cells in a 15-cm dish were placed under selection with either 100 nM selumetinib or 5 nM trametinib for 20 days. A portion of pre-selection cells were harvested as a control. Cells were passaged every 3 days to ensure they were maintained at <70% confluency. After selection, cells were harvested, and genomic DNA was extracted using QuickExtract (Lucigen). The MEK1 exons were amplified with exon-specific primers (Table 5) using Phusion U Hot Start Master Mix. Concurrently, RNA was harvested from selected cells using the Qiagen RNeasy Mini Plus Kit (Cat 74134). The cDNA was generated by reverse transcription using Maxima H Minus Reverse Transcriptase (Thermo Fisher). Sequencing libraries for cDNA were generated using the modified Nextera XT Kit protocol described in the “High-throughput DNA sequencing of genomic DNA samples” section above. All libraries were sequenced on a NextSeq (Illumina) with paired-end reads (read1, 160 bp; index1, 8 bp; index2, 8 bp; read2, 160 bp). The mutation rate (allele frequency) for each base of the MEK1 sequence was calculated for both pre- and post-selection samples. Significant mutations were identified by comparing the base counts between pre- and post-selection samples using a Fisher's exact test (Table 9). The mutation rate was compared between RNA and DNA samples and it was found that they had a high correlation.

### SRE Reporter Assay

pEF1a-MEK1 wild type, pEF1a-MEK1G128D, pEF1a-MEK1G202E, and pEF1a-MEK1E203K were generated using Gibson assembly. Sequences of MEK1-derived constructs are available in Table 10. The SRE reporter assay was performed using the SRE reporter kit (BPS Biosciences) according to the manufacturer's protocols. In brief, ˜10,000 HEK293FT cells in 100 μl of growth medium were seeded in 96-well white opaque assay plates. The cells were transfected with 60 ng of reporter plasmid and 40 ng of respective MEK1 plasmids. The culture medium was replaced 6 hours post-transfection with 50 μl of trametinib-containing medium with 0.5% FBS. After 12 hours, the cells were washed and incubated with 50 μl of 0.5% FBS-containing culture medium supplemented with recombinant human epidermal growth factor protein (Life Technologies) at a final concentration of 10 ng/ml. After 6 hours of incubation, the reporter activity was assayed using a dual luciferase (Firefly-Renilla) assay system (BPS Bioscience) according to the manufacturer's instructions using a SpectraMax M5 (Molecular Devices) plate reader. The ratio between Firefly luminescence and Renilla luminescence intensity was calculated for each well after background subtraction.

Design of SF3B1 splicing minigene reporter

The minigene reporter to probe SF3B1 function was constructed by Gibson assembly of a synthetic minigene sequence (synthesized by Twist Biosciences) into a custom bicistronic mCherry/GFP reporter plasmid. To construct the minigene reporter, the VCP exon 10 sequence and 150 bp of its immediate downstream intron were fused with DLST exon 6 and 97 bp of its immediate upstream intron. Also an “ATG” start codon was appended at the beginning of the sequence. The open reading frame was adjusted such that correct splicing in wild-type cells will result in pre-mature termination before the GFP. In contrast, the alternative 3′ splice-site usage in SF3B1 mutant cells will result in full-length GFP expression. The minigene reporter sequences are annotated in Table 11.

### SF3B1 Mis-Splicing Screen

HEK293FT cells were diversified for 3 days by co-transfection of HE variants AID-PcrA M6-UGI and TadA-PcrA M6-UGI, nCas9 D10A, sgRNAs targeting SF3B1 exons 13-17, and splicing mingene reporter. Cells transfected with only minigene reporter were used as undiversified control. The experiment was performed in triplicate, with ˜10 million cells transfected per replicate. After diversification, cells were prepared for flow sorting by washing and resuspending in 1×PBS with 2% BSA. Cells were sorted using a SONY MA900 sorter, where mCherry-positive cells were sorted into a GFP⁻ and GFP⁺ bin. At least 1 million cells were collected for each cell population. After flow sorting, the RNA of the cells was extracted using the Qiagen RNeasy Mini Plus Kit. The cDNA was generated by reverse transcription using Maxima H Minus Reverse Transcriptase. Sequencing libraries for cDNA were generated using the modified Nextera XT Kit protocol and sequenced on a NextSeq. Fold enrichment was calculated by dividing the mutation rate in GFP⁺ by that of GFP⁻ samples. The significant mutations were identified using a Fisher's exact test and are shown in Table 12. The clinical mutations that are observed in SF3B1 were retrieved from COSMIC. A mutation was considered high frequency if there were at least 3 observations in the dataset.

### CD69 Enhancer Tiling for Functional Bases

K562 cells were nucleofected with 2.5 μg of HE and 2.5 ug of nCas9 and sgRNA plasmids using the SF Cell Line 4D-Nucleofector X Kit L (Lonza V4XC-2024), following the manufacturer's protocol. Each plasmid contained a fluorescent protein reporter (sgRNA mCherry, nCas9 GFP, HE BFP). Approximately 1.5-2×10⁶ cells were used per nucleofection reaction. After 24 hours, cells were sorted using either SONY SH800 or BD Aria flow cytometry sorter to isolate cells expressing all plasmid components.

On day 7 post-nucleofection, cells were stimulated with PMA/Ionomycin for 2-3 hours. The cells were stained with the antibody cocktail in the staining buffer of a 1:1 mix of PBS and Brilliant Staining Buffer (BD 566349) at room temperature for 20 mins or at 4° C. for 30 mins. The following antibodies and dyes from BioLegend were used: Brilliant Violet 510 anti-human CD69 Antibody (310936); APC anti-human CD69 Antibody (310910); Zombie NIR Fixable Viability Kit (423106). Cells were washed once in PBS with 1% FBS and then resuspended in the same buffer to prepare for flow sorting. Subsequently, the top 40% of cells showing high CD69 expression (CD69^{high}) and the bottom 20% with low CD69 expression (CD69^{low}) were sorted using the SONY SH800 flow cytometer. A minimum of 100,000 cells were collected per tube.

Genomic DNA was then isolated from these cells either by using the QIAGEN DNA micro isolation kit (Cat #56304) or by lysis buffer (0.5% Triton X-100, 0.1 AU/ml QIAGEN Protease (Cat 19157) in H₂O). The lysis process involved incubation at 56° C. for 20 minutes and at 72° C. for 20 minutes at 600 rpm on a thermo shaker. Amplicon PCR for the genomic DNA was processed using the KAPA HiFi HotStart ReadyMix PCR Kit (Roche, KR0370). The following program was used: 95° C. for 5 min; 30 cycles of 95° C. for 30 s, 60° C. for 30 s, 72° C. for 30 s; 72° C. for 5 min; 4° C. forever. The amplicon libraries were sequenced on a NextSeq.

To identify enriched bases, the % C→T or % G→A of each group were first calculated for both CD69^{high} and CD69^{low} groups (% high or % low). Then the log 2 odds ratio of CD69^{high} versus CD69^{low} was calculated as log₂OR=log 2[(% high/(1−% high))/(% low/(1−% low))]. The correlation of technical replicates was plotted using GraphPad Prism 10.0. The top hits are recorded in Table 13.

### Base Editing Validation

The following base editors are used in this study: pRDA_478 (Addgene 179096), pRDA_479 (Addgene 179099), pCAG-CBE4max-SpG-P2A-EGFP (Addgene: RTW4552/139998), pCAG-CBE4max-SpRY-P2A-EGFP (Addgene: RTW5133/139999), pCMV-T7-ABE8.20m-nSpCas9-NG-P2A-EGFP (Addgene: KAC1164/185919), pCMV-T7-ABE8.20m-nSpRY-P2A-EGFP (Addgene: KAC1335/185917). The validation sgRNAs are listed in Table 14. The sgRNA sequences were cloned into pCMV-BFP-U6-sgRNA.

(Addgene: 196725, gift from Bernhard Schmierer) or directly into pRDA_478 or pRDA_479. Mutations for sgRNAs and bystander editing rates were quantified using CRISPResso2 (6).

To validate MEK1 variants, 67 ng of cytidine base editor and 33 ng sgRNA plasmids were transfected into A375 cells per well in a 96-well format. A non-targeting sgRNA was used as a control. Post-diversification for 3 days, the cells were selected with either 100 nM selumetinib or 5 nM trametinib for 14 days. The mutation rate pre- and post-selection were analyzed by amplicon sequencing. All experiments were conducted in triplicates.

For validation of SF3B1 variants, HEK293FT cells in a 96-well format were transfected with 67 ng of the base editor-sgRNA plasmid and 33 ng of the minigene splicing reporter per well. A non-targeting sgRNA was used as a control. Cells were diversified for 3 days, then the GFP:mCherry ratio in each well was quantified by confocal microscopy using a custom cell segmentation and quantification pipeline. Briefly, individual cells were segmented via watershed segmentation using the mCherry channel. For each segmented cell, the total pixel area and mean intensity of the pixels were computed for GFP (488 nm) and mCherry (561 nm) channels to obtain a “pseudo-flow cytometry” dataset. The fluorescence background for each channel was subtracted from all conditions in that channel, and aggregated values for each condition were divided by area to obtain average fluorescence intensity. Standard deviation was computed by comparing average values in three technical transfection replicates. Editing at each sgRNA was quantified by amplicon sequencing of genomic DNA samples from each well. All experiments were conducted in triplicates.

For validation of CD69 variants, 2 μg of the base editor plasmid and 2 μg of the sgRNA plasmid were nucleofected into 1.5×10⁶ K562 cells using the SF Cell Line 4D-Nucleofector X Kit L according to the manufacturer's protocol. At 24 h post nucleofection, the cells co-expressing base editor and sgRNA were sorted based on reporter expression. On day 4 post-nucleofection, cells were stimulated with PMA/ionomycin for 2-3 hours, and the top 40% of CD69 high expression cells and bottom 20% of CD69 low expression cells were sorted using a Sony SH800 flow cytometer, collecting at least 10,000 cells per tube. Genomic DNA was isolated from the sorted cells using the QIAgen DNA Micro Kit (Cat #56304) and prepared for amplicon sequencing using the protocol described in the “High-throughput DNA sequencing of genomic DNA samples” section. The mutation rate at each locus was quantified using CRISPResso2.

### Prime Editing Validation

The following prime editor plasmids are used in this study: pCMV-PEmax-P2A-hMLH1dn (Addgene: 174828), pCMV-PEmax-P2A-GFP (Addgene: 180020), pEFla-hMLH1dn (Addgene: 174824). Desired pegRNA and nickase sgRNA sequences were designed using PrimeDesign. The epegRNA overhang was designed using pegLIT. Sequences of pegRNAs are shown in Table 15.

For prime editing validation of SF3B1 variants, HEK293FT cells in a 96-well format were transfected with 150 ng of PEmax, 50 ng of epegRNA, 25 ng of nicking sgRNA, and 50 ng of minigene splicing reporter using 0.5 uL of TransIT-LT1 per well. Cells were diversified for 3 days, then the GFP:mCherry ratio in each well was quantified by confocal microscopy as described above. Editing at each sgRNA was quantified by amplicon sequencing of genomic DNA samples from each well using CRISPResso2.

For prime editing validations for CD69 enhancer variants in K562 cells, 2 ug of the prime editor plasmid, 1 ug of hMLH1dn plasmid and 1 ug of epegRNA plasmid, and 0.5 ug of nickase sgRNA plasmid were nucleofected in 1.5×10⁶ cells using SF Cell Line 4D-Nucleofector X Kit L according to the manufacturer's protocols. After 24 hours, the cells that were positive for both prime editor and epegRNA were sorted based on the GFP and mCherry reporters and cultured in regular complete RPMI media. A second round of nucleofection and sorting was performed 4 days post-transfection to increase prime editing efficiency. On day 5 post the second nucleofection, CD69 expression levels were quantified by flow cytometry. Genomic DNA was harvested from CD69^{high} (top 40%) and CD69^{low} (bottom 20%) cells, and the editing efficiency was quantified by performing amplicon sequencing using the protocols described above. The mutation rate at each locus was quantified using CRISPResso2.

TABLE 4

| sgRNA | Target gene/ | | |
|---|---|---|---|
| name | region | Experiment | Spacer sequence |
| HEK3.1 | HEK3 | Initial validation | GGCCCAGACTGAGCACGTGA |
| | | (FIG. 1 and 2) | (SEQ ID NO: 44) |
| TNF.1 | TNF | Initial validation | TGAAAGCATGATCCGGGACG |
| | | (FIG. 1 and 2) | (SEQ ID NO: 45) |
| IL6.1 | IL6 | Initial validation | TGAAAGCAGCAAAGAGGCAC |
| | | (FIG. 1 and 2) | (SEQ ID NO: 46) |
| MAP2K1.1 | MAP2K1 | Initial validation | GAAAGAAAGCTCCAGGTCTG |
| | | (FIG. 1 and 2) | (SEQ ID NO: 47) |
| DNMT1.1 | DNMT1 | Initial validation | GATTCCTGGTGCCAGAAACA |
| | | (FIG. 1 and 2) | (SEQ ID NO: 48) |
| VEGFA.1 | VEGFA | Initial validation | GATGTCTGCAGGCCAGATGA |
| | | (FIG. 1 and 2) | (SEQ ID NO: 49) |
| CD209.1 | CD209 | Initial validation | GCCCTCCACTAGGGCAAGGGT |
| | | (FIG. 1 and 2) | (SEQ ID NO: 50) |

HACE sgRNA sequences for multiplex editing
Set 1

| | | | |
|---|---|---|---|
| CD209-1 | CD209 | Initial validation | GCCCTCCACTAGGGCAAGGGT |
| | | (FIG. 1 and 2) | (SEQ ID NO: 51) |
| RUNX1-1 | RUNX1 | Initial validation | ATGAAGCACTGTGGGTACGA |
| | | (FIG. 1 and 2) | (SEQ ID NO: 52) |
| VEGFA-1 | VEGFA | Initial validation | GATGTCTGCAGGCCAGATGA |
| | | (FIG. 1 and 2) | (SEQ ID NO: 53) |

Set 2

| | | | |
|---|---|---|---|
| DNMT1-1 | DNMT1 | Initial validation | GATTCCTGGTGCCAGAAACA |
| | | (FIG. 1 and 2) | (SEQ ID NO: 54) |
| HEK3-1 | HEK3 | Initial validation | GGCCCAGACTGAGCACGTGA |
| | | (FIG. 1 and 2) | (SEQ ID NO: 55) |
| IL6-1 | IL6 | Initial validation | TGAAAGCAGCAAAGAGGCAC |
| | | (FIG. 1 and 2) | (SEQ ID NO: 56) |

sgRNA used for MEK1 screen

| | | | |
|---|---|---|---|
| MEK1i1 | MAP2K1 | MEKli resistance | GAAAGAAAGCTCCAGGTCTG |
| | | screen (FIG. 3) | (SEQ ID NO: 57) |
| MEK1i2 | MAP2K1 | MEKli resistance | AAGCTCTTTAAGGTAGAGGG |
| | | screen (FIG. 3) | (SEQ ID NO: 58) |
| MEK115 | MAP2K1 | MEKli resistance | ACACCCCCGTCCGCCATCAG |
| | | screen (FIG. 3) | (SEQ ID NO: 59) |

sgRNA used for SF3B1 screen

| | | | |
|---|---|---|---|
| SF3B1_1 | SF3B1 | SF3B1 missplicing | ATAAGAAATTTAGAATTATC |
| | | screen (FIG. 4) | (SEQ ID NO: 60) |
| SF3B1_2 | SF3B1 | SF3B1 missplicing | AAGAAAGGACAGTCATGAGT |
| | | screen (FIG. 4) | (SEQ ID NO: 61) |
| SF3B1_3 | SF3B1 | SF3B1 missplicing | CCAGACTGGACTCAAACCTT |
| | | screen (FIG. 4) | (SEQ ID NO: 62) |
| SF3B1_13 | SF3B1 | SF3B1 missplicing | TGTTCACATTAAACAAAATT |
| | | screen (FIG. 4) | (SEQ ID NO: 63) |
| SF3B1_5 | SF3B1 | SF3B1 missplicing | ATTTATCTTCATTAAAGTTA |
| | | screen (FIG. 4) | (SEQ ID NO: 64) |
| SF3B1_6 | SF3B1 | SF3B1 missplicing | CCAGATTTGTTAATGTAAAC |
| | | screen (FIG. 4) | (SEQ ID NO: 65) |
| SF3B1_7 | SF3B1 | SF3B1 missplicing | TATATGTGCTTGATTATGAA |
| | | screen (FIG. 4) | (SEQ ID NO: 66) |

sgRNA used for CD69 enhancer screen

| | | | |
|---|---|---|---|
| CD69_A | CD69 enhancer | CD69 enhancer | CAAAGTGTGCAGAGAAGGTG |
| | | screen (FIG. 5) | (SEQ ID NO: 67) |
| CD69_B | CD69 enhancer | CD69 enhancer | TGTCTTAGGTCGGAAGTCTG |
| | | screen (FIG. 5) | (SEQ ID NO: 68) |
| CD69_C | CD69 enhancer | CD69 enhancer | GACTTGAAGAGGACAGAAGG |
| | | screen (FIG. 5) | (SEQ ID NO: 69) |

TABLE 5
Table of primer sequence used for sequencing

| | | |
|---|---|---|
| ID | Target | Sequence |

Amplicon Sequencing

| | | |
|---|---|---|
| DC1200 | HEK3 | TCGTCGGCAGCGTCAGATGTGTATAAGAGA |
| | | CAGGAGCTGCACATACTAGCCCC (SEQ ID |
| | | NO: 70) |
| DC1201 | HEK3 | GTCTCGTGGGCTCGGAGATGTGTATAAGAG |
| | | ACAGAGGGAGCTTGGCATGAGAAA (SEQ |
| | | ID NO: 71) |
| DC642 | TNF | TCGTCGGCAGCGTCAGATGTGTATAAGAGA |
| | | CAGNNNNNNGCAGAGGACCAGCTAAGAG |
| | | G (SEQ ID NO: 72) |
| DC643 | TNF | GTCTCGTGGGCTCGGAGATGTGTATAAGAG |
| | | ACAGCCCAGTCACTCCAAAGTGCAGCAGG |
| | | (SEQ ID NO: 73) |
| DC640 | IL6 | TCGTCGGCAGCGTCAGATGTGTATAAGAGA |
| | | CAGNNNNNNTGCCAGGATGCCAATGAGTT |
| | | (SEQ ID NO: 74) |
| DC641 | IL6 | GTCTCGTGGGCTCGGAGATGTGTATAAGAG |
| | | ACAGCCCAGCACTGCATGCAAGAGGGAGA |
| | | (SEQ ID NO: 75) |
| DC381 | MEK1 | TCGTCGGCAGCGTCAGATGTGTATAAGAGA |
| | | CAGNNNNNTGGTGATAGTCATCCCGGGT |
| | | (SEQ ID NO: 76) |
| DC497 | MEK1 | GTCTCGTGGGCTCGGAGATGTGTATAAGAG |
| | | ACAGCCCAGCGTCATCCTTCAGTTCTCCC |
| | | (SEQ ID NO: 77) |
| DC1198 | DNMT1 | TCGTCGGCAGCGTCAGATGTGTATAAGAGA |
| | | CAGTGTTCCCCAGAGTGACTTTTCC (SEQ |
| | | ID NO: 78) |
| DC1199 | DNMT1 | GTCTCGTGGGCTCGGAGATGTGTATAAGAG |
| | | ACAGTTCCACTCATACAGTGGTAGATTT |
| | | (SEQ ID NO: 79) |
| DC1206 | VEGFA | TCGTCGGCAGCGTCAGATGTGTATAAGAGA |
| | | CAGGGGTTTTGCCAGACTCCACA (SEQ ID |
| | | NO: 80) |
| DC1207 | VEGFA | GTCTCGTGGGCTCGGAGATGTGTATAAGAG |
| | | ACAGTTGGGACTGGAGTTGCTTCA (SEQ ID |
| | | NO: 81) |
| DC1208 | CD209 | TCGTCGGCAGCGTCAGATGTGTATAAGAGA |
| | | CAGAACAGGAAGTTGGGTAGGGA (SEQ ID |
| | | NO: 82) |
| DC1209 | CD209 | GTCTCGTGGGCTCGGAGATGTGTATAAGAG |
| | | ACAGGAGGACAGCAGCAGCTCAAA (SEQ |
| | | ID NO: 83) |
| DC317 | RUNX1 | TCGTCGGCAGCGTCAGATGTGTATAAGAGA |
| | | CAGNNNNNACAAACAAGACAGGGAACTG |
| | | G (SEQ ID NO: 84) |
| DC318 | RUNX1 | GTCTCGTGGGCTCGGAGATGTGTATAAGAG |
| | | ACAGCCCAGCTAGAGGGGTGAGGCTGAAA |
| | | C (SEQ ID NO: 85) |
| DC1238 | HEK3 long range | AACTAAACAGTCCCACTCCATCC (SEQ ID |
| | | NO: 86) |
| DC1239 | HEK3 long range | GTATCTTCATGCATTCTCCACGCC (SEQ ID |
| | | NO: 87) |
| DC895 | TNF long range | GGAGAAACAGAGACAGGCCC (SEQ ID NO: |
| | | 88) |
| DC896 | TNF long range | CCAGGTTTCGAAGTGGTGGT (SEQ ID NO: 89) |
| DC891 | IL6 long range | CCCACCGGGAACGAAAGAGA (SEQ ID NO: |
| | | 90) |
| DC892 | IL6 long range | GTCTCCCATTAGACCACAAGCA (SEQ ID NO: |
| | | (91) |

MEK1 screen

| | | |
|---|---|---|
| GW_73 | MEK1 exon 2 | TCGTCGGCAGCGTCAGATGTGTATAAGAGA |
| | | CAGNNNNNNNNNNTTGTGCTCCCCACTTTG |
| | | GAA (SEQ ID NO: 92) |
| GW_74 | MEK1 exon 2 | GTCTCGTGGGCTCGGAGATGTGTATAAGAG |
| | | ACAGNNNNNNNNNNCCTGTTAATCAAGGC |
| | | AAACTCACC (SEQ ID NO: 93) |
| GW_77 | MEK1 exon 3 | TCGTCGGCAGCGTCAGATGTGTATAAGAGA |
| | | CAGNNNNNNNNNNGACTATATCTTTCATCC |
| | | CTTCCTCCC (SEQ ID NO: 94) |
| GW_78 | MEK1 exon 3 | GTCTCGTGGGCTCGGAGATGTGTATAAGAG |
| | | ACAGNNNNNNNNNNCAACTCTTAAGGCCA |
| | | TTGCTCC (SEQ ID NO: 95) |
| GW_81 | MEK1 exon 6 | TCGTCGGCAGCGTCAGATGTGTATAAGAGA |
| | | CAGNNNNNNNNNNCCCAATCTACCTGTGT |
| | | CAGTTCC (SEQ ID NO: 96) |
| GW_82 | MEK1 exon 6 | GTCTCGTGGGCTCGGAGATGTGTATAAGAG |
| | | ACAGNNNNNNNNNNCCTACCCAGCACAAG |
| | | ACTCTG (SEQ ID NO: 97) |
| DC1247 | MEK1 CDS | GGAGTTGGAAGCGCGTTAC (SEQ ID NO: 98) |
| DC1248 | MEK1 CDS | CAAAAGCGACATGGCAAACC (SEQ ID NO: |
| | | 99 |

SF3B1 screen

| | | |
|---|---|---|
| DC1510 | SF3B1 exon 12 | TCGTCGGCAGCGTCAGATGTGTATAAGAGA |
| | | CAGTCTGCTCTTTTTCCCAGGCT (SEQ ID |
| | | NO: 100) |
| DC1511 | SF3B1 exon 12 | GTCTCGTGGGCTCGGAGATGTGTATAAGAG |
| | | ACAGATTAAGGAGAACAAACCTTATGCAC |
| | | (SEQ ID NO: 101) |
| DC1512 | SF3B1 exon 13 | TCGTCGGCAGCGTCAGATGTGTATAAGAGA |
| | | CAGCTCGTGGTCATTGAACCGCT (SEQ ID |
| | | NO: 102) |
| DC1513 | SF3B1 exon 13 | GTCTCGTGGGCTCGGAGATGTGTATAAGAG |
| | | ACAGAGAAAGGACAGTCATGAGTTGGT |
| | | SEQ ID NO: 103) |

TABLE 6
Table of helicase constructs tested

| Helicase | Species | Directionality |
|---|---|---|
| BLM(9) | Human | 3′ to 5′ |
| Ns3h(10) | Hepatitis C Virus | 3′ to 5′ |
| PcrA(11) | Bacillus | 3′ to 5′ |
| PcrA M6(12) | G. stearothermophilus, engineered | 3′ to 5′ |
| TraI(13) | E. coli | 5′ to 3′ |
| UvrD(14) | E. coli | 3′ to 5′ |

TABLE 7
Table of helicase sequences

| Helicase | Sequence |
|---|---|
| BLM | ATGGCTGCTGTTCCTCAAAATAATCTACAGGAGCAACTAGAACGTCACTC |
| | AGCCAGAACACTTAATAATAAATTAAGTCTTTCAAAACCAAAATTTTCAG |
| | GTTTCACTTTTAAAAAGAAAACATCTTCAGATAACAATGTATCTGTAACTA |
| | ATGTGTCAGTAGCAAAAACACCTGTATTAAGAAATAAAGATGTTAATGTTA |
| | CCGAAGACTTTTCCTTCAGTGAACCTCTACCCAACACCACAAATCAGCAA |
| | AGGGTCAAGGACTTCTTTAAAAATGCTCCAGCAGGACAGGAAACACAGA |
| | GAGGTGGATCAAAATCATTATTGCCAGATTTCTTGCAGACTCCGAAGGAA |
| | GTTGTATGCACTACCCAAAACACACCAACTGTAAAGAAATCCCGGGATACT |
| | GCTCTCAAGAAATTAGAATTTAGTTCTTCACCAGATTCTTTAAGTACCATC |
| | AATGATTGGGATGATATGGATGACTTTGATACTTCTGAGACTTCAAAATCA |
| | TTTGTTACACCACCCCAAAGTCACTTTGTAAGAGTAAGCACTGCTCAGAA |
| | ATCAAAAAAGGGTAAGAGAAACTTTTTTAAAGCACAGCTTTATACAACAA |
| | ACACAGTAAAGACTGACTTGCCTCCACCCTCCTCTGAAAGCGAGCAAATA |
| | GATTTGACTGAGGAACAGAAGGATGACTCAGAATGGTTAAGCAGCGATGT |
| | GATTTGCATCGATGATGGCCCCATTGCTGAAGTGCATATAAATGAAGATGC |
| | TCAGGAAAGTGACTCTCTGAAAACTCATTTGGAAGATGAAAGAGATAATA |
| | GCGAAAAGAAGAAGAATTTGGAAGAAGCTGAATTACATTCAACTGAGAA |
| | AGTTCCATGTATTGAATTTGATGATGATGATTATGATACGGATTTTGTTCCA |
| | CCTTCTCCAGAAGAAATTATTTCTGCTTCTTCTTCCTCTTCAAAATGCCTTA |
| | GTACGTTAAAGGACCTTGACACCTCTGACAGAAAAGAGGATGTTCTTAGC |
| | ACATCAAAAGATCTTTTGTCAAAACCTGAGAAAATGAGTATGCAGGAGCT |
| | GAATCCAGAAACCAGCACAGACTGTGACGCTAGACAGATAAGTTTACAGO |
| | AGCAGCTTATTCATGTGATGGAGCACATCTGTAAATTAATTGATACTATTC |
| | CTGATGATAAACTGAAACTTTTGGATTGTGGGAACGAACTGCTTCAGCAGC |
| | GGAACATAAGAAGGAAACTTCTAACGGAAGTAGATTTTAATAAAAGTGAT |
| | GCCAGTCTTCTTGGCTCATTGTGGAGATACAGGCCTGATTCACTTGATGGC |
| | CCTATGGAGGGTGATTCCTGCCCTACAGGGAATTCTATGAAGGAGTTAAA |
| | TTTTTCACACCTTCCCTCAAATTCTGTTTCTCCTGGGGACTGTTTACTGACT |
| | ACCACCCTAGGAAAGACAGGATTCTCTGCCACCAGGAAGAATCTTTTTGA |
| | AAGGCCTTTATTCAATACCCATTTACAGAAGTCCTTTGTAAGTAGCAACTG |
| | GGCTGAAACACCAAGACTAGGAAAAAAAAATGAAAGCTCTTATTTCCCAG |
| | GAAATGTTCTCACAAGCACTGCTGTGAAAGATCAGAATAAACATACTGCT |
| | TCAATAAATGACTTAGAAAGAGAAACCCAACCTTCCTATGATATTGATAA |
| | TTTTGACATAGATGACTTTGATGATGATGATGACTGGGAAGACATAATGCA |
| | TAATTTAGCAGCCAGCAAATCTTCCACAGCTGCCTATCAACCCATCAAGGA |
| | AGGTCGGCCAATTAAATCAGTATCAGAAAGACTTTCCTCAGCCAAGACAG |
| | ACTGTCTTCCAGTGTCATCTACTGCTCAAAATATAAACTTCTCAGAGTCAA |
| | TTCAGAATTATACTGACAAGTCAGCACAAAATTTAGCATCCAGAAATCTG |
| | AAACATGAGCGTTTCCAAAGTCTTAGTTTTCCTCATACAAAGGAAATGATG |
| | AAGATTTTTCATAAAAAATTTGGCCTGCATAATTTTAGAACTAATCAGCTAG |
| | AGGCGATCAATGCTGCACTGCTTGGTGAAGACTGTTTTATCCTGATGCCGA |
| | CTGGAGGTGGTAAGAGTTTGTGTTACCAGCTCCCTGCCTGTGTTTCTCCTG |
| | GGGTCACTGTTGTCATTTCTCCCTTGAGATCACTT (SEQ ID NO: 104) |
| | ATCGTAGATCAAGTCCAAAAGCTGACTTCCTTGGATATTCCAGCTACATA |
| | TCTGACAGGTGATAAGACTGACTCAGAAGCTACAAATATTTACCTCCAGT |
| | TATCAAAAAAAGACCCAATCATAAAACTCCTATATGTCACTCCAGAAAA |
| | GATCTGTGCAAGTAACAGACTCATTTCTACTCTGGAGAATCTCTATGAGA |
| | GGAAGCTCTTGGCACGTTTTGTTATTGATGAAGCACATTGTGTCAGTCAG |
| | TGGGGACATGATTTTCGTCAAGATTACAAAAGAATGAATATGCTTCGCCA |
| | GAAGTTTCCTTCTGTTCCGGTGATGGCTCTTACGGCCACAGCTAATCCCA |
| | GGGTACAGAAGGACATCCTGACTCAGCTGAAGATTCTCAGACCTCAGGT |
| | GTTTAGCATGAGCTTTAACAGACATAATCTGAAATACTATGTATTACCGA |
| | AAAAGCCTAAAAAGGTGGCATTTGATTGCCTAGAATGGATCAGAAAGCA |
| | CCACCCATATGATTCAGGGATAATTTACTGCCTCTCCAGGCGAGAATGTG |
| | ACACCATGGCTGACACGTTACAGAGAGATGGGCTCGCTGCTCTTGCTTAC |
| | CATGCTGGCCTCAGTGATTCTGCCAGAGATGAAGTGCAGCAGAAGTGGA |
| | TTAATCAGGATGGCTGTCAGGTTATCTGTGCTACAATTGCATTTGGAATG |
| | GGGATTGACAAACCGGACGTGCGATTTGTGATTCATGCATCTCTCCCTAA |
| | ATCTGTGGAGGGTTACTACCAAGAATCTGGCAGAGCTGGAAGAGATGGG |
| | GAAATATCTCACTGCCTGCTTTTCTATACCTATCATGATGTGACCAGACTG |
| | AAAAGACTTATAATGATGGAAAAAGATGGAAACCATCATACAAGAGAAA |
| | CTCACTTCAATAATTTGTATAGCATGGTACATTACTGTGAAAATATAACA |
| | GAATGCAGGAGAATACAGCTTTTGGCCTACTTTGGTGAAAATGGATTTAA |
| | TCCTGATTTTTGTAAGAAACACCCAGATGTTTCTTGTGATAATTGCTGTAA |
| | AACAAAGGATTATAAAACAAGAGATGTGACTGACGATGTGAAAAGTATT |
| | GTAAGATTTGTTCAAGAACATAGTTCATCACAAGGAATGAGAAATATAA |
| | AACATGTAGGTCCTTCTGGAAGATTTACTATGAATATGCTGGTCGACATT |
| | TTCTTGGGGAGTAAGAGTGCAAAAATCCAGTCAGGTATATTTGGAAAAG |
| | GATCTGCTTATTCACGACACAATGCCGAAAGACTTTTTAAAAAGCTGATA |
| | CTTGACAAGATTTTGGATGAAGACTTATATATCAATGCCAATGACCAGGC |
| | GATCGCTTATGTGATGCTCGGAAATAAAGCACAAACTGTACTAAATGGCA |
| | ATTTAAAGGTAGACTTTATGGAAACAGAAAATTCCAGCAGTGTGAAAAA |
| | ACAAAAAGCGTTAGTAGCAAAAGTGTCTCAGAGGGAAGAGATGGTTAAA |
| | AAATGTCTTGGAGAACTTACAGAAGTCTGCAAATCTCTGGGGAAAGTTTT |
| | TGGTGTCCATTACTTCAATATTTTTAATACCGTCACTCTCAAGAAGCTTGC |
| | AGAATCTTTATCTTCTGATCCTGAGGTTTTGCTTCAAATTGATGGTGTTAC |
| | TGAAGACAAACTGGAAAAATATGGTGCGGAAGTGATTTCAGTATTACAG |
| | AAATACTCTGAATGGACATCGCCAGCTGAAGACAGTTCCCCAGGGATAA |
| | GCCTGTCCAGCAGCAGAGGCCCCGGAAGAAGTGCCGCTGAGGAGCTTGA |
| | CGAGGAAATACCCGTATCTTCCCACTACTTTGCAAGTAAAACCAGAAATG |
| | AAAGGAAGAGGAAAAAGATGCCAGCCTCCCAAAGGTCTAAGAGGAGAA |
| | AAACTGCTTCCAGTGGTTCCAAGGCAAAGGGGGGGTCTGCCACATGTAG |
| | AAAGATATCTTCCAAAACGAAATCCTCCAGCATCATTGGATCCAGTTCAG |
| | CCTCACATACTTCTCAAGCGACATCAGGAGCCAATAGCAAATTGGGGATT |
| | ATGGCTCCACCGAAGCCTATAAATAGACCGTTTCTTAAGCCTTCATATGC |
| | ATTCTCA (SEQ ID NO: 105) |
| Ns3h | TGCCGCCACCTTAGGGTTTGGGGCGTATATGTCCAAGGCACACGGTATC |
| | GACCCTAACATCAGAACTGGGGTAAGGACCATTACCACGGGCGGCTCCA |
| | TTACGTACTCCACCTATGGCAAGTTCCTTGCCGACGGTGGCTGTTCTGGG |
| | GGCGCCTATGACATCATAATATGTGATGAGTGCCACTCAACTGACTCGAC |
| | TACCATCTTGGGCATCGGCACAGTCCTGGACCAAGCGGAGACGGCTGGA |
| | GCGCGGCTCGTCGTGCTCGCCACCGCTACACCTCCGGGATCGGTTACCGT |
| | GCCACACCCCAATATCGAGGAAATAGGCCTGTCCAACAATGGAGAGATC |
| | CCCTTCTATGGCAAAGCCATCCCCATTGAGGCCATCAAGGGGGGGAGGC |
| | ATCTCATTTTCTGCCATTCCAAGAAGAAATGTGACGAGCTCGCCGCAAA |
| | GCTGACAGGCCTCGGACTGAACGCTGTAGCATATTACCGGGGCCTTGAT |
| | GTGTCCGTCATACCGCCTATCGGAGACGTCGTTGTCGTGGCAACAGACG |
| | CTCTAATGACGGGTTTCACCGGCGATTTTGACTCAGTGATCGACTGCAAT |
| | ACATGTGTCACCCAGACAGTCGACTTCAGCTTGGATCCCACCTTCACCAT |
| | TGAGACGACGACCGTGCCCCAAGACGCGGTGTCGCGCTCGCAACGGCGA |
| | GGTAGAACTGGCAGGGGTAGGAGTGGCATCTACAGGTTTGTGACTCCAG |
| | GAGAACGGCCCTCGGGCATGTTCGATTCTTCGGTCCTGTGTGAGTGCTAT |
| | GACGCGGGCTGTGCTTGGTATGAGCTCACGCCCGCTGAGACCTCGGTTA |
| | GGTTGCGGGCTTACCTAAATACACCAGGGTTGCCCGTCTGCCAGGACCA |
| | TCTGGAGTTCTGGGAGAGCGTCTTCACAGGCCTCACCCACATAGATGCC |
| | CACTTCCTGTCCCAGACTAAACAGGCAGGAGACAACTTTCCTTACCTGGT |
| | GGCATATCAAGCTACAGTGTGCGCCAGGGCTCAAGCTCCACCTCCATCG |
| | TGGGACCAAATGTGGAAGTGTCTCATACGGCTGAAACCTACACTGCACG |
| | GGCCAACACCCCTGCTGTATAGGCTAGGAGCCGTCCAAAATGAGGTCAT |
| | CCTCACACACCCCATAACTAAATACATCATGGCATGCATGTCGGCTGACC |
| | TGGAGGTCGTCACT (SEQ ID NO: 106) |
| PcrA | ATGAATGCCCTGCTGAACCATATGAATACAGAACAATCCGAGGCGGTAAA |
| | GACCACAGAAGGCCCCTTGTTGATCATGGCGGGGGCTGGGAGTGGTAAA |
| | ACGAGGGTCCTTACTCACCGAATAGCGTACTTGCTTGACGAAAAGGACG |
| | TGAGTCCATATAACGTGCTTGCCATTACCTTCACAAACAAGGCTGCTAGA |
| | GAGATGAAGGAAAGAGTCCAAAAACTTGTCGGTGACCAAGCGGAGGTC |
| | ATTTGGATGTCTACCTTCCATTCTATGTGCGTTCGCATACTTCGGCGAGAC |
| | GCGGATAGGATTGGGATCGAACGGAACTTCACGATAATAGATCCTACAGA |
| | TCAAAAGTCTGTAATAAAAGATGTTCTCAAAAATGAGAATATAGATAGCA |
| | AAAAATTTGAACCCCGAATGTTCATAGGTGCCATATCAAACTTGAAGAA |
| | CGAACTCAAAACACCTGCGGACGCACAAAAGGAAGCAACAGACTACCA |
| | CAGTCAGATGGTCGCAACTGTTTATTCCGGCTACCAACGACAGCTGAGTC |
| | GGAATGAAGCACTGGATTTTGATGATCTGATCATGACTACTATTAACCTTT |
| | TTGAAAGAGTACCGGAAGTGTTGGAATATTACCAAAACAAATTTCAATA |
| | TATCCACGTTGATGAATACCAAGATACTAATAAGGCACAGTATACATTGGT |
| | AAAGCTGCTGGCGTCAAAGTTTAAAAATCTTTGCGTGGTCGGGGATAGTG |
| | ACCAGAGCATATACGGTTGGCGCGGCGCCGACATACAGAATATCTTGTC |
| | CTTCGAGAAAGATTATCCTGAGGCGAATACAATCTTCCTTGAGCAGAATTA |
| | TAGATCTACAAAAACTATTTTGAACGCGGCTAACGAAGTAATAAAAAATA |
| | ATAGTGAGCGAAAGCCTAAAGGTCTGTGGACAGCTAACACAAATGGTGA |
| | AAAGATTCATTACTACGAAGCAATGACTGAACGAGACGAAGCGGAGTTC |
| | GTCATCCGGGAAATAATGAAACACCAACGCAACGGCAAGAAATACCAAG |
| | ACATGGCAATTCTGTACAGGACCAATGCGCAATCCAGAGTTCTCGAAGA |
| | AACCTTTATGAAGAGCAATATGCCATACACGATGGTTGGAGGCCAAAAAT |
| | TCTATGATAGGAAAGAGATCAAAGACCTGCTGAGCTACCTCCGAATCATT |
| | GCCAACAGTAACGACGACATCTCACTTCAACGGATTATTAACGTACCGAA |
| | ACGCGGGGTTGGACCCTCATCAGTTGAGAAAGTTCAAAACTATGCGTTGC |
| | AGAACAATATTTCCATGTTTGACGCTCTTGGAGAAGCTGATTTTATCGGC |
| | TTGTCAAAGAAAGTAACCCAGGAGTGTCTTAACTTTTACGAACTGATACA |
| | AAGCCTGATAAAGGAACAGGAATTCCTTGAGATCCACGAGATCGTAGAT |
| | GAAGTTCTGCAAAAATCCGGCTATCGGGAAATGTTGGAAAGGGAGAACA |
| | CGCTCGAAAGTAGGTCAAGACTCGAGAACATAGATGAGTTCATGTCAGT |
| | GCCCAAAGACTATGAGGAGAATACGCCCCTTGAAGAGCAGTCATTGATCA |
| | ATTTCCTTACTGACCTGTCACTCGTTGCCGATATTGACGAAGCTGACACT |
| | GAGAATGGGGTAACATTGATGACGATGCACAGTGCTAAGGGATTGGAGT |
| | TTCCCATAGTCTTCATCATGGGTATGGAGGAGTCCCTCTTTCCACACATTC |
| | GGGCAATCAAATCCGAGGATGATCATGAGATGCAAGAGGAGCGCAGAAT |
| | CTGTTACGTTGCGATTACACGAGCGGAGGAGGTTCTTTATATTACACACG |
| | CAACGTCTCGGATGCTCTTTGGACGCCCACAGTCAAACATGCCCTCTAGG |
| | TTCCTTAAGGAAATACCCGAGAGCCTGCTGGAGAACCATAGCTCAGGTA |
| | AGCGGCAGACCATACAACCAAAAGCTAAGCCGTTCGCCAAACGCGGCTT |
| | CTCACAGCGCACGACTAGCACGAAGAAGCAGGTGCTCTCCAGCGATTGG |
| | AATGTTGGCGATAAGGTTATGCACAAAGCTTGGGGAGAGGGCATGGTTTC |
| | CAATGTGAATGAGAAAAATGGATCCATAGAGCTCGACATCATCTTTAAGA |
| | GCCAGGGCCCAAAGCGCCTGCTCGCTCAGTTCGCTCCAATCGAGAAAAA |
| | AGAAGAC (SEQ ID NO: 107) |
| PcrA M6 | ATGAACTTCCTGTCTGAGCAACTGCTGGCACACCTGAACAAGGAGCAGC |
| | AAGAAGCTGTGCGGACCACCGAGGGACCTCTGCTGATCATGGCCGGCG |
| | CTGGAAGCGGAAAAACAAGAGTGCTGACACACCGCATCGCCTACCTGA |
| | TGGCTGAGAAGCACGTGGCCCCTTGGAACATCCTGGCCATTACCTTTAC |
| | AAACAAGGCCGCTAGAGAGATGAGAGAAAGAGTGCAGAGCCTCCTGGG |
| | AGGCGCCGCCGAGGACGTGTGGATCAGCACCTTCGCCAGCATGGCCGTG |
| | CGGATCCTGAGAAGAGATATCGACAGAATCGGCATCAACCGGAACTTCA |
| | GCATCCTGGACCCAACAGACCAGCTGAGCGTGATGAAAACCATCCTGA |
| | AAGAAAAGAACATCGACCCCAAGAAATTCGAGCCTAGAACAATCCTGG |
| | GCACAATCAGCGCCGCCAAGAATGAACTGCTGCCTCCAGAACAGTTTGC |
| | GAAGCGGGCCTCCACCTACTATGAGAAAGTGGTGTCTGACGTCTACCAG |
| | GAGTATCAGCAGCGGCTGCTCAGGTGTCACAGCCTTGACTTCGATGATC |
| | TGATCATGACCACAATCCAGCTGTTTGACCGGGTCCCCGACGTGCTCCA |
| | CTACTACCAATACAAGTTTCAGTACATCCACATCGATGAGTACCAGGAC |
| | ACAAACAGAGCCCAATACACCCTGGTGAAAAAGCTGGCTGAGCGGTTC |
| | CAGAACATCGCCGCCGTGGGCGACGCCGATCAGTCTATCTACAGATGGC |
| | GGGGCGCCGACATCCAGAACATCCTGAGCTTCGAAAGAGATTACCCCAA |
| | CGCCAAAGTGATCCTGCTGGAGCAAAATTACCGGAGCACGAAGCGCATC |
| | CTGCAGGCCGCAAACGAGGTGATCGAGCACAACGTGAACAGAAAGCCT |
| | AAGCGGATCTGGACCGAGAATCCTGAGGGCAAGCCCATCCTGTACTACG |
| | AGGCCATGAACGAAGCCGACGAGGCCCAGTTCGTGGCCGGCAGAATCA |
| | GAGAGGCCGTGGAGCGCGGCGAGAGAAGATACCGAGACTTCGCCGTGC |
| | TGTACAGAACCAATGCCCAGTCCAGAGTCATGGAAGAGATGCTGCTGA |
| | AGGCCAACATCCCTTACCAGATAGTGGGCGGCGTGAAGTTCTACGACAG |
| | AAAGGAAATCAAGGA (SEQ ID NO: 108) |
| TraI | ATGGCGAAGATCCACATGGTCCTTCAGGGTAAAGGTGGGGTCGGAAAAA |
| | GCGCAATCGCGGCGATCATAGCCCAGTACAAGATGGACAAAGGTCAGAC |
| | GCCGCTTTGTATAGATACAGATCCAGTCAATGCCACGTTTGAGGGATATAA |
| | GGCCCTTAATGTACGACGCCTTAACATCATGGCTGGGGATGAGATCAACA |
| | GCCGCAACTTTGATACACTGGTCGAGCTCATCGCGCCCACCAAAGATGAC |
| | GTAGTAATCGACAACGGTGCCTCATCTTTTGTTCCTCTGTCACACTATCTC |
| | ATATCAAATCAAGTACCGGCACTCCTCCAGGAGATGGGACATGAGCTCGT |
| | GATACATACCGTGGTAACTGGCGGCCAGGCATTGCTTGACACTGTAAGTG |
| | GGTTCGCCCAGCTTGCCAGCCAGTTTCCAGCTGAAGCTCTGTTTGTCGTG |
| | TGGCTGAATCCGTACTGGGGTCCAATTGAACACGAAGGGAAGTCATTCG |
| | AGCAAATGAAAGCTTACACTGCTAATAAGGCTAGGGTCTCAAGCATTATC |
| | CAAATCCCAGCCCTTAAGGAGGAGACATACGGACGAGACTTCTCCGATAT |
| | GCTGCAAGAACGGTTGACTTTCGACCAGGCCCTCGCAGACGAATCTTTGA |
| | CTATAATGACCCGGCAGAGGCTTAAAATAGTGAGACGCGGCCTTTTCGAA |
| | CAATTGGACGCCGCAGCCGTTCTG (SEQ ID NO: 109) |
| UvrD | ATGGACGTATCTTACCTTTTGGATTCATTGAATGACAAACAGCGCGAAGC |
| | TGTAGCTGCCCCAAGATCCAACCTCTTGGTGTTGGCGGGTGCTGGCTCAG |
| | GGAAGACCCGAGTTCTTGTACACAGAATCGCGTGGCTTATGTCTGTTGAA |
| | AATTGCAGCCCTTACTCTATAATGGCCGTCACGTTTACGAATAAGGCAGCT |
| | GCGGAAATGCGCCATCGAATTGGGCAGCTTATGGGCACTTCACAAGGTGG |
| | CATGTGGGTGGGAACTTTTCACGGGCTCGCTCATCGGCTGTTGCGCGCAC |
| | ATCACATGGACGCGAACCTGCCTCAAGATTTTCAGATCCTCGATTCCGAA |
| | GATCAATTGCGCCTTCTGAAAAGACTGATCAAGGCAATGAACCTGGATGA |
| | AAAGCAATGGCCACCCCGACAGGCAATGTGGTACATAAACAGCCAAAAA |
| | GACGAGGGTCTTCGACCACATCATATCCAAAGTTATGGTAACCCTGTTGA |
| | ACAAACATGGCAGAAGGTTTATCAGGCGTACCAGGAAGCCTGTGACCGA |
| | GCGGGTCTTGTCGATTTTGCAGAACTGCTCCTCAGAGCACATGAGTTGTG |
| | GCTCAATAAACCTCACATCCTTCAGCATTATAGGGAAAGATTCACGAATAT |
| | ACTGGTTGATGAGTTTCAGGATACAAACAATATCCAATATGCTTGGATTA |
| | GACTTCTCGCAGGAGACACGGGTAAGGTGATGATCGTCGGTGACGATGA |
| | CCAATCAATTTACGGATGGCGGGGTGCCCAGGTGGAAAACATACAAAGA |
| | TTTCTTAACGACTTCCCTGGGGCGGAAACGATTAGGTTGGAGCAGAATTA |
| | TCGGAGTACTTCTAACATACTCAGTGCAGCTAACGCACTCATCGAGAACA |
| | ACAACGGCCGACTGGGAAAGAAGCTTTGGACAGATGGCGCTGACGGTGA |
| | GCCGATATCACTGTATTGCGCGTTTAACGAGCTTGACGAGGCCCGCTTCGT |
| | CGTCAATAGAATAAAAACCTGGCAAGATAATGGGGGGGCGCTCGCTGAG |
| | TGCGCTATTTTGTATCGGAGTAACGCGCAAAGCCGGGTTTTGGAGGAAGC |
| | ACTTCTTCAGGCATCTATGCCATACCGGATATATGGAGGAATGCGATTTTTT |
| | GAACGCCAGGAGATCAAAGATGCGCTTAGTTATCTTCGACTTATTGCGAAT |
| | AGAAATGATGATGCGGCCTTTGAGCGGGTTGTCAATACACCCACACGCGG |
| | GATAGGTGACAGAACTCTGGATGTTGTCAGGCAAACATCTAGAGACCGG |
| | CAACTCACCCTTTGGCAGGCATGTAGAGAACTGCTCCAGGAAAAAGCAC |
| | TGGCTGGCCGCGCGGCATCAGCACTGCAAAGGTTTATGGAGCTGATTGA |
| | CGCTCTTGCACAGGAAACAGCGGACATGCCCTTGCATGTCCAAACGGAT |
| | AGAGTGATCAAAGATTCAGGTCTTCGCACGATGTATGAACAAGAAAAAG |
| | GGGAGAAAGGGCAGACTAGGATAGAGAACCTCGAAGAATTGGTTACAG |
| | CTACCCGCCAGTTTTCATACAATGAGGAAGACGAGGACCTGATGCCACTC |
| | CAAGCTTTCTTGTCCCACGCTGCGCTCGAAGCCGGCGAGGGTCAAGCTGA |
| | TACCTGGCAGGATGCGGTACAACTGATGACCCTCCACTCAGCCAAGGGT |
| | CTGGAATTCCCTCAAGTGTTTATCGTCGGGATGGAGGAGGGTATGTTCCC |
| | ATCCCAGATGTCTTTGGACGAAGGAGGACGACTCGAGGAGGAGCGGCGA |
| | CTCGCCTATGTTGGTGTAACCCGAGCGATGCAGAAACTCACGTTGACGTA |
| | TGCAGAGACGCGCAGGTTGTATGGGAAGGAGGTGTACCACCGACCCTCT |
| | AGGTTCATTGGCGAACTTCCTGAAGAATGTGTGGAAGAAGTACGCCTGC |
| | GGGCTACGGTATCTAGGCCCGTTAGCCATCAACGGATGGGGACTCCAATG |
| | GTGGAGAATGACTCAGGCTATAAGCTGGGCCAAAGGGTCCGCCACGCGA |
| | AATTTGGTGAGGGCACCATCGTAAACATGGAAGGAAGTGGTGAACATTC |
| | AAGGCTTCAAGTAGCCTTTCAGGGACAAGGGATTAAGTGGCTTGTGGCC |
| | GCATACGCGCGACTCGAGAGCGTG (SEQ ID NO: 110) |

TABLE 8
Table of significant exome off-target sites

| Genomic bin | Sample |
|---|---|
| chr1: 44100001-44200000 | AID |
| chr1: 145300001-145400000 | AID-BLM-UGI |
| chr1: 152100001-152200000 | AID |
| chr1: 152200001-152300000 | AID, AID-Ns3h-UGI |
| chr1: 152300001-152400000 | AID |
| chr10: 128100001-128200000 | AID |
| chr11: 1100001-1200000 | AID |
| chr11: 1200001-1300000 | AID |
| chr11: 62500001-62600000 | AID, AID-Ns3h-UGI |
| chr14: 104900001-105000000 | AID |
| chr4: 9200001-9300000 | AID |
| chr5: 140800001-140900000 | AID |
| chr5: 141300001-141400000 | AID |
| chr6: 26100001-26200000 | AID-Ns3h-UGI, AID-PcrA M6-UGI |
| chr6: 27100001-27200000 | AID-Ns3h-UGI, AID-PcrA M6-UGI |
| chr6: 31000001-31100000 | AID |
| chr6: 31800001-31900000 | AID-Ns3h-UGI, AID-PcrA-UGI |
| chr7: 73100001-73200000 | AID-PcrA-UGI |
| chr7: 101000001-101100000 | AID |
| chr8: 143800001-143900000 | AID |
| chrX: 115100001-115200000 | AID |

TABLE 9
Table of hit candidates identified in MEK1 Resistance Screens

| | CDS base | Reference | Alternate | Pheno- | Adjusted |
|---|---|---|---|---|---|
| Drug | position | Base | Base | type | p-value |
| Selumetinib | 170 | A | T | K57M | 1.37E−45 |
| Selumetinib | 360 | G | A | Silent | 1.18E−30 |
| Selumetinib | 383 | G | A | G128D | 5.51E−56 |
| Selumetinib | 607 | G | A | E203K | 0 |
| Selumetinib | 681 | G | A | Silent | 6.66E−24 |
| Trametinib | 383 | G | A | G128D | 0.00E+00 |
| Trametinib | 605 | G | A | G202E | 0.00E+00 |
| Trametinib | 607 | G | A | E203K | 3.98E−190 |
| Trametinib | 630 | G | A | Silent | 0.00E+00 |
| Trametinib | 654 | C | T | Silent | 0.04652943 |

TABLE 10
CDS sequence for MEK 1 mutations

| Name | Sequence |
|---|---|
| MEK 1 WT | ATGCCCAAGAAGAAGCCGACGCCCATCCAGCTGAACCCGGCCCCC |
| | GACGGCTCTGCAGTTAACGGGACCAGCTCTGCGGAGACCAACTTG |
| | GAGGCCTTGCAGAAGAAGCTGGAGGAGCTAGAGCTTGATGAGCAG |
| | CAGCGAAAGCGCCTTGAGGCCTTTCTTACCCAGAAGCAGAAGGTG |
| | GGAGAACTGAAGGATGACGACTTTGAGAAGATCAGTGAGCTGGGG |
| | GCTGGCAATGGCGGTGTGGTGTTCAAGGTCTCCCACAAGCCTTCTG |
| | GCCTGGTCATGGCCAGAAAGCTAATTCATCTGGAGATCAAACCCG |
| | CAATCCGGAACCAGATCATAAGGGAGCTGCAGGTTCTGCATGAGT |
| | GCAACTCTCCGTACATCGTGGGCTTCTATGGTGCGTTCTACAGCGA |
| | TGGCGAGATCAGTATCTGCATGGAGCACATGGATGGAGGTTCTCTG |
| | GATCAAGTCCTGAAGAAAGCTGGAAGAATTCCTGAACAAATTTTA |
| | GGAAAAGTTAGCATTGCTGTAATAAAAGGCCTGACATATCTGAGG |
| | GAGAAGCACAAGATCATGCACAGAGATGTCAAGCCCTCCAACATC |
| | CTAGTCAACTCCCGTGGGGAGATCAAGCTCTGTGACTTTGGGGTCA |
| | GCGGGCAGCTCATCGACTCCATGGCCAACTCCTTCGTGGGCACAA |
| | GGTCCTACATGTCGCCAGAAAGACTCCAGGGGACTCATTACTCTGT |
| | GCAGTCAGACATCTGGAGCATGGGACTGTCTCTGGTAGAGATGGC |
| | GGTTGGGAGGTATCCCATCCCTCCTCCAGATGCCAAGGAGCTGGA |
| | GCTGATGTTTGGGTGCCAGGTGGAAGGAGATGCGGCTGAGACCCC |
| | ACCCAGGCCAAGGACCCCCGGGAGGCCCCTTAGCTCATACGGAAT |
| | GGACAGCCGACCTCCCATGGCAATTTTTGAGTTGTTGGATTACATA |
| | GTCAACGAGCCTCCTCCAAAACTGCCCAGTGGAGTGTTCAGTCTGG |
| | AATTTCAAGATTTTGTGAATAAATGCTTAATAAAAAACCCCGCAG |
| | AGAGAGCAGATTTGAAGCAACTCATGGTTCATGCTTTTATCAAGA |
| | GATCTGATGCTGAGGAAGTGGATTTTGCAGGTTGGCTCTGCTCCAC |
| | CATCGGCCTTAACCAGCCCAGCACACCAACCCATGCTGCTGGCGTC |
| | TAA (SEQ ID NO: 111) |
| MEK1 G128D | ATGCCCAAGAAGAAGCCGACGCCCATCCAGCTGAACCCGGCCCC |
| | CGACGGCTCTGCAGTTAACGGGACCAGCTCTGCGGAGACCAACTT |
| | GGAGGCCTTGCAGAAGAAGCTGGAGGAGCTAGAGCTTGATGAGC |
| | AGCAGCGAAAGCGCCTTGAGGCCTTTCTTACCCAGAAGCAGAAG |
| | GTGGGAGAACTGAAGGATGACGACTTTGAGAAGATCAGTGAGCT |
| | GGGGGCTGGCAATGGCGGTGTGGTGTTCAAGGTCTCCCACAAGCC |
| | TTCTGGCCTGGTCATGGCCAGAAAGCTAATTCATCTGGAGATCAA |
| | ACCCGCAATCCGGAACCAGATCATAAGGGAGCTGCAGGTTCTGC |
| | ATGAGTGCAACTCTCCGTACATCGTGGACTTCTATGGTGCGTTCTA |
| | CAGCGATGGCGAGATCAGTATCTGCATGGAGCACATGGATGGAGG |
| | TTCTCTGGATCAAGTCCTGAAGAAAGCTGGAAGAATTCCTGAACA |
| | AATTTTAGGAAAAGTTAGCATTGCTGTAATAAAAGGCCTGACATA |
| | TCTGAGGGAGAAGCACAAGATCATGCACAGAGATGTCAAGCCCT |
| | CCAACATCCTAGTCAACTCCCGTGGGGAGATCAAGCTCTGTGACT |
| | TTGGGGTCAGCGGGCAGCTCATCGACTCCATGGCCAACTCCTTCG |
| | TGGGCACAAGGTCCTACATGTCGCCAGAAAGACTCCAGGGGACT |
| | CATTACTCTGTGCAGTCAGACATCTGGAGCATGGGACTGTCTCTGG |
| | TAGAGATGGCGGTTGGGAGGTATCCCATCCCTCCTCCAGATGCCA |
| | AGGAGCTGGAGCTGATGTTTGGGTGCCAGGTGGAAGGAGATGCG |
| | GCTGAGACCCCACCCAGGCCAAGGACCCCCGGGAGGCCCCTTAG |
| | CTCATACGGAATGGACAGCCGACCTCCCATGGCAATTTTTGAGTT |
| | GTTGGATTACATAGTCAACGAGCCTCCTCCAAAACTGCCCAGTGG |
| | AGTGTTCAGTCTGGAATTTCAAGATTTTGTGAATAAATGCTTAATA |
| | AAAAACCCCGCAGAGAGAGCAGATTTGAAGCAACTCATGGTTCAT |
| | GCTTTTATCAAGAGATCTGATGCTGAGGAAGTGGATTTTGCAGGTT |
| | GGCTCTGCTCCACCATCGGCCTTAACCAGCCCAGCACACCAACCC |
| | ATGCTGCTGGCGTCTAA (SEQ ID NO: 112) |
| MEK1 G202E | ATGCCCAAGAAGAAGCCGACGCCCATCCAGCTGAACCCGGCCCC |
| | CGACGGCTCTGCAGTTAACGGGACCAGCTCTGCGGAGACCAACTT |
| | GGAGGCCTTGCAGAAGAAGCTGGAGGAGCTAGAGCTTGATGAGC |
| | AGCAGCGAAAGCGCCTTGAGGCCTTTCTTACCCAGAAGCAGAAG |
| | GTGGGAGAACTGAAGGATGACGACTTTGAGAAGATCAGTGAGCT |
| | GGGGGCTGGCAATGGCGGTGTGGTGTTCAAGGTCTCCCACAAGCC |
| | TTCTGGCCTGGTCATGGCCAGAAAGCTAATTCATCTGGAGATCAA |
| | ACCCGCAATCCGGAACCAGATCATAAGGGAGCTGCAGGTTCTGC |
| | ATGAGTGCAACTCTCCGTACATCGTGGGCTTCTATGGTGCGTTCTA |
| | CAGCGATGGCGAGATCAGTATCTGCATGGAGCACATGGATGGAGG |
| | TTCTCTGGATCAAGTCCTGAAGAAAGCTGGAAGAATTCCTGAACA |
| | AATTTTAGGAAAAGTTAGCATTGCTGTAATAAAAGGCCTGACATA |
| | TCTGAGGGAGAAGCACAAGATCATGCACAGAGATGTCAAGCCCT |
| | CCAACATCCTAGTCAACTCCCGTGAGGAGATCAAGCTCTGTGACT |
| | TTGGGGTCAGCGGGCAGCTCATCGACTCCATGGCCAACTCCTTCG |
| | TGGGCACAAGGTCCTACATGTCGCCAGAAAGACTCCAGGGGACT |
| | CATTACTCTGTGCAGTCAGACATCTGGAGCATGGGACTGTCTCTG |
| | GTAGAGATGGCGGTTGGGAGGTATCCCATCCCTCCTCCAGATGCC |
| | AAGGAGCTGGAGCTGATGTTTGGGTGCCAGGTGGAAGGAGATGC |
| | GGCTGAGACCCCACCCAGGCCAAGGACCCCCGGGAGGCCCCTTA |
| | GCTCATACGGAATGGACAGCCGACCTCCCATGGCAATTTTTGAGT |
| | TGTTGGATTACATAGTCAACGAGCCTCCTCCAAAACTGCCCAGTG |
| | GAGTGTTCAGTCTGGAATTTCAAGATTTTGTGAATAAATGCTTAA |
| | TAAAAAACCCCGCAGAGAGAGCAGATTTGAAGCAACTCATGGTT |
| | CATGCTTTTATCAAGAGATCTGATGCTGAGGAAGTGGATTTTGCA |
| | GGTTGGCTCTGCTCCACCATCGGCCTTAACCAGCCCAGCACACCA |
| | ACCCATGCTGCTGGCGTCTAA (SEQ ID NO: 113) |
| MEK1 E203K | ATGCCCAAGAAGAAGCCGACGCCCATCCAGCTGAACCCGGCCCC |
| | CGACGGCTCTGCAGTTAACGGGACCAGCTCTGCGGAGACCAACTT |
| | GGAGGCCTTGCAGAAGAAGCTGGAGGAGCTAGAGCTTGATGAGC |
| | AGCAGCGAAAGCGCCTTGAGGCCTTTCTTACCCAGAAGCAGAAG |
| | GTGGGAGAACTGAAGGATGACGACTTTGAGAAGATCAGTGAGCT |
| | GGGGGCTGGCAATGGCGGTGTGGTGTTCAAGGTCTCCCACAAGCC |
| | TTCTGGCCTGGTCATGGCCAGAAAGCTAATTCATCTGGAGATCAA |
| | ACCCGCAATCCGGAACCAGATCATAAGGGAGCTGCAGGTTCTGCA |
| | TGAGTGCAACTCTCCGTACATCGTGGGCTTCTATGGTGCGTTCTAC |
| | AGCGATGGCGAGATCAGTATCTGCATGGAGCACATGGATGGAGGT |
| | TCTCTGGATCAAGTCCTGAAGAAAGCTGGAAGAATTCCTGAACAA |
| | ATTTTAGGAAAAGTTAGCATTGCTGTAATAAAAGGCCTGACATAT |
| | CTGAGGGAGAAGCACAAGATCATGCACAGAGATGTCAAGCCCTC |
| | CAACATCCTAGTCAACTCCCGTGGGAAGATCAAGCTCTGTGACTT |
| | TGGGGTCAGCGGGCAGCTCATCGACTCCATGGCCAACTCCTTCGT |
| | GGGCACAAGGTCCTACATGTCGCCAGAAAGACTCCAGGGGACTC |
| | ATTACTCTGTGCAGTCAGACATCTGGAGCATGGGACTGTCTCTGG |
| | TAGAGATGGCGGTTGGGAGGTATCCCATCCCTCCTCCAGATGCCA |
| | AGGAGCTGGAGCTGATGTTTGGGTGCCAGGTGGAAGGAGATGCG |
| | GCTGAGACCCCACCCAGGCCAAGGACCCCCGGGAGGCCCCTTAG |
| | CTCATACGGAATGGACAGCCGACCTCCCATGGCAATTTTTGAGTT |
| | GTTGGATTACATAGTCAACGAGCCTCCTCCAAAACTGCCCAGTGG |
| | AGTGTTCAGTCTGGAATTTCAAGATTTTGTGAATAAATGCTTAAT |
| | AAAAAACCCCGCAGAGAGAGCAGATTTGAAGCAACTCATGGTTC |
| | ATGCTTTTATCAAGAGATCTGATGCTGAGGAAGTGGATTTTGCAG |
| | GTTGGCTCTGCTCCACCATCGGCCTTAACCAGCCCAGCACACCAA |
| | CCCATGCTGCTGGCGTCTAA (SEQ ID NO: 114) |

TABLE 11
Sequence for SF3B1 splicing minigene reporter

| | |
|---|---|
| DLST | (1 Start)ATGGGTCGCTTTGACAGGGAGGTAGATATTGGAATTCCTGATGCTA |
| | CAGGACGCTTAGAGATTCTTCAGATCCATACCAAGAACATGAAGCTGGC |
| | AGATGAGTGGACCTGGAACAG(1 End)(2 Start)GTGAAGTGATGATGATG |
| | GCTGACCAGGCGTTACAGTGTCTCTAGGCAGTTGCTGGGAACTGGCTAG |
| | AGACATAAGGTTAAGATGTGAGGAGATGGGTTTTGATTTCTGGACAGGG |
| | GAAAGGAAGTAATCTGAGATGAATCCAGGAAATG(2 End)AAGCTTCG |
| | ACACCGAGCTCG(3 Start)CCTTTGACAACTGTCTTGTTACAAATGTTTGGT |
| | CCTTGCTTCTTTAACCTCTTAGTAAAG(3End)[[CCTTTGATTGTCTTTTCA |
| | G]](4 Start)CTGTTGGAGACACAGTTGCAGAAGATGAAGTGGTTTGTGAGA |
| | TTGAAACTGACAAG(4 End)CAAGGCAGC(5 start)GAGGGCAGAGGAAGTC |
| | TTCTAACATGCGG(5 end)TGACGTGGAGGAGAATCCCGGCCCT(6 start)GT |
| | CAGTAAAGGTGAAGAACTGTTCACCGGGGTGGTGCCCATCCTGGTCGAGC |
| | TGGACGGCGACGTAAACGGCCACAAGTTCAGCGTGTCCGGCGAGGGCGAG |
| | GGCGATGCCACCTACGGCAAGCTGACCCTGAAGTTCATCTGCACCACCGG |
| | CAAGCTGCCCGTGCCCTGGCCCACCCTCGTGACCACCCTGACCTACGGCGT |
| | GCAGTGCTTCAGCCGCTACCCCGACCACATGAAGCAGCACGACTTCTTCAA |
| | GTCCGCCATGCCCGAAGGCTACGTCCAGGAGCGCACCATCTTCTTCAAGG |
| | ACGACGGCAACTACAAGACCCGCGCCGAGGTGAAGTTCGAGGGCGACACC |
| | CTGGTGAACCGCATCGAGCTGAAGGGCATCGACTTCAAGGAGGACGGCAA |
| | CATCCTGGGGCACAAGCTGGAGTACAACTACAACAGCCACAACGTCTATA |
| | TCATGGCCGACAAGCAGAAGAACGGCATCAAGGTGAACTTCAAGATCCGC |
| | CACAACATCGAGGACGGCAGCGTGCAGCTCGCCGACCACTACCAGCAGAA |
| | CACCCCCATCGGCGACGGCCCCGTGCTGCTGCCCGACAACCACTACCTGA |
| | GCACCCAGTCCGCCCTGAGCAAAGACCCCAACGAGAAGCGCGATCACATG |
| | GTCCTGCTGGAGTTCGTGACCGCCGCCGGGATCACTCTCGGTATGGATGAA |
| | TTATATAAATAA(6 end) (SEQ ID NO: 115) |
| PLAC4 | (1 Start)ATGGGTCGCTTTGACAGGGAGGTAGATATTGGAATTCCTGATGCTAC |
| | AGGACGCTTAGAGATTCTTCAGATCCATACCAAGAACATGAAGCTGGCAG |
| | ATGATGTGGACCTGGAACAG(1 end)(2 start)GTGAAGTGATGATGATGGCT |
| | GACCAGGCGTTACAGTGTCTCTAGGCAGTTGCTGGGAACTGGCTAGAGAC |
| | ATAAGGTTAAGATGTGAGGAGATGGGTTTTGATTTCTGGACAGGGGAAAG |
| | GAAGTAATCTGAGATTGAATCCAGGAAATG(2end)AAGCTTCGACACCGAG |
| | CTCG[(3 start))CCTTTGACAACTGTCTTGTTACAAATGTTTGGTCCTTGCTTC |
| | TTTAACCTCTTAGTAAAG(3 end)[[TTTGTGTATTCTAG]](4 start)ATTACCA |
| | CAGTTCCAGAGACAATGCTGGCACAAGGCTTCCAGCCCATCCTGTCACACT |
| | GACACGGAGAATGAAATCGTCCTGCCTCTGGGCTCCTTAGATCAG(4end)CA |
| | AGGCAGC(5 start)GAGGGCAGAGGAAGTCTTCTAACATGCGGTGACGTGGA |
| | GGAGAATCCCGG CCCT(5 end)(6 start)GTCAGTAAAGGTGAAGAACTGTT |
| | CACCGGGGTGGTGCCCATCCTGGTCGAGCTGGACGGCGACGTAAACGGCC |
| | ACAAGTTCAGCGTGTCCGGCGAGGGCGAGGGCGATGCCACCTACGGCAAG |
| | CTGACCCTGAAGTTCATCTGCACCACCGGCAAGCTGCCCGTGCCCTGGCCC |
| | ACCCTCGTGACCACCCTGACCTACGGCGTGCAGTGCTTCAGCCGCTACCCC |
| | GACCACATGAAGCAGCACGACTTCTTCAAGTCCGCCATGCCCGAAGGCTA |
| | CGTCCAGGAGCGCACCATCTTCTTCAAGGACGACGGCAACTACAAGACCC |
| | GCGCCGAGGTGAAGTTCGAGGGCGACACCCTGGTGAACCGCATCGAGCTG |
| | AAGGGCATCGACTTCAAGGAGGACGGCAACATCCTGGGGCACAAGCTGGA |
| | GTACAACTACAACAGCCACAACGTCTATATCATGGCCGACAAGCAGAAGA |
| | ACGGCATCAAGGTGAACTTCAAGATCCGCCACAACATCGAGGACGGCAGC |
| | GTGCAGCTCGCCGACCACTACCAGCAGAACACCCCCATCGGCGACGGCCC |
| | CGTGCTGCTGCCCGACAACCACTACCTGAGCACCCAGTCCGCCCTGAGCAA |
| | AGACCCCAACGAGAAGCGCGATCACATGGTCCTGCTGGAGTTCGTGACCG |
| | CCGCCGGGATCACTCTCGGTATGGATGAATTATATAAATAA(6 end). (SEQ ID |
| | NO: 116) |

1-Upstream Exon
2-Upstream intron
3-Downstream intron
[[Alternative 3′ splice inclusion]]
4-Downstream exon
5-T2A linker
6-EGFP

TABLE 12
Table of hit candidates identified for SF3B1 Sequences

| Base | ref_{—} | alt_{—} | Pheno- | Mutation | log_fold_{—} | Mutation |
|---|---|---|---|---|---|---|
| Position | base | base | type | Type | change | Type |
| 1627 | A | G | T543A | Missense | 1.38663646 | Non-clinical |
| 1629 | A | G | NA | Silent | 2.53851962 | Non-clinical |
| 1653 | A | G | NA | Silent | 1.93565474 | Non-clinical |
| 1666 | A | G | I556V | Missense | 1.45809111 | Non-clinical |
| 1677 | A | G | I559M | Missense | 1.7797861 | Non-clinical |
| 1682 | A | G | Y561C | Missense | 1.81357408 | Non-clinical |
| 1698 | A | G | NA | Silent | 1.69377311 | Non-clinical |
| 1709 | A | G | Y570C | Missense | 1.24133574 | Clinical |
| 1743 | A | G | NA | Silent | 1.15100255 | Non-clinical |
| 1751 | A | G | D584G | Missense | 1.00724764 | Non-clinical |
| 1757 | A | G | D586G | Missense | 1.72523491 | Clinical |
| 1760 | A | G | Y587C | Missense | 2.29587436 | Non-clinical |
| 1763 | A | G | Y588C | Missense | 1.7345498 | Non-clinical |
| 1768 | A | G | R590G | Missense | 1.17442273 | Non-clinical |
| 1789 | A | G | I597V | Missense | 1.10438786 | \|Non-clinical |
| 1795 | A | G | N599D | Missense | 1.93523264 | Non-clinical |
| 1807 | G | A | A603T | Missense | 1.01810563 | Non-clinical |
| 1822 | A | G | T608A | Missense | 1.17303987 | Non-clinical |
| 1837 | A | G | M613V | Missense | 1.14329741 | Non-clinical |
| 1849 | A | G | I617V | Missense | 2.46217362 | Non-clinical |
| 1851 | A | G | I617M | Missense | 1.63338506 | Non-clinical |
| 1855 | A | G | N619D | Missense | 1.44132924 | Non-clinical |
| 1868 | A | G | Y623C | Missense | 2.08956152 | Clinical |
| 1890 | A | G | NA | Silent | 1.92804782 | Non-clinical |
| 1932 | A | G | NA | Silent | 1.58256239 | Non-clinical |
| 1944 | A | G | NA | Silent | 1.92578678 | Non-clinical |
| 1946 | A | G | K649R | Missense | 1.2510653 | Non-clinical |
| 1985 | A | G | H662R | Missense | 1.30651587 | Clinical |
| 1987 | A | G | T663A | Missense | 1.26105039 | Clinical |
| 1993 | A | G | I665V | Missense | 2.07577179 | Non-clinical |

TABLE 13
Table of hit candidates identified for CD69 enhancer screen.

| | | | | Potential motif | |
|---|---|---|---|---|---|
| Base Position | ref_base | alt_base | Phenotype | targeting | log2_fold_change |
| Chr12: 9764879 | G | A | Reduced CD69 | Affecting ETS | −2.375045886 |
| | | | expression(validated) | motif | |
| Chr12: 9764880 | G | A | Reduced CD69 | Affecting ETS | −2.639362938 |
| | | | expression(validated) | motif | |
| Chr12: 9764930 | G | A | Reduced CD69 | Unclear | −3.28869167 |
| | | | expression(screening) | | |
| Chr12: 9764976 | G | A | Reduced CD69 | Unclear | −2.203083513 |
| | | | expression(screening) | | |
| Chr12: 9764996 | C | T | Reduced CD69 | Affecting RUNX | −3.085492338 |
| | | | expression(validated) | motif | |
| Chr12: 9765002 | C | T | Reduced CD69 | Affecting RUNX | −2.710291572 |
| | | | expression(screening) | motif | |
| Chr12: 9765072 | G | A | Reduced CD69 | Affecting | −2.963576938 |
| | | | expression(screening) | ZSCAN4 motif | |
| Chr12: 9765102 | C | T | Reduced CD69 | Affecting STAT6 | −2.123218172 |
| | | | expression(screening) | motif | |
| Chr12: 9765256 | G | A | Reduced CD69 | Affecting RelB | −2.421260686 |
| | | | expression(screening) | motif | |

Below are the additional manually selected ones from FIG. 5c

| | | | | | |
|---|---|---|---|---|---|
| Ch12: 9764948 | C | T | Reduced CD69 | Affecting GATA | −1.148686331 |
| | | | expression(validated) | motif | |
| Ch12: 9764995 | C | T | Reduced CD69 | Affecting RUNX | −1.553088638 |
| | | | expression(validated) | motif | |
| Ch12: 9764998 | C | T | Reduced CD69 | Affecting RUNX | −1.442156962 |
| | | | expression(validated) | motif | |

TABLE 14
Table of base editor validation sgRNAs

| sgRNA | Target | | | |
|---|---|---|---|---|
| name | gene/region | Experiment | sgRNA sequence | Target function |
| sgCtrl | AAVS1 | NA | GGGGCCACTAG | Safe harbor locus |
| | | | GGACAGGAT | |
| | | | (SEQ ID NO: 117) | |
| sg383 | MEK1 | Validation for CDS | TAGAAGCCCAC | |
| | | base 383 (G > A) | GATGTACGG | |
| | | | (SEQ ID NO: 118) | |
| sg607-1 | MEK1 | Validation for CDS | CCCACGGGAGT | |
| | | base 607 (G > A) | TGACTAGGA | |
| | | | (SEQ ID NO: 119) | |
| sg607-2 | MEK1 | Validation for CDS | CTTGATCTCCCC | |
| | | base 607 (G > A) | ACGGGAGT | |
| | | | (SEQ ID NO: 120) | |
| sg1682 | SF3B1 | Validation for CDS | CTGTACAAACT | |
| | | base 1849 (A > G) | TGATGACTT | |
| | | | (SEQ ID NO: | |
| | | | 121) | |
| sg1849 | SF3B1 | Validation for CDS | ATAGATAACAT | |
| | | base 1849 (A > G) | GGATGAGTA | |
| | | | (SEQ ID NO: 122) | |
| sg1868 | SF3B1 | Validation for CDS | GAGTATGTCCGT | |
| | | base 1868 (A > G) | AACACAAC | |
| | | | (SEQ ID NO: 123) | |
| sg1996-1 | SF3B1 | Validation for CDS | ATTAAGATTGTA | |
| | | base 1996 (A > G) | CAACAGAT (SEQ | |
| | | | ID NO: 124) | |
| sg1996-2 | SF3B1 | Validation for CDS | TGGTATTAAGAT | |
| | | base 1996 (A > G) | TGTACAAC | |
| | | | (SEQ ID NO: 125) | |
| sgK700E-1 | SF3B1 | Validation positive | CAGAAAGTTCG | |
| | | control for K700E | GACCATCAG | |
| | | | (SEQ ID NO: 126) | |
| sgK700E-2 | SF3B1 | Validation positive | AGCAGAAAGTT | |
| | | control for K700E | CGGACCATC | |
| | | | (SEQ ID NO: 127) | |
| sg4948 | CD69 | Validation for | TCCTTTCTGACG | destroy GATA |
| | enhancer | chr12:9764948 | TCTCACCC (SEQ | motif |
| | | C > T (FIG. S6) | ID NO: 128) | |
| sg4879 | CD69 | Validation for | TATCAGACAGCT | IRF/STAT or |
| | enhancer | chr12:9764879/80 | GCAGCAGC (SEQ ID NO: | destroy potential |
| | | G > A (FIG. S6) | 129) | IRF/ETS motif |
| sg4995 | CD69 | Validation for | GACCACAGACT | destroy RUNX |
| | enhancer | chr12:9764995/6/8 | TCCGACCTA | motif, potential |
| | | C > T (FIG. 5 and S7) | (SEQ ID NO: 130) | gain GATA motif |

TABLE 15
Table of prime editor validation sgRNAs

| pegRNA | Target | | | Target |
|---|---|---|---|---|
| name | gene/region | Experiment | pegRNA sequence | function |
| peg1682-1 | SF3B1 | Validation for | GAACAAACCTTATGCACAT | |
| | | CDS base | AGTTTTAGAGCTAGAAATA | |
| | | 1682 (A > G) | GCAAGTTAAAATAAGGCT | |
| | | | A | |
| | | | GTCCGTTATCAACTTGAAA | |
| | | | AAGTGGCACCGAGTCGGT | |
| | | | G | |
| | | | CTgCAAACTTGATGACTTA | |
| | | | G | |
| | | | TTCGTCCATATGTGCATAA | |
| | | | G | |
| | | | GTTCCGAAAGATTGACGC | |
| | | | G | |
| | | | GTTCTATCTAGTTACGCGT | |
| | | | T | |
| | | | AAACCAACTAGAAATTTTT | |
| | | | T (SEQ ID NO: 131) | |
| peg1682-2 | SF3B1 | Validation for | GTACTTGTGAAAGTTATTG | |
| | | CDS base | ATGTTTTAGAGCTAGAAATA | |
| | | 1682 (A > G) | GCAAGTTAAAATAAGGCTA | |
| | | | GTCCGTTATCAACTTGAAA | |
| | | | AAGTGGCACCGAGTCGGTG | |
| | | | CGTTTGCACAGTATCCTATC | |
| | | | AATAACTTTCACTCCCTTTA | |
| | | | TTGACGCGGTTCTATCTAGT | |
| | | | TACGCGTTAAACCAACTAG | |
| | | | AAATTTTTT (SEQ ID NO: | |
| | | | 132) | |
| peg1849 | SF3B1 | Validation for | GAAGCTCTAGCTGTTGTGT | |
| | | CDS base | TAGTTTTAGAGCTAGAAATA | |
| | | 1849 (A > G) | GCAAGTTAAAATAAGGCTA | |
| | | | GTCCGTTATCAACTTGAAA | |
| | | | AAGTGGCACCGAGTCGGTG | |
| | | | CACCTGATgTAGATAACATG | |
| | | | GATGAGTATGTCCGTAACA | |
| | | | CAACAGCTAGATAATAACA | |
| | | | TTGACGCGGTTCTATCTAGT | |
| | | | TACGCGTTAAACCAACTAG | |
| | | | AAATTTTTT (SEQ ID NO: | |
| | | | 133) | |
| peg1849.51- | SF3B1 | Validation for | GAAGCTCTAGCTGTTGTGT | |
| 1 | | CDS base | TAGTTTTAGAGCTAGAAATA | |
| | | 1849 and 1951 | GCAAGTTAAAATAAGGCTA | |
| | | (A > G) | GTCCGTTATCAACTTGAAA | |
| | | | AAGTGGCACCGAGTCGGTG | |
| | | | CGATgTgGATAACATGGATG | |
| | | | AGTATGTCCGTAACACAAC | |
| | | | AGCTAGAGCGAAATATTTT | |
| | | | GACGCGGTTCTATCTAGTTA | |
| | | | CGCGTTAAACCAACTAGAA | |
| | | | ATTTTTT (SEQ ID NO: | |
| | | | 134) | |
| peg1849.51- | SF3B1 | Validation for | GAAGCTCTAGCTGTTGTGT | |
| 2 | | CDS base | TAGTTTTAGAGCTAGAAATA | |
| | | 1849 and 1951 | GCAAGTTAAAATAAGGCTA | |
| | | (A > G) | GTCCGTTATCAACTTGAAA | |
| | | | AAGTGGCACCGAGTCGGTG | |
| | | | CACCTGATgTgGATAACATG | |
| | | | GATGAGTATGTCCGTAACA | |
| | | | CAACAGCTAGATAAATATTT | |
| | | | TGACGCGGTTCTATCTAGTT | |
| | | | ACGCGTTAAACCAACTAGA | |
| | | | AATTTTTT (SEQ ID NO: | |
| | | | 135) | |
| peg1851-1 | SF3B1 | Validation for | GAAGCTCTAGCTGTTGTGT | |
| | | CDS base | TAGTTTTAGAGCTAGAAATA | |
| | | 1851 (A > G) | GCAAGTTAAAATAAGGCTA | |
| | | | GTCCGTTATCAACTTGAAA | |
| | | | AAGTGGCACCGAGTCGGTG | |
| | | | CGATATgGATAACATGGATG | |
| | | | AGTATGTCCGTAACACAAC | |
| | | | AGCTAGAGCACAGAACCTT | |
| | | | GACGCGGTTCTATCTAGTTA | |
| | | | CGCGTTAAACCAACTAGAA | |
| | | | ATTTTTT (SEQ ID NO: | |
| | | | 136) | |
| peg1851-2 | SF3B1 | Validation for | GAAGCTCTAGCTGTTGTGT | |
| | | CDS base | TAGTTTTAGAGCTAGAAATA | |
| | | 1851 (A > G) | GCAAGTTAAAATAAGGCTA | |
| | | | GTCCGTTATCAACTTGAAA | |
| | | | AAGTGGCACCGAGTCGGTG | |
| | | | CACCTGATATgGATAACATG | |
| | | | GATGAGTATGTCCGTAACA | |
| | | | CAACAGCTAGAAACAACA | |
| | | | CTTGACGCGGTTCTATCTAG | |
| | | | TTACGCGTTAAACCAACTA | |
| | | | GAAATTTTTT (SEQ ID NO: | |
| | | | 137) | |
| peg1868 | SF3B1 | Validation for | GAAGCTCTAGCTGTTGTGT | |
| | | CDS base | TAGTTTTAGAGCTAGAAATA | |
| | | 1868 (A > G) | GCAAGTTAAAATAAGGCTA | |
| | | | GTCCGTTATCAACTTGAAA | |
| | | | AAGTGGCACCGAGTCGGTG | |
| | | | CTGAGTgTGTCCGTAACAC | |
| | | | AACAGCTAGTAACAAATTT | |
| | | | GACGCGGTTCTATCTAGTTA | |
| | | | CGCGTTAAACCAACTAGAA | |
| | | | ATTTTTT (SEQ ID NO: | |
| | | | 138) | |
| peg1996-1 | SF3B1 | Validation for | GTCCTGGCAAGCGAGACAC | |
| | | CDS base | ACGTTTTAGAGCTAGAAAT | |
| | | 1996 (A > G) | AGCAAGTTAAAATAAGGCT | |
| | | | AGTCCGTTATCAACTTGAA | |
| | | | AAAGTGGCACCGAGTCGGT | |
| | | | GCATCTTAAcACCAGTGTGT | |
| | | | CTCGCTTGCAACCCTTCTT | |
| | | | GACGCGGTTCTATCTAGTTA | |
| | | | CGCGTTAAACCAACTAGAA | |
| | | | ATTTTTT (SEQ ID NO: | |
| | | | 139) | |
| peg1996-2 | SF3B1 | Validation for | GTCCTGGCAAGCGAGACAC | |
| | | CDS base | ACGTTTTAGAGCTAGAAAT | |
| | | 1996 (A > G) | AGCAAGTTAAAATAAGGCT | |
| | | | AGTCCGTTATCAACTTGAA | |
| | | | AAAGTGGCACCGAGTCGGT | |
| | | | GCATCTcAAcACCAGTGTGT | |
| | | | CTCGCTTGCAATTCTTCTTG | |
| | | | ACGCGGTTCTATCTAGTTAC | |
| | | | GCGTTAAACCAACTAGAAA | |
| | | | TTTTTT (SEQ ID NO: 140) | |
| peg1996-3 | SF3B1 | Validation for | GTGTGCAAAAGCAAGAAG | |
| | | CDS base | TCCGTTTTAGAGCTAGAAA | |
| | | 1996 (A > G) | TAGCAAGTTAAAATAAGGC | |
| | | | TAGTCCGTTATCAACTTGAA | |
| | | | AAAGTGGCACCGAGTCGGT | |
| | | | GCATCTTAAcACCAGTGTGT | |
| | | | CTCGCTTGCCAGGACTTCT | |
| | | | TGCTTTTGCTTCTAAATTTG | |
| | | | ACGCGGTTCTATCTAGTTAC | |
| | | | GCGTTAAACCAACTAGAAA | |
| | | | TTTTTT (SEQ ID NO: 141) | |
| pegWT | CD69 | Validation | TGTCTTAGGTCGGAAGTCT | none |
| | enhancer | Control for | GGTTTTAGAGCTAGAAATA | |
| | | chr12: | GCAAGTTAAAATAAGGCTA | |
| | | 9764995/6/8 | GTCCGTTATCAACTTGAAA | |
| | | C->T (FIG. 5 | AAGTGGCACCGAGTCGGTG | |
| | | and S7) | CAACAGGGACCACAGACTT | |
| | | | CCGACCTAAG (SEQ ID NO: | |
| | | | 142) | |
| peg4995 | CD69 | Validation for | TGTCTTAGGTCGGAAGTCT | destroy RUNX |
| | enhancer | chr12:9764995 | GGTTTTAGAGCTAGAAATA | motif |
| | | C->T (FIG. 5 | GCAAGTTAAAATAAGGCTA | |
| | | and S7) | GTCCGTTATCAACTTGAAA | |
| | | | AAGTGGCACCGAGTCGGTG | |
| | | | CAACAGGGATCACAGACTT | |
| | | | CCGACCTAAG (SEQ ID NO: | |
| | | | 143) | |
| peg4996 | CD69 | Validation for | TGTCTTAGGTCGGAAGTCT | destroy RUNX |
| | enhancer | chr12:9764996 | GGTTTTAGAGCTAGAAATA | motif |
| | | C->T (FIG. 5 | GCAAGTTAAAATAAGGCTA | |
| | | and S7) | GTCCGTTATCAACTTGAAA | |
| | | | AAGTGGCACCGAGTCGGTG | |
| | | | CAACAGGGACTACAGACTT | |
| | | | CCGACCTAAG (SEQ ID NO: | |
| | | | 144) | |
| peg4998 | CD69 | Validation for | TGTCTTAGGTCGGAAGTCT | destroy RUNX |
| | enhancer | chr12:9764998 | GGTTTTAGAGCTAGAAATA | motif |
| | | C->T (FIG. 5 | GCAAGTTAAAATAAGGCTA | |
| | | and S7) | GTCCGTTATCAACTTGAAA | |
| | | | AAGTGGCACCGAGTCGGTG | |
| | | | CAACAGGGACCATAGACTT | |
| | | | CCGACCTAAG (SEQ ID NO: | |
| | | | 145) | |
| peg4995/6/ | CD69 | Validation for | TGTCTTAGGTCGGAAGTCT | destroy RUNX |
| 8 | enhancer | chr12:9764995/ | GGTTTTAGAGCTAGAAATA | motif, gain |
| | | 6/8 | GCAAGTTAAAATAAGGCTA | GATA motif |
| | | C->T (FIG. 5 | GTCCGTTATCAACTTGAAA | |
| | | and S7) | AAGTGGCACCGAGTCGGTG | |
| | | | CAACAGGGATTATAGACTT | |
| | | | CCGACCTAAG (SEQ ID NO: | |
| | | | 146) | |

Nicking guides

| ID | Paired pegRNA | Spacer sequence |
|---|---|---|
| DC737 | peg1682-1 | GCTGATGTCTCCTACACTTG (SEQ ID NO: 147) |
| DC738 | peg1682-2 | GAACAAACCTTATGCACATA (SEQ ID NO: 148) |
| DC740 | peg1849 | TGCTGTTGTAGCCTCTGCCC (SEQ ID NO: 149) |
| DC740 | peg1849.51-1 | TGCTGTTGTAGCCTCTGCCC (SEQ ID NO: 150) |
| DC740 | peg1849.51-2 | TGCTGTTGTAGCCTCTGCCC (SEQ ID NO: 151) |
| DC740 | peg1851-1 | TGCTGTTGTAGCCTCTGCCC (SEQ ID NO: 152) |
| DC740 | peg1851-2 | TGCTGTTGTAGCCTCTGCCC (SEQ ID NO: 153) |
| DC743 | peg1868 | GCTGTTGTAGCCTCTGCCCT (SEQ ID NO: 154) |
| DC745 | peg1996-1 | ACTTCTAAGATGTGGCAAGA (SEQ ID NO: 155) |
| DC745 | peg1996-2 | ACTTCTAAGATGTGGCAAGA (SEQ ID NO: 156) |
| DC748 | peg1996-3 | GCAATAAAGAAGGAATGCCC (SEQ ID NO: 157 |

TABLE 16
Comparison of HACE to state-of-the-art
methods for nucleotide diversification

| | | | | Editing |
|---|---|---|---|---|
| Method | Species | Target | Editing rate | window |
| TRACE | Mammalian | Exogenous | ~2 kbp⁻¹ per | ~2000 bp per |
| | | overexpression | 3 days | T7 promoter |
| TRIDENT | Mammalian | Exogenous | ~2 kbp⁻¹ per | ~2000 bp per |
| | | overexpression | 3 days | T7 promoter |
| CRISPR-X | Mammalian | Endogenous | ~1 kbp⁻¹ | 10-50 bp per |
| | | | per ~12 days | sgRNA |
| TAM | Mammalian | Endogenous | ~1 kbp⁻¹ | 10-50 bp per |
| | | | per ~7 days | sgRNA |
| EvolvR | E. coli | Endogenous | ~0.05 kbp⁻¹ | ~50 bp per |
| | | | per day | sgRNA |
| OrthoRep | Yeast | Exogenous | ~0.16 kbp⁻¹ | At least 5000 |
| | | overexpression | per day | bp |
| VEGAS | Mammalian | Exogenous | ~1 kbp⁻¹ per | ~6000 bp |
| | | overexpression | day | |
| HACE | Human | Endogenous | ~2 kbp⁻¹ per | >1000 bp per |
| | | | 3 days | sgRNA |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth. Citation or identification of any document in this application is not an admission that such a document is available as prior art to the present invention.

1. A composition for targeted mutagenesis comprising:
(a) one or more programmable nickases configured to introduce a single-strand nick in double-stranded DNA (dsDNA) at one or more targeted nick sites;
(b) one or more helicases configured to unwind a portion of the dsDNA at the one or more targeted nick sites to form a portion of unwound dsDNA; and
(c) one or more deaminases configured to introduce one or more base edits within the portion of unwound dsDNA.

2. The composition of claim 1, wherein the one or more programmable nickases comprise:
(a) a Cas nickase (nCas); and
(b) one or more guide molecules capable of forming a complex with the nCas and directing sequence-specific binding of the complex to the one or more targeted nick sites.

3. (canceled)

4. The composition of claim 1, wherein the one or more programmable nickases comprise:
(a) an OMEGA nickase; and
(b) one or more @RNA molecules capable of forming a complex with the OMEGA nickase and directing sequence-specific binding of the complex to the one or more targeted nick sites.

5. The composition of claim 4, wherein the OMEGA nickase comprises an IscB nickase, an IsrB nickase, an IshB nickase, a TnpB nickase, or a Fanzor nickase.

6. The composition of claim 1, wherein the one or more helicases exhibit a processivity range of greater than or equal to 200 base pairs.

7. (canceled)

8. The composition of claim 1, wherein the one or more helicases exhibits a processivity range of less than 200 base pairs.

9. (canceled)

10. The composition of claim 1, wherein the deaminase is linked to or otherwise capable of associating with the one or more helicases.

11. (canceled)

12. The composition of claim 1, wherein the deaminase functions as a cytidine deaminase, an adenosine deaminase, or both.

13. The composition of claim 12, wherein:
the deaminase is a cytidine deaminase selected from the group comprising AID, APOBEC, and TadA; and/or
the composition further comprises a uracil DNA glycosylase inhibitor (UGI).

14-17. (canceled)

18. A vector system comprising one or more polynucleotides encoding the one or more programmable nickases, one or more helicases, and one or more deaminases of claim 1.

19. A delivery system comprising the composition of claim 1.

20-23. (canceled)

24. A method of targeted continuous mutagenesis comprising:
delivering a composition to a cell population, the composition of comprising: (i) one or more programmable nickases; (ii) one or more helicases; and (iii) one or more deaminases,
wherein the one or more programmable nickases introduce a single strand nick in double-stranded DNA (dsDNA) at one or more targeted nick sites at one or more genomic regions to be diversified by continuous mutagenesis,
wherein the one or more helicases unwinds a portion of the dsDNA starting at the targeted nick site to form a portion of unwound DNA, and
the one or more deaminases introduce point mutations via base edits in the portion of unwound DNA.

25. The method of claim 24, wherein:
(a) the one or more helicases unwinds a portion of dsDNA between approximately 1000 bp-5000 bp from the one or more targeted nick sites, and wherein multiple point mutations are made within the portion of unwound dsDNA;
(b) the method further comprises sequencing DNA isolated from the cell population to identify mutations introduced in the one or more genomic regions; and/or
(c) the one or more genomic regions to be diversified comprise one or more exons of a protein or encode a functional polynucleotide, and the method further comprises functionally screening the protein or the functional polynucleotide to select for a change in one or more functions

26-27. (canceled)

28. The method of claim 25, wherein the one or more functions comprise enhanced stability, increased catalytic efficiency, new catalytic activity, altered substrate specificity, improved substrate binding affinity, new enzymatic activity, or a combination thereof.

29.-31. (canceled)

32. A method for identifying mutations conferring resistance to therapeutic agents comprising:
diversifying one or more target regions by delivering to a sample cell population a composition comprising: (i) one or more programmable nickases configured to introduce a single-strand nick in double-stranded DNA (dsDNA) at one or more targeted nick sites; (ii) one or more helicases configured to unwind a portion of the dsDNA at the one or more targeted nick sites to form a portion of unwound dsDNA; and (iii) one or more deaminases configured to introduce one or more base edits within the portion of unwound dsDNA;
selecting for one or more resistance mutations by exposing the sample cell population to one or more therapeutic agents to be screened;
isolating DNA from cells surviving the selecting step, and
identifying one or more resistance mutations by sequencing.

33. The method of claim 32, comprising further validating the one or more resistance mutations by:
generating a modified cell population by introducing the one or more resistance mutations into a wildtype cell population;
exposing the cell population to the one or more therapeutic agents; and
selecting for enriched allele frequencies of the one or more resistance mutations after exposure to the one or more therapeutic agents to define a final set of one or more resistance mutations.

34. A method for identifying mutations associated with incorrect splicing events comprising:
introducing into a sample cell population a splicing reporter configured to produce a detectable signal in the presence of an alternative splicing event;
diversifying one or more target regions by introducing into a sample cell population the composition of claim 1;
selecting cells having alternative splicing event(s) from the sample cell population based on expression of the detectable signal from the splicing reporter;
isolating DNA from cells having an alternative splicing event; and
sequencing the one or more target regions to identify a set of candidate mutations associated with the alternative splicing event.

35. The method of claim 34, wherein the splicing reporter comprises a portion of an endogenous intron and downstream exon fused to a constant upstream exon, and a downstream fluorescent protein reporter, such that correct splicing results in a frameshift in an opening reading of the fluorescent protein reporter suppressing fluorescence, while an incorrect splicing event permits expression of the fluorescent protein reporter.

36. (canceled)

37. A method of identifying one or more functional variant within non-coding gene regulatory elements comprising:
diversifying one or more non-coding gene regulatory elements by delivering to a sample cell population the composition of claim 1;
inducing expression of one or more genes regulated by the one or more non-coding gene regulatory elements;
selecting cells from the sample cell population exhibiting increased expression of the one or more genes;
sequencing DNA from the cells exhibiting increased expression of the one or more genes to identify a set of candidate mutations associated with functional variants within non-coding gene regulatory elements.

38. The method of claim 37, comprising further validating the one or more functional variants comprising:
introducing the set of candidate mutations into a population of wild-type cells;
selecting for cells enriched in expression of the one or more genes; and
sequencing DNA from cells enriched in expression of the one or more genes to define a validated set of functional variants.
